(19) United States
(12) Patent Application Publication
(10) Pub. No.: US 2024/0065099 A1
(43) Pub. Date: Feb. 22, 2024

(54) ORGANIC ELECTROLUMINESCENT COMPOUND, ORGANIC ELECTROLUMINESCENT MATERIAL, AND ORGANIC ELECTROLUMINESCENT DEVICE COMPRISING THE SAME

(71) Applicant: ROHM AND HAAS ELECTRONIC MATERIALS KOREA LTD., Chungcheongnam-do (KR)

(72) Inventors: Hee-Ryong KANG, Gyeonggi-do (KR); So-Young JUNG, Gyeonggi-do (KR); Eun-Joung CHOI, Gyeonggi-do (KR); Sang-Hee CHO, Gyeonggi-do (KR); So-Mi PARK, Gyeonggi-do (KR)

(21) Appl. No.: 18/352,549

(22) Filed: Jul. 14, 2023

(30) Foreign Application Priority Data
Aug. 3, 2022 (KR) 10-2022-0096764

(51) Int. Cl. H10K 85/60; H10K 50/11

(52) U.S. Cl. CPC H10K 85/654; H10K 85/6574; H10K 85/6572; H10K 85/656; H10K 50/11

(57) ABSTRACT

The present disclosure relates to an organic electroluminescent compound represented by formula 1, an organic electroluminescent material comprising the same and an organic electroluminescent device. By comprising an organic electroluminescent compound according to the present disclosure as a host material or an electron buffer material, or a specific combination of compounds as host materials, an organic electroluminescent device having improved drive voltage, luminous efficiency, and/or lifespan characteristics can be provided.

### TECHNICAL FIELD

The present disclosure relates to an organic electroluminescent compound, organic electroluminescent material, and organic electroluminescent device comprising the same.

### BACKGROUND ART

The TPD/Alq₃ bilayer small molecule organic electroluminescent device (OLED) consisting of a light-emitting layer and a charge transport layer, was first developed by Tang, et al., of Eastman Kodak in 1987. Thereafter, the studies on an OLED have been rapidly effected, and OLEDs have been commercialized. At present, an organic electroluminescent device mainly includes phosphorescent materials having excellent luminous efficiency in panel realization. For prolonged use and high resolution of the display, an OLED having high luminous efficiency is necessary.

Korean Patent Application Laid-open No. 2022-0051794 A discloses a plurality of host materials comprising phenanthrene derivative compounds. However, said reference does not specifically disclose specific compounds as described in the present disclosure. Further, there is a need to develop light-emitting materials having improved performance, such as higher luminous efficiency and/or improved lifespan characteristics, compared to combinations of specific compounds disclosed in said reference.

### DISCLOSURE OF THE INVENTION

### Problems to be Solved

The object of the present disclosure is to provide an organic electroluminescent compound with a novel structure suitable for applying to an organic electroluminescent device. Another object of the present disclosure is to provide an improved organic electroluminescent material that can provide an organic electroluminescent device with improved driving voltage, luminous efficiency, and/or lifespan characteristics. The other object of the present disclosure is to provide an organic electroluminescent device having improved driving voltage, luminous efficiency, and/or lifespan characteristics by comprising a compound of the present disclosure as a host material or an electron buffer material, or a specific combination of compounds according to the present disclosure as host materials.

### Solution to Problem

The present inventors found that the aforementioned objective can be achieved by a compound represented by the following formula 1, so that the present invention was completed.

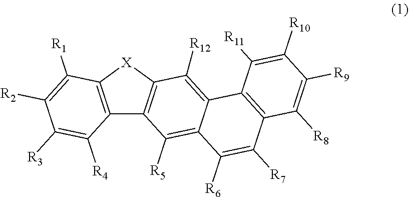

In formula 1,

X represents —C(R′)(R″)- or —Se—;

R′ and R″ each independently represent a substituted or unsubstituted (C1-C30)alkyl, a substituted or unsubstituted (C6-C30)aryl, or a substituted or unsubstituted (3- to 30-membered)heteroaryl; or R′ and R″ may be linked together to form a ring, and R′ and R″ may be the same or different from each other;

R₁ to R₁₂ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of (C3-C30) aliphatic ring and (C6-C30) aromatic ring, or *-L-HAr; or may be linked to an adjacent substituent(s) to form a ring(s);

with a proviso that any one of R₁ to R₁₂ represents *-L-HAr;

L represents a single bond, a substituted or unsubstituted (C6-C30)arylene, or a substituted or unsubstituted (3- to 30-membered)heteroarylene; and

HAr represents a substituted or unsubstituted (3- to 30-membered)heteroaryl containing at least one nitrogen atom(s).

### ADVANTAGEOUS EFFECTS OF INVENTION

An organic electroluminescent compound according to the present disclosure and an organic electroluminescent material comprising thereof exhibit performance suitable for use in an organic electroluminescent device. Further, by including an organic electroluminescent compound according to the present disclosure as a host material or an electron buffer material, or a specific combination of compounds according to the present disclosure as host materials, an organic electroluminescent device having improved driving voltage, luminous efficiency, and/or lifespan properties can be provided.

### EMBODIMENTS OF THE INVENTION

Hereinafter, the present disclosure will be described in detail. However, the following description is intended to explain the present disclosure, and is not meant to restrict the scope of the present disclosure.

Herein, the term “organic electroluminescent compound” in the present disclosure means a compound that may be used in an organic electroluminescent device, and may be comprised in any layer constituting an organic electroluminescent device, as necessary.

Herein, the term “organic electroluminescent material” in the present disclosure means a material that may be used in an organic electroluminescent device, and may comprise at least one compound. The organic electroluminescent material may be comprised in any layer constituting an organic electroluminescent device, as necessary. For example, the organic electroluminescent material may be a hole injection material, a hole transport material, a hole auxiliary material, a light-emitting auxiliary material, an electron blocking material, a light-emitting material (including a host material and a dopant material), an electron buffer material, a hole blocking material, an electron transport material, an electron injection material, etc.

Herein, the term “a plurality of organic electroluminescent materials” in the present disclosure means an organic electroluminescent material comprising a combination of at least two compounds, which may be comprised in any layer constituting an organic electroluminescent device. It may mean both a material before being comprised in an organic electroluminescent device (for example, before vapor deposition) and a material after being comprised in an organic electroluminescent device (for example, after vapor deposition). For example, a plurality of organic electroluminescent materials may be a combination of at least two compounds, which may be comprised in at least one layer of a hole injection layer, a hole transport layer, a hole auxiliary layer, a light-emitting auxiliary layer, an electron blocking layer, a light-emitting layer, an electron buffer layer, a hole blocking layer, an electron transport layer, and an electron injection layer. Such at least two compounds may be comprised in the same layer or in different layers, and may be mixture-evaporated or co-evaporated, or may be individually evaporated.

Herein, the term “a plurality of host materials” means host materials comprising a combination of at least two compounds, which may be comprised in any light-emitting layer constituting an organic electroluminescent device. It may mean both a material before being comprised in an organic electroluminescent device (e.g., before vapor deposition) and a material after being comprised in an organic electroluminescent device (e.g., after vapor deposition). For example, a plurality of host materials of the present disclosure may be a combination of at least two host materials, and optionally, it may further include a conventional material included in the organic electroluminescent material. The at least two compounds comprised in a plurality of host materials of the present disclosure may be comprised together in one light-emitting layer through methods used in the art, or may each be comprised in separate light-emitting layers. For example, such at least two compounds may be mixture-evaporated or co-evaporated, or may be individually evaporated.

Herein, the term “(C1-C30)alkyl” is meant to be a linear or branched alkyl having 1 to 30 carbon atoms constituting the chain, in which the number of carbon atoms is preferably 1 to 10, and more preferably 1 to 6. The above alkyl may include methyl, ethyl, n-propyl, isopropyl, n-butyl, isobutyl, tent-butyl, sec-butyl, etc. The term “(C3-C30)cycloalkyl” is meant to be a monocyclic or polycyclic hydrocarbon having 3 to 30 ring backbone carbon atoms, and preferably 3 to 20 ring backbone carbon atoms, more preferably 3 to 7 ring backbone carbon atoms. Examples of the cycloalkyl may include cyclopropyl, cyclobutyl, cyclopentyl, cyclohexyl, cyclopentylmethyl, cyclohexylmethyl, etc. The term “(3- to 7-membered)heterocycloalkyl” in the present disclosure is meant to be a cycloalkyl having 3 to 7 ring backbone atoms and containing at least one heteroatom(s) selected from the group consisting of B, N, O, S, Si, P, Se, Te, and Ge, preferably the group consisting of O, S, and N. The above heterocycloalkyl includes tetrahydrofuran, pyrrolidine, thiolane, tetrahydropyran, etc. The term “(C6-C30)aryl(ene)” in the present disclosure is meant to be a monocyclic or fused ring radical derived from an aromatic hydrocarbon having 6 to 30 ring backbone carbon atoms. The above aryl may be partially saturated, and may comprise a spiro structure. The above aryl may include phenyl, biphenyl, terphenyl, naphthyl, binaphthyl, phenylnaphthyl, naphthylphenyl, fluorenyl, phenylfluorenyl, diphenylfluorenyl, benzofluorenyl, dibenzofluorenyl, phenanthrenyl, phenylphenanthrenyl, benzophenanthrenyl, anthracenyl, indenyl, triphenylenyl, pyrenyl, tetracenyl, perylenyl, chrysenyl, naphthacenyl, fluoranthenyl, spirobifluorenyl, spiro[fluorene-benzofluorene]yl, spiro[cyclopentene-fluorene]yl, spiro[dihydroindene-fluorene]yl, azulenyl, tetramethyldihydrophenanthrenyl, etc. More specifically, the aryl may include phenyl, 1-naphthyl, 2-naphthyl, 1-anthryl, 2-anthryl, 9-anthryl, benzanthryl, 1-phenanthryl, 2-phenanthryl, 3-phenanthryl, 4-phenanthryl, 9-phenanthryl, naphthacenyl, pyrenyl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 1-fluorenyl, 2-fluorenyl, 3-fluorenyl, 4-fluorenyl, 9-fluorenyl, benzo[a]fluorenyl, benzo[b]fluorenyl, benzo[c]fluorenyl, dibenzofluorenyl, 2-biphenylyl, 3-biphenylyl, 4-biphenylyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 3-fluoranthenyl, 4-fluoranthenyl, 8-fluoranthenyl, 9-fluoranthenyl, benzofluoranthenyl, o-tolyl, m-tolyl, p-tolyl, 2,3-xylyl, 3,4-xylyl, 2,5-xylyl, mesityl, o-cumenyl, m-cumenyl, p-cumenyl, p-tert-butylphenyl, p-(2-phenylpropyl)phenyl, 4′-methylbiphenyl, 4″-tert-butyl-p-terphenyl-4-yl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-di methyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-di phenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9, 9, 10, 10-tetramethyl-9, 10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

Herein, the term “(3- to 30-membered)heteroaryl(ene)” in the present disclosure is meant to be an aryl or arylene having 3 to 30 ring backbone atoms and including at least one heteroatom selected from the group consisting of B, N, O, S, Si, P, Se, Te, and Ge. The number of heteroatoms is preferably from 1 to 4, and may be a monocyclic ring or a fused ring condensed with at least one benzene ring, and may be partially saturated. In addition, the above heteroaryl or heteroarylene comprises one formed by linking at least one heteroaryl or aryl group to a heteroaryl group via a single bond(s), and may comprise a spiro structure. The above heteroaryl may include a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, di benzothiophenyl, di benzoselenophenyl, naphthobenzofuranyl, naphthobenzothiophenyl, benzofuroquinolyl, benzofuroquinazolinyl, benzofuronaphthyridinyl, benzofuropyrimidinyl, naphthofuropyri midi nyl, benzothienoquinolyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphtofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, benzocarbazolyl, dibenzocarbazolyl, phenoxazinyl, phenanthridinyl, benzodioxolyl, dihydroacridinyl, benzotriazolyl, phenazinyl, imidazopyridyl, chromenoquinazolinyl, thiochromenoquinazolinyl, dimethylbenzopyrimidinyl, indolocarbazolyl, indenocarbazolyl, etc. More specifically, the heteroaryl may include 1-pyrrolyl, 2-pyrrolyl, 3-pyrrolyl, pyrazinyl, 2-pyridyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolidinyl, 2-indolidinyl, 3-indolidinyl, 5-indolidinyl, 6-indolidinyl, 7-indolidinyl, 8-indolidinyl, 2-imidazopyridyl, 3-imidazopyridyl, 5-imidazopyridyl, 6-imidazopyridyl, 7-imidazopyridyl, 8-imidazopyridyl, 3-pyridyl, 4-pyridyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazolyI-1-yl, azacarbazolyl-2-yl, azacarbazolyl-3-yl, azacarbazolyl-4-yl, azacarbazolyl-5-yl, azacarbazolyl-6-yl, azacarbazolyl-7-yl, azacarbazolyl-8-yl, azacarbazolyl-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-tert-butylpyrrol-4-yl, 3-(2-phenylpropyl)pyrrol-1-yl, 2-methyl-1-indolyl, 4-methyl-1-indolyl, 2-methyl-3-indolyl , 4-methyl-3-indolyl, 2-tert-butyl-1-indolyl, 4-tert-butyl-1-indolyl, 2-tent-butyl-3-indolyl, 4-tert-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho-[1,2-b]-benzofuranyl, 2-naphtho-[1,2-b]-benzofuranyl, 3-naphtho-[1,2-b]-benzofuranyl, 4-naphtho-[1,2-b]-benzofuranyl, 5-naphtho-[1,2-b]-benzofuranyl, 6-naphtho-[1,2-b]-benzofuranyl, 7-naphtho-[1,2-b]-benzofuranyl, 8-naphtho-[1,2-b]-benzofuranyl, 9-naphtho-[1,2-b]-benzofuranyl, 10-naphtho-[1,2-b]-benzofuranyl, 1-naphtho-[2,3-b]-benzofuranyl, 2-naphtho-[2,3-b]-benzofuranyl, 3-naphtho-[2,3-b]-benzofuranyl, 4-naphtho-[2,3-b]-benzofuranyl, 5-naphtho-[2,3-b]-benzofuranyl, 6-naphtho-[2,3-b]-benzofuranyl, 7-naphtho-[2, 3-b]-benzofuranyl, 8-naphtho-[2,3-b]-benzofuranyl, 9-naphtho-[2,3-b]-benzofuranyl, 10-naphtho-[2,3-b]-benzofuranyl, 1-naphtho-[2,1-b]-benzofuranyl, 2-naphtho-[2,1-b]-benzofuranyl, 3-naphtho-[2,1-b]-benzofuranyl, 4-naphtho-[2,1-b]-benzofuranyl, 5-naphtho-[2,1-b]-benzofuranyl, 6-naphtho-[2,1-b]-benzofuranyl, 7-naphtho-[2,1-b]-benzofuranyl, 8-naphtho-[2,1-b]-benzofuranyl, 9-naphtho-[2,1-b]-benzofuranyl, 10-naphtho-[2,1-b]-benzofuranyl, 1-naphtho-[1,2-b]-benzothiophenyl, 2-naphtho-[1,2-b]-benzothiophenyl, 3-naphtho-[1,2-b]-benzothiophenyl, 4-naphtho-[1,2-b]-benzothiophenyl, 5-naphtho-[1,2-b]-benzothiophenyl, 6-naphtho-[1,2-b]-benzothiophenyl, 7-naphtho-[1,2-b]-benzothiophenyl, 8-naphtho-[1,2-b]-benzothiophenyl, 9-naphtho-[1,2-b]-benzothiophenyl, 10-naphtho-[1,2-b]-benzothiophenyl, 1-naphtho-[2,3-b]-benzothiophenyl, 2-naphtho-[2,3-b]-benzothiophenyl, 3-naphtho-[2,3-b]-benzothiophenyl, 4-naphtho-[2,3-b]-benzothiophenyl, 5-naphtho-[2,3-b]-benzothiophenyl, 1-naphtho-[2,1-b]-benzothiophenyl, 2-naphtho-[2,1-b]-benzothiophenyl, 3-naphtho-[2,1-b]-benzothiophenyl, 4-naphtho-[2,1-b]-benzothiophenyl, 5-naphtho-[2,1-b]-benzothiophenyl, 6-naphtho-[2,1-b]-benzothiophenyl, 7-naphtho-[2,1-b]-benzothiophenyl, 8-naphtho-[2,1-b]-benzothiophenyl, 9-naphtho-[2,1-b]-benzothiophenyl, 10-naphtho-[2,1-b]-benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. In the present disclosure, the term “halogen” includes F, CI, Br, and I.

In addition, “ortho (o-),” “meta (m-),” and “para (p-)” are prefixes, which represent the relative positions of substituents, respectively. Ortho indicates that two substituents are adjacent to each other, and for example, when two substituents in a benzene derivative occupy positions 1 and 2, it is called an ortho position. Meta indicates that two substituents are at positions 1 and 3, and for example, when two substituents in a benzene derivative occupy positions 1 and 3, it is called a meta position. Para indicates that two substituents are at positions 1 and 4, and for example, when two substituents in a benzene derivative occupy positions 1 and 4, it is called a para position.

Herein, the term “substituted” in the expression “substituted or unsubstituted” means that a hydrogen atom in a certain functional group is replaced with another atom or another functional group (i.e., a substituent), and also includes that the hydrogen atom is substituted with a group to which two or more substituents of the above substituents are connected. For example, “a substituent to which two or more substituents are connected” may be pyridine-triazine. That is, pyridine-triazine may be interpreted as one heteroaryl substituent, or as substituents in which two heteroaryls are linked. Herein, the substituent(s) of the substituted alkyl, the substituted aryl, the substituted arylene, the substituted heteroaryl, the substituted heteroarylene, the substituted cycloalkyl, the substituted alkoxy, the substituted trialkylsilyl, the substituted dialkylarylsilyl, the substituted alkyldiarylsilyl, the substituted triarylsilyl, and the substituted fused ring of an aliphatic ring and an aromatic ring, each independently are at least one selected from the group consisting of deuterium; halogen; cyano; carboxyl; nitro; hydroxyl; phosphine oxide; (C1-C30)alkyl; halo(C1-C30)alkyl; (C2-C30)alkenyl unsubstituted or substituted with at least one of (C6-C30)aryl(s); (C2-C30)alkynyl; (C1-C30)alkoxy; (C1-C30)alkylthio; (C3-C30)cycloalkyl; (C3-C30)cycloalkenyl; (3- to 7-membered) heterocycloalkyl; (C6-C30)aryloxy; (C6-C30)arylthio; (3- to 30-membered)heteroaryl unsubstituted or substituted with at least one of deuterium and (C6-C30)aryl(s); (C6-C30)aryl unsubstituted or substituted with at least one of deuterium, cyano, (C1-C30)alkyl, and (3- to 30-membered)heteroaryl ; tri(C1-C30)alkylsi lyl ; tri(C6-C30)arylsi lyl ; di(C1-C30)alkyl(C6-C30)arylsilyl; (C1-C30)alkyldi(C6-C30)arylsilyl; a fused ring of (C3-C30) aliphatic ring and (C6-C30) aromatic ring; (C1-C30)alkylcarbonyl; (C1-C30)alkoxycarbonyl; (C6-C30)arylcarbonyl ; di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl. According to one embodiment of the present disclosure, the substituent(s) each independently are at least one selected from the group consisting of deuterium; cyano; (C1-C20)alkyl; C5-C25)cycloalkyl; (5- to 25-membered)heteroaryl unsubstituted or substituted with at least one of deuterium and (C6-C25)aryl; (C6-C25)aryl unsubstituted or substituted with at least one of deuterium, cyano, (C1-C20)alkyl, and (C6-C25)aryl; tri(C1-C20)alkylsilyl; and tri(C6-C25)arylsilyl. According to another embodiment of the present disclosure, the substituent(s) each independently are at least one selected from the group consisting of deuterium; cyano; (C1-C10)alkyl; (C5-C12)cycloalkyl; (5- to 20-membered)heteroaryl unsubstituted or substituted with at least one of deuterium and (C6-C18)aryl; (C6-C18)aryl unsubstituted or substituted with at least one of deuterium, cyano, (C1-C10)alkyl, and (C6-C18)aryl; tri(C1-C10)alkylsilyl; and tri(C6-C18)arylsilyl. For example, the substituent(s) may be at least one selected from the group consisting of deuterium; phenyl unsubstituted or substituted with at least one of cyano and methyl; naphthyl; naphthylphenyl; phenylnapthyl; biphenyl; terphenyl; fluorenyl substituted with phenyl; pyridyl; dibenzofuranyl; dibenzothiophenyl; benzoxazolyl substituted with phenyls; cyclopentadecanyl; triphenylsilyl; and trimethylsilyl. Further, they may be further substituted with deuterium.

In the present disclosure, a ring formed by a linkage of an adjacent substituent(s) means that at least two adjacent substituents are linked or fused to each other to form a substituted or unsubstituted, mono- or polycyclic, (3- to 30-membered)alicyclic or aromatic ring, or the combination thereof. The ring may be preferably a substituted or unsubstituted, mono- or polycyclic, (3- to 26-membered)alicyclic or aromatic ring, or the combination thereof, and more preferably a mono- or polycyclic, (5- to 25-membered) aromatic ring unsubstituted or substituted with at least one of (C6-C18)aryl and (3- to 20-membered)heteroaryl. In addition, the formed ring may contain at least one heteroatom selected from the group consisting of B, N, O, S, Si, P, Se, Te, and Ge, preferably at least one heteroatom selected from the group consisting of N, O, S, and Se. For example, the ring may be a benzene ring, a cyclopentane ring, an indan ring, a fluorene ring, a phenanthrene ring, an indole ring, a xanthene ring, etc.

In the present disclosure, a heteroaryl, a heteroarylene, and a heterocycloalkyl each independently may include at least one heteroatom(s) selected from the group consisting of B, N, O, S, Si, P, Se, Te, and Ge. In addition, the heteroatom may be bonded to at least one selected from the group consisting of hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted mono- or di-(C1-C30)alkylamino, a substituted or unsubstituted mono- or di-(C2-C30)alkenylamino, a substituted or unsubstituted mono- or di-(C6-C30)arylamino, a substituted or unsubstituted mono- or di-(3- to 30-membered)heteroarylamino, a substituted or unsubstituted (C1-C30)alkyl(C2-C30)alkenylamino, a substituted or unsubstituted (C1-C30)alkyl(C6-C30)arylamino, a substituted or unsubstituted (C1-C30)alkyl(3- to 30-membered)heteroarylami no, a substituted or unsubstituted (C2-C30)alkenyl(C6-C30)arylamino, a substituted or unsubstituted (C2-C30)alkenyl(3- to 30-membered)heteroarylamino, and a substituted or unsubstituted (C6-C30)aryl(3- to 30-membered)heteroarylamino.

The compound represented by formula 1 of the present disclosure can be more specifically described as follows.

In formula 1, X represents —C(R′)(R″)- or —Se—.

In formula 1, R′ and R″ each independently represent a substituted or unsubstituted (C1-C30)alkyl, a substituted or unsubstituted (C6-C30)aryl, or a substituted or unsubstituted (3- to 30-membered)heteroaryl; or R′ and R″ may be linked to each other to form a ring, and R′ and R″ may be the same as or different from each other. According to one embodiment of the present disclosure, R′ and R″ each independently may be a substituted or unsubstituted (C1-C20)alkyl, or a substituted or unsubstituted (C6-C25)aryl; or R′ and R″ may be linked together to form a ring. According to another embodiment of the present disclosure, R′ and R″ each independently may be (C1-C10)alkyl unsubstituted or substituted with deuterium, or (C6-C18)aryl unsubstituted or substituted with deuterium, or R′ and R″ may be linked together to form a spiro ring. For example, R′ and R″ each independently may be methyl, phenyl, or naphthyl, which are unsubstituted or substituted with deuterium, or R′ and R″ may be linked together to form a spirofluorene ring.

In formula 1, R₁ to R₁₂ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of a (C3-C30) aliphatic ring and a (C6-C30) aromatic ring, or *-L-HAr; or may be linked to an adjacent substituent(s) to form a ring. According to one embodiment of the present disclosure, R₁ to R₁₂ each independently may be hydrogen, deuterium, a substituted or unsubstituted (C6-C25)aryl, a substituted or unsubstituted (5- to 25-membered)heteroaryl, or *-L-HAr; or may be linked to an adjacent substituent(s) to form a ring. According to another embodiment of the present disclosure, R₁ to R₁₂ each independently may be hydrogen, deuterium, (C6-C18)aryl unsubstituted or substituted with deuterium, or a (5- to 20-membered)heteroaryl unsubstituted or substituted with at least one of deuterium and (C6-C18)aryl, or *-L-HAr; or may be linked to an adjacent substituent(s) to form a ring. For example, R₁ to R₁₂ each independently may be hydrogen, deuterium, phenyl, naphthyl, biphenyl, phenyl-substituted benzoxazole, or dibenzofuranyl; or may be linked to an adjacent substituent(s) to form a benzene ring. The substituents may be further substituted with deuterium.

In formula 1, at least one of R₁ to R₁₂ represents *-L-HAr. According to one embidiment of the present disclosure, any one of R₁ to R₁₂ is *-L-HAr.

In formula 1, L represents a single bond, a substituted or unsubstituted (C6-C30)arylene, or a substituted or unsubstituted (3- to 30-membered)heteroarylene. According to one embodiment of the present disclosure, L represents a single bond or a substituted or unsubstituted (C6-C25)arylene. According to another embodiment of the present disclosure, L represents a single bond or (C6-C18)arylene unsubstituted or substituted with deuterium. For example, L may be a single bond, phenylene, naphthylene, etc. The substituents may be substituted with deuterium.

In formula 1, HAr represents a substituted or unsubstituted (3- to 30-membered)heteroaryl containing at least one nitrogen atom(s). According to one embodiment of the present disclosure, HAr represents a substituted or unsubstituted (5-to 25-membered)heteroaryl containing one or more nitrogen atoms. According to another embodiment of the present disclosure, HAr represents a substituted or unsubstituted (5-to 20-membered)heteroaryl containing one or more nitrogen atoms. The substituent on said heteroaryl may be at least one selected from the group consisting of deuterium, cyano, (C1-C30)alkyl, (C6-C30)aryl, (3- to 30-membered)heteroaryl, (C6-C30)cycloalkyl, tri(C1-C30)alkylsilyl, and tri(C6-C30)arylsilyl. Specifically, HAr may be a substituted or unsubstituted, pyridyl, pyrimidinyl, triazinyl, quinazolinyl, benzoquinazolinyl, quinoxalinyl, benzoquinoxalinyl, quinolyl, benzoquinolinyl, isoquinolyl, benzoisoquinolyl, triazolyl, pyrazolyl, naphthyridinyl, benzofuropyrimidinyl, benzothienopyrimidinyl, carbazolyl, or pyridopyrazinyl. For example, HAr may be a substituted triazinyl, a substituted pyrimidinyl, a substituted quinoxalinyl, a substituted benzoquinoxalinyl, etc., wherein the substituent thereof is phenyl unsubstituted or substituted with at least one of cyano and methyl, naphthyl, naphthylphenyl, phenylnaphthyl, biphenyl, terphenyl, fluorenyl substituted with phenyl, pyridyl, dibenzofuranyl, dibenzothiophenyl, benzoxazolyl substituted with phenyl, triphenylsilyl, cyclodecanyl; trimethylsilyl, etc. They may be further substituted with deuterium.

According to one embodiment of the present disclosure, the formula 1 above may be represented by any one of the following formulas 1-1 to 1-12.

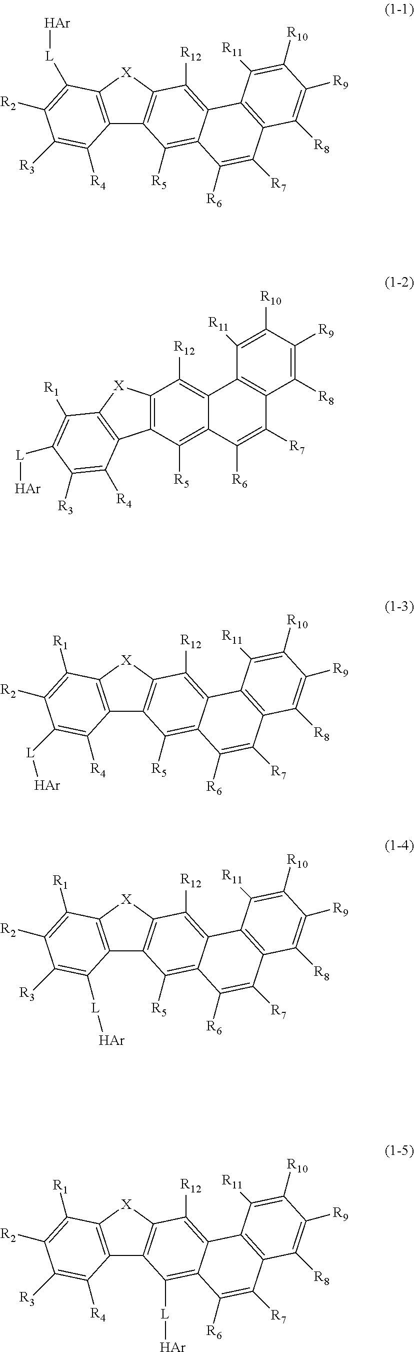

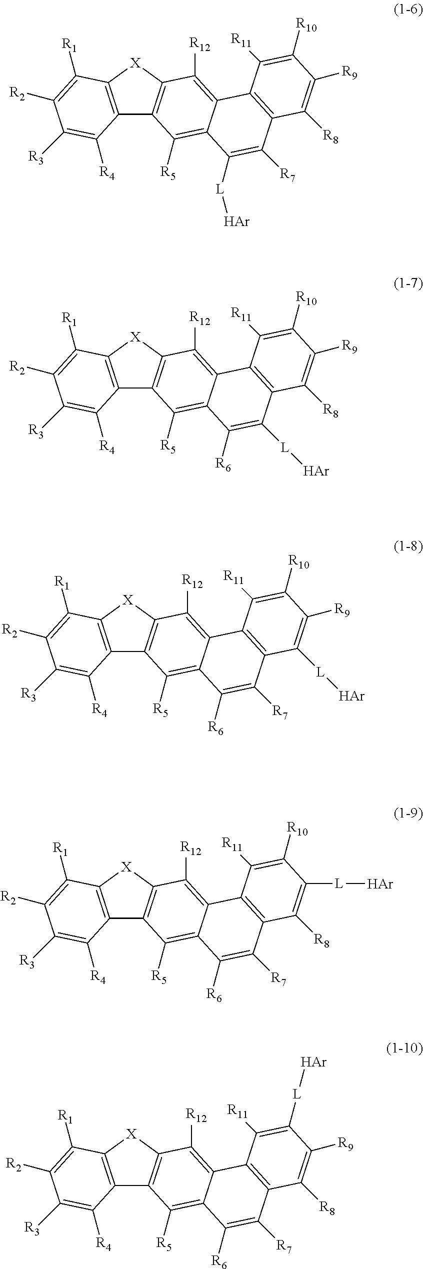

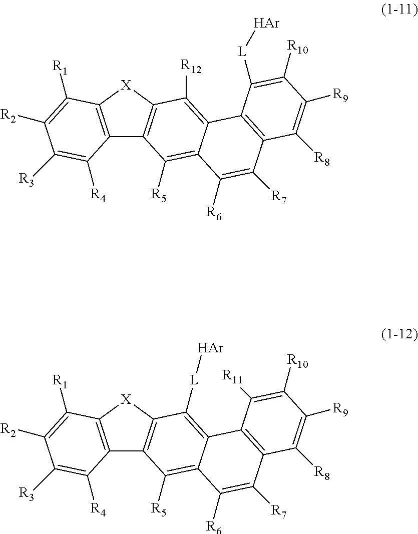

In formulas 1-1 to 1-12, R₁ to R₁₂, X, L, and HAr are each as defined in formula 1.

The compound represented by the formula 1 may be at least one selected from the group consisting of the following compounds, but is not limited thereto.

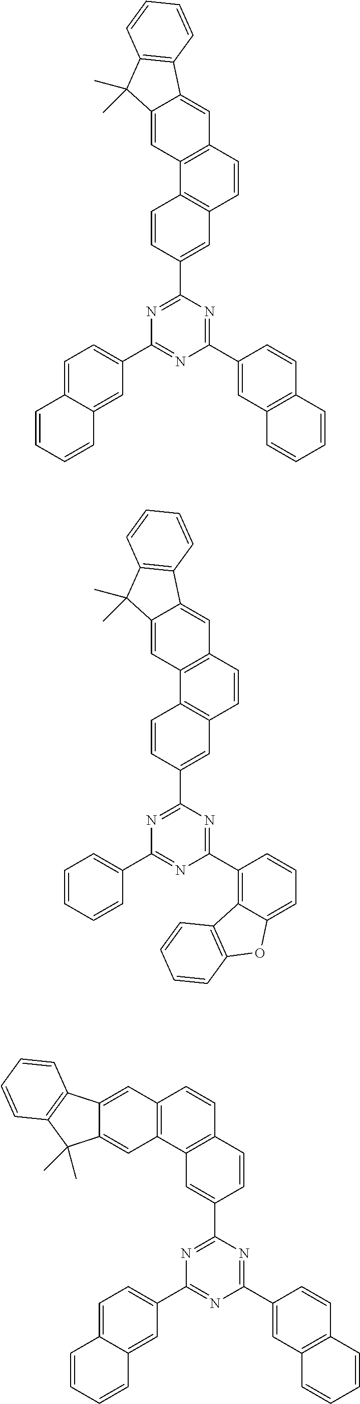 [SMILES: CC1(C)c2ccccc2-c2cc3ccc4cc(-c5nc(-c6ccc7ccccc7c6)nc(-c6ccc7ccccc7c6)n5)ccc4c3cc21 ; CC1(C)c2ccccc2-c2cc3ccc4cc(-c5nc(-c6ccccc6)nc(-c6cccc7oc8ccccc8c67)n5)ccc4c3cc21 ; CC1(C)c2ccccc2-c2cc3ccc4ccc(-c5nc(-c6ccc7ccccc7c6)nc(-c6ccc7ccccc7c6)n5)cc4c3cc21]

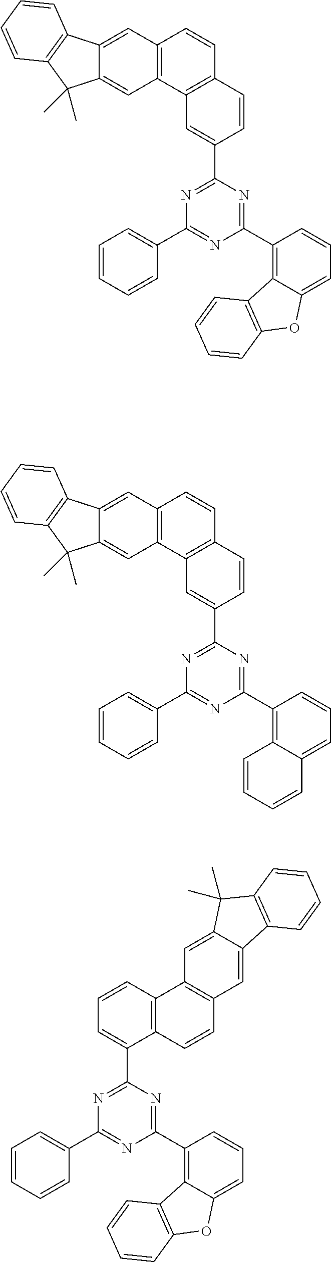 [SMILES: CC1(C)c2ccccc2-c2cc3ccc4ccc(-c5nc(-c6ccccc6)nc(-c6cccc7oc8ccccc8c67)n5)cc4c3cc21 ; CC1(C)c2ccccc2-c2cc3ccc4ccc(-c5nc(-c6ccccc6)nc(-c6cccc7ccccc67)n5)cc4c3cc21 ; CC1(C)c2ccccc2-c2cc3ccc4c(-c5nc(-c6ccccc6)nc(-c6cccc7oc8ccccc8c67)n5)cccc4c3cc21]

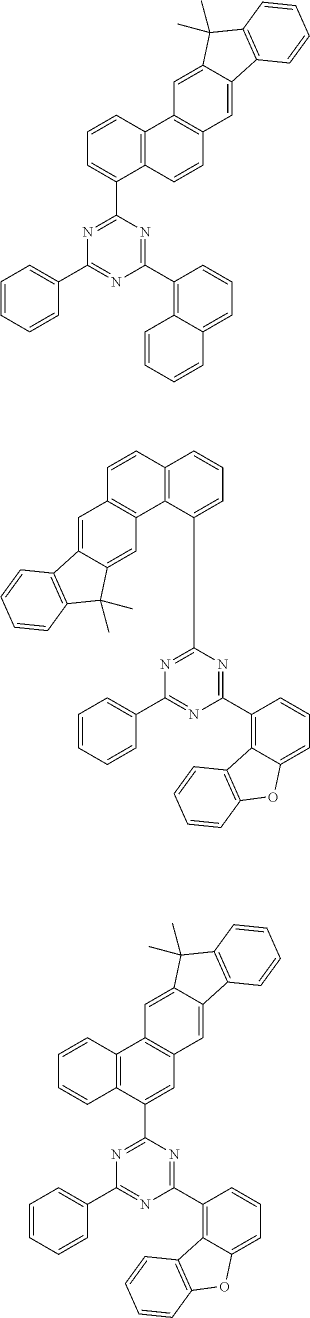 [SMILES: CC1(C)c2ccccc2-c2cc3ccc4c(-c5nc(-c6ccccc6)nc(-c6cccc7ccccc67)n5)cccc4c3cc21 ; CC1(C)c2ccccc2-c2cc3ccc4cccc(-c5nc(-c6ccccc6)nc(-c6cccc7oc8ccccc8c67)n5)c4c3cc21 ; CC1(C)c2ccccc2-c2cc3cc(-c4nc(-c5ccccc5)nc(-c5cccc6oc7ccccc7c56)n4)c4ccccc4c3cc21]

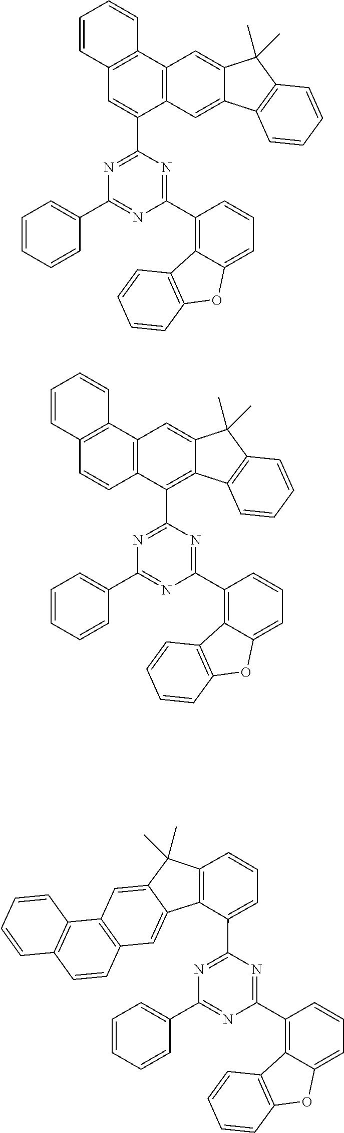 [SMILES: CC1(C)c2ccccc2-c2cc3c(-c4nc(-c5ccccc5)nc(-c5cccc6oc7ccccc7c56)n4)cc4ccccc4c3cc21 ; CC1(C)c2ccccc2-c2c1cc1c(ccc3ccccc31)c2-c1nc(-c2ccccc2)nc(-c2cccc3oc4ccccc4c23)n1 ; CC1(C)c2cc3c(ccc4ccccc43)cc2-c2c(-c3nc(-c4ccccc4)nc(-c4cccc5oc6ccccc6c45)n3)cccc21]

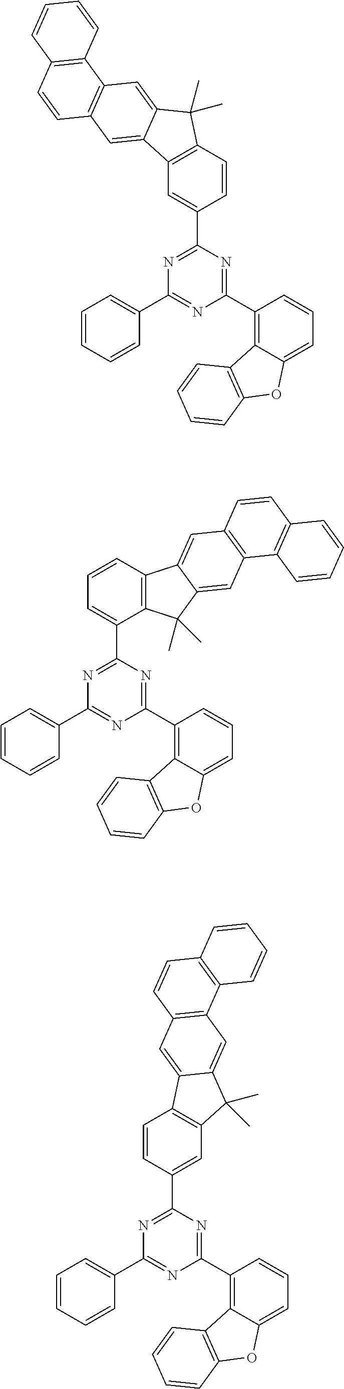 [SMILES: CC1(C)c2ccc(-c3nc(-c4ccccc4)nc(-c4cccc5oc6ccccc6c45)n3)cc2-c2cc3ccc4ccccc4c3cc21 ; CC1(C)c2cc3c(ccc4ccccc43)cc2-c2cccc(-c3nc(-c4ccccc4)nc(-c4cccc5oc6ccccc6c45)n3)c21 ; CC1(C)c2cc(-c3nc(-c4ccccc4)nc(-c4cccc5oc6ccccc6c45)n3)ccc2-c2cc3ccc4ccccc4c3cc21]

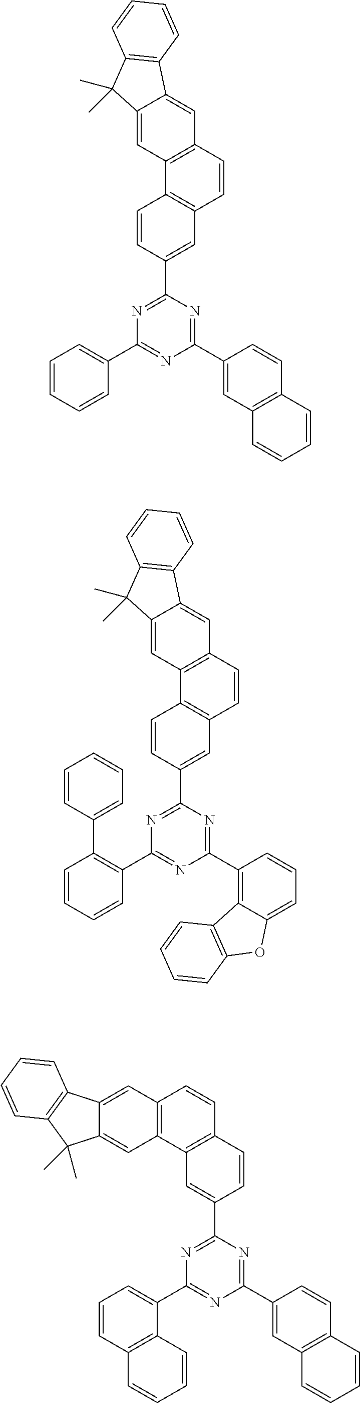 [SMILES: CC1(C)c2ccccc2-c2cc3ccc4cc(-c5nc(-c6ccccc6)nc(-c6ccc7ccccc7c6)n5)ccc4c3cc21 ; CC1(C)c2ccccc2-c2cc3ccc4cc(-c5nc(-c6ccccc6-c6ccccc6)nc(-c6cccc7oc8ccccc8c67)n5)ccc4c3cc21 ; CC1(C)c2ccccc2-c2cc3ccc4ccc(-c5nc(-c6ccc7ccccc7c6)nc(-c6cccc7ccccc67)n5)cc4c3cc21]

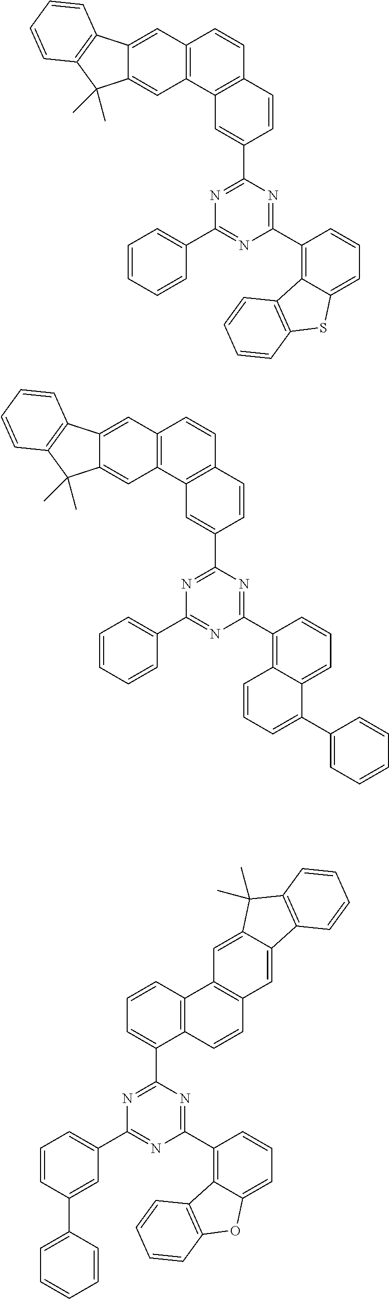 [SMILES: CC1(C)c2ccccc2-c2cc3ccc4ccc(-c5nc(-c6ccccc6)nc(-c6cccc7sc8ccccc8c67)n5)cc4c3cc21 ; CC1(C)c2ccccc2-c2cc3ccc4ccc(-c5nc(-c6ccccc6)nc(-c6cccc7c(-c8ccccc8)cccc67)n5)cc4c3cc21 ; CC1(C)c2ccccc2-c2cc3ccc4c(-c5nc(-c6cccc(-c7ccccc7)c6)nc(-c6cccc7oc8ccccc8c67)n5)cccc4c3cc21]

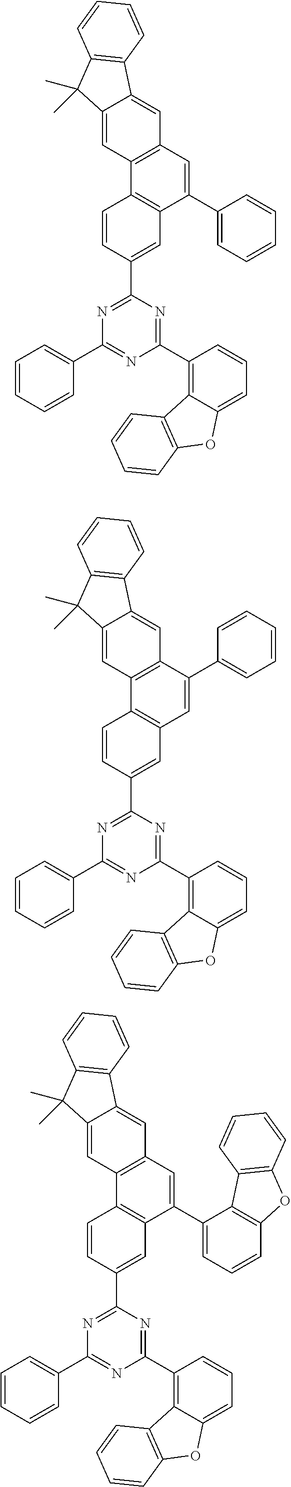

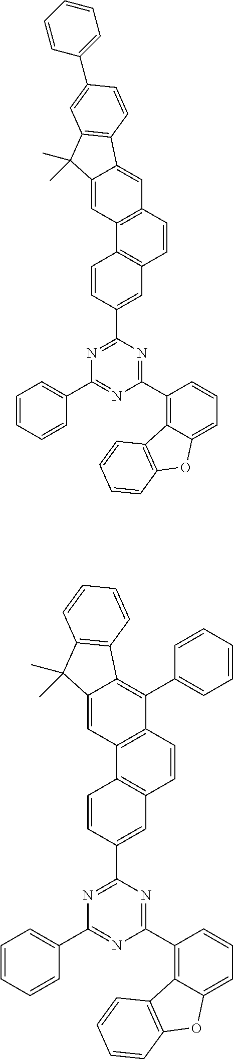 [SMILES: CC1(C)c2cc(-c3ccccc3)ccc2-c2cc3ccc4cc(-c5nc(-c6ccccc6)nc(-c6cccc7oc8ccccc8c67)n5)ccc4c3cc21 ; CC1(C)c2ccccc2-c2c1cc1c(ccc3cc(-c4nc(-c5ccccc5)nc(-c5cccc6oc7ccccc7c56)n4)ccc31)c2-c1ccccc1]

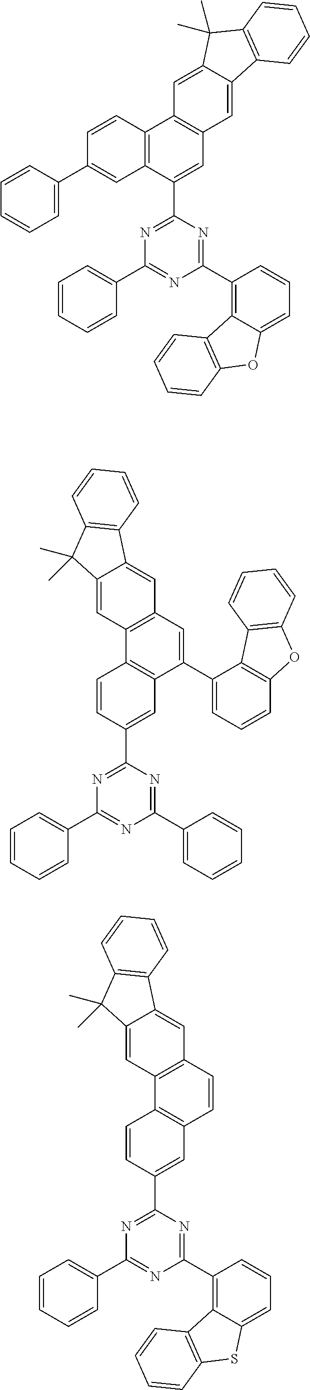 [SMILES: CC1(C)c2ccccc2-c2cc3cc(-c4nc(-c5ccccc5)nc(-c5cccc6oc7ccccc7c56)n4)c4cc(-c5ccccc5)ccc4c3cc21 ; CC1(C)c2ccccc2-c2cc3cc(-c4cccc5oc6ccccc6c45)c4cc(-c5nc(-c6ccccc6)nc(-c6ccccc6)n5)ccc4c3cc21 ; CC1(C)c2ccccc2-c2cc3ccc4cc(-c5nc(-c6ccccc6)nc(-c6cccc7sc8ccccc8c67)n5)ccc4c3cc21]

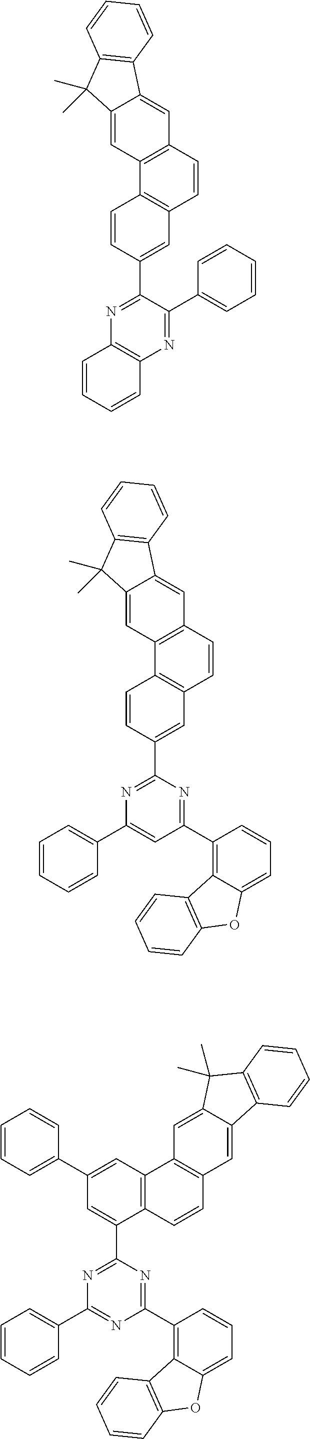 [SMILES: CC1(C)c2ccccc2-c2cc3ccc4cc(-c5nc6ccccc6nc5-c5ccccc5)ccc4c3cc21 ; CC1(C)c2ccccc2-c2cc3ccc4cc(-c5nc(-c6ccccc6)cc(-c6cccc7oc8ccccc8c67)n5)ccc4c3cc21 ; CC1(C)c2ccccc2-c2cc3ccc4c(-c5nc(-c6ccccc6)nc(-c6cccc7oc8ccccc8c67)n5)cc(-c5ccccc5)cc4c3cc21]

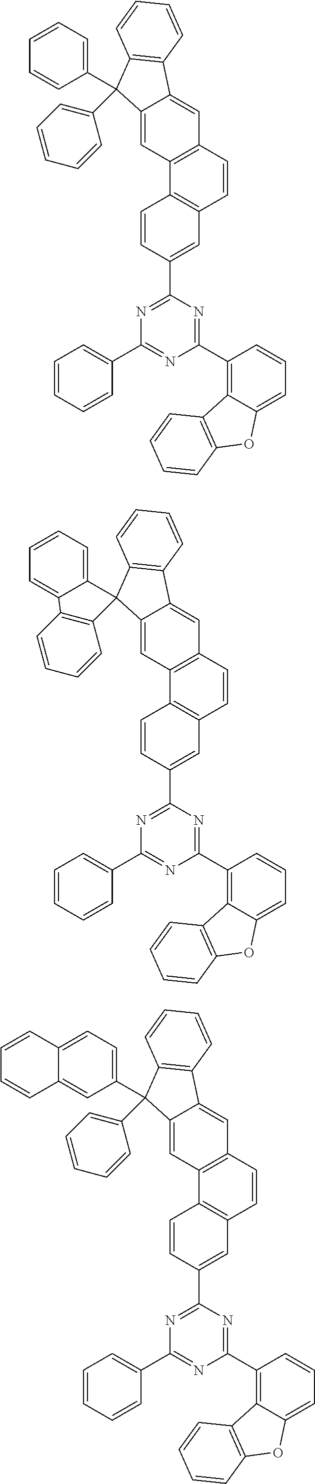

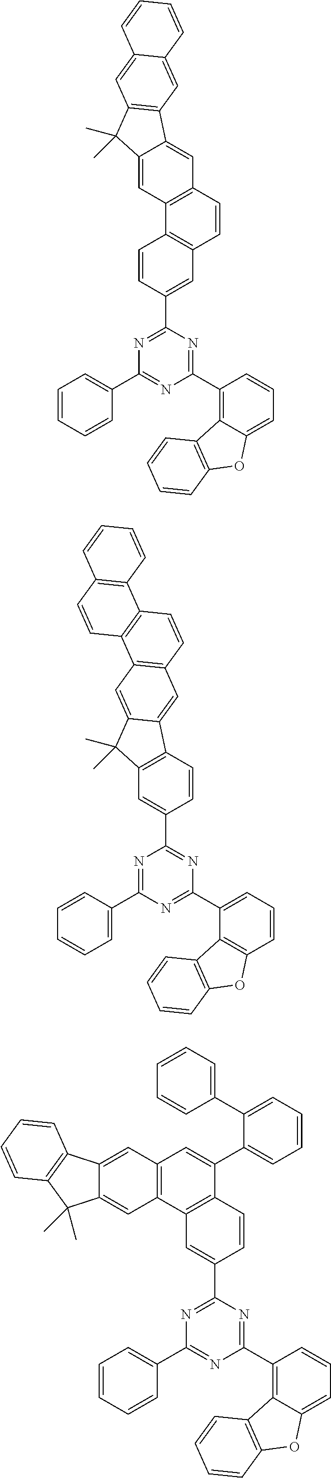

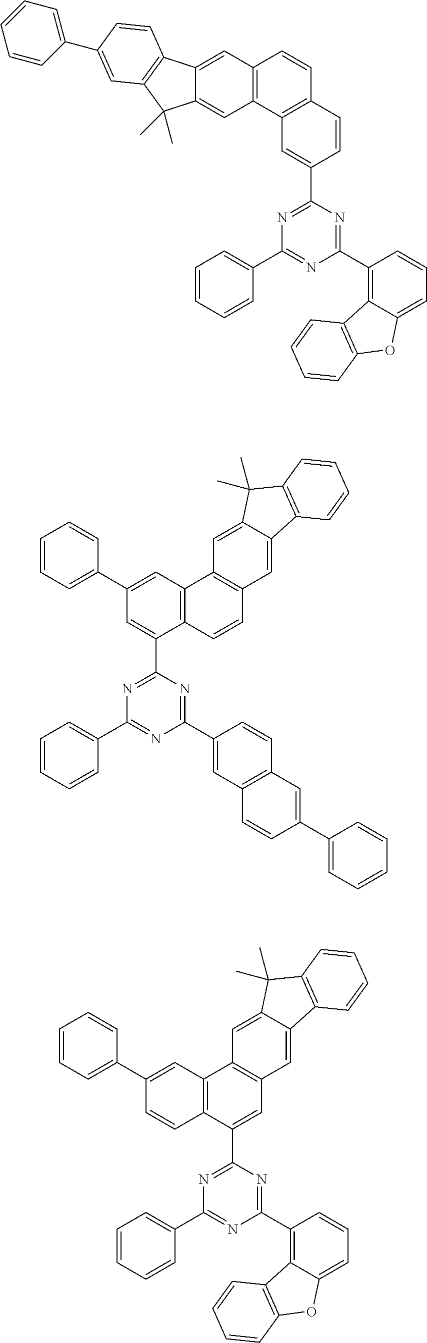 [SMILES: CC1(C)c2cc(-c3ccccc3)ccc2-c2cc3ccc4ccc(-c5nc(-c6ccccc6)nc(-c6cccc7oc8ccccc8c67)n5)cc4c3cc21 ; CC1(C)c2ccccc2-c2cc3ccc4c(-c5nc(-c6ccccc6)nc(-c6ccc7cc(-c8ccccc8)ccc7c6)n5)cc(-c5ccccc5)cc4c3cc21 ; CC1(C)c2ccccc2-c2cc3cc(-c4nc(-c5ccccc5)nc(-c5cccc6oc7ccccc7c56)n4)c4ccc(-c5ccccc5)cc4c3cc21]

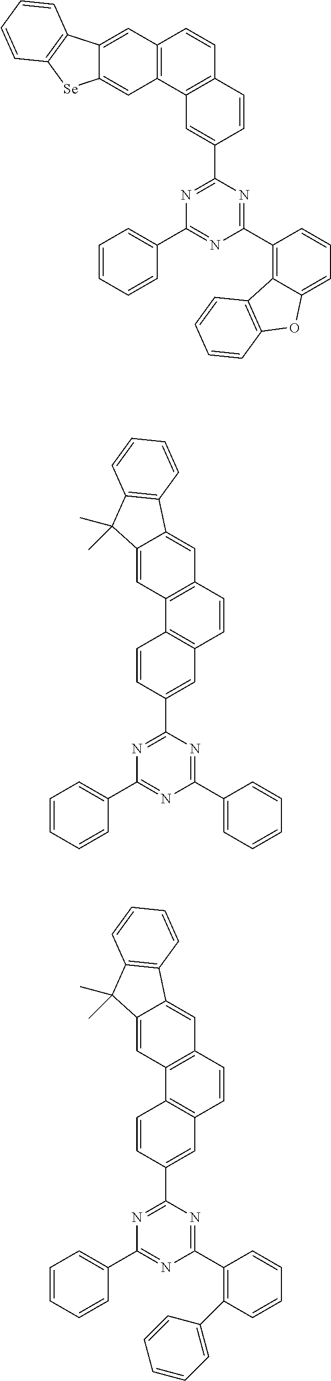 [SMILES: c1ccc(-c2nc(-c3ccc4ccc5cc6c(cc5c4c3)[se]c3ccccc36)nc(-c3cccc4oc5ccccc5c34)n2)cc1 ; CC1(C)c2ccccc2-c2cc3ccc4cc(-c5nc(-c6ccccc6)nc(-c6ccccc6)n5)ccc4c3cc21 ; CC1(C)c2ccccc2-c2cc3ccc4cc(-c5nc(-c6ccccc6)nc(-c6ccccc6-c6ccccc6)n5)ccc4c3cc21]

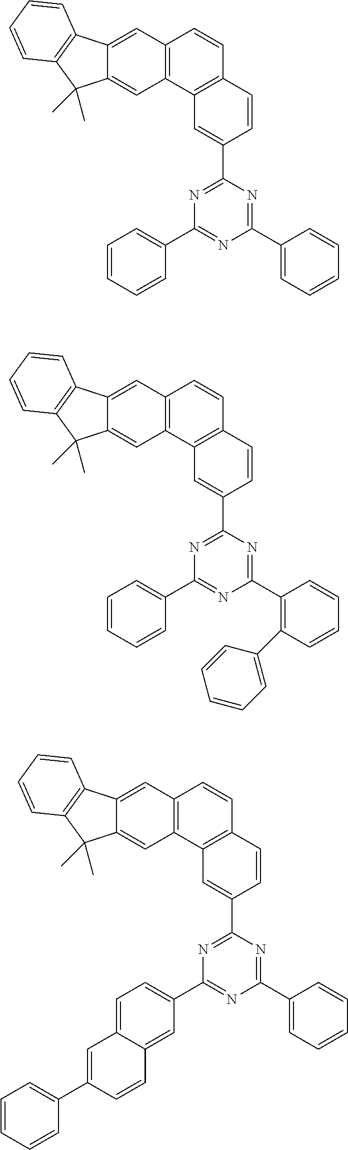 [SMILES: CC1(C)c2ccccc2-c2cc3ccc4ccc(-c5nc(-c6ccccc6)nc(-c6ccccc6)n5)cc4c3cc21 ; CC1(C)c2ccccc2-c2cc3ccc4ccc(-c5nc(-c6ccccc6)nc(-c6ccccc6-c6ccccc6)n5)cc4c3cc21 ; CC1(C)c2ccccc2-c2cc3ccc4ccc(-c5nc(-c6ccccc6)nc(-c6ccc7cc(-c8ccccc8)ccc7c6)n5)cc4c3cc21]

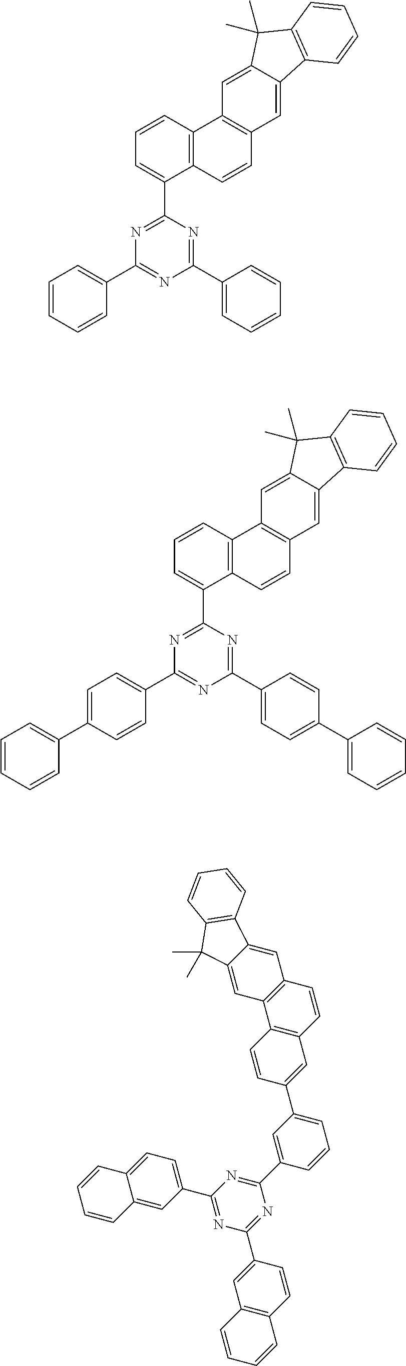 [SMILES: CC1(C)c2ccccc2-c2cc3ccc4c(-c5nc(-c6ccccc6)nc(-c6ccccc6)n5)cccc4c3cc21 ; CC1(C)c2ccccc2-c2cc3ccc4c(-c5nc(-c6ccc(-c7ccccc7)cc6)nc(-c6ccc(-c7ccccc7)cc6)n5)cccc4c3cc21 ; CC1(C)c2ccccc2-c2cc3ccc4cc(-c5cccc(-c6nc(-c7ccc8ccccc8c7)nc(-c7ccc8ccccc8c7)n6)c5)ccc4c3cc21]

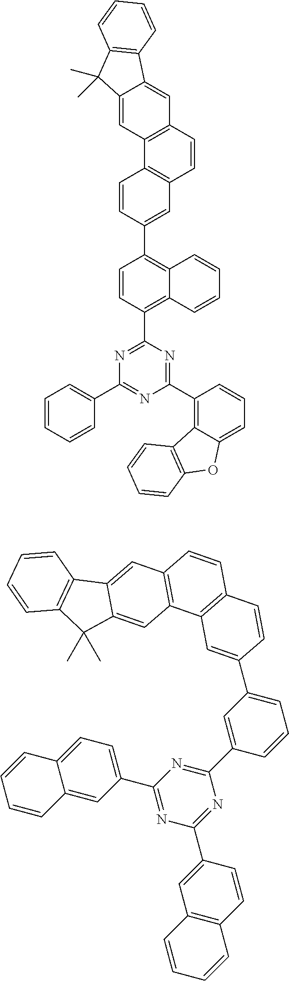 [SMILES: CC1(C)c2ccccc2-c2cc3ccc4cc(-c5ccc(-c6nc(-c7ccccc7)nc(-c7cccc8oc9ccccc9c78)n6)c6ccccc56)ccc4c3cc21 ; CC1(C)c2ccccc2-c2cc3ccc4ccc(-c5cccc(-c6nc(-c7ccc8ccccc8c7)nc(-c7ccc8ccccc8c7)n6)c5)cc4c3cc21]

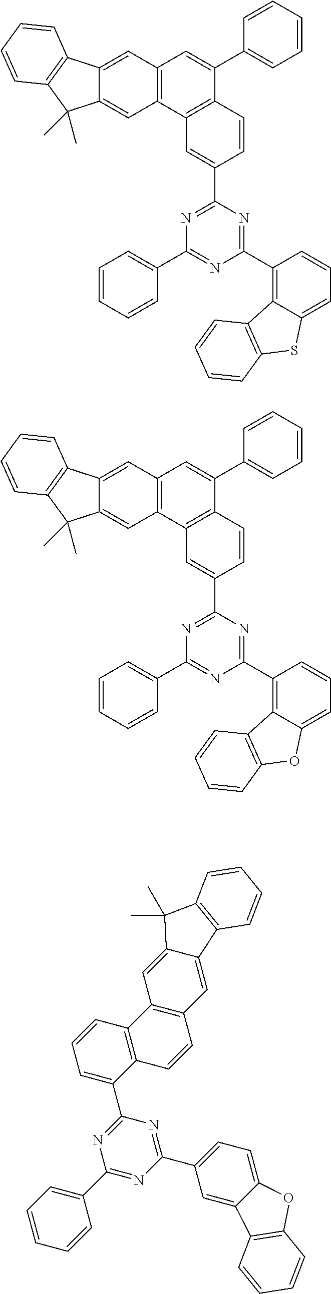

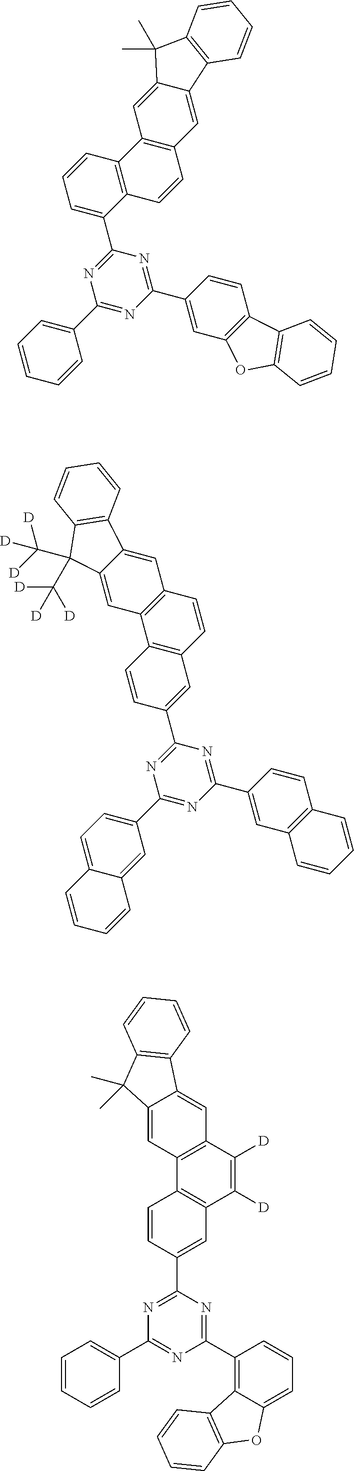 [SMILES: CC1(C)c2ccccc2-c2cc3ccc4c(-c5nc(-c6ccccc6)nc(-c6ccc7c(c6)oc6ccccc67)n5)cccc4c3cc21 ; [2H]C([2H])([2H])C1(C([2H])([2H])[2H])c2ccccc2-c2cc3ccc4cc(-c5nc(-c6ccc7ccccc7c6)nc(-c6ccc7ccccc7c6)n5)ccc4c3cc21 ; [2H]c1c([2H])c2cc3c(cc2c2ccc(-c4nc(-c5ccccc5)nc(-c5cccc6oc7ccccc7c56)n4)cc12)C(C)(C)c1ccccc1-3]

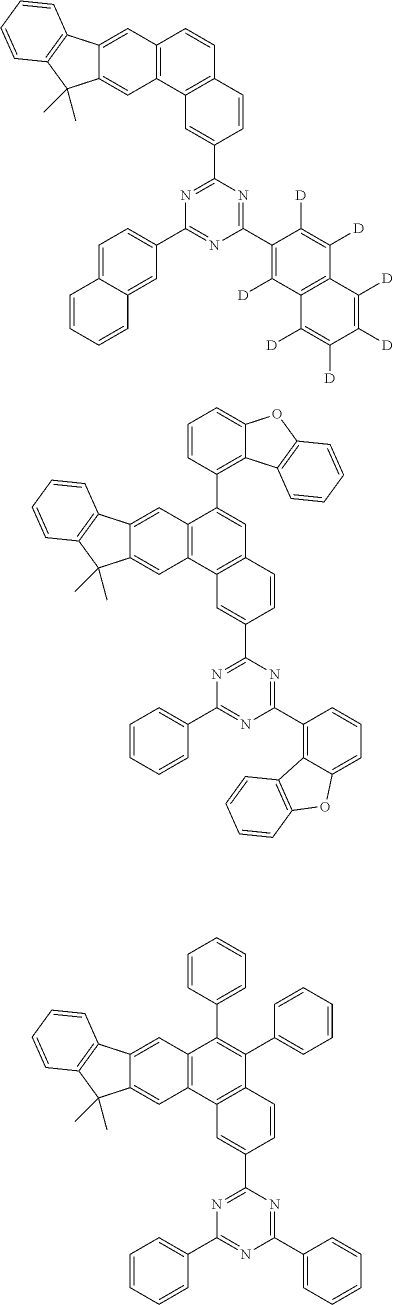 [SMILES: [2H]c1c([2H])c([2H])c2c([2H])c(-c3nc(-c4ccc5ccccc5c4)nc(-c4ccc5ccc6cc7c(cc6c5c4)C(C)(C)c4ccccc4-7)n3)c([2H])c([2H])c2c1[2H] ; CC1(C)c2ccccc2-c2cc3c(-c4cccc5oc6ccccc6c45)cc4ccc(-c5nc(-c6ccccc6)nc(-c6cccc7oc8ccccc8c67)n5)cc4c3cc21 ; CC1(C)c2ccccc2-c2cc3c(-c4ccccc4)c(-c4ccccc4)c4ccc(-c5nc(-c6ccccc6)nc(-c6ccccc6)n5)cc4c3cc21]

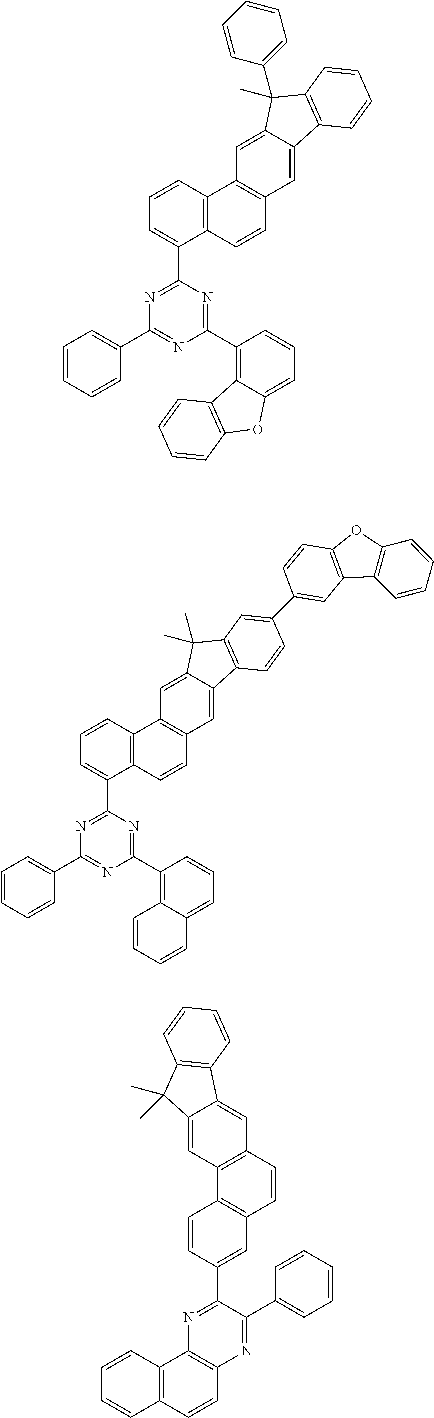 [SMILES: CC1(c2ccccc2)c2ccccc2-c2cc3ccc4c(-c5nc(-c6ccccc6)nc(-c6cccc7oc8ccccc8c67)n5)cccc4c3cc21 ; CC1(C)c2cc(-c3ccc4oc5ccccc5c4c3)ccc2-c2cc3ccc4c(-c5nc(-c6ccccc6)nc(-c6cccc7ccccc67)n5)cccc4c3cc21 ; CC1(C)c2ccccc2-c2cc3ccc4cc(-c5nc6c(ccc7ccccc76)nc5-c5ccccc5)ccc4c3cc21]

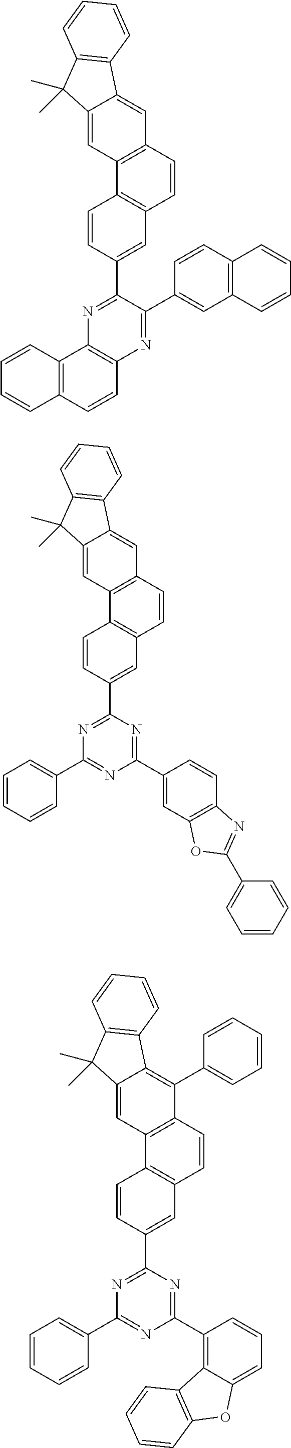

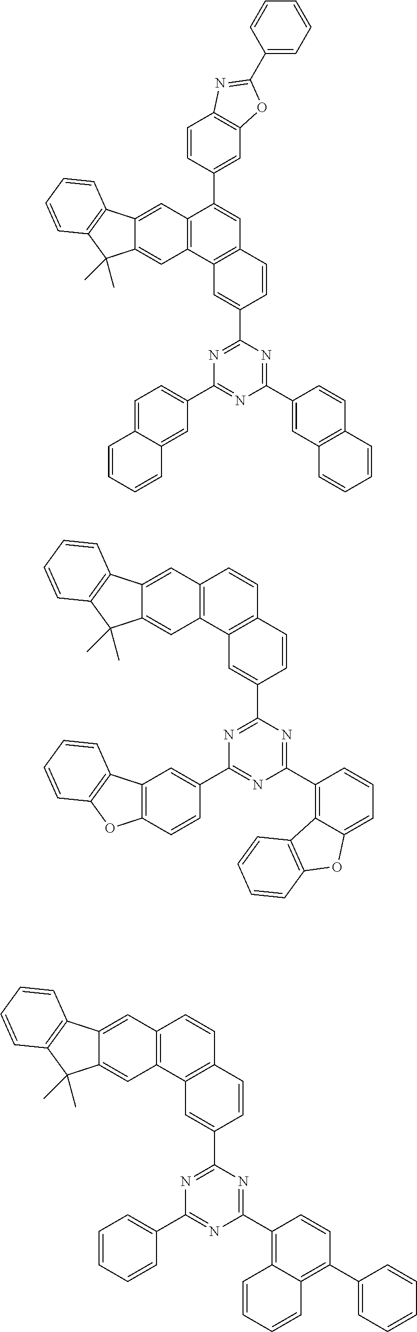 [SMILES: CC1(C)c2ccccc2-c2cc3c(-c4ccc5nc(-c6ccccc6)oc5c4)cc4ccc(-c5nc(-c6ccc7ccccc7c6)nc(-c6ccc7ccccc7c6)n5)cc4c3cc21 ; CC1(C)c2ccccc2-c2cc3ccc4ccc(-c5nc(-c6ccc7oc8ccccc8c7c6)nc(-c6cccc7oc8ccccc8c67)n5)cc4c3cc21 ; CC1(C)c2ccccc2-c2cc3ccc4ccc(-c5nc(-c6ccccc6)nc(-c6ccc(-c7ccccc7)c7ccccc67)n5)cc4c3cc21]

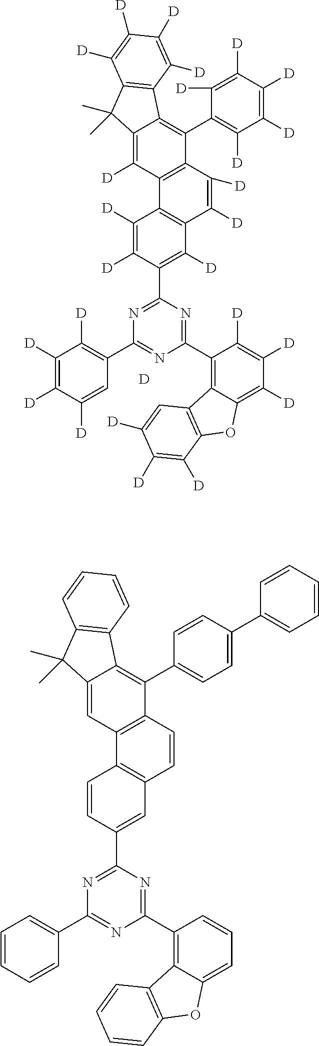

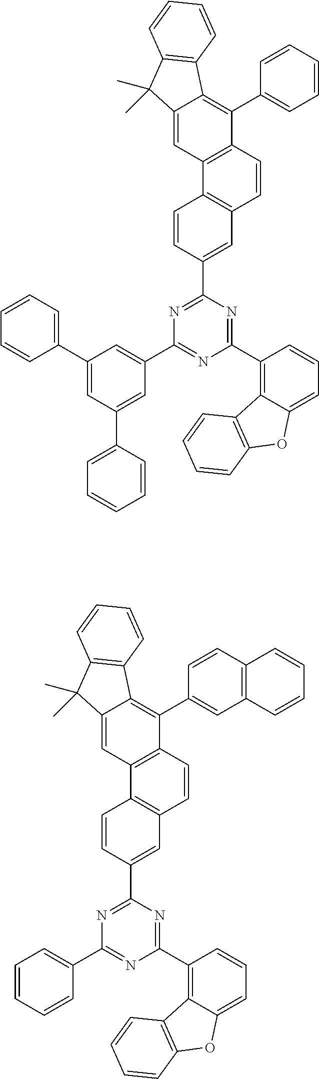 [SMILES: CC1(C)c2ccccc2-c2c1cc1c(ccc3cc(-c4nc(-c5cc(-c6ccccc6)cc(-c6ccccc6)c5)nc(-c5cccc6oc7ccccc7c56)n4)ccc31)c2-c1ccccc1 ; CC1(C)c2ccccc2-c2c1cc1c(ccc3cc(-c4nc(-c5ccccc5)nc(-c5cccc6oc7ccccc7c56)n4)ccc31)c2-c1ccc2ccccc2c1]

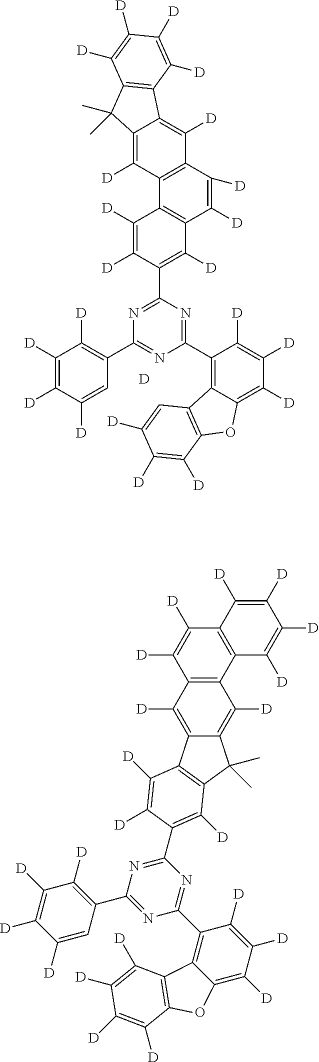

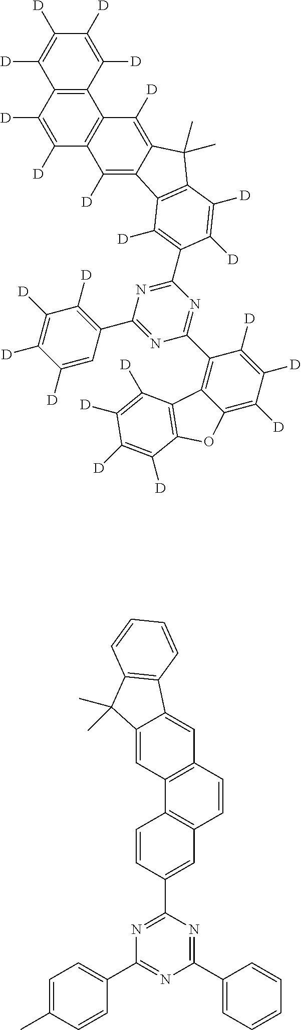 [SMILES: [2H]c1cc(-c2nc(-c3c([2H])c([2H])c4c(c3[2H])-c3c(c([2H])c5c(c3[2H])c([2H])c([2H])c3c([2H])c([2H])c([2H])c([2H])c35)C4(C)C)nc(-c3c([2H])c([2H])c([2H])c4oc5c([2H])c([2H])c([2H])c([2H])c5c34)n2)c([2H])c([2H])c1[2H] ; Cc1ccc(-c2nc(-c3ccccc3)nc(-c3ccc4c(ccc5cc6c(cc54)C(C)(C)c4ccccc4-6)c3)n2)cc1]

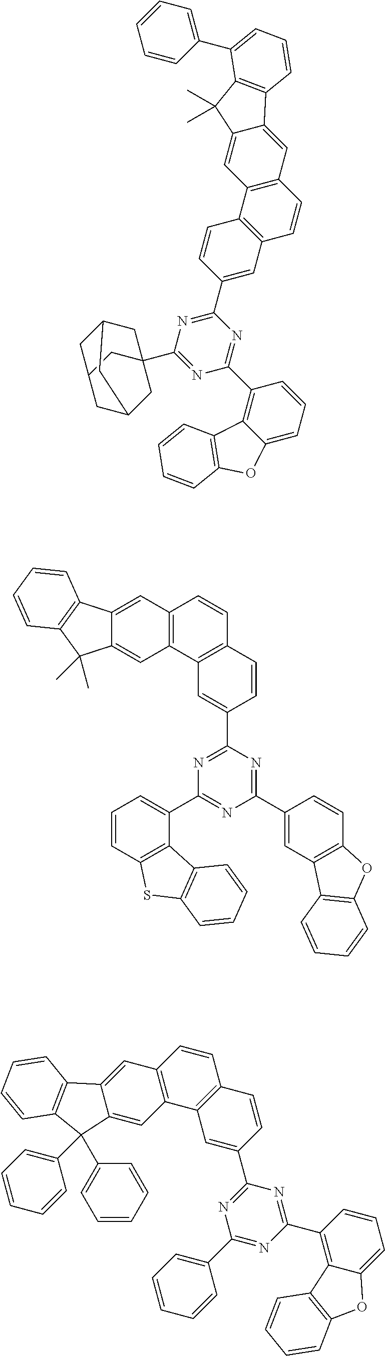 [SMILES: CC1(C)c2cc3c(ccc4cc(-c5nc(-c6cccc7oc8ccccc8c67)nc(C67CC8CC(CC(C8)C6)C7)n5)ccc43)cc2-c2cccc(-c3ccccc3)c21 ; CC1(C)c2ccccc2-c2cc3ccc4ccc(-c5nc(-c6ccc7oc8ccccc8c7c6)nc(-c6cccc7sc8ccccc8c67)n5)cc4c3cc21 ; c1ccc(-c2nc(-c3ccc4ccc5cc6c(cc5c4c3)C(c3ccccc3)(c3ccccc3)c3ccccc3-6)nc(-c3cccc4oc5ccccc5c34)n2)cc1]

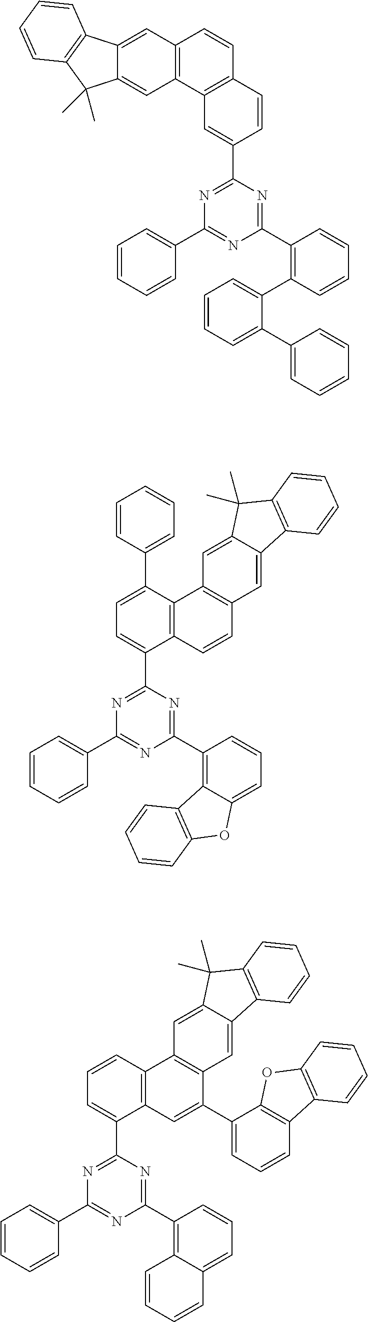 [SMILES: CC1(C)c2ccccc2-c2cc3ccc4ccc(-c5nc(-c6ccccc6)nc(-c6ccccc6-c6ccccc6-c6ccccc6)n5)cc4c3cc21 ; CC1(C)c2ccccc2-c2cc3ccc4c(-c5nc(-c6ccccc6)nc(-c6cccc7oc8ccccc8c67)n5)ccc(-c5ccccc5)c4c3cc21 ; CC1(C)c2ccccc2-c2cc3c(-c4cccc5c4oc4ccccc45)cc4c(-c5nc(-c6ccccc6)nc(-c6cccc7ccccc67)n5)cccc4c3cc21]

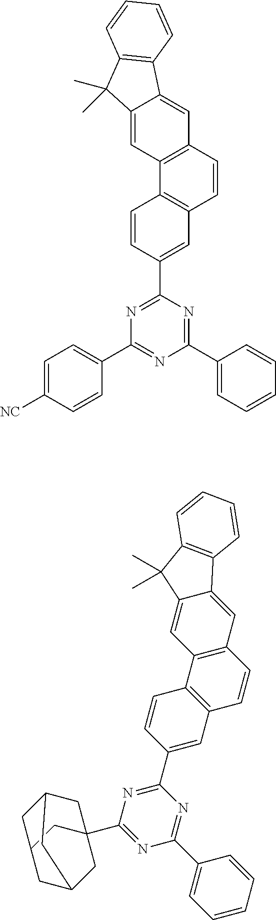 [SMILES: CC1(C)c2ccccc2-c2cc3ccc4cc(-c5nc(-c6ccccc6)nc(-c6ccc(C#N)cc6)n5)ccc4c3cc21 ; CC1(C)c2ccccc2-c2cc3ccc4cc(-c5nc(-c6ccccc6)nc(C67CC8CC(CC(C8)C6)C7)n5)ccc4c3cc21]

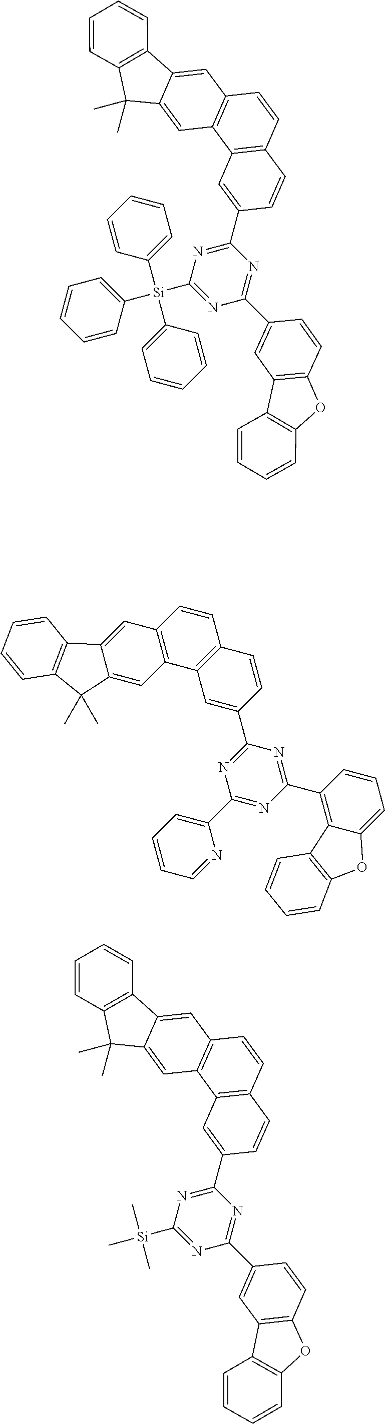 [SMILES: CC1(C)c2ccccc2-c2cc3ccc4ccc(-c5nc(-c6ccc7oc8ccccc8c7c6)nc([Si](c6ccccc6)(c6ccccc6)c6ccccc6)n5)cc4c3cc21 ; CC1(C)c2ccccc2-c2cc3ccc4ccc(-c5nc(-c6ccccn6)nc(-c6cccc7oc8ccccc8c67)n5)cc4c3cc21 ; CC1(C)c2ccccc2-c2cc3ccc4ccc(-c5nc(-c6ccc7oc8ccccc8c7c6)nc([Si](C)(C)C)n5)cc4c3cc21]

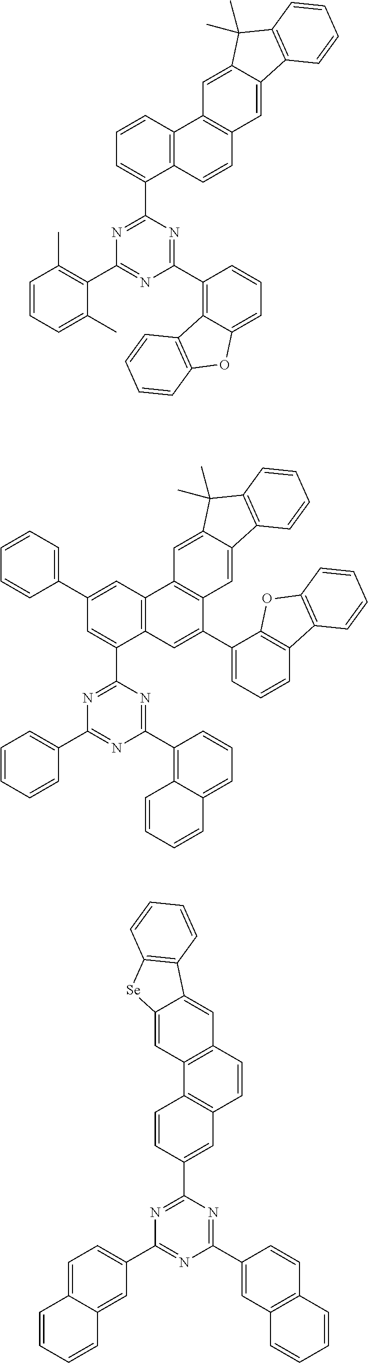 [SMILES: Cc1cccc(C)c1-c1nc(-c2cccc3c2ccc2cc4c(cc23)C(C)(C)c2ccccc2-4)nc(-c2cccc3oc4ccccc4c23)n1 ; CC1(C)c2ccccc2-c2cc3c(-c4cccc5c4oc4ccccc45)cc4c(-c5nc(-c6ccccc6)nc(-c6cccc7ccccc67)n5)cc(-c5ccccc5)cc4c3cc21 ; c1ccc2cc(-c3nc(-c4ccc5ccccc5c4)nc(-c4ccc5c(ccc6cc7c(cc65)[se]c5ccccc57)c4)n3)ccc2c1]

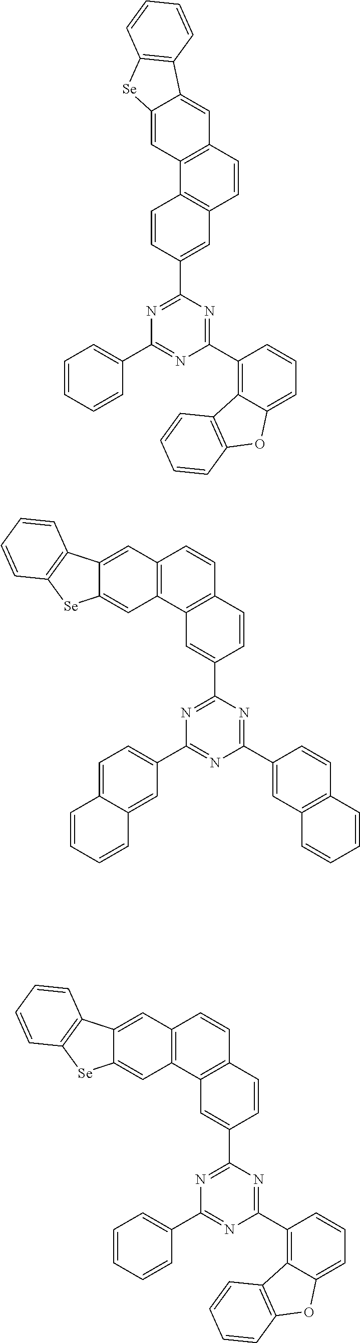 [SMILES: c1ccc(-c2nc(-c3ccc4c(ccc5cc6c(cc54)[se]c4ccccc46)c3)nc(-c3cccc4oc5ccccc5c34)n2)cc1 ; c1ccc2cc(-c3nc(-c4ccc5ccccc5c4)nc(-c4ccc5ccc6cc7c(cc6c5c4)[se]c4ccccc47)n3)ccc2c1 ; c1ccc(-c2nc(-c3ccc4ccc5cc6c(cc5c4c3)[se]c3ccccc36)nc(-c3cccc4oc5ccccc5c34)n2)cc1]

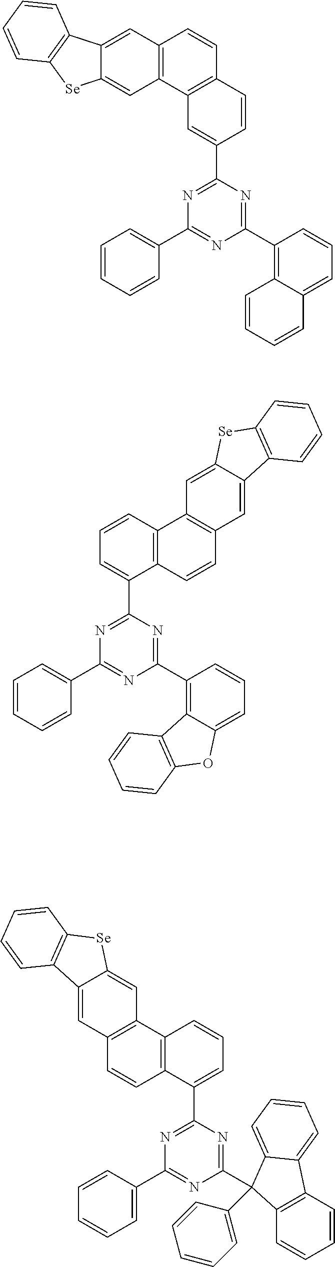 [SMILES: c1ccc(-c2nc(-c3ccc4ccc5cc6c(cc5c4c3)[se]c3ccccc36)nc(-c3cccc4ccccc34)n2)cc1 ; c1ccc(-c2nc(-c3cccc4c3ccc3cc5c(cc34)[se]c3ccccc35)nc(-c3cccc4oc5ccccc5c34)n2)cc1 ; c1ccc(-c2nc(-c3cccc4c3ccc3cc5c(cc34)[se]c3ccccc35)nc(C3(c4ccccc4)c4ccccc4-c4ccccc43)n2)cc1]

The compound represented by formula 1 according to the present disclosure may be prepared by synthetic methods known to a person skilled in the art, for example by reference to the following Reaction Scheme, but not limited thereto.

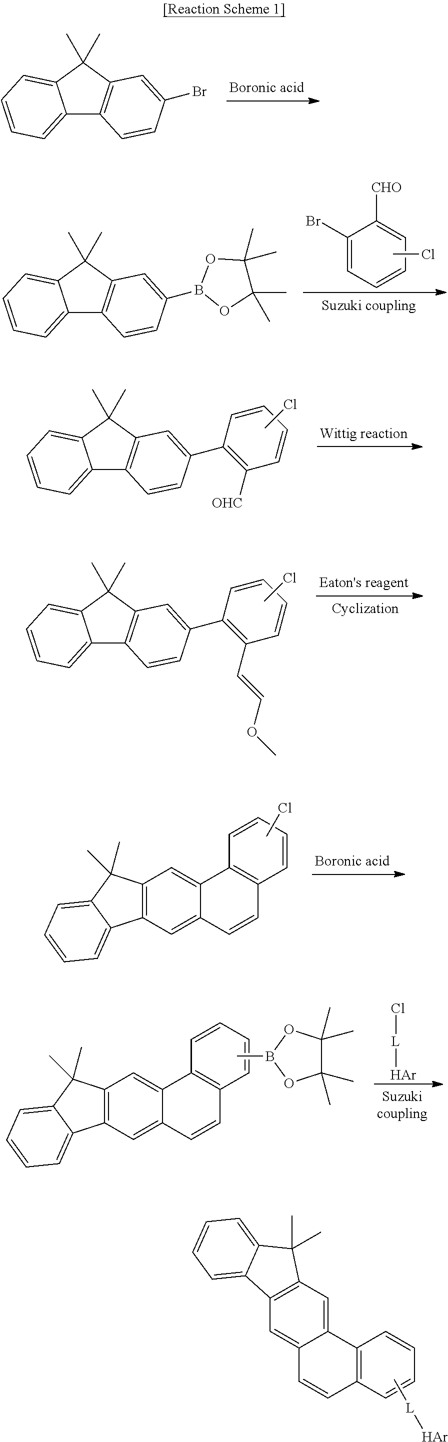

In Reaction Scheme 1, L and HAr are as defined in formula 1.

Although illustrative synthesis examples of the compound represented by formula 1 are described above, a person skilled in the art will be able to readily understand that all of them are based on a Buchwald-Hartwig cross coupling reaction, an N-arylation reaction, a H-mont-mediated etherification reaction, a Miyaura borylation reaction, a Suzuki cross-coupling reaction, a Wittig reaction, an Intramolecular acid-induced cyclization reaction, a Pd(II)-catalyzed oxidative cyclization reaction, a Grignard reaction, a Heck reaction, a Cyclic Dehydration reaction, a SN₁ substitution reaction, a SN₂ substitution reaction, a Phosphine-mediated reductive cyclization reaction, etc, and it will be readily understood by a person skilled in the art that the above reactions proceed even when substituents defined in formula 1 other than the substituents specified in the specific synthesis examples are bonded.

The organic electroluminescent material of the present disclosure includes at least one compound represented by formula 1. The organic electroluminescent material may be a host material or an electron buffer material, and may be a single host material or a plurality of host materials, but is not limited thereto. The material may consist of said organic electroluminescent compound alone or may further comprise conventional substances included in organic electroluminescent materials. The host material of the present disclosure may further comprise an organic electroluminescent compound other than the organic electroluminescent compound represented by formula 1 (first host material) as a second host material. Wherein, the weight ratio of the first host material to the second host material may be in the range of about 1:99 to about 99:1. When two or more materials are included in one layer, they may be mixture-deposited to form the layer, or they may be separately and simultaneously co-deposited to form the layer.

The present disclosure provides an organic electroluminescent material or a plurality of host materials comprising the compound represented by formula 1 above as a first host and a compound represented by formula 2 below as a second host.

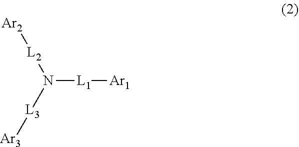

In formula 2,

L₁ to L₃ each independently represent a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted (C3-C30)cycloalkylene;

Ar₁ to Ar₃ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of (C3-C30) aliphatic ring and (C6-C30) aromatic ring, or -Lₐ-N(Arₐ)(Ar_{b});

Lₐ each independently represents a single bond, a substituted or unsubstituted (C6-C30)arylene, or a substituted or unsubstituted (3- to 30-membered)heteroarylene;

Arₐ and Ar_{b} each independently represent hydrogen, a substituted or unsubstituted (C1-C30)alkyl, a substituted or unsubstituted (C2-C30)alkenyl, a substituted or unsubstituted fused ring of (C3-C30) aliphatic ring and (C6-C30) aromatic ring, a substituted or unsubstituted (C6-C30)aryl, or a substituted or unsubstituted (3- to 30-membered)heteroaryl; and

provided that the compound where all of L₁ to L₃ are a single bond and all of Ar₁ to Ar₃ are hydrogen is excluded.

In formula 2 above, according to one embodiment of the present disclosure, L₁ to L₃ each independently represent single bond, or a substituted or unsubstituted (C6-C12)arylene. According to another embodiment of the present disclosure, L₁ to L₃ each independently represent a single bond, or an unsubstituted (C6-C12)arylene. For example, L₁ to L₃ each independently may be a single bond, phenylene, naphthylene, etc.

In formula 2 above, according to one embodiment of the present disclosure, Ar₁ to Ar₃ each independently represent a substituted or unsubstituted (C6-C30)aryl, a substituted or unsubstituted (3- to 30-membered)heteroaryl, a substituted or unsubstituted (C3-C10)cycloalkyl, or -Lₐ-N(Arₐ)(Ar_{b}). According to another embodiment of the present disclosure, Ar₁ to Ar₃ each independently represent (C6-C30)aryl unsubstituted or substituted with at least one of deuterium, (C1-C6)alkyl, and (C6-C20)aryl; a (3- to 30-membered)heteroaryl unsubstituted or substituted with at least one (C6-C15)aryl; an unsubstituted (C3-C10)cycloalkyl; or -Lₐ-N(Arₐ)(Ar_{b}). For example, Ar₁ to Ar₃ each independently may be phenyl, phenyl substituted with deuterium, phenyl substituted with methyl, phenyl substituted with tent-butyl, naphthyl, phenylnaphthyl, biphenyl, terphenyl, anthracenyl, phenanthrenyl, fluoranthenyl, tetramethyltetrahydrophenanthrenyl, di methylfl uorenyl, methylphenylfluorenyl, diphenylfluorenyl, dimethylbenzofluorenyl, spirobifluorenyl, (C22)aryl, phenylpyridyl, benzofuranyl, benzothiophenyl, benzimidazolyl substituted with phenyl, dibenzofuranyl, dibenzothiophenyl, dibenzofuranyl substituted with phenyl, carbazolyl substituted with phenyl, dibenzocarbazolyl, benzonaphthofuranyl, benzonaphthothiophenyl, phenoxazinyl, phenanthroxazolyl, phenanthroxazolyl substituted with phenyl, phenanthroxazolyl substituted with biphenyl, phenanthroxazolyl substituted with pyridyl, phenanthrothiazolyl substituted with phenyl, phenanthrothiazolyl substituted with biphenyl, benzene-fused phenanthroxazolyl substituted with phenyl, (14-membered) heteroaryl containing nitrogen substituted with methyl, (23-membered) heteroaryl containing nitrogen unsubstituted or substituted with phenyl, benzene-fused (23-membered) heteroaryl containing nitrogen, (26-membered) heteroaryl containing nitrogen unsubstituted or substituted with phenyl, benzene-fused (26-membered)-heteroaryl containing nitrogen, diphenylamino, etc.

According to one embodiment of the present disclosure, Lₐ each independently represents a single bond.

According to one embodiment of the present disclosure, Arₐ and Ar_{b} each independently represent a substituted or unsubstituted (C6-C12)aryl. According to another embodiment of the present disclosure, Arₐ and Ar_{b} each independently represent an unsubstituted (C6-C12)aryl. For example, Arₐ and Ar_{b} each independently may be phenyl, etc.

According to one embodiment of the present disclosure, the formula 2 may be represented by any one of the following formulas 2-1 to 2-12.

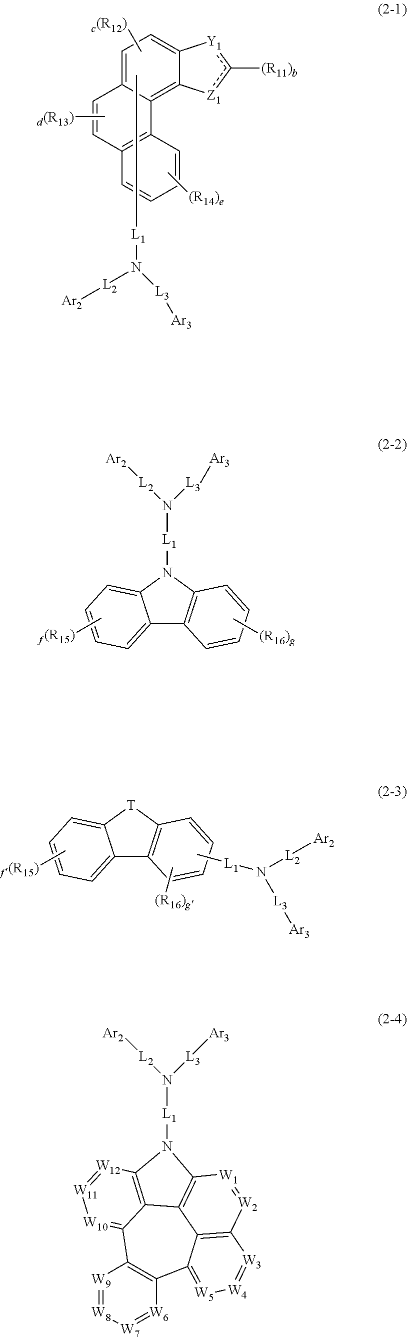

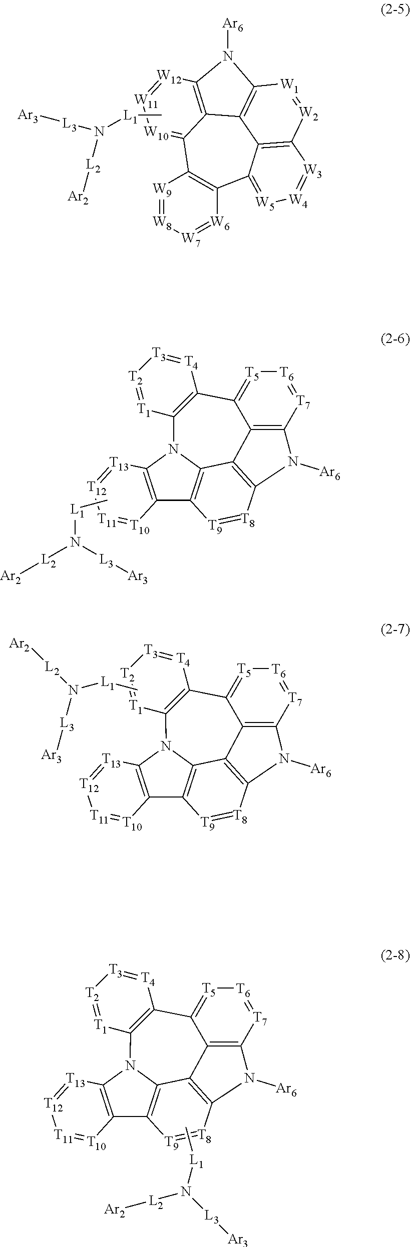

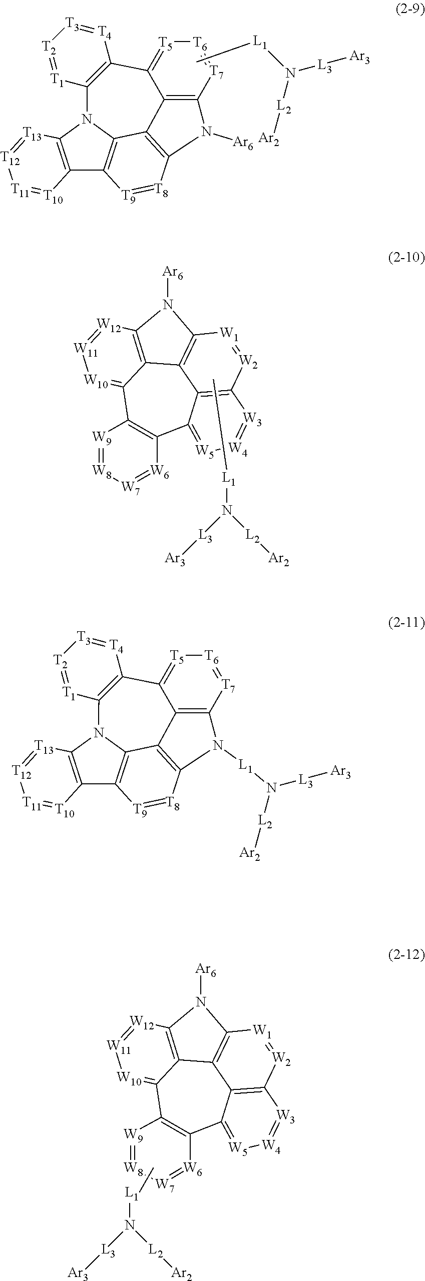

In the formulas 2-1 to 2-12,

Y₁ and Z₁ each independently represent -N═, —N(R₂₁)—, —O—, or —S—, with a proviso that any one of Y₁ and Z₁ represents —N═ and the other represents —N(R₂₁)—, —O—, or —S—;

T represents C(R₂₂)(R₂₃), N(R₂₄), O, or S;

T₁ to T₁₃ and W₁ to W₁₂ each independently represent N or C(V₁);

R₁₁ represents a substituted or unsubstituted (C6-C30)aryl, or substituted or unsubstituted (3- to 30-membered)heteroaryl;

R₁₂ to R₁₆, R₂₁ to R₂₄, and V₁ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of a (C3-C30) aliphatic ring and a (C6-C30) aromatic ring, or -L_{c}-N(Arₑ)(Ar_{f}); or may be linked to an adjacent substituent(s) to form a ring;

L, each independently represents single bond, a substituted or unsubstituted (C6-C30)arylene, or substituted or unsubstituted (3- to 30-membered)heteroarylene;

Arₑ and Ar_{f} each independently represent hydrogen, a substituted or unsubstituted (C1-C30)alkyl, a substituted or unsubstituted (C2-C30)alkenyl, a fused ring of substituted or unsubstituted (C3-C30) aliphatic ring and (C6-C30) aromatic ring, a substituted or unsubstituted (C6-C30)aryl, or a substituted or unsubstituted (3- to 30-membered)heteroaryl;

Ar₆ represents a substituted or unsubstituted (C6-C30)aryl, or a substituted or unsubstituted (3- to 30-membered)heteroaryl;

b represents 1, c and d each independently represent 1 or 2, e, f, g and f′ each independently represent an integer of 1 to 4, and g′ represents an integer of 1 to 3, when c to g, f′ and g′ are 2 or more, each of R₁₂ to R₁₆ may be the same or different from each other; and

Arₑ, Arₐ, and L₁ to L₃ are as defined in formula 2.

The compound represented by the formula 2 may be at least one selected from the group consisting of the following compounds, but is not limited thereto.

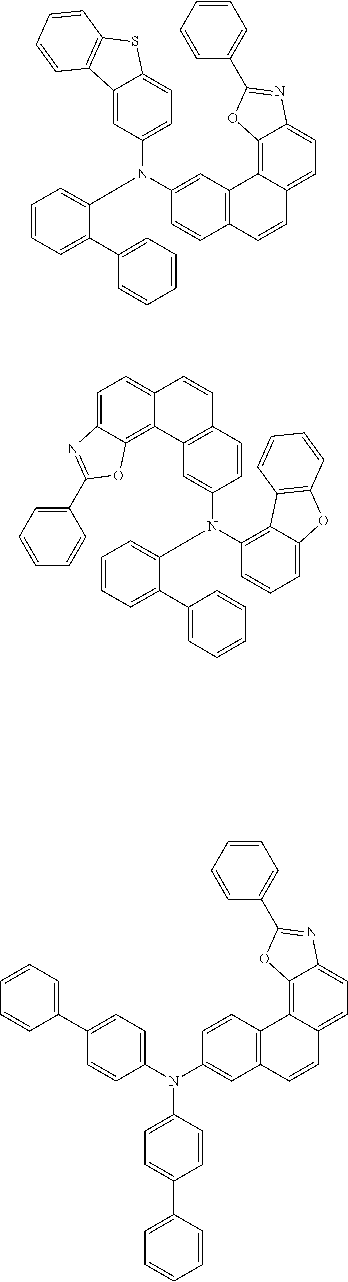 [SMILES: c1ccc(-c2nc3ccc4ccc5ccc(N(c6ccc7sc8ccccc8c7c6)c6ccccc6-c6ccccc6)cc5c4c3o2)cc1 ; c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1]

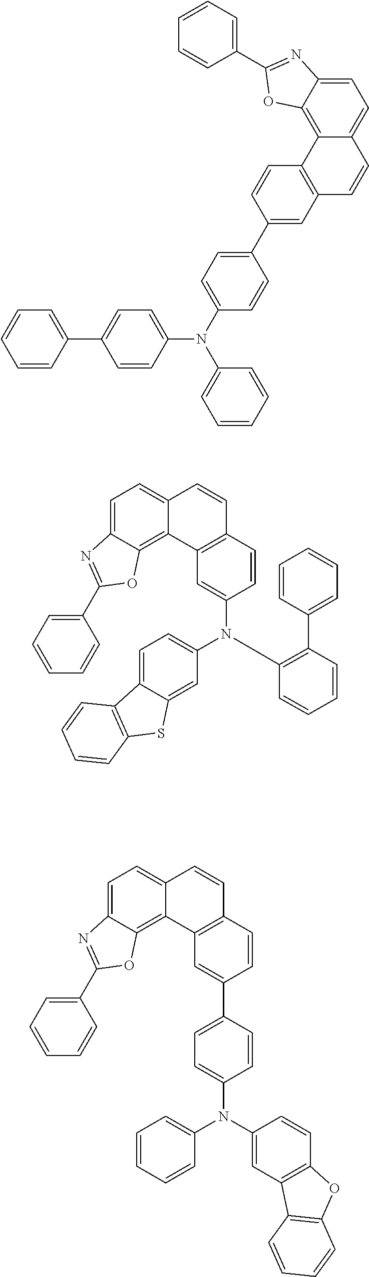 [SMILES: c1ccc(-c2ccc(N(c3ccccc3)c3ccc(-c4ccc5c(ccc6ccc7nc(-c8ccccc8)oc7c65)c4)cc3)cc2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1]

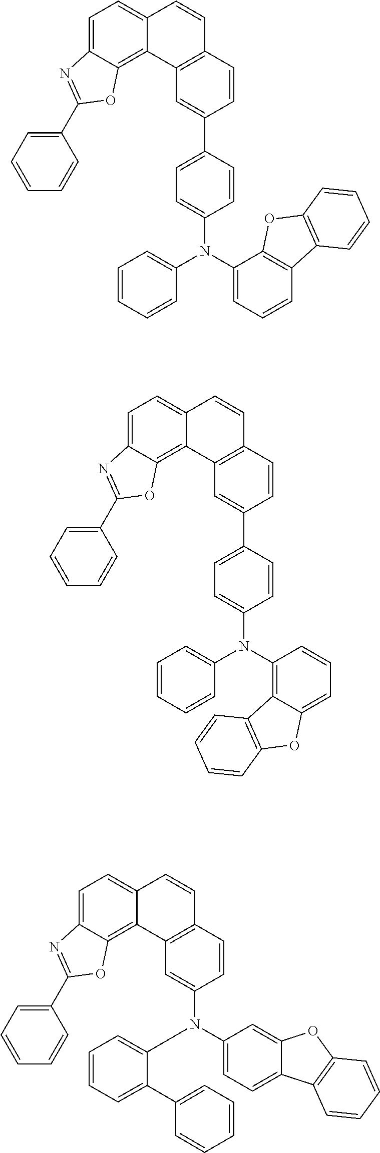 [SMILES: c1ccc(-c2nc3ccc4ccc5ccc(-c6ccc(N(c7ccccc7)c7cccc8c7oc7ccccc78)cc6)cc5c4c3o2)cc1 ; c1ccc(-c2nc3ccc4ccc5ccc(-c6ccc(N(c7ccccc7)c7cccc8oc9ccccc9c78)cc6)cc5c4c3o2)cc1 ; c1ccc(-c2nc3ccc4ccc5ccc(N(c6ccc7c(c6)oc6ccccc67)c6ccccc6-c6ccccc6)cc5c4c3o2)cc1]

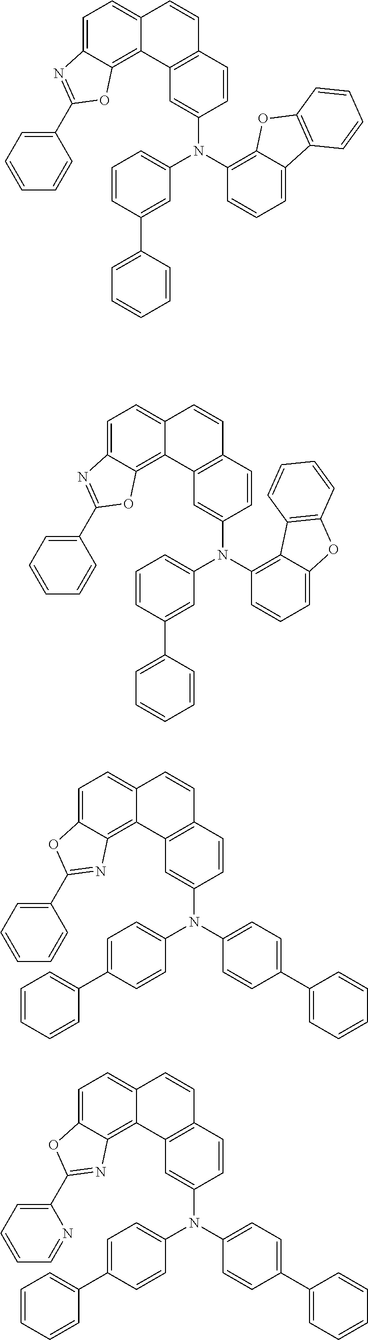 [SMILES: c1ccc(-c2cccc(N(c3ccc4ccc5ccc6nc(-c7ccccc7)oc6c5c4c3)c3cccc4c3oc3ccccc34)c2)cc1 ; c1ccc(-c2cccc(N(c3ccc4ccc5ccc6nc(-c7ccccc7)oc6c5c4c3)c3cccc4oc5ccccc5c34)c2)cc1 ; c1ccc(-c2ccc(N(c3ccc(-c4ccccc4)cc3)c3ccc4ccc5ccc6oc(-c7ccccc7)nc6c5c4c3)cc2)cc1 ; c1ccc(-c2ccc(N(c3ccc(-c4ccccc4)cc3)c3ccc4ccc5ccc6oc(-c7ccccn7)nc6c5c4c3)cc2)cc1]

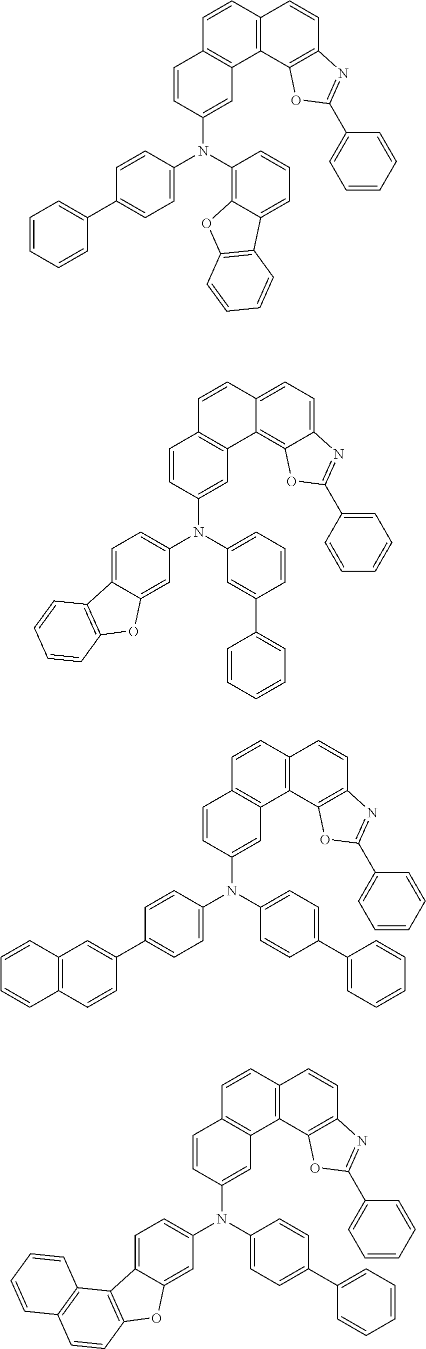

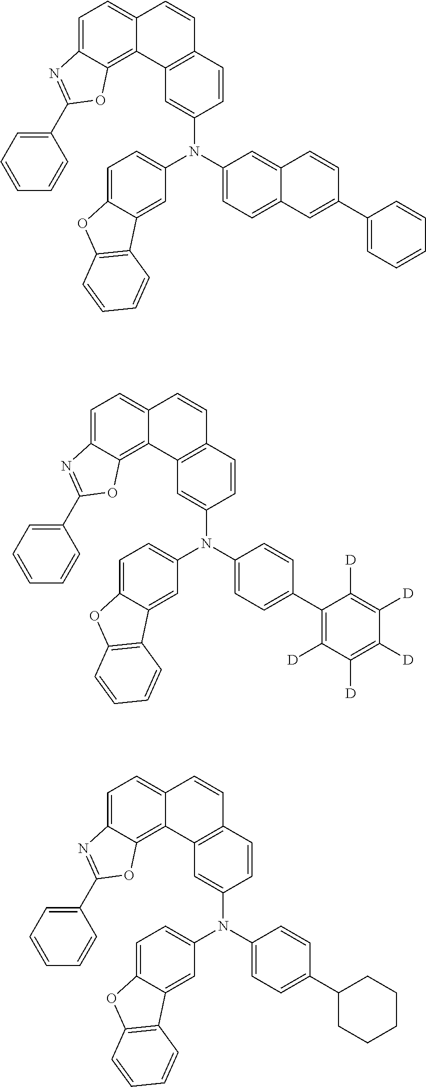 [SMILES: c1ccc(-c2ccc3cc(N(c4ccc5oc6ccccc6c5c4)c4ccc5ccc6ccc7nc(-c8ccccc8)oc7c6c5c4)ccc3c2)cc1 ; [2H]c1c([2H])c([2H])c(-c2ccc(N(c3ccc4oc5ccccc5c4c3)c3ccc4ccc5ccc6nc(-c7ccccc7)oc6c5c4c3)cc2)c([2H])c1[2H] ; c1ccc(-c2nc3ccc4ccc5ccc(N(c6ccc(C7CCCCC7)cc6)c6ccc7oc8ccccc8c7c6)cc5c4c3o2)cc1]

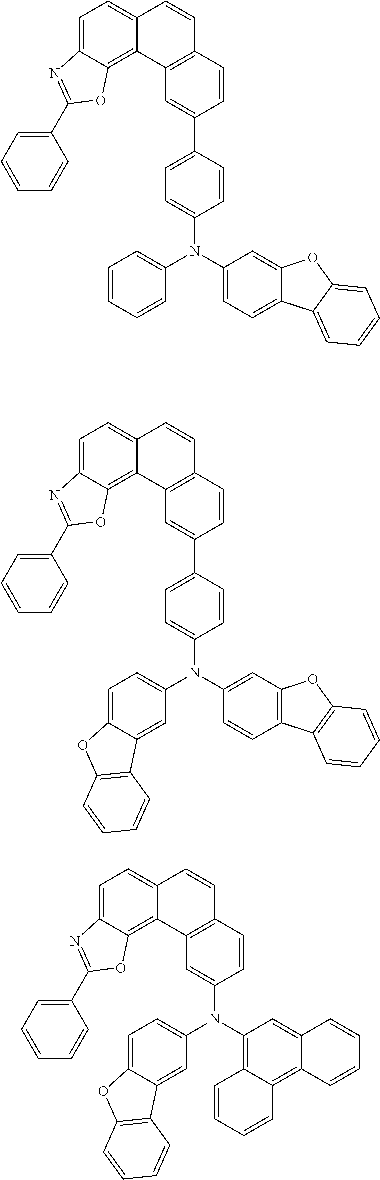

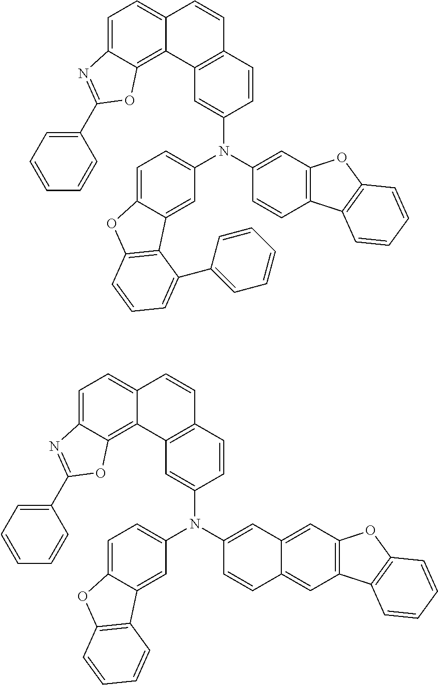 [SMILES: c1ccc(-c2nc3ccc4ccc5ccc(N(c6ccc7c(c6)oc6ccccc67)c6ccc7oc8cccc(-c9ccccc9)c8c7c6)cc5c4c3o2)cc1 ; c1ccc(-c2nc3ccc4ccc5ccc(N(c6ccc7cc8c(cc7c6)oc6ccccc68)c6ccc7oc8ccccc8c7c6)cc5c4c3o2)cc1]

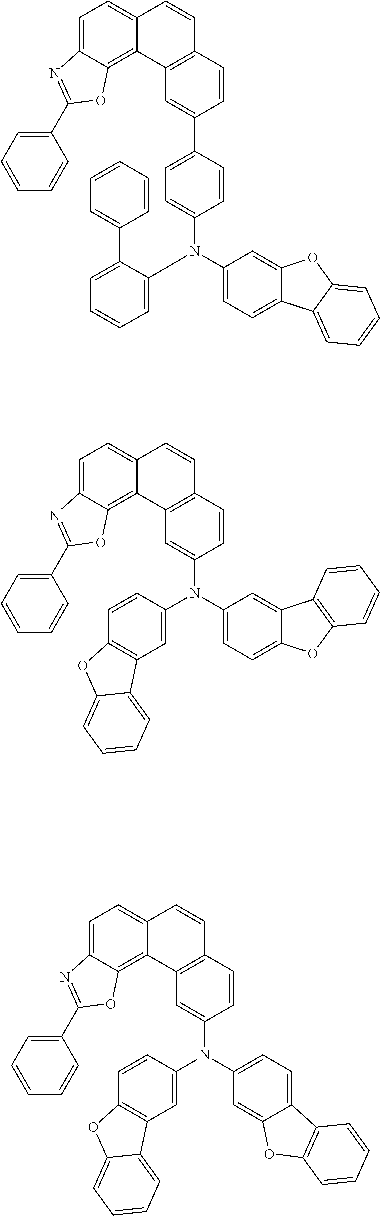 [SMILES: c1ccc(-c2nc3ccc4ccc5ccc(-c6ccc(N(c7ccc8c(c7)oc7ccccc78)c7ccccc7-c7ccccc7)cc6)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3o2)cc1]

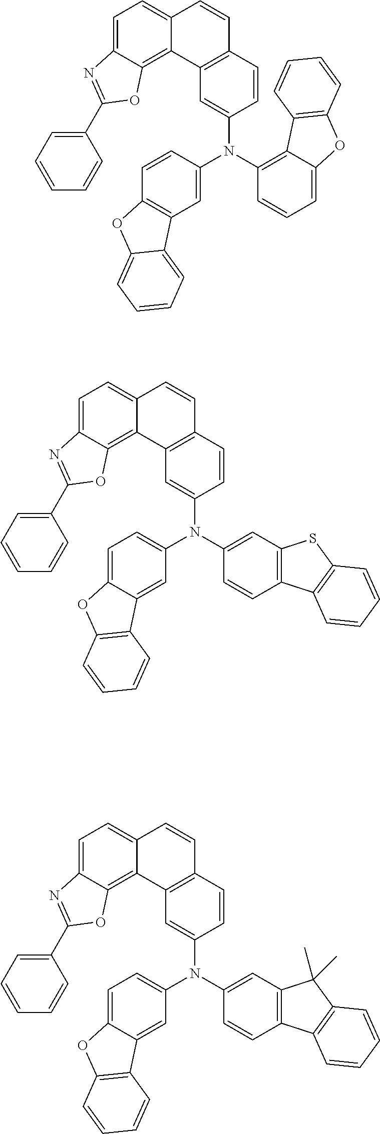 [SMILES: c1ccc(-c2nc3ccc4ccc5ccc(N(c6ccc7oc8ccccc8c7c6)c6cccc7oc8ccccc8c67)cc5c4c3o2)cc1 ; c1ccc(-c2nc3ccc4ccc5ccc(N(c6ccc7c(c6)sc6ccccc67)c6ccc7oc8ccccc8c7c6)cc5c4c3o2)cc1 ; CC1(C)c2ccccc2-c2ccc(N(c3ccc4oc5ccccc5c4c3)c3ccc4ccc5ccc6nc(-c7ccccc7)oc6c5c4c3)cc21]

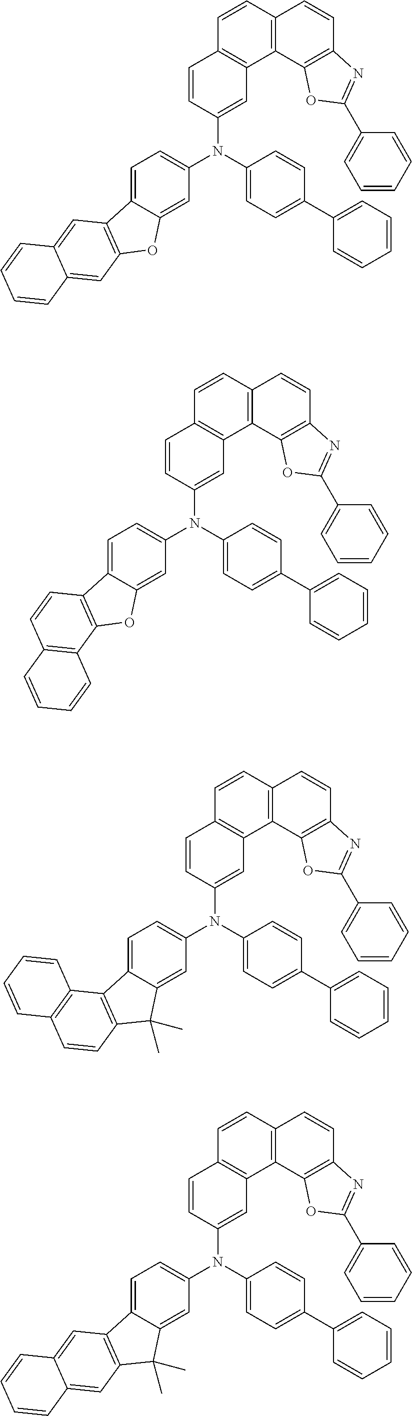 [SMILES: c1ccc(-c2ccc(N(c3ccc4c(c3)oc3cc5ccccc5cc34)c3ccc4ccc5ccc6nc(-c7ccccc7)oc6c5c4c3)cc2)cc1 ; c1ccc(-c2ccc(N(c3ccc4c(c3)oc3c5ccccc5ccc43)c3ccc4ccc5ccc6nc(-c7ccccc7)oc6c5c4c3)cc2)cc1 ; CC1(C)c2cc(N(c3ccc(-c4ccccc4)cc3)c3ccc4ccc5ccc6nc(-c7ccccc7)oc6c5c4c3)ccc2-c2c1ccc1ccccc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21]

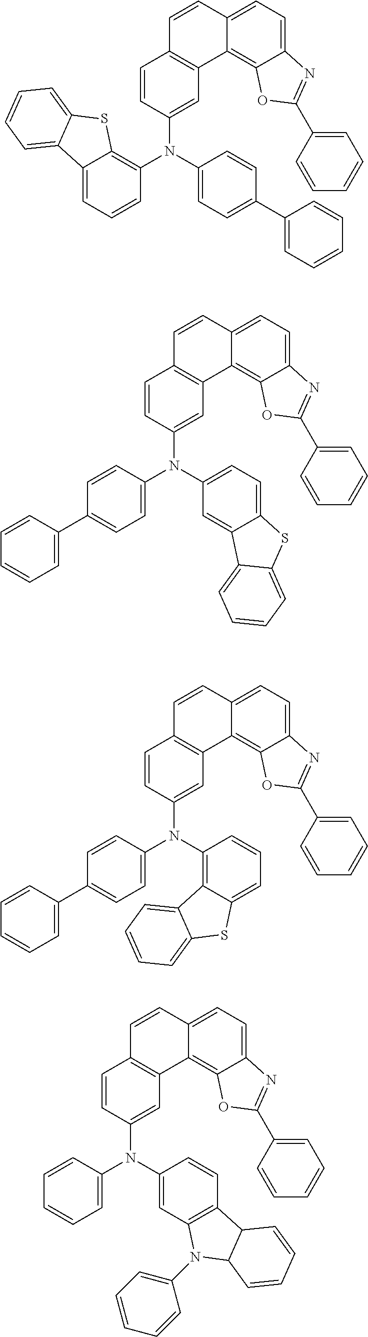

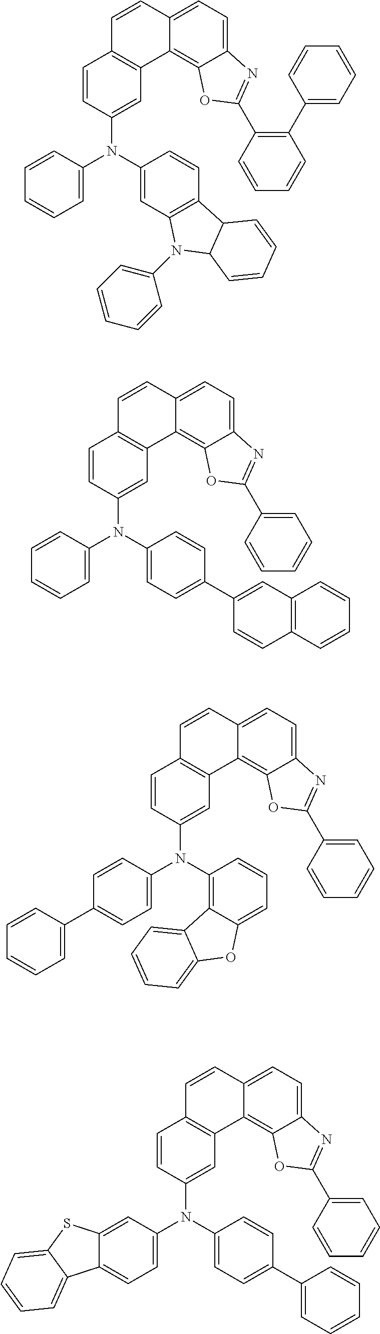 [SMILES: C1=CC2c3ccc(N(c4ccccc4)c4ccc5ccc6ccc7nc(-c8ccccc8-c8ccccc8)oc7c6c5c4)cc3N(c3ccccc3)C2C=C1 ; c1ccc(-c2nc3ccc4ccc5ccc(N(c6ccccc6)c6ccc(-c7ccc8ccccc8c7)cc6)cc5c4c3o2)cc1 ; c1ccc(-c2ccc(N(c3ccc4ccc5ccc6nc(-c7ccccc7)oc6c5c4c3)c3cccc4oc5ccccc5c34)cc2)cc1 ; c1ccc(-c2ccc(N(c3ccc4c(c3)sc3ccccc34)c3ccc4ccc5ccc6nc(-c7ccccc7)oc6c5c4c3)cc2)cc1]

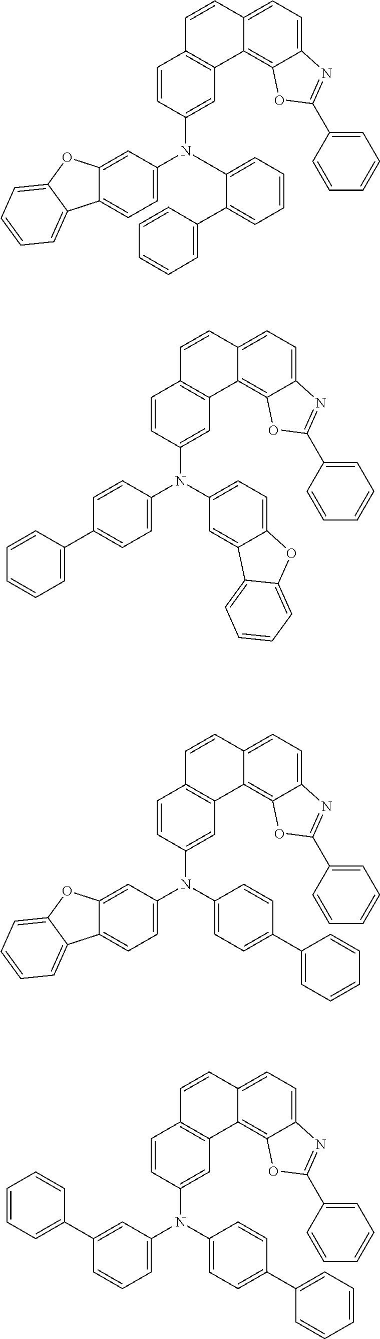 [SMILES: c1ccc(-c2nc3ccc4ccc5ccc(N(c6ccc7c(c6)oc6ccccc67)c6ccccc6-c6ccccc6)cc5c4c3o2)cc1 ; c1ccc(-c2ccc(N(c3ccc4oc5ccccc5c4c3)c3ccc4ccc5ccc6nc(-c7ccccc7)oc6c5c4c3)cc2)cc1 ; c1ccc(-c2ccc(N(c3ccc4c(c3)oc3ccccc34)c3ccc4ccc5ccc6nc(-c7ccccc7)oc6c5c4c3)cc2)cc1 ; c1ccc(-c2ccc(N(c3cccc(-c4ccccc4)c3)c3ccc4ccc5ccc6nc(-c7ccccc7)oc6c5c4c3)cc2)cc1]

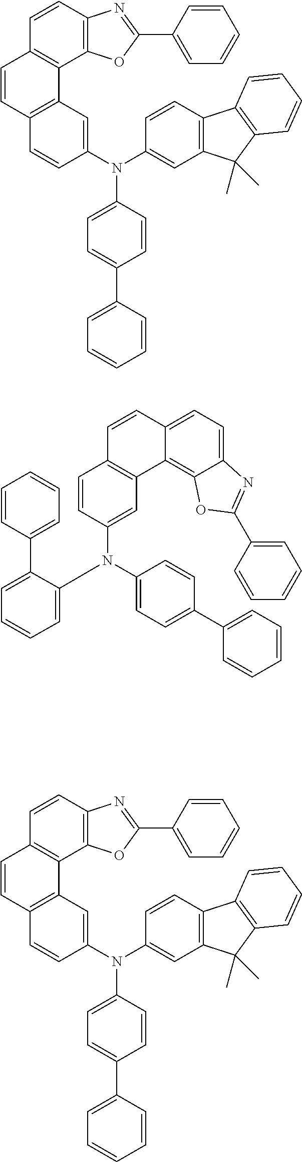 [SMILES: CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc4ccc5ccc6nc(-c7ccccc7)oc6c5c4c3)c3ccccc3-c3ccccc3)cc2)cc1 ; CC1(C)c2ccccc2-c2ccc(N(c3ccc(-c4ccccc4)cc3)c3ccc4ccc5ccc6nc(-c7ccccc7)oc6c5c4c3)cc21]

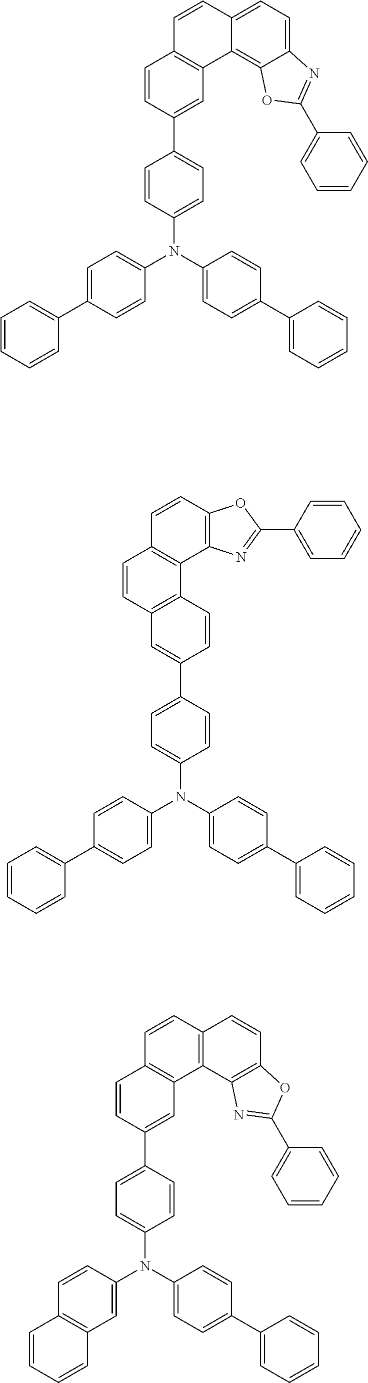 [SMILES: c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1]

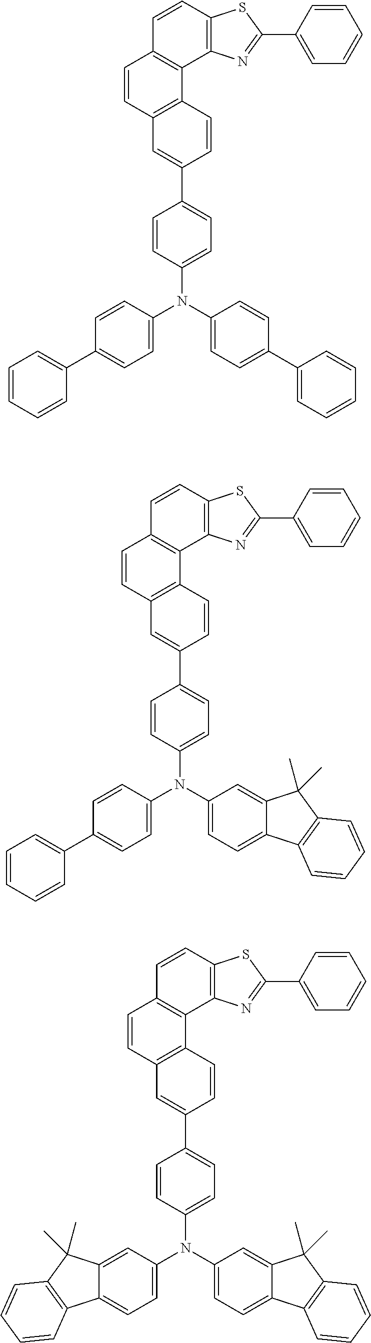 [SMILES: c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21]

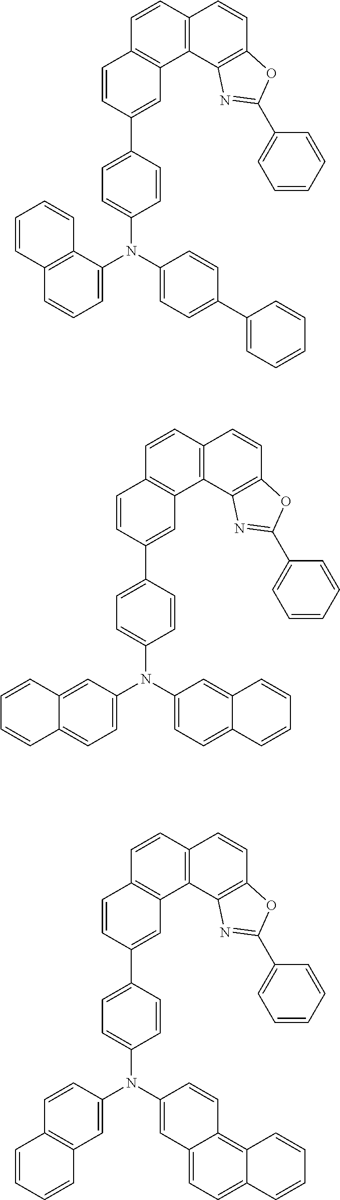 [SMILES: c1ccc(-c2ccc(N(c3ccc(-c4ccc5ccc6ccc7oc(-c8ccccc8)nc7c6c5c4)cc3)c3cccc4ccccc34)cc2)cc1 ; c1ccc(-c2nc3c(ccc4ccc5ccc(-c6ccc(N(c7ccc8ccccc8c7)c7ccc8ccccc8c7)cc6)cc5c43)o2)cc1 ; c1ccc(-c2nc3c(ccc4ccc5ccc(-c6ccc(N(c7ccc8ccccc8c7)c7ccc8c(ccc9ccccc98)c7)cc6)cc5c43)o2)cc1]

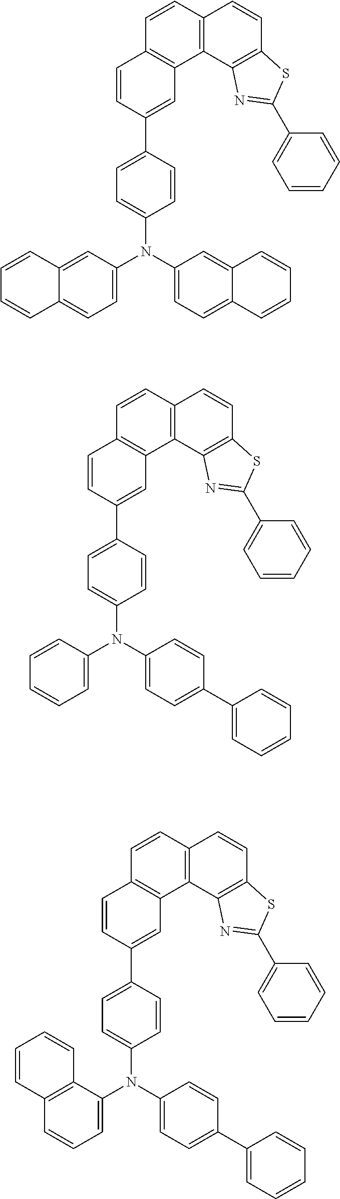 [SMILES: c1ccc(-c2nc3c(ccc4ccc5ccc(-c6ccc(N(c7ccc8ccccc8c7)c7ccc8ccccc8c7)cc6)cc5c43)s2)cc1 ; c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1]

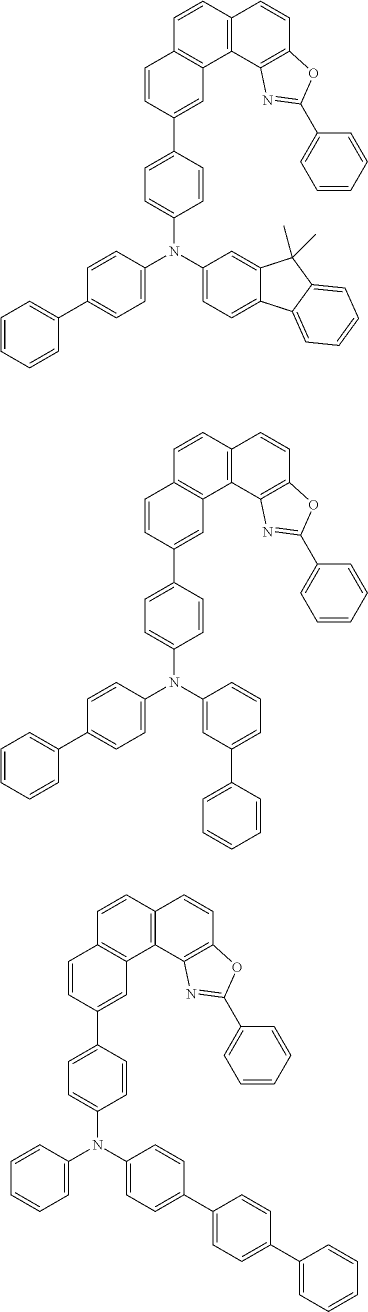 [SMILES: CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1]

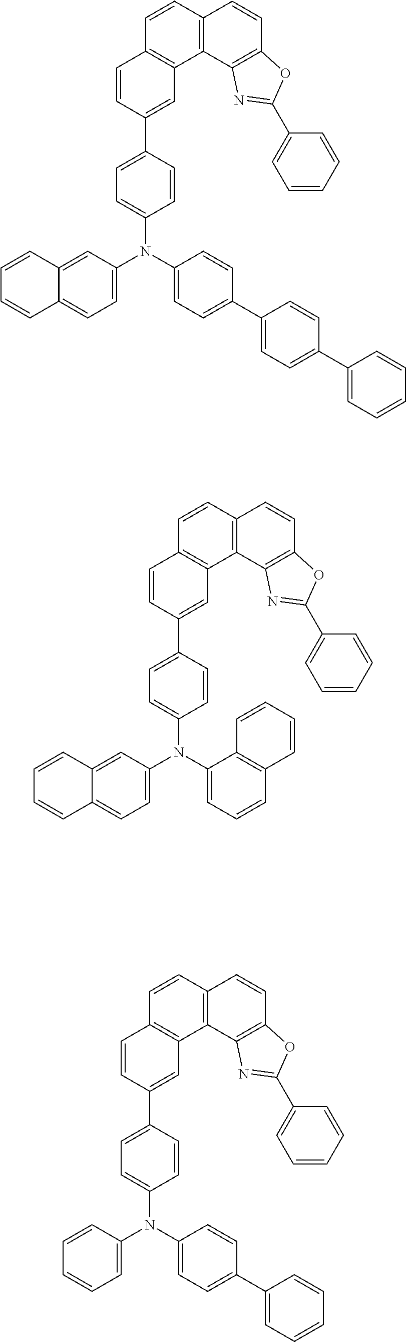 [SMILES: c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(N(c3ccccc3)c3ccc(-c4ccc5ccc6ccc7oc(-c8ccccc8)nc7c6c5c4)cc3)cc2)cc1]

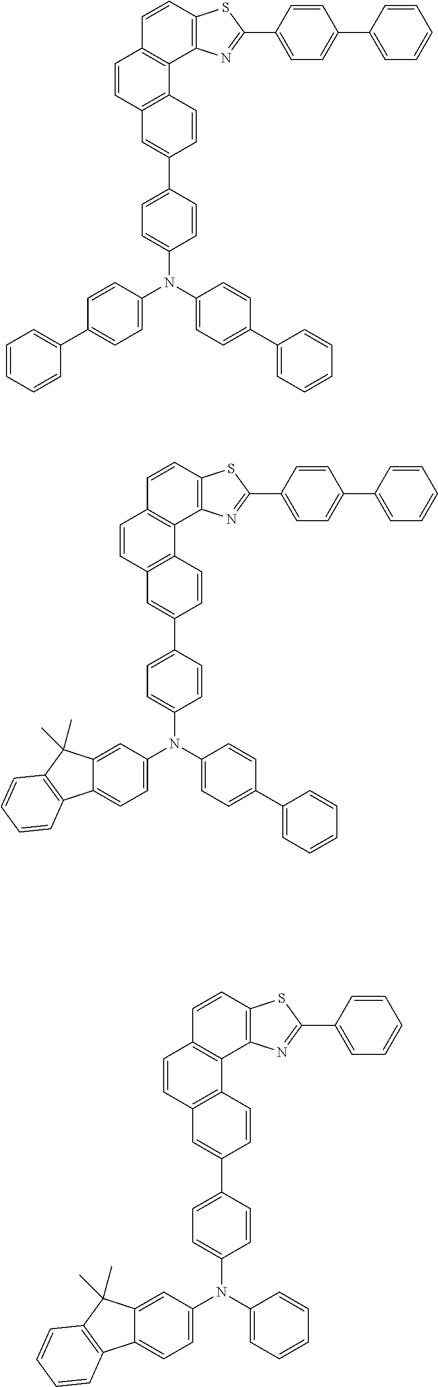 [SMILES: c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; CC1(C)c2ccccc2-c2ccc(N(c3ccccc3)c3ccc(-c4ccc5c(ccc6ccc7sc(-c8ccccc8)nc7c65)c4)cc3)cc21]

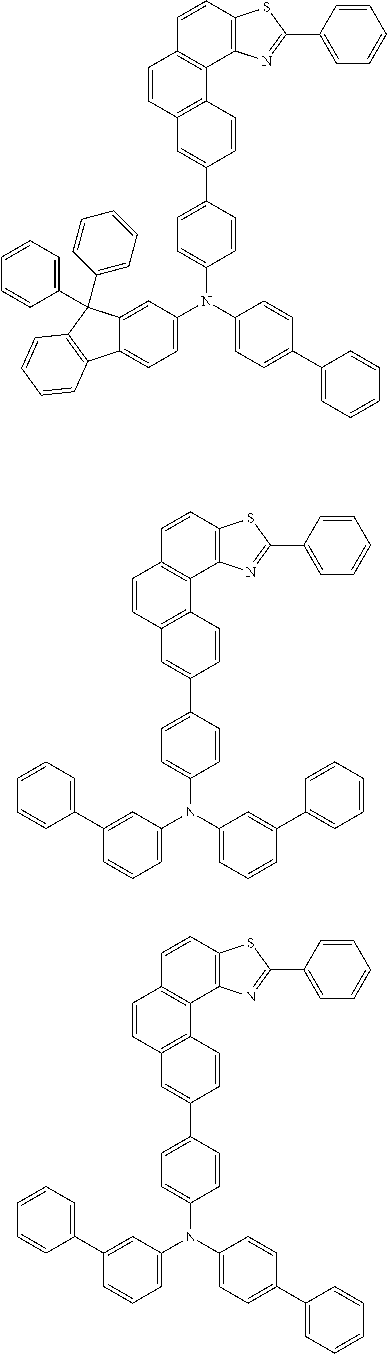 [SMILES: c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1]

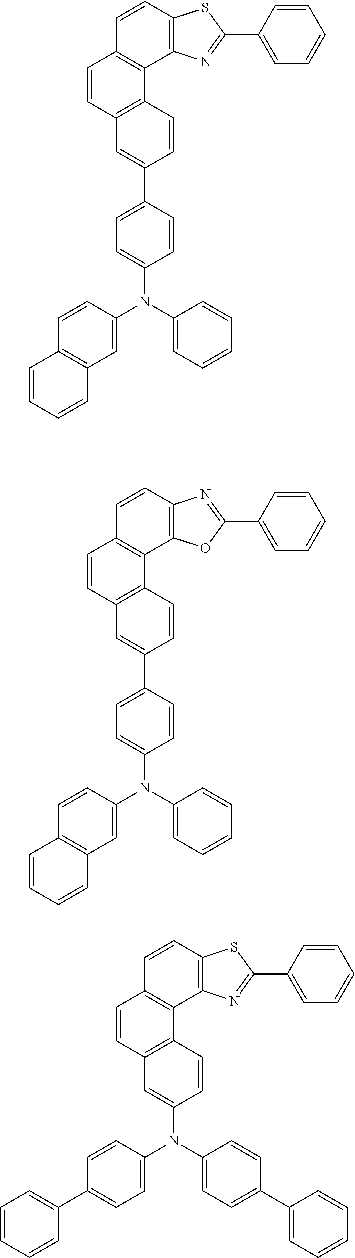 [SMILES: c1ccc(-c2nc3c(ccc4ccc5cc(-c6ccc(N(c7ccccc7)c7ccc8ccccc8c7)cc6)ccc5c43)s2)cc1 ; c1ccc(-c2nc3ccc4ccc5cc(-c6ccc(N(c7ccccc7)c7ccc8ccccc8c7)cc6)ccc5c4c3o2)cc1 ; c1ccc(-c2ccc(N(c3ccc(-c4ccccc4)cc3)c3ccc4c(ccc5ccc6sc(-c7ccccc7)nc6c54)c3)cc2)cc1]

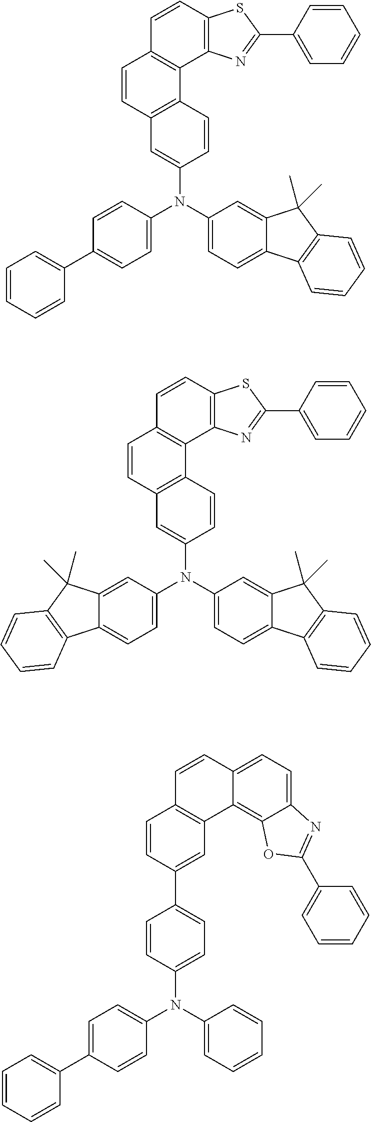 [SMILES: CC1(C)c2ccccc2-c2ccc(N(c3ccc(-c4ccccc4)cc3)c3ccc4c(ccc5ccc6sc(-c7ccccc7)nc6c54)c3)cc21 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5ccc6ccc7nc(-c8ccccc8)oc7c6c5c4)cc3)cc2)cc1]

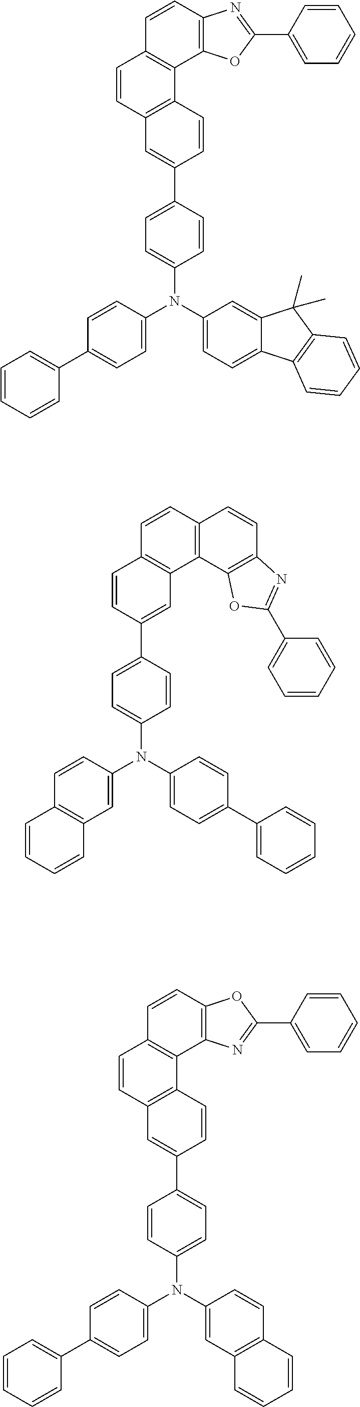 [SMILES: CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21 ; c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1]

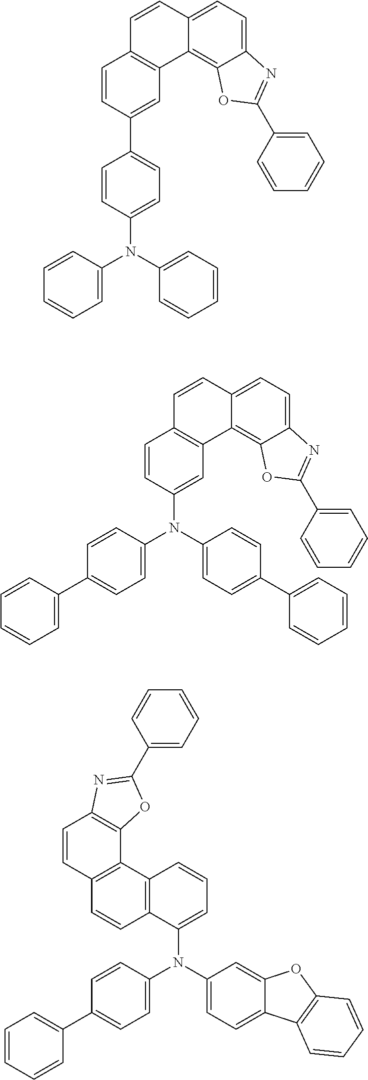 [SMILES: c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1 ; c1ccc(-c2ccc(N(c3ccc4c(c3)oc3ccccc34)c3cccc4c3ccc3ccc5nc(-c6ccccc6)oc5c34)cc2)cc1]

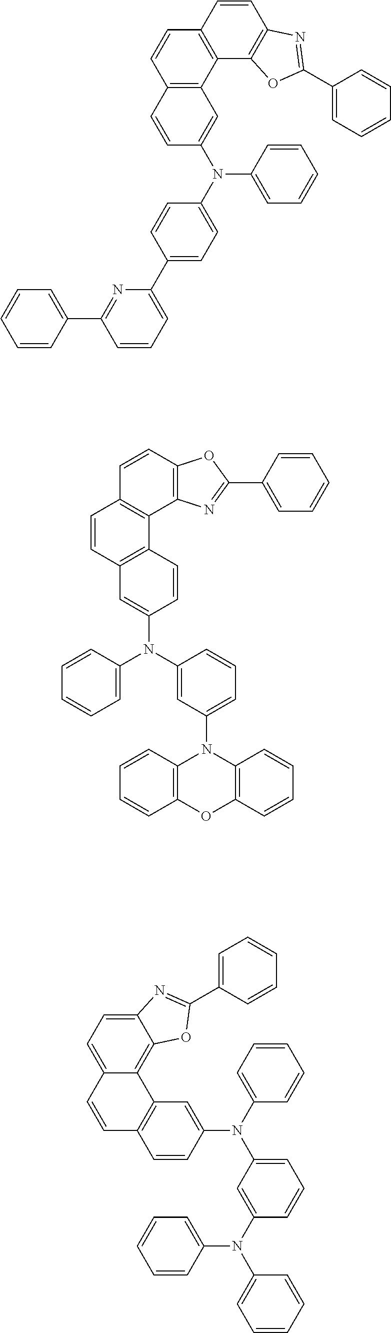 [SMILES: c1ccc(-c2cccc(-c3ccc(N(c4ccccc4)c4ccc5ccc6ccc7nc(-c8ccccc8)oc7c6c5c4)cc3)n2)cc1 ; c1ccc(-c2nc3c(ccc4ccc5cc(N(c6ccccc6)c6cccc(N7c8ccccc8Oc8ccccc87)c6)ccc5c43)o2)cc1 ; c1ccc(-c2nc3ccc4ccc5ccc(N(c6ccccc6)c6cccc(N(c7ccccc7)c7ccccc7)c6)cc5c4c3o2)cc1]

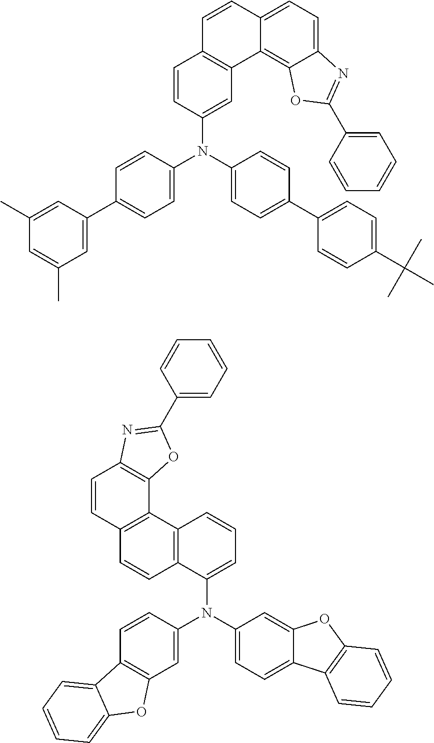 [SMILES: Cc1cc(C)cc(-c2ccc(N(c3ccc(-c4ccc(C(C)(C)C)cc4)cc3)c3ccc4ccc5ccc6nc(-c7ccccc7)oc6c5c4c3)cc2)c1 ; c1ccc(-c2nc3ccc4ccc5c(N(c6ccc7c(c6)oc6ccccc67)c6ccc7c(c6)oc6ccccc67)cccc5c4c3o2)cc1]

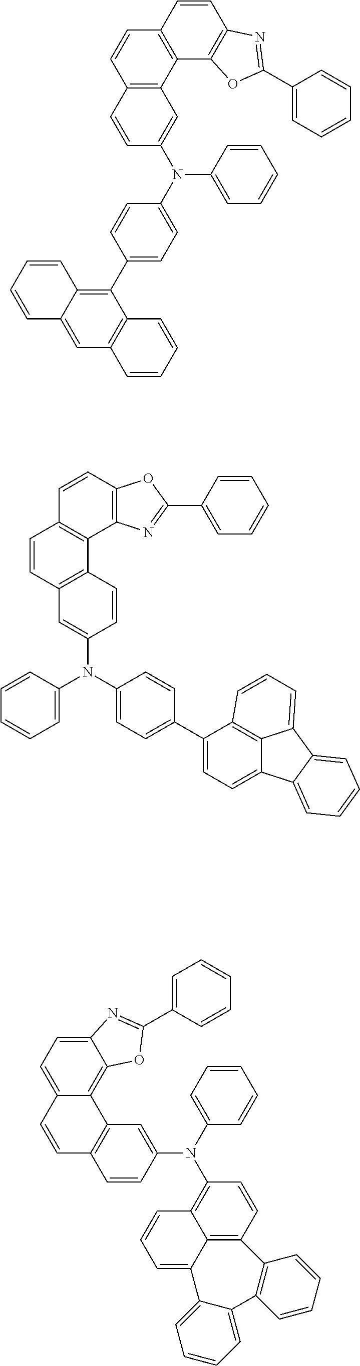 [SMILES: c1ccc(-c2nc3ccc4ccc5ccc(N(c6ccccc6)c6ccc(-c7c8ccccc8cc8ccccc78)cc6)cc5c4c3o2)cc1 ; c1ccc(-c2nc3c(ccc4ccc5cc(N(c6ccccc6)c6ccc(-c7ccc8c9c(cccc79)-c7ccccc7-8)cc6)ccc5c43)o2)cc1 ; c1ccc(-c2nc3ccc4ccc5ccc(N(c6ccccc6)c6ccc7c8c(cccc68)-c6ccccc6-c6ccccc6-7)cc5c4c3o2)cc1]

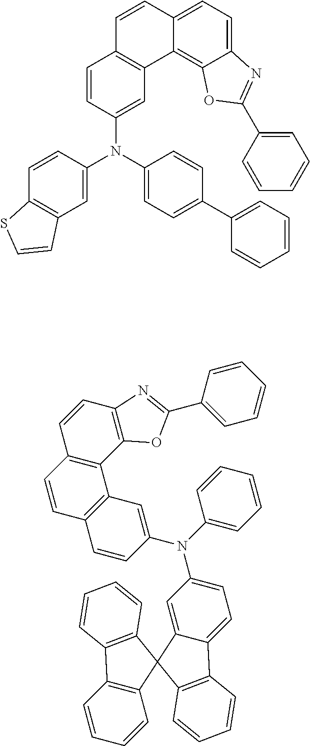 [SMILES: c1ccc(-c2ccc(N(c3ccc4sccc4c3)c3ccc4ccc5ccc6nc(-c7ccccc7)oc6c5c4c3)cc2)cc1 ; c1ccc(-c2nc3ccc4ccc5ccc(N(c6ccccc6)c6ccc7c(c6)C6(c8ccccc8-c8ccccc86)c6ccccc6-7)cc5c4c3o2)cc1]

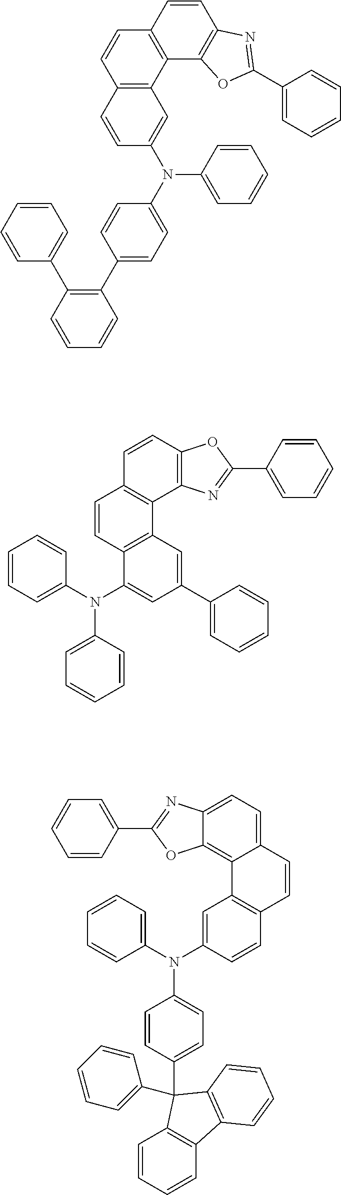 [SMILES: c1ccc(-c2nc3ccc4ccc5ccc(N(c6ccccc6)c6ccc(-c7ccccc7-c7ccccc7)cc6)cc5c4c3o2)cc1 ; c1ccc(-c2cc(N(c3ccccc3)c3ccccc3)c3ccc4ccc5oc(-c6ccccc6)nc5c4c3c2)cc1 ; c1ccc(-c2nc3ccc4ccc5ccc(N(c6ccccc6)c6ccc(C7(c8ccccc8)c8ccccc8-c8ccccc87)cc6)cc5c4c3o2)cc1]

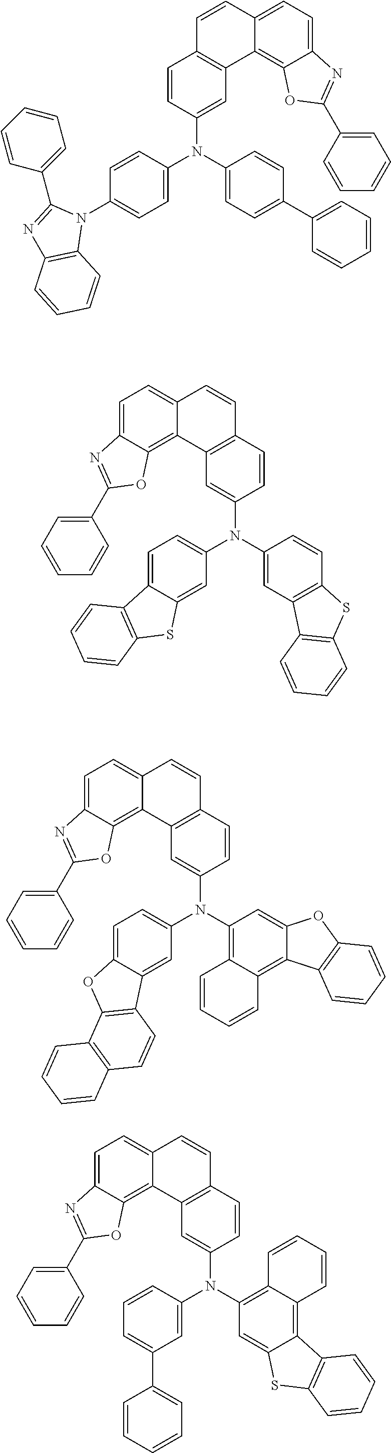

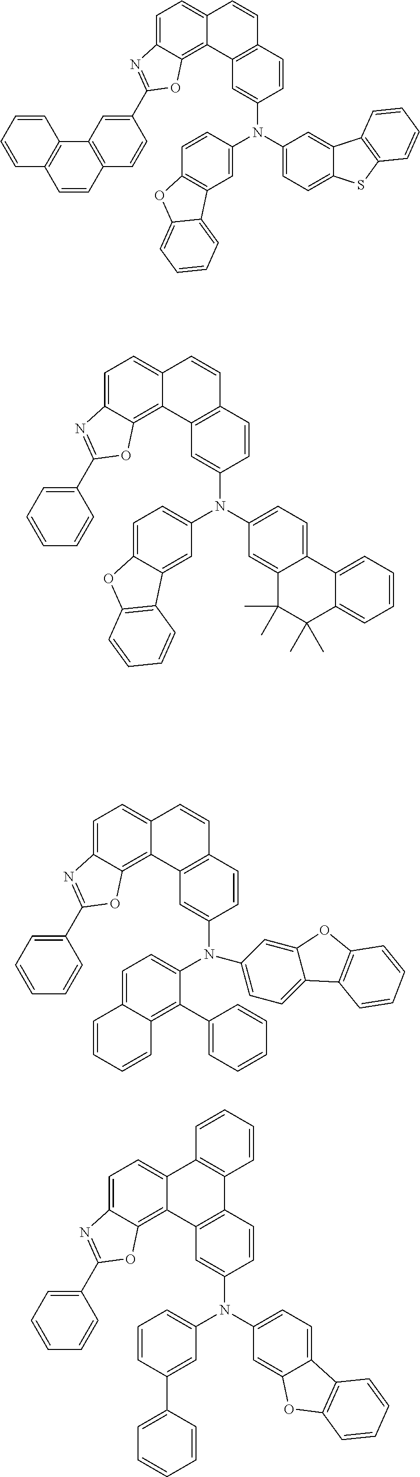

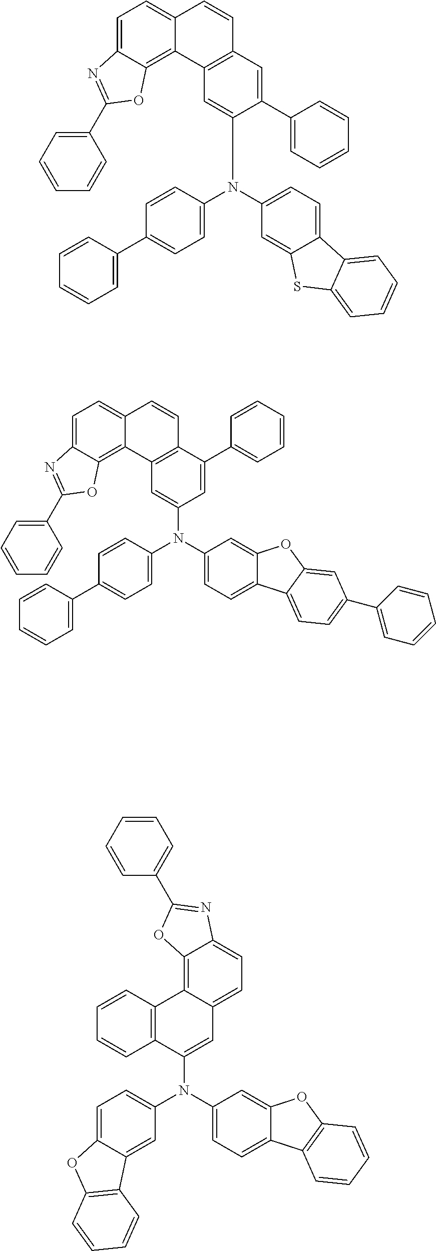 [SMILES: c1ccc(-c2ccc(N(c3ccc4c(c3)sc3ccccc34)c3cc4c(ccc5ccc6nc(-c7ccccc7)oc6c54)cc3-c3ccccc3)cc2)cc1 ; c1ccc(-c2ccc(N(c3ccc4c(c3)oc3cc(-c5ccccc5)ccc34)c3cc(-c4ccccc4)c4ccc5ccc6nc(-c7ccccc7)oc6c5c4c3)cc2)cc1 ; c1ccc(-c2nc3ccc4cc(N(c5ccc6c(c5)oc5ccccc56)c5ccc6oc7ccccc7c6c5)c5ccccc5c4c3o2)cc1]

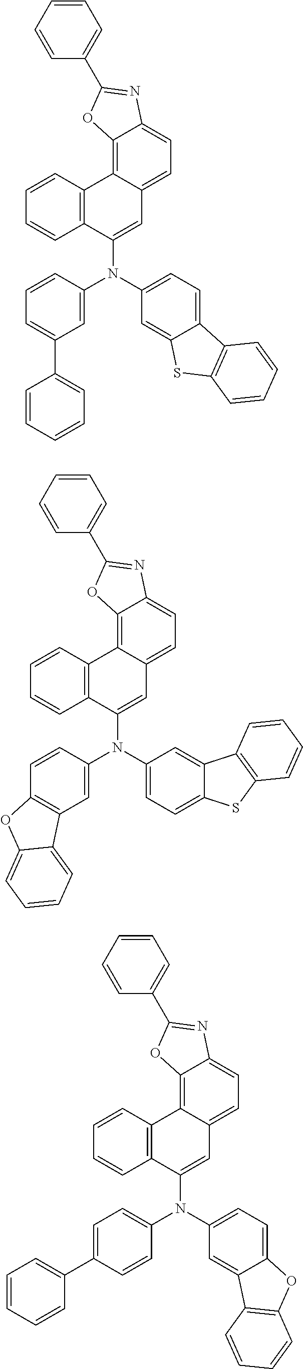

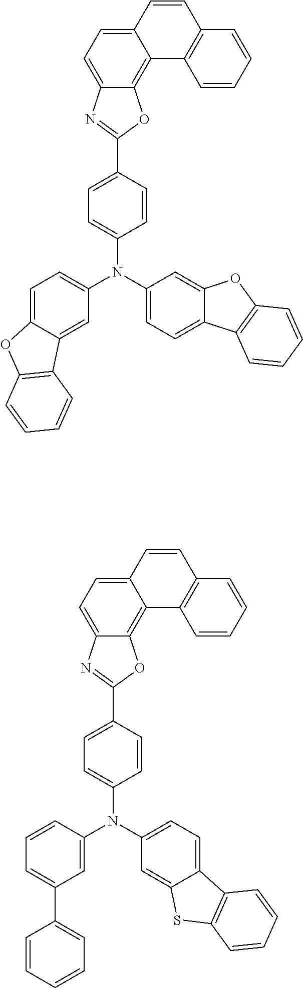 [SMILES: c1ccc2c(c1)ccc1ccc3nc(-c4ccc(N(c5ccc6c(c5)oc5ccccc56)c5ccc6oc7ccccc7c6c5)cc4)oc3c12 ; c1ccc(-c2cccc(N(c3ccc(-c4nc5ccc6ccc7ccccc7c6c5o4)cc3)c3ccc4c(c3)sc3ccccc34)c2)cc1]

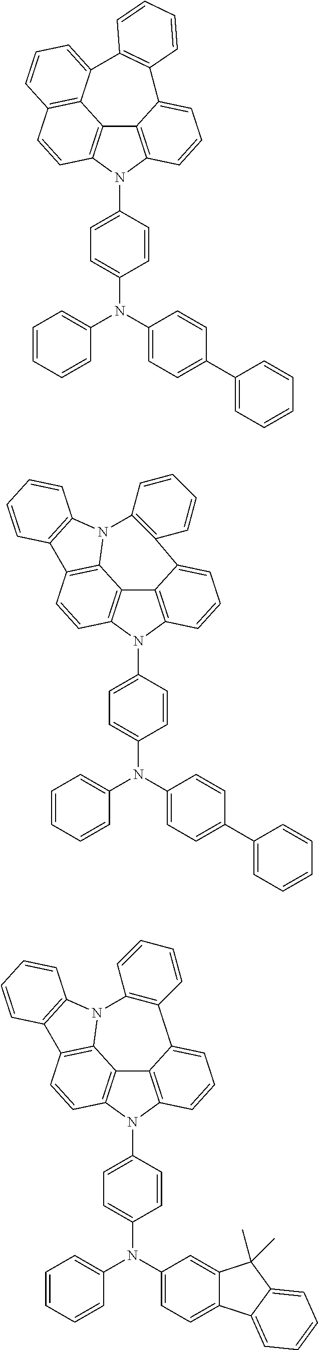 [SMILES: c1ccc(-c2ccc(N(c3ccccc3)c3ccc(-n4c5cccc6c5c5c7c(cccc7ccc54)-c4ccccc4-6)cc3)cc2)cc1 ; c1ccc(-c2ccc(N(c3ccccc3)c3ccc(-n4c5cccc6c7ccccc7n7c8ccccc8c8ccc4c(c65)c87)cc3)cc2)cc1 ; CC1(C)c2ccccc2-c2ccc(N(c3ccccc3)c3ccc(-n4c5cccc6c7ccccc7n7c8ccccc8c8ccc4c(c65)c87)cc3)cc21]

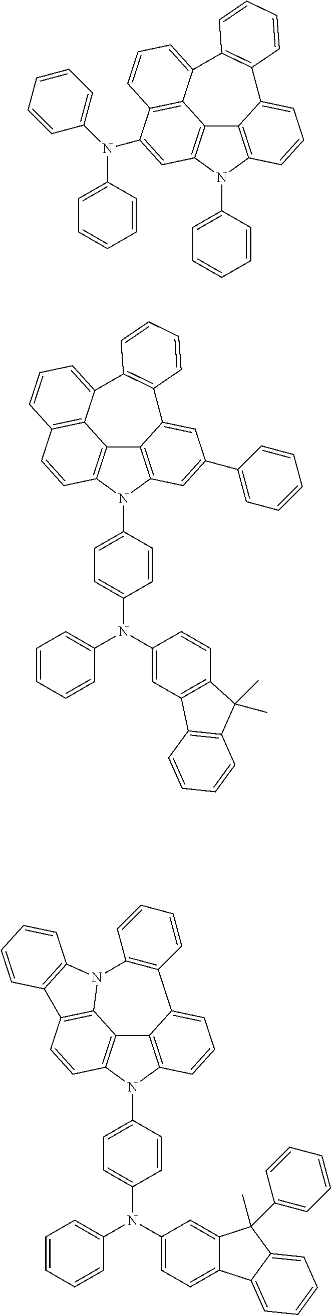 [SMILES: c1ccc(N(c2ccccc2)c2cc3c4c5c(cccc25)-c2ccccc2-c2cccc(c24)n3-c2ccccc2)cc1 ; CC1(C)c2ccccc2-c2cc(N(c3ccccc3)c3ccc(-n4c5cc(-c6ccccc6)cc6c5c5c7c(cccc7ccc54)-c4ccccc4-6)cc3)ccc21 ; CC1(c2ccccc2)c2ccccc2-c2ccc(N(c3ccccc3)c3ccc(-n4c5cccc6c7ccccc7n7c8ccccc8c8ccc4c(c65)c87)cc3)cc21]

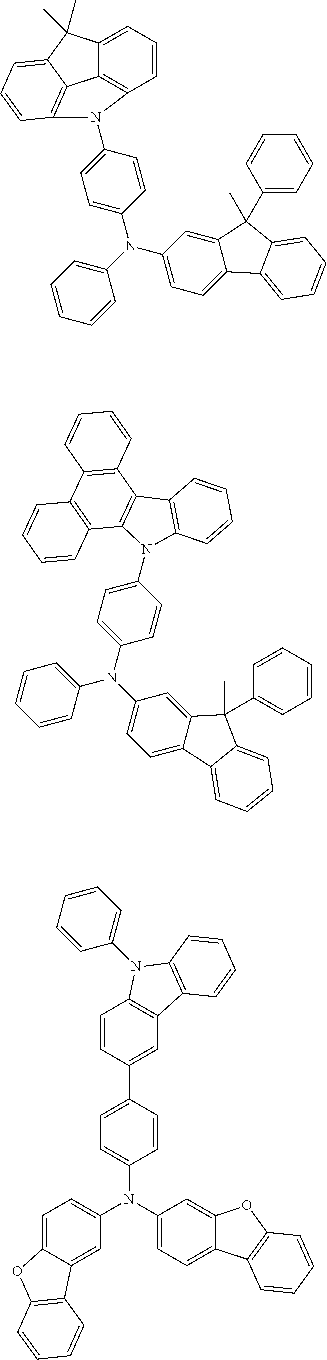 [SMILES: CC1(C)c2cccc3c2c2c1cccc2n3-c1ccc(N(c2ccccc2)c2ccc3c(c2)C(C)(c2ccccc2)c2ccccc2-3)cc1 ; CC1(c2ccccc2)c2ccccc2-c2ccc(N(c3ccccc3)c3ccc(-n4c5ccccc5c5c6ccccc6c6ccccc6c54)cc3)cc21 ; c1ccc(-n2c3ccccc3c3cc(-c4ccc(N(c5ccc6c(c5)oc5ccccc56)c5ccc6oc7ccccc7c6c5)cc4)ccc32)cc1]

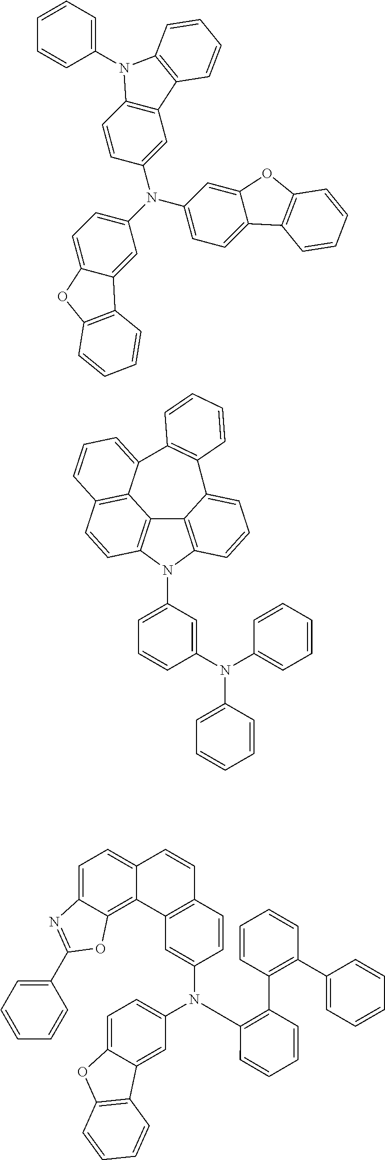 [SMILES: c1ccc(-n2c3ccccc3c3cc(N(c4ccc5c(c4)oc4ccccc45)c4ccc5oc6ccccc6c5c4)ccc32)cc1 ; c1ccc(N(c2ccccc2)c2cccc(-n3c4cccc5c4c4c6c(cccc6ccc43)-c3ccccc3-5)c2)cc1 ; c1ccc(-c2nc3ccc4ccc5ccc(N(c6ccc7oc8ccccc8c7c6)c6ccccc6-c6ccccc6-c6ccccc6)cc5c4c3o2)cc1]

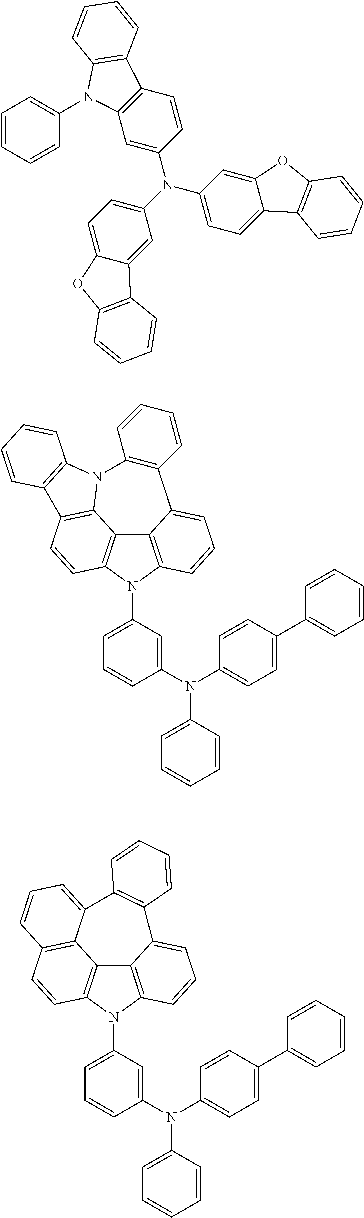

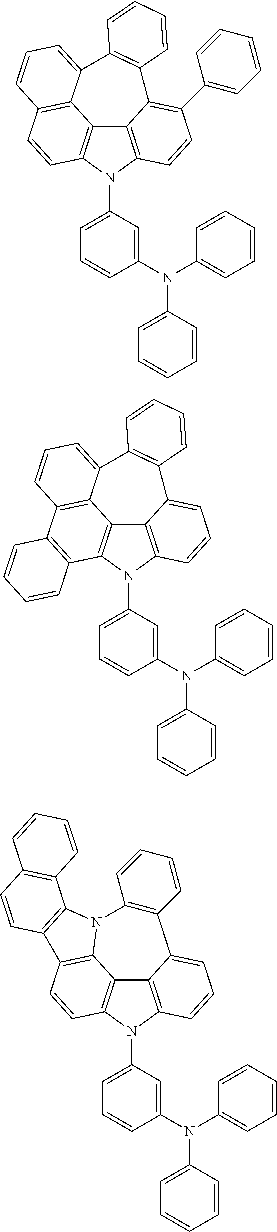

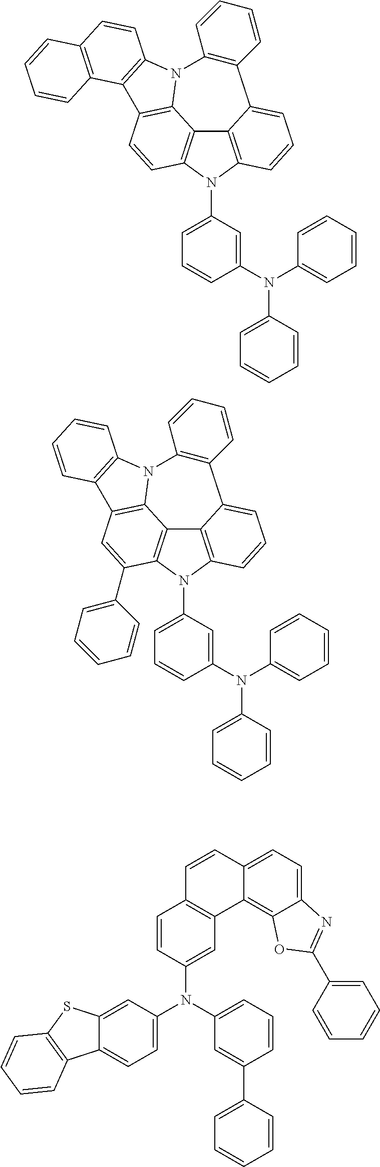

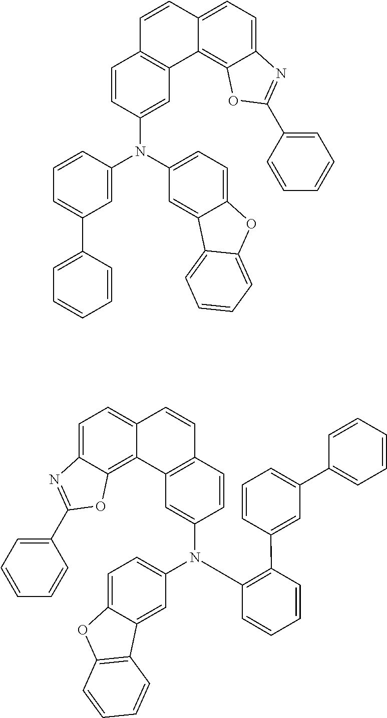 [SMILES: c1ccc(-c2cccc(N(c3ccc4oc5ccccc5c4c3)c3ccc4ccc5ccc6nc(-c7ccccc7)oc6c5c4c3)c2)cc1 ; c1ccc(-c2cccc(-c3ccccc3N(c3ccc4oc5ccccc5c4c3)c3ccc4ccc5ccc6nc(-c7ccccc7)oc6c5c4c3)c2)cc1]

The compounds represented by formula 2 according to the present disclosure may be prepared by synthetic methods known to a person skilled in the art, for example by reference to Korean Patent Application Laid-open No. 2022-0051794 A (published Apr. 26, 2022), but is not limited thereto.

The dopants comprised in the organic electroluminescent device of the present disclosure may be at least one phosphorescent or fluorescent dopant, and are preferably a phosphorescent dopant. The phosphorescent dopant materials applied to the organic electroluminescent device of the present disclosure are not particularly limited, but may be a complex compound of a metal atom selected from iridium (Ir), osmium (Os), copper (Cu), and platinum (Pt), preferably ortho-metallated complex compounds of a metal atom selected from iridium (Ir), osmium (Os), copper (Cu), and platinum (Pt), and more preferably ortho-metallated iridium complex compounds.

A compound represented by the following formula 101 may be used as a dopant included in the organic electroluminescent device of the present disclosure, but is not limited thereto.

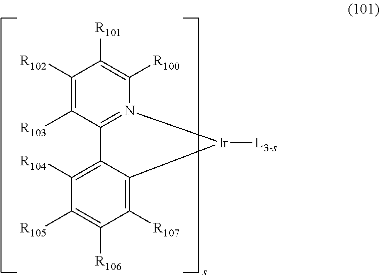

In formula 101,

L is any one selected from the following structures 1 to 3:

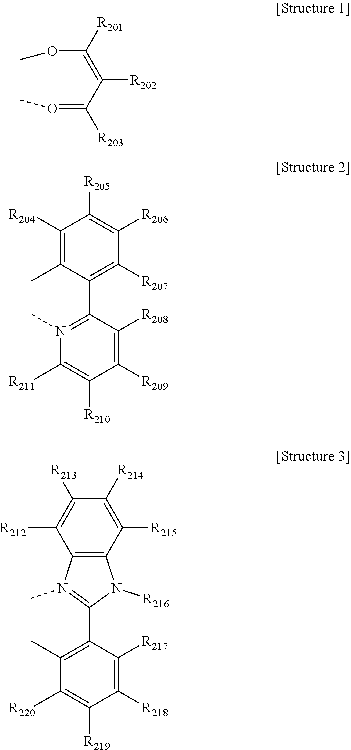

R₁₀₀ to R₁₀₃ each independently represent hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, cyano, a substituted or unsubstituted (3- to 30-membered)heteroaryl, or a substituted or unsubstituted (C1-C30)alkoxy; R₁₀₀ to R₁₀₃ or may be linked to an adjacent substituent(s) to form a ring, for example, a substituted or unsubstituted quinoline, a substituted or unsubstituted benzofuropyridine, a substituted or unsubstituted benzothienopyridine, a substituted or unsubstituted indenopyridine, a substituted or unsubstituted benzofuroquinoline, a substituted or unsubstituted benzothienoquinoline, or a substituted or unsubstituted indenoquinoline, together with pyridine;

R₁₀₄ to R₁₀₇ each independently represent hydrogen, deuterium, halogen, (C1-C30)alkyl unsubstituted or substituted with deuterium and/or halogen, a substituted or unsubstituted (C3-C30)cycloalkyl, a substituted or unsubstituted (C6-C30)aryl, a substituted or unsubstituted (3- to 30-membered)heteroaryl, cyano, or a substituted or unsubstituted (C1-C30)alkoxy; or R₁₀₄ to R₁₀₇ may be linked to an adjacent substituent(s) to form a ring, for example, a substituted or unsubstituted naphthalene, a substituted or unsubstituted fluorene, a substituted or unsubstituted dibenzothiophene, a substituted or unsubstituted dibenzofuran, a substituted or unsubstituted indenopyridine, a substituted or unsubstituted benzofuropyridine, or a substituted or unsubstituted benzothienopyridine, together with benzene;

R₂₀₁ to R₂₂₀ each independently represent hydrogen, deuterium, halogen, a (C1-C30)alkyl unsubstituted or substituted with deuterium and/or halogen, a substituted or unsubstituted (C3-C30)cycloalkyl, or a substituted or unsubstituted (C6-C30)aryl; or R₂₀₁ to R₂₂₀ may be linked to an adjacent substituent(s) to form a substituted or unsubstituted ring; and

s is an integer of 1 to 3.

The specific examples of the dopant compound are as follows, but are not limited thereto.

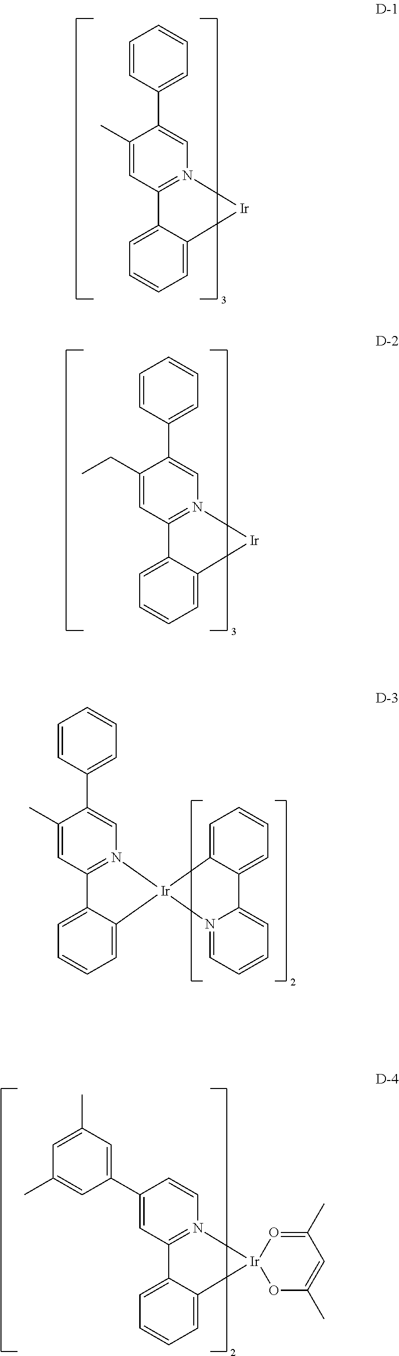

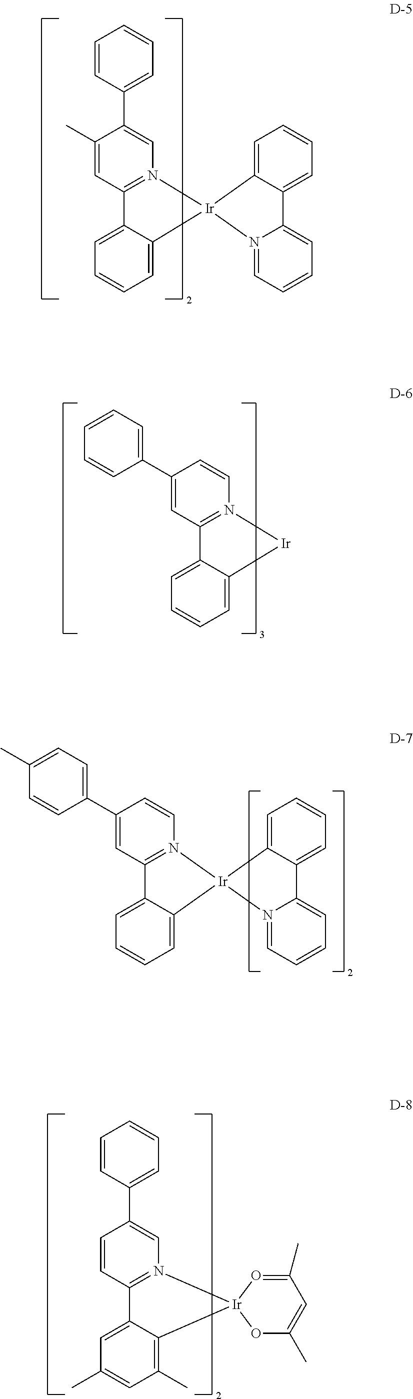

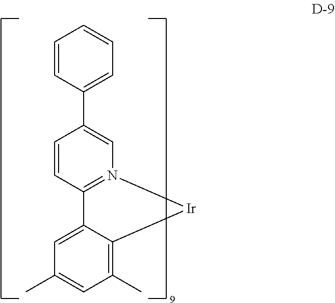

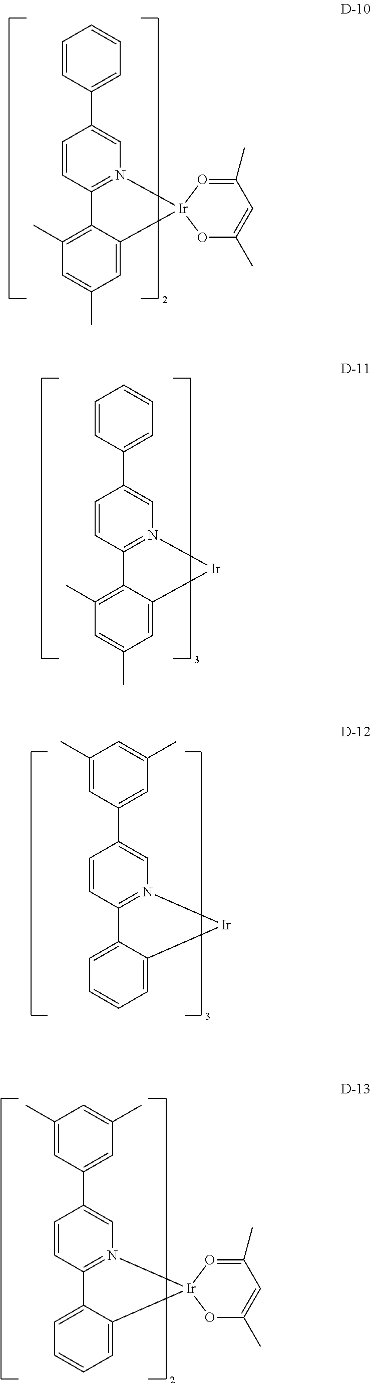

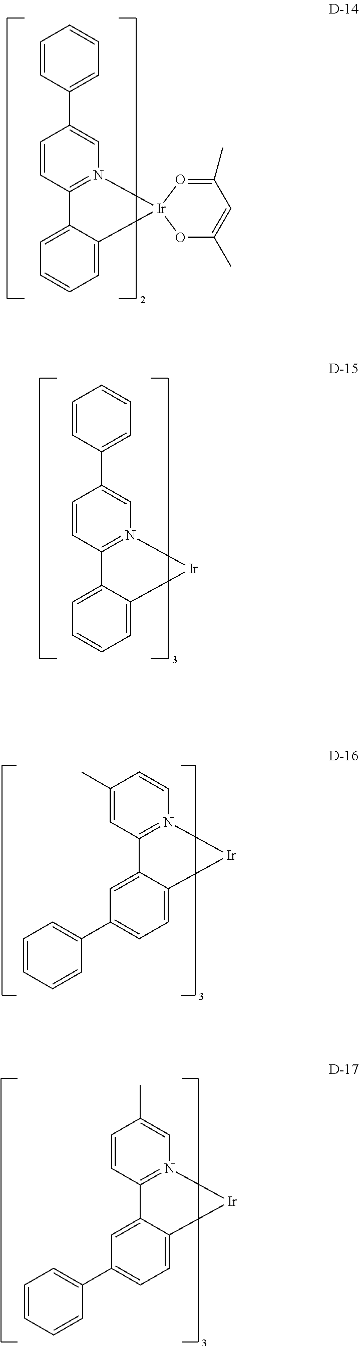

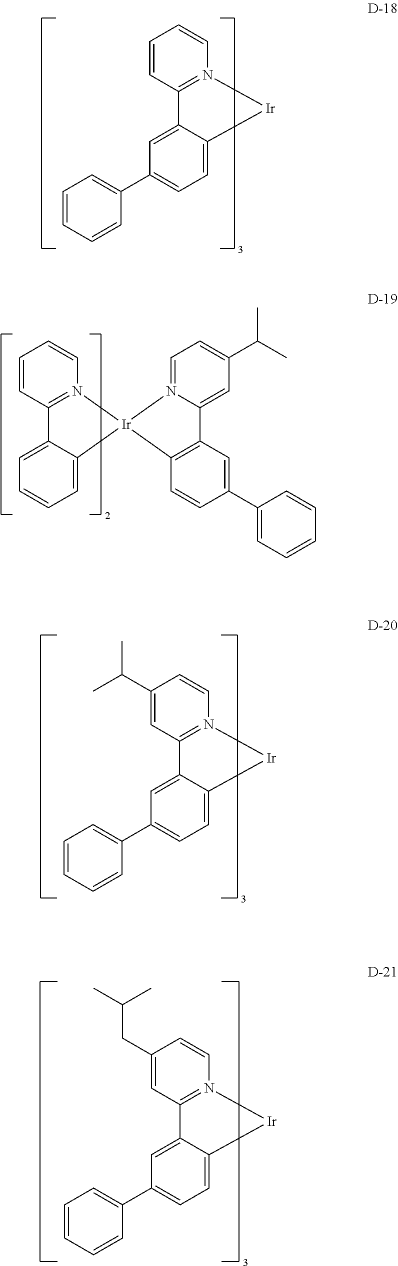

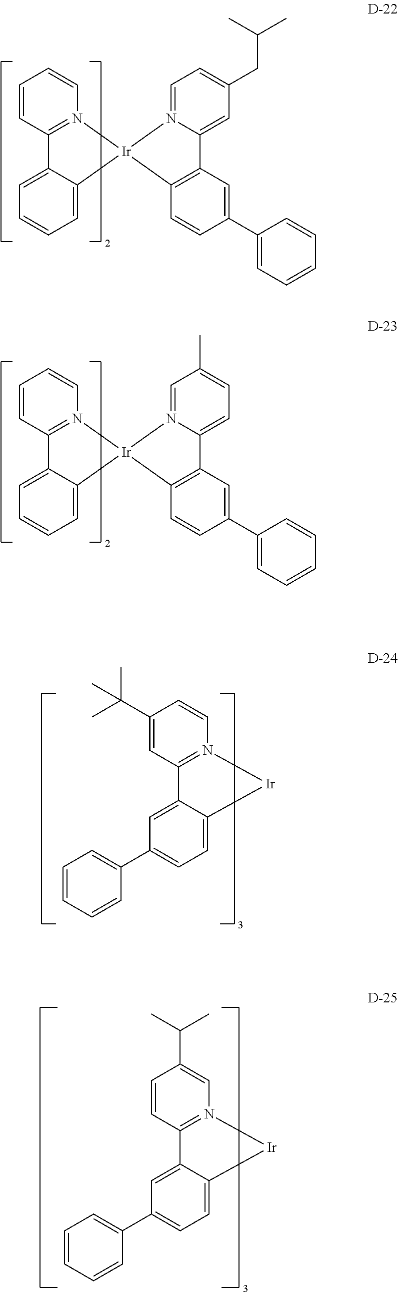

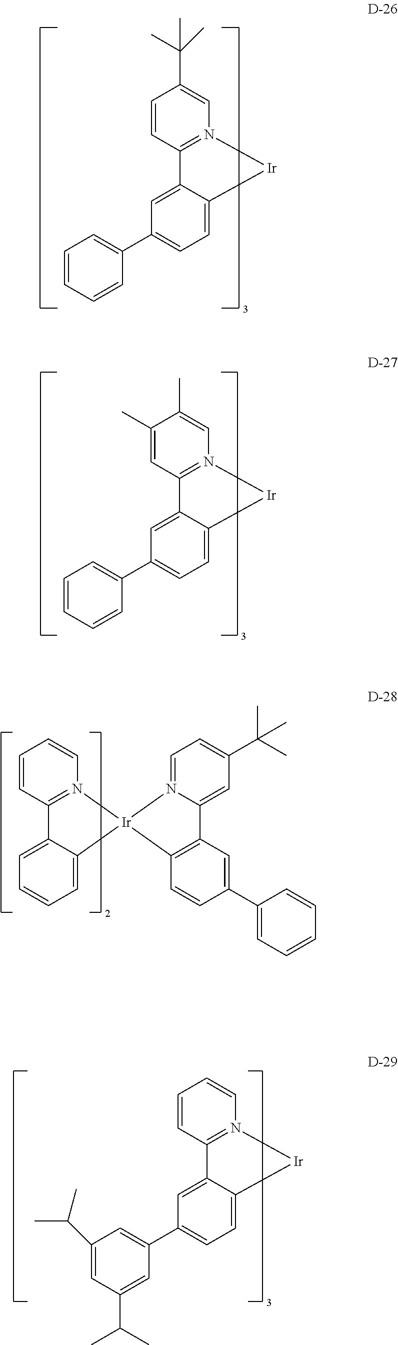

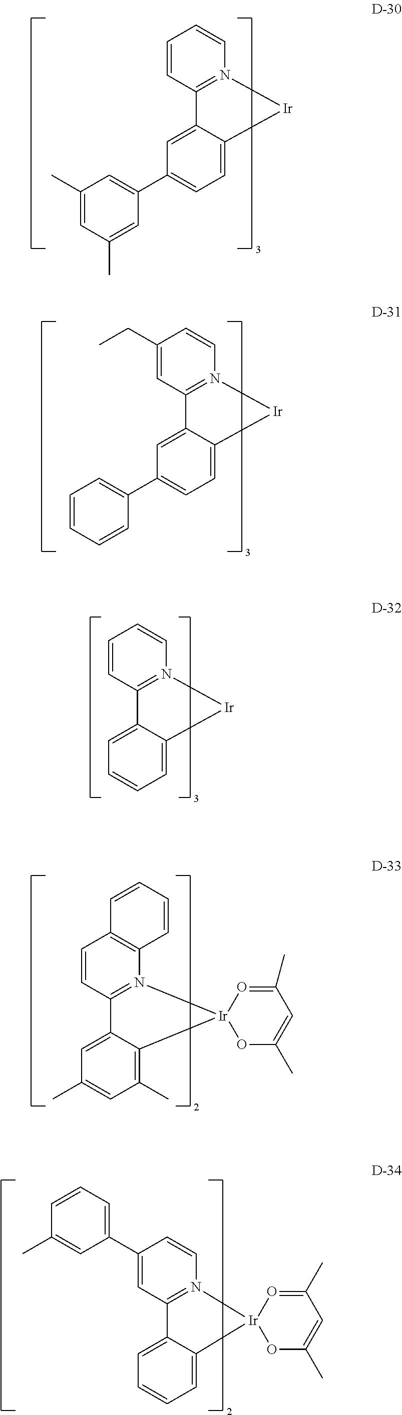

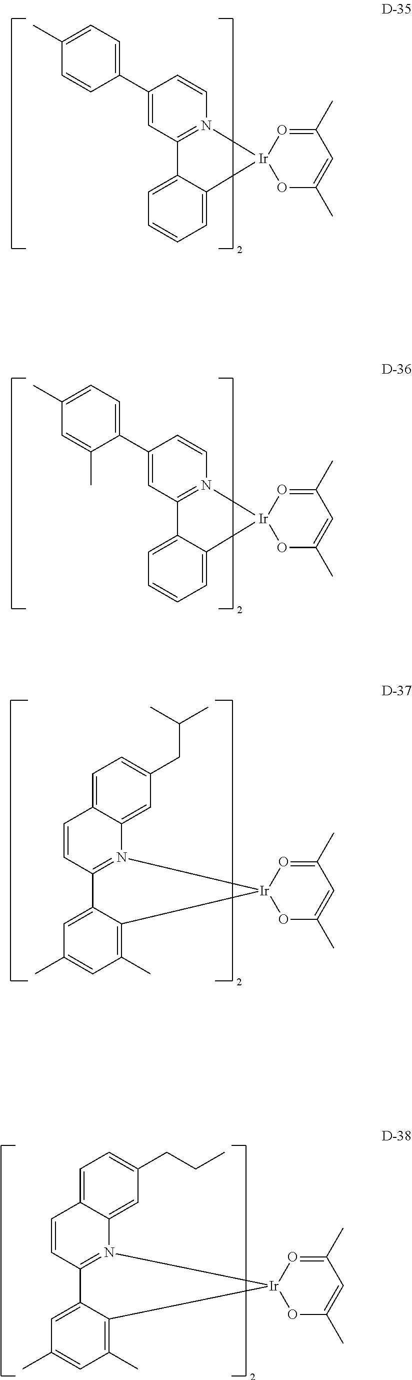

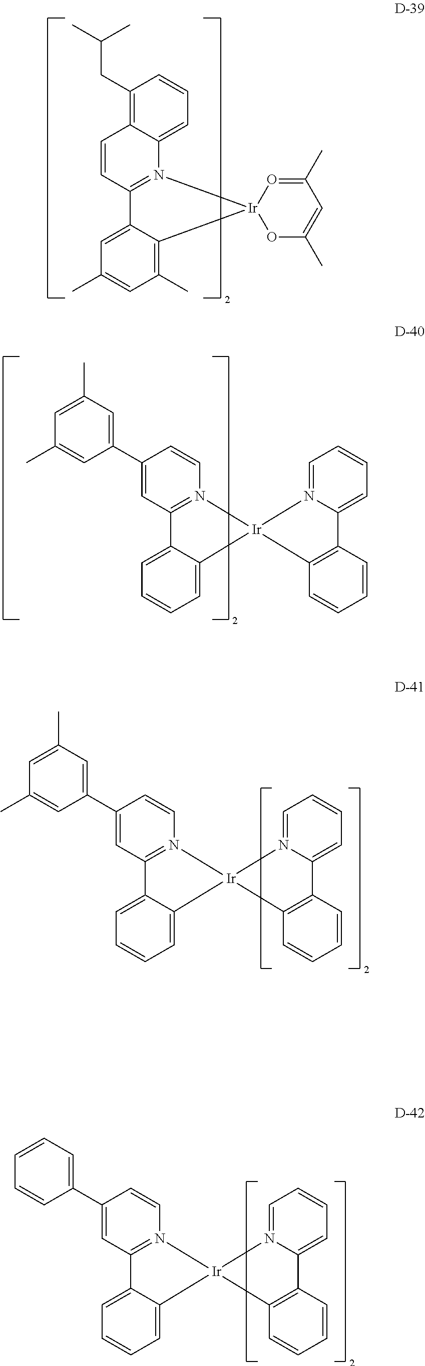

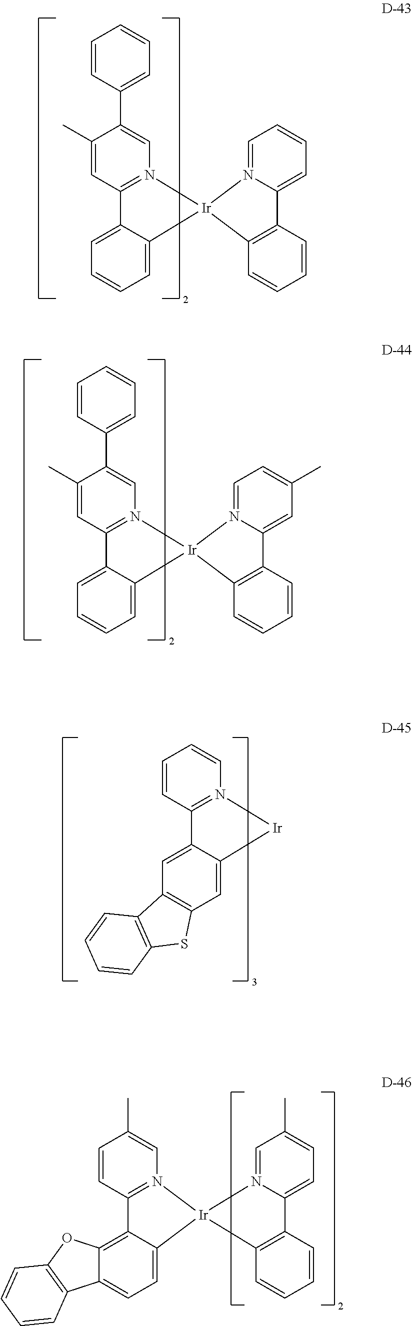

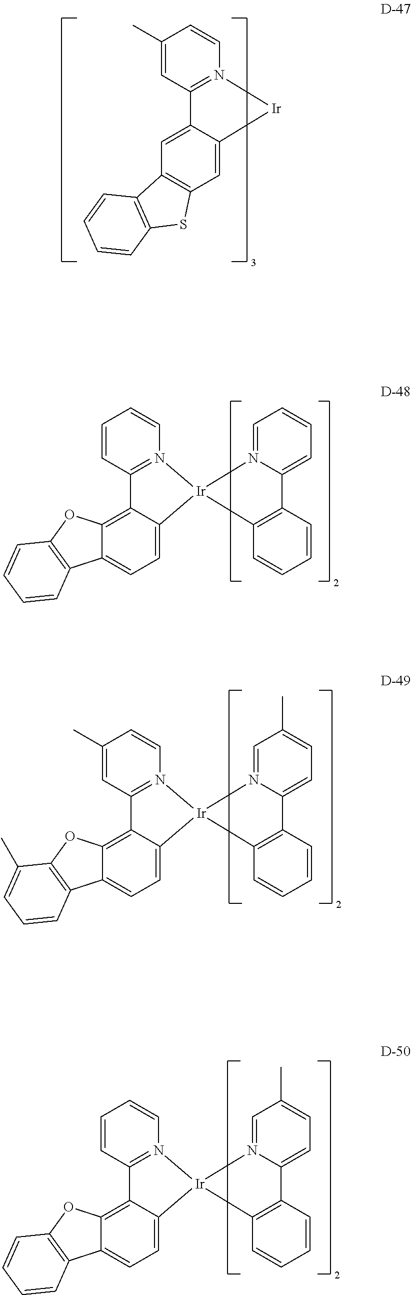

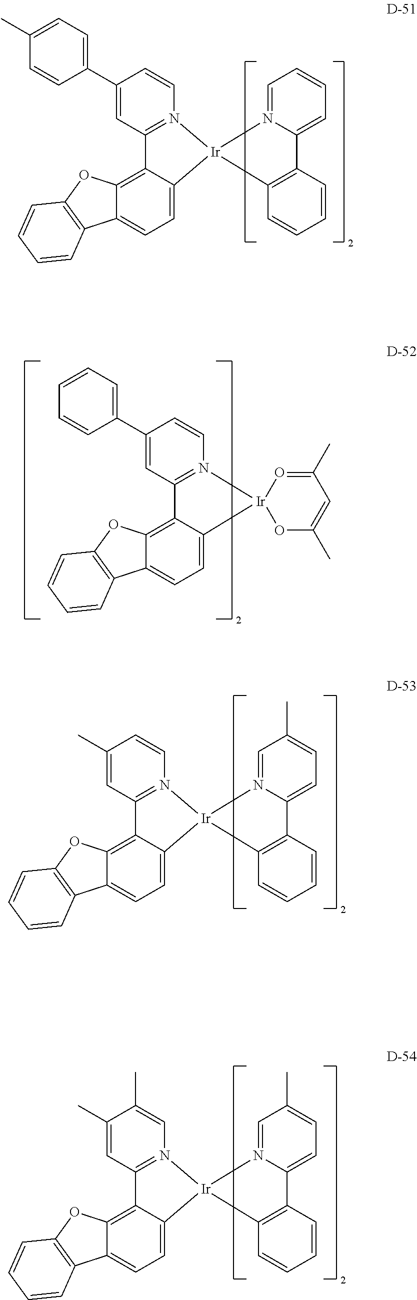

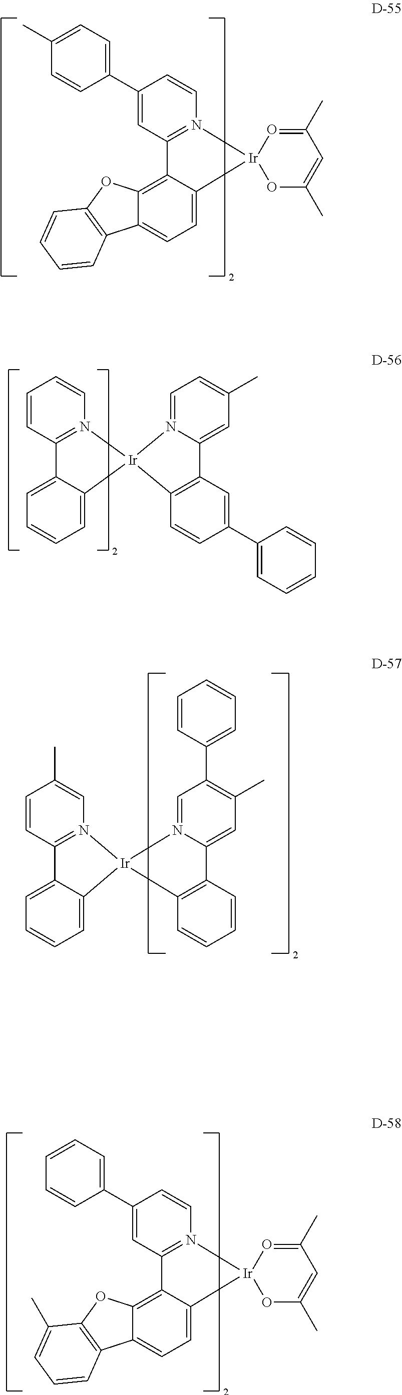

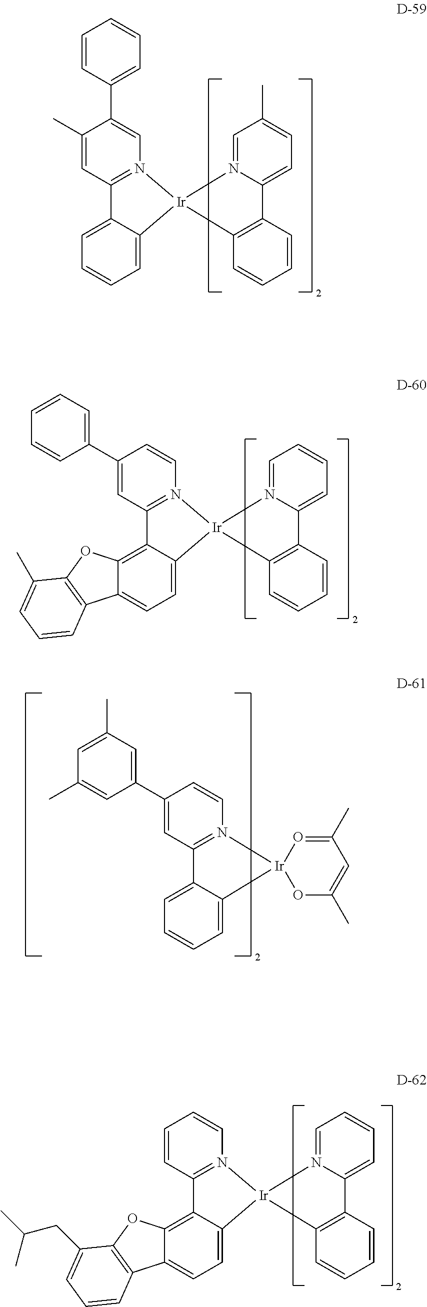

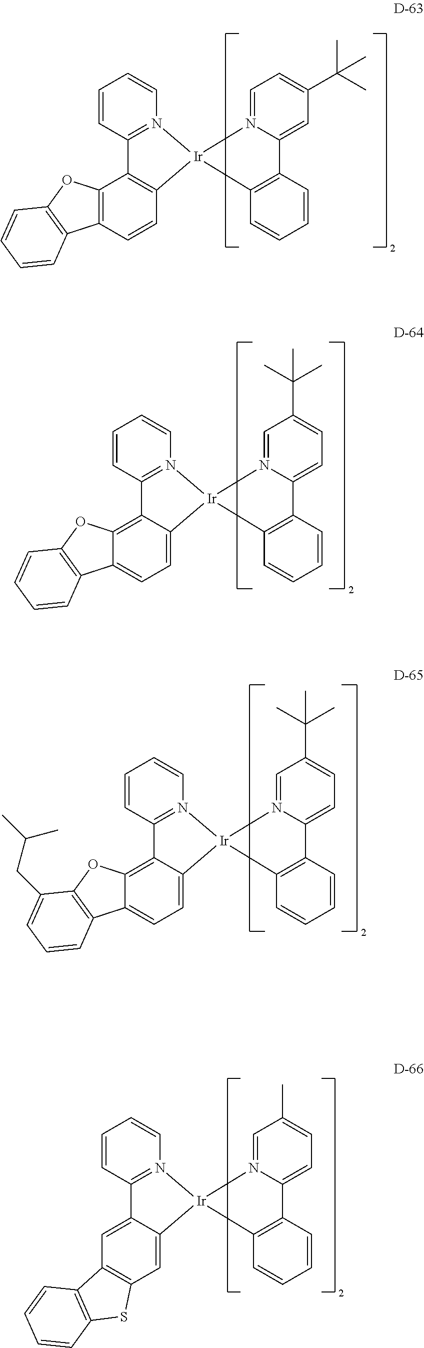

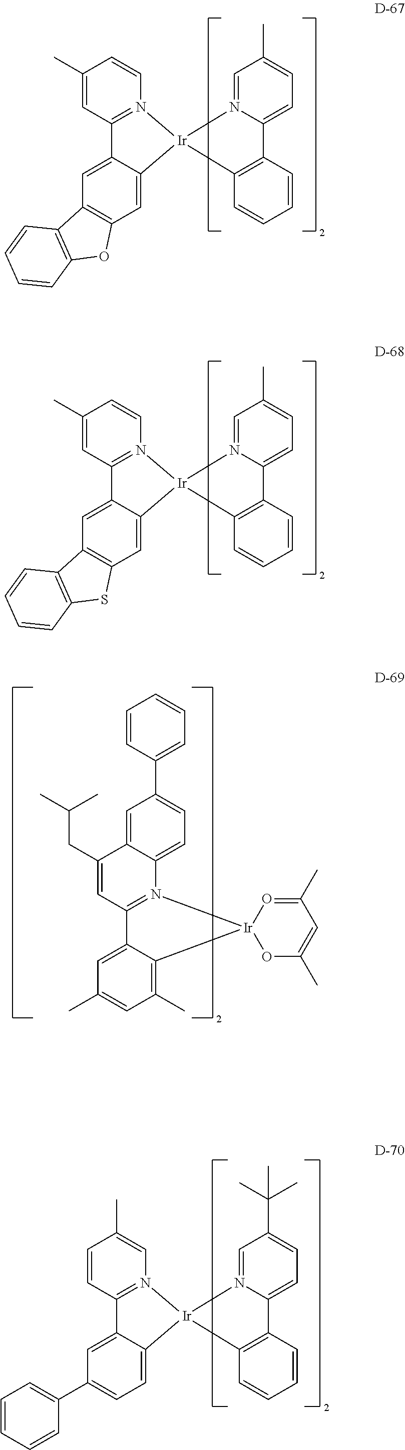

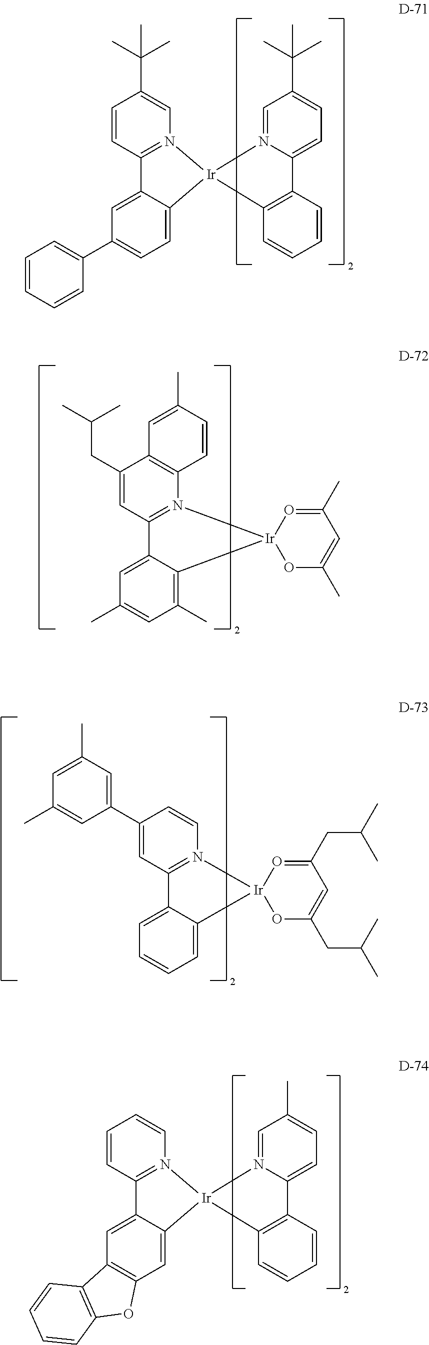

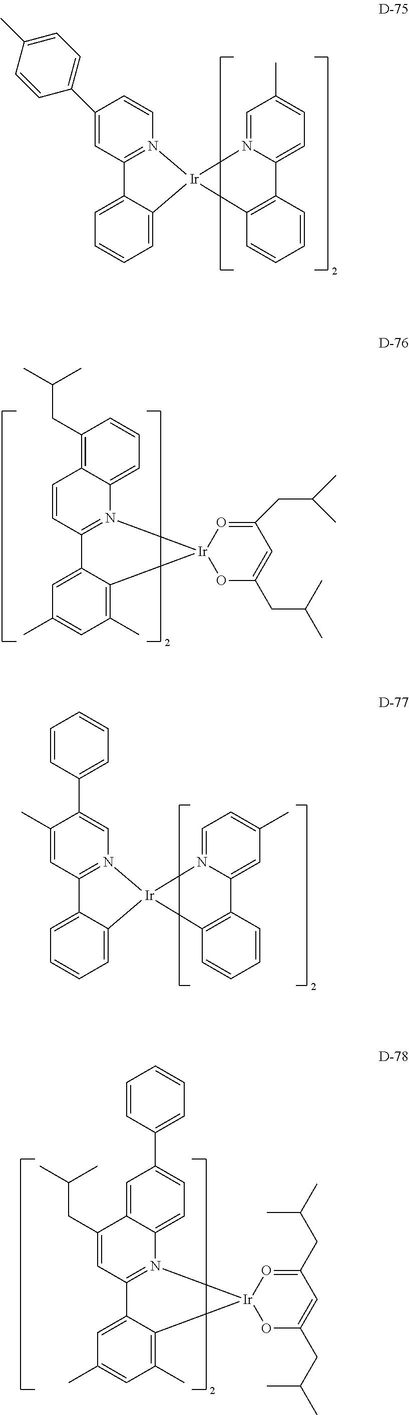

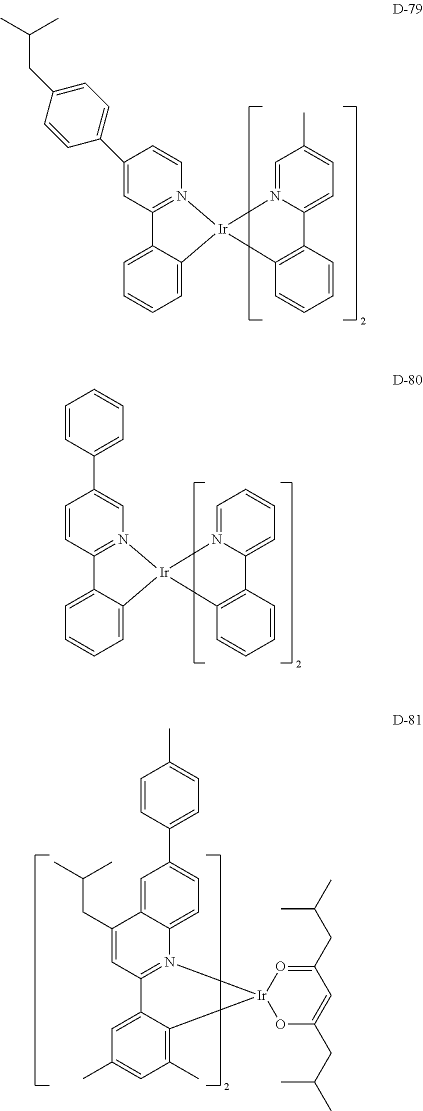

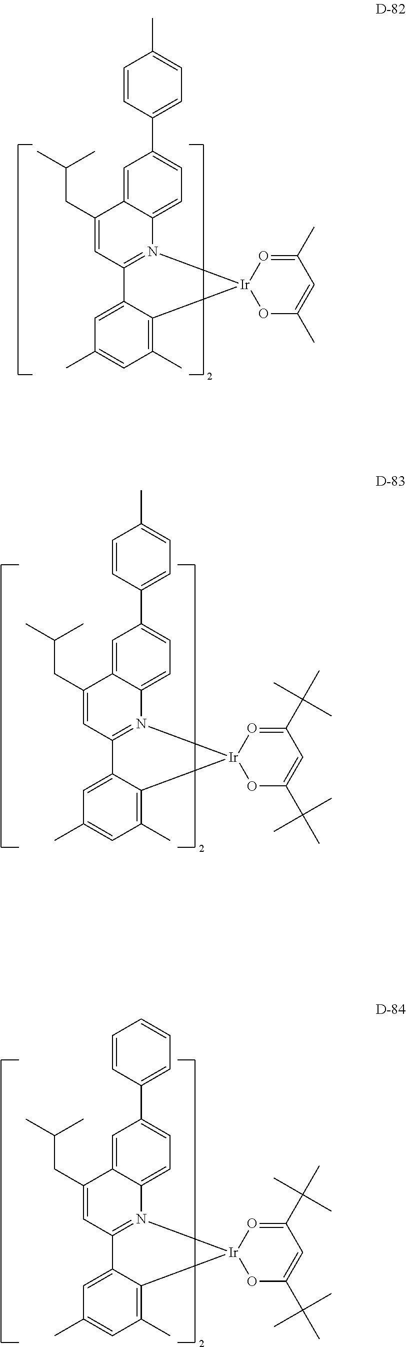

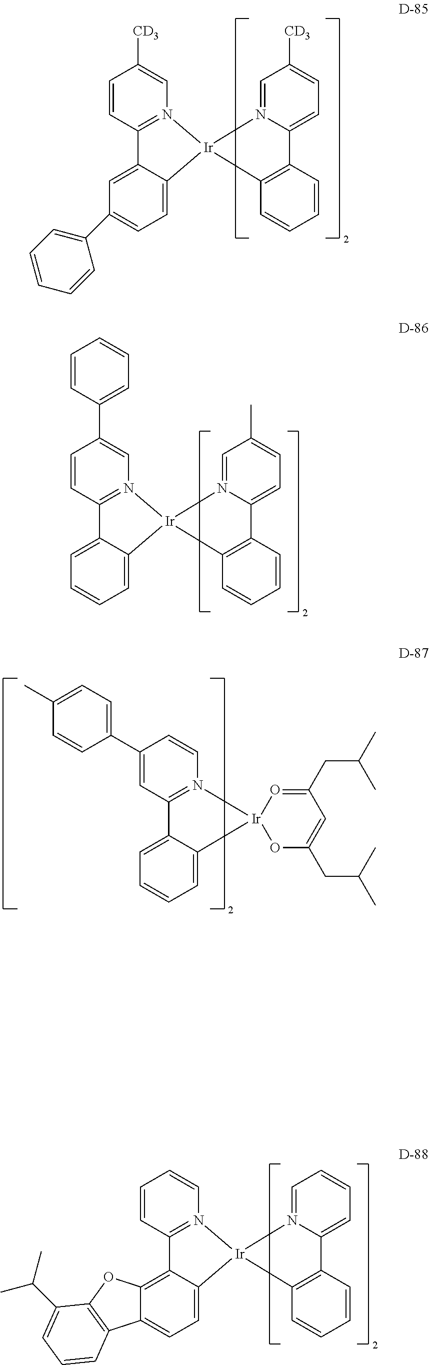

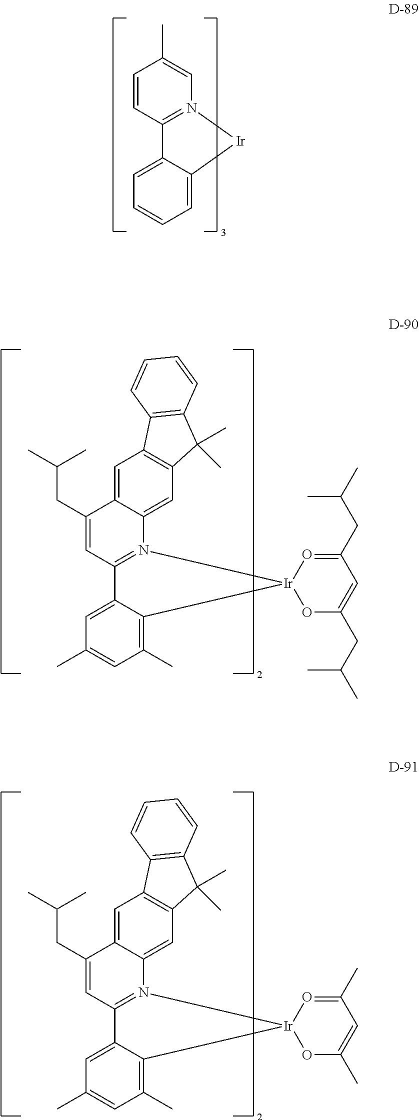

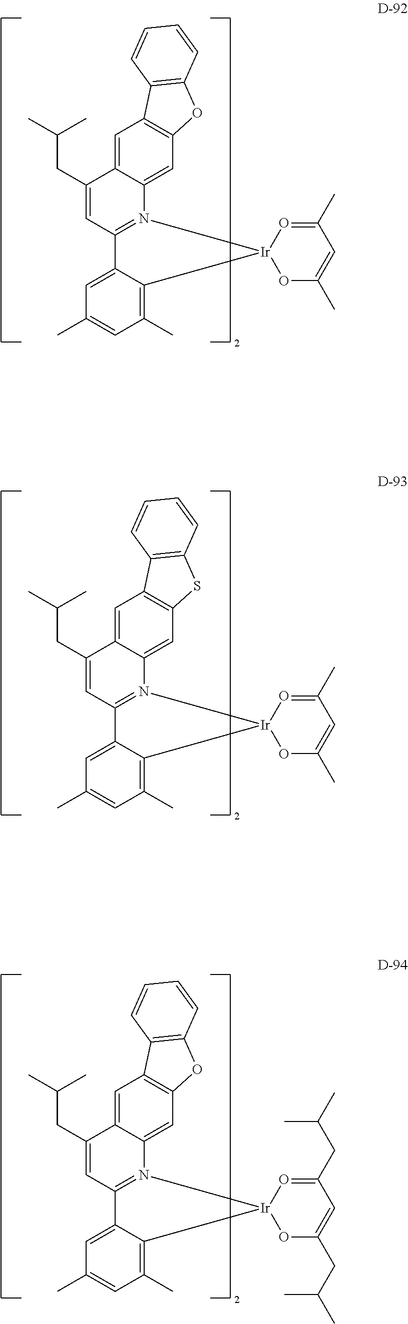

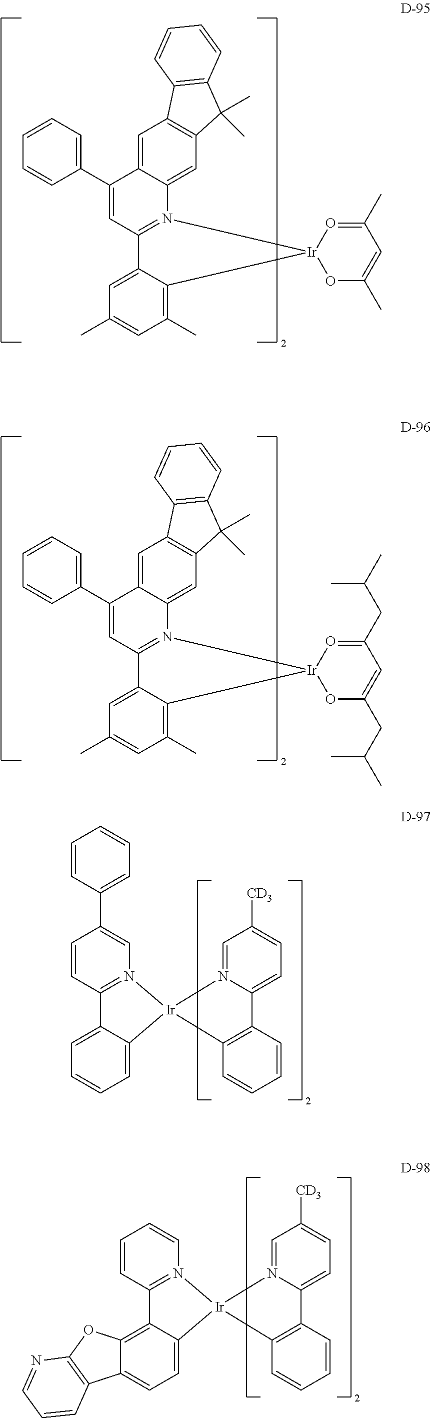

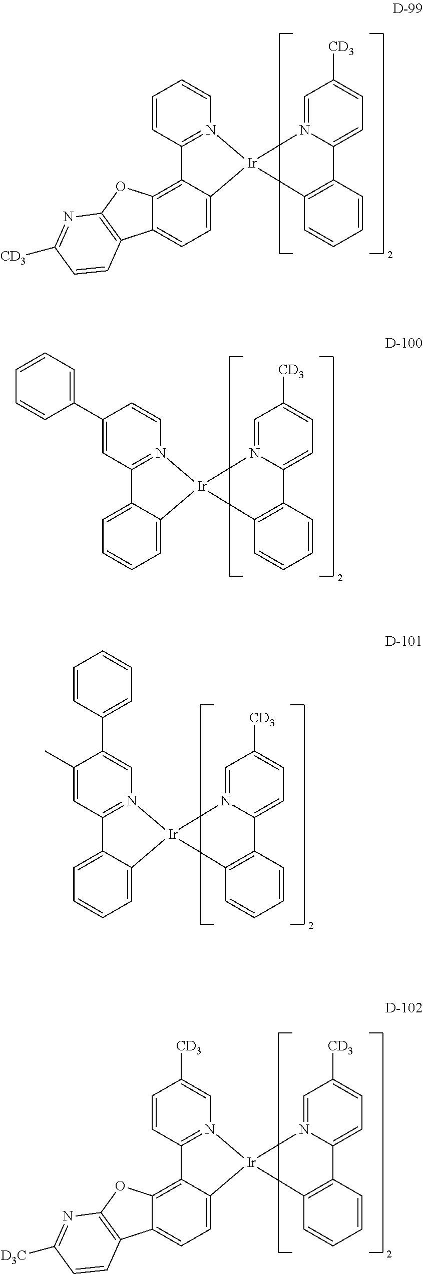

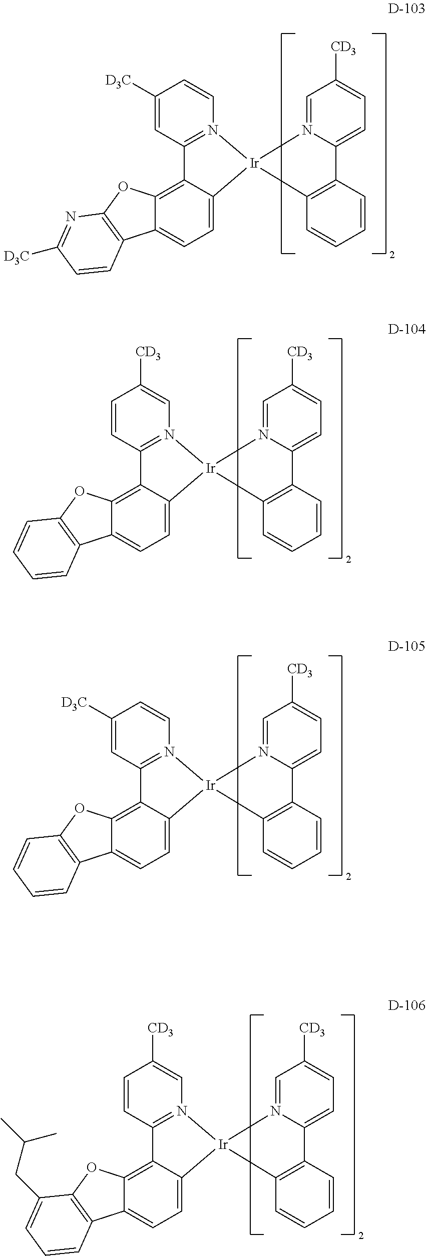

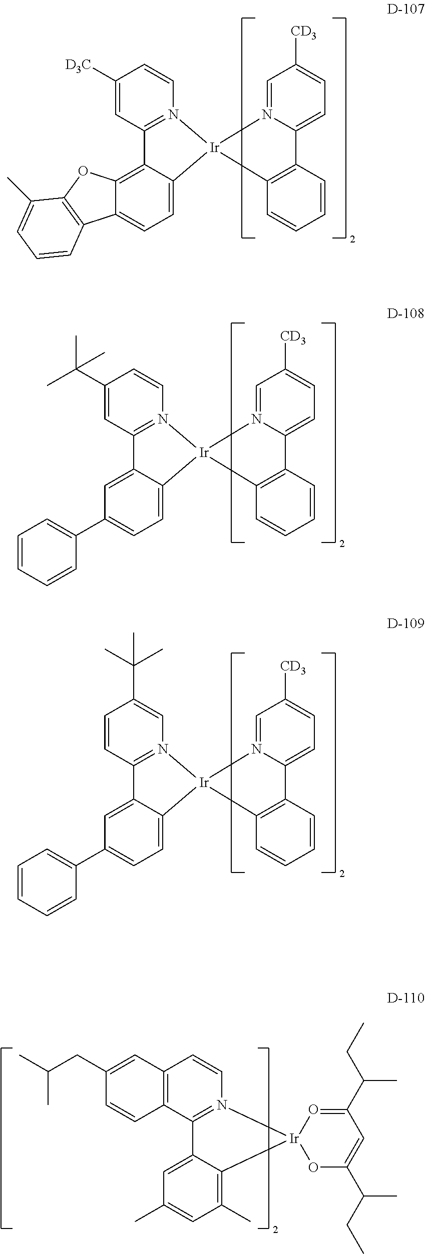

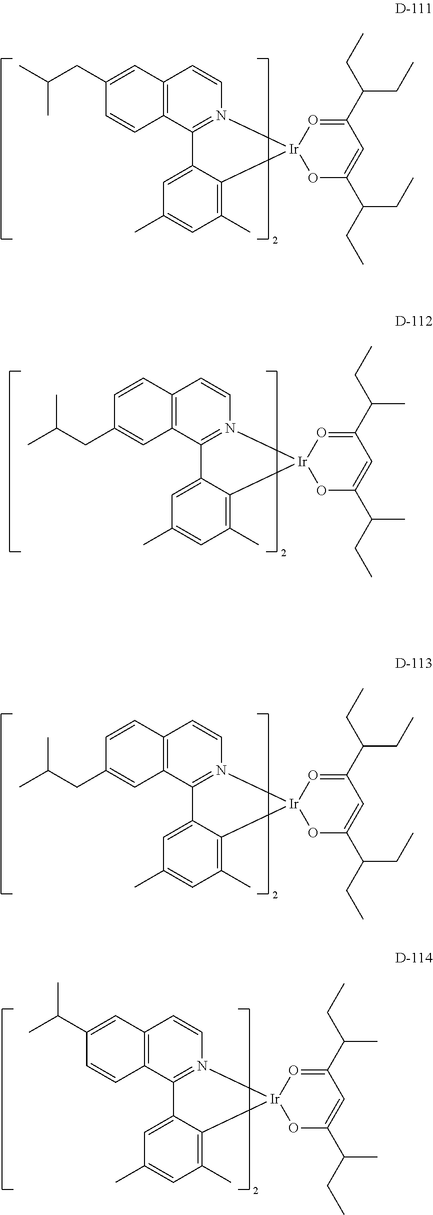

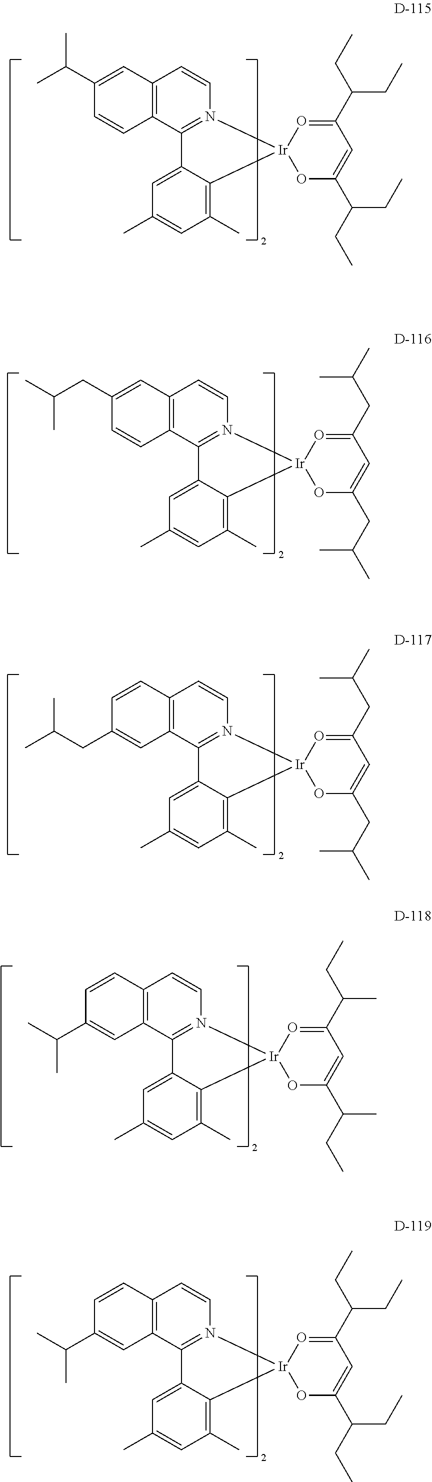

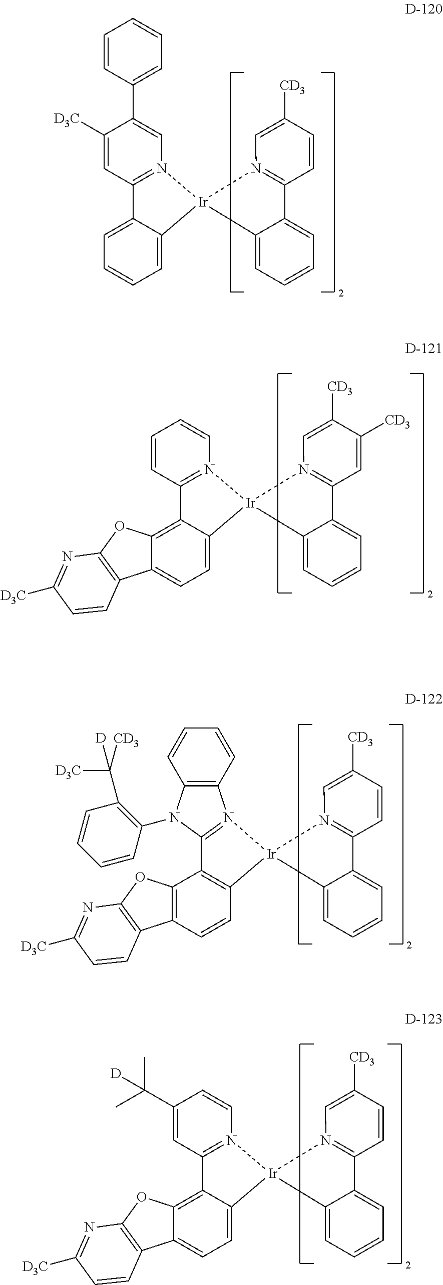

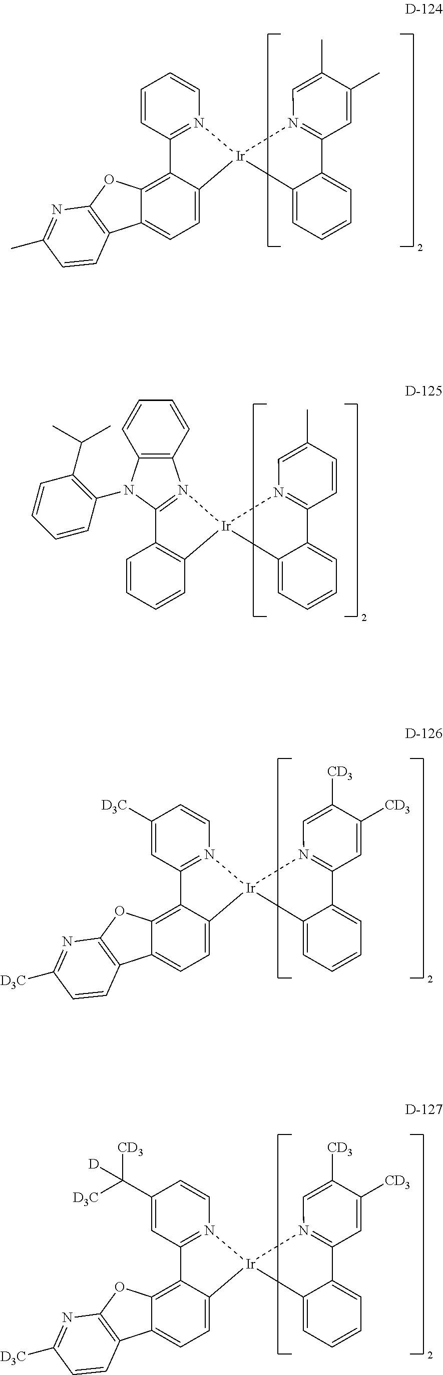

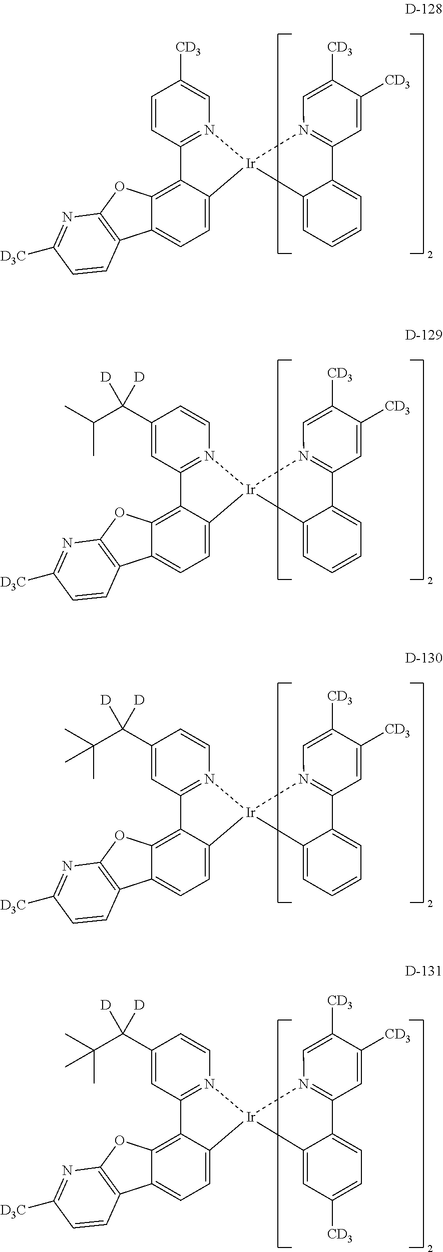

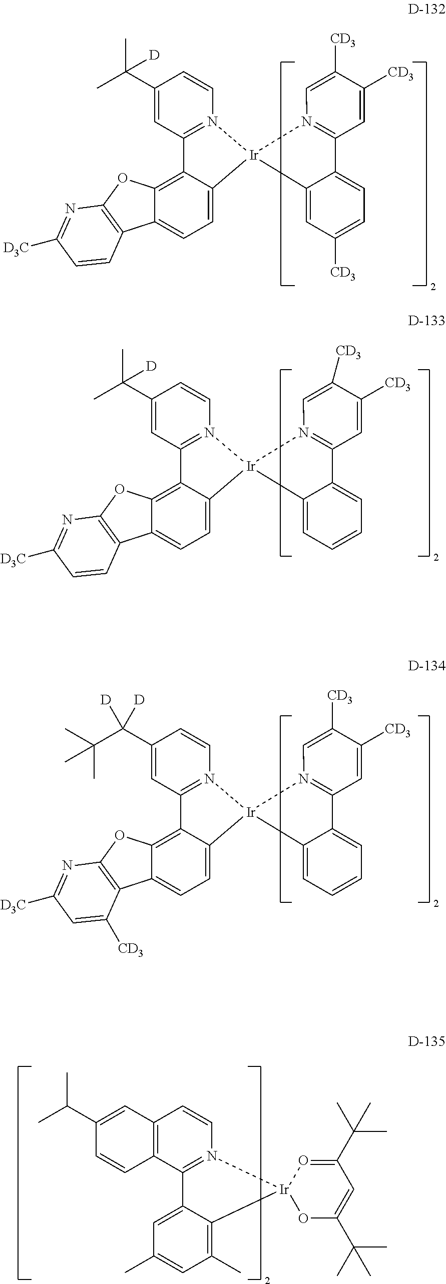

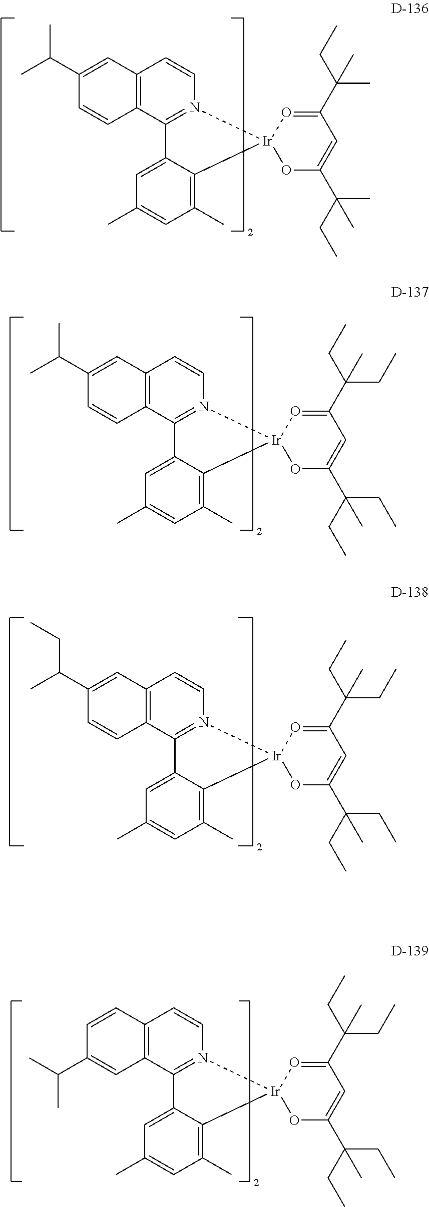

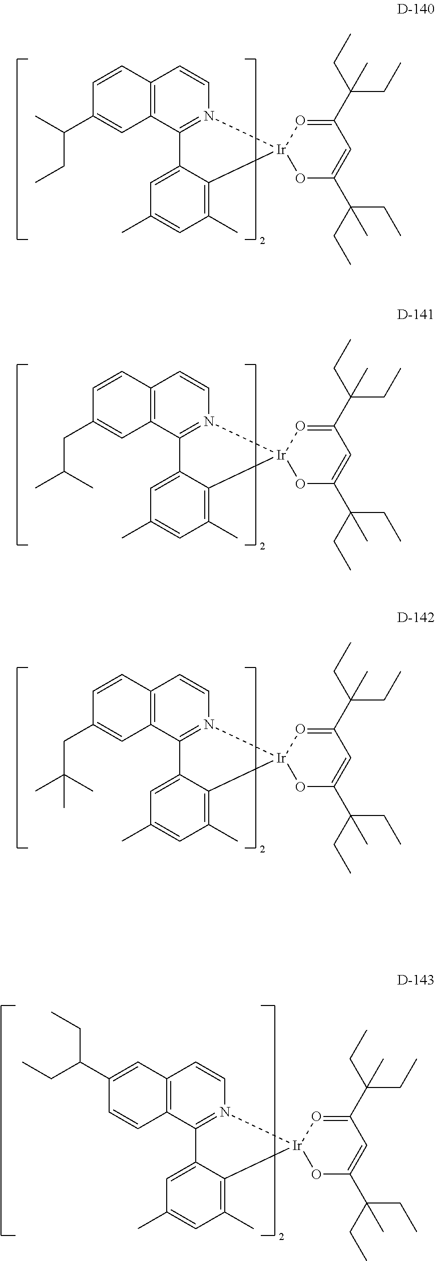

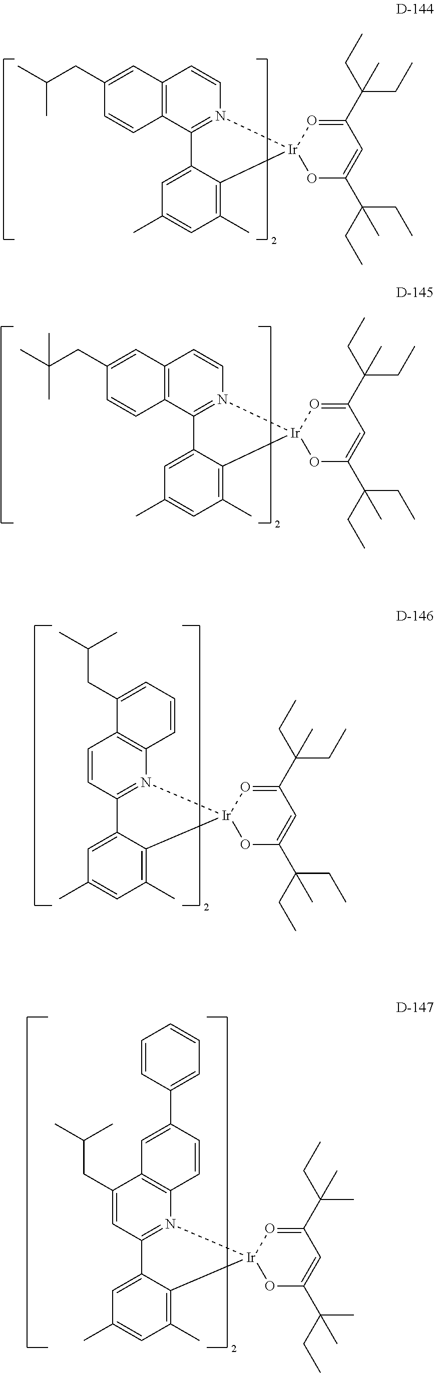

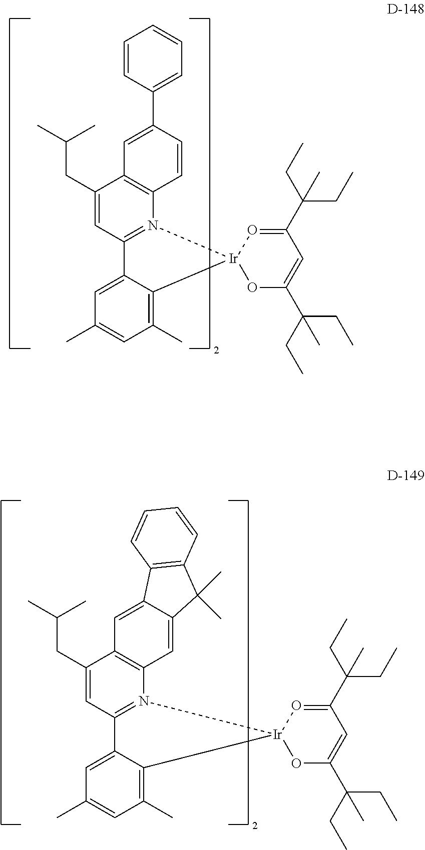

The present disclosure provides an organic electroluminescent device comprising an organic electroluminescent compound represented by formula 1, or an organic electroluminescent device comprising an organic electroluminescent material according to the present disclosure. Herein, the organic electroluminescent compound of the present disclosure may be included in a light-emitting layer, an electron transport layer, or an electron buffer layer, but is not limited thereto. The organic electroluminescent device according to the present disclosure includes a first electrode; a second electrode; and at least one organic layer(s) interposed between the first electrode and the second electrode.

One of the first electrode and the second electrode may be an anode and the other may be a cathode. The organic layer may comprise a light-emitting layer and may further comprise at least one layer selected from a hole injection layer, a hole transport layer, a hole auxiliary layer, a light-emitting auxiliary layer, an electron transport layer, an electron buffer layer, an electron injection layer, an interlayer, a hole blocking layer, and an electron blocking layer. Each of the above layers may further comprise multiple layers.

Each of the first electrode and the second electrode may be formed of a transparent conductive material, or a transflective or reflective conductive material. The organic electroluminescent device may be a top emission type, a bottom emission type, or a both-sides emission type depending on the types of materials forming the first electrode and the second electrode. In addition, the hole injection layer may further be doped with a p-dopant, and the electron injection layer may further be doped with an n-dopant.

The organic layer may further comprise one or more compounds selected from the group consisting of arylamine-based compounds and styrylarylamine-based compounds.

Further, the organic layer may further comprise at least one metal selected from the group consisting of metals of Group 1, metals of Group 2, transition metals of the 4^{th} period, transition metals of the 5^{th} period, lanthanides, and organic metals of the d-transition elements of the Periodic Table, or at least one complex compound comprising such a metal.

Further, the organic electroluminescent device of the present disclosure may be made to emit white light by further including one or more light-emitting layers comprising blue, red, or green light-emitting compounds known in the art in addition to the compounds of the present disclosure. Furthermore, if desired, a yellow or orange light-emitting layer may be further included.

In the organic electroluminescent device of the present disclosure, preferably, at least one inner surface layer selected from a chalcogenide layer, a metal halide layer, and a metal oxide layer (hereinafter referred to as “surface layers”) may be placed on an inner surface(s) of at least one of the pair of electrodes. Specifically, a chalcogenide (including oxides) layer of silicon and aluminum is preferably placed on an anode surface of an electroluminescent medium layer, and a halogenated metal layer or a metal oxide layer is preferably placed on a cathode surface of a light-emitting medium layer. The operation stability for the organic electroluminescent device may be obtained by the surface layer. Preferred examples of the chalcogenide include SiOₓ (1≤X≤2), AlOₓ (1≤X≤1.5), SiON, SiAlON, etc; preferred examples of the metal halide include LiF, MgF₂, CaF₂, rare earth fluoride metal, etc; and preferred examples of the metal oxide include Cs₂O, Li₂O, MgO, SrO, BaO, CaO, etc.

A hole-injection layer, a hole-transport layer, or an electron-blocking layer, or a combination thereof, may be used between the anode and the light-emitting layer. For the hole-injection layer, a plurality of layers may be used for the purpose of lowering the hole-injection barrier (or hole-injection voltage) from the anode to the hole-transport layer or electron-blocking layer, and each layer may have two compounds used simultaneously. The hole transport layer or electron blocking layer may also have a plurality of layers.

An electron buffer layer, a hole blocking layer, an electron transport layer, an electron injection layer, or a combination thereof can be used between the light-emitting layer and the cathode. The electron buffer layer may be multi-layers in order to control the injection of the electron and improve the interfacial properties between the light-emitting layer and the electron injection layer, wherein each of the multi-layers may use two compounds simultaneously. A plurality of layers may be used for the hole blocking layer or electron transport layer, and a plurality of compounds may be used for each layer.

The light-emitting auxiliary layer may be placed between the anode and the light-emitting layer, or between the cathode and the light-emitting layer. When the light-emitting auxiliary layer is placed between the anode and the light-emitting layer, it can be used for promoting the hole injection and/or the hole transport, or for preventing the overflow of electrons. When the light-emitting auxiliary layer is placed between the cathode and the light-emitting layer, it can be used for promoting the electron injection and/or the electron transport, or for preventing the overflow of holes. In addition, the hole auxiliary layer may be placed between the hole transport layer (or hole injection layer) and the light-emitting layer, and may be effective to promote or block the hole transport rate (or the hole injection rate), thereby enabling the charge balance to be controlled. Also, the electron blocking layer may be placed between the hole transport layer (or hole injection layer) and the light-emitting layer, and can confine the excitons within the light-emitting layer by blocking the overflow of electrons from the light-emitting layer to prevent a light-emitting leakage. When the hole transport layer is included by two or more, the additional layers may be used for the hole auxiliary layer or the electron blocking layer. The light-emitting auxiliary layer, hole auxiliary layer or electron blocking layer has the effect of improving the efficiency and/or lifespan of the organic electroluminescent device.

In addition, in the organic electroluminescent device of the present disclosure, preferably, a mixed region of an electron transport compound and a reductive dopant, or a mixed region of a hole transport compound and an oxidative dopant may be placed on at least one surface of a pair of electrodes. In this case, the electron transport compound is reduced to an anion, and thus it becomes easier to inject and transport electrons from the mixed region to an electroluminescent medium. Furthermore, the hole transport compound is oxidized to a cation, and thus it becomes easier to inject and transport holes from the mixed region to the electroluminescent medium. Preferably, the oxidative dopant includes various Lewis acids and acceptor compounds, and the reductive dopant includes alkali metals, alkali metal compounds, alkaline earth metals, rare-earth metals, and mixtures thereof. In addition, a reductive dopant layer may be employed as a charge generating layer to prepare an organic electroluminescent device having two or more light-emitting layers and emitting white light.

In addition, an organic electroluminescent material according to one embodiment may be used as light-emitting materials for a white organic light-emitting device. The white organic light-emitting device has been suggested to have various structures such as a side-by-side structure or a stacking structure depending on the arrangement of R (red), G (green) or YG (yellow green), and B (blue) light-emitting parts, or color conversion material (CCM) method, etc. In addition, the organic electroluminescent material according to one embodiment may also be applied to the organic electroluminescent device comprising a quantum dot (QD).

In order to form each layer of the organic electroluminescent device of the present disclosure, dry film-forming methods such as vacuum evaporation, sputtering, plasma, ion plating methods, etc, or wet film-forming methods such as inkjet printing, nozzle printing, slot coating, spin coating, dip coating, flow coating methods, etc, may be used. When forming a layer by the compound of the present disclosure, the layer can be formed by co-deposition or mixed-deposition.

When using a wet film-forming method, a thin film may be formed by dissolving or diffusing materials forming each layer into any suitable solvent such as ethanol, chloroform, tetrahydrofuran, dioxane, etc. The solvent may be any solvent where the materials forming each layer can be dissolved or diffused, and where there are no problems in film-formation capability.

In addition, it is possible to manufacture a display system, such as a display system for a smartphone, tablet, laptop, PCs, TVs, or vehicle, or a lighting system, such as an outdoor or indoor lighting system, using the organic electroluminescent device of the present disclosure.

Hereinafter, the preparation method of the compound according to the present disclosure and the physical properties thereof will be explained with reference to the representative compounds of the present disclosure. However, the present disclosure is not limited to the following examples.

### Example 1: Preparation of Compound C-1

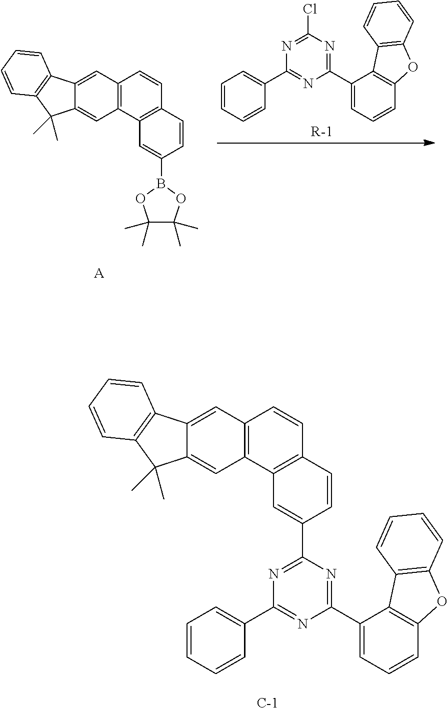

In a flask, compound A (5.9 g, 14.1 mmol), compound R-1 (5.6 g, 15.5 mmol), potassium carbonate (3.9 g, 28.2 mmol), and Pd(PPh₃)₄ (0.81 g, 0.7 mmol) were dissolved in 70 mL of toluene, 30 mL of ethanol, and 30 mL of water and refluxed at 120° C. for 2 hours. After completion of the reaction, the organic layer was extracted with ethyl acetate, and the residual moisture was removed using magnesium sulfate, followed by dryng. It was then separated by column chromatography to obtain compound C-1 (1.3 g, yield: 15%).

| Compound | MW | M.P. |
|---|---|---|
| C-1 | 615.74 | 230° C. |

### Example 2: Preparation of Compound C-2

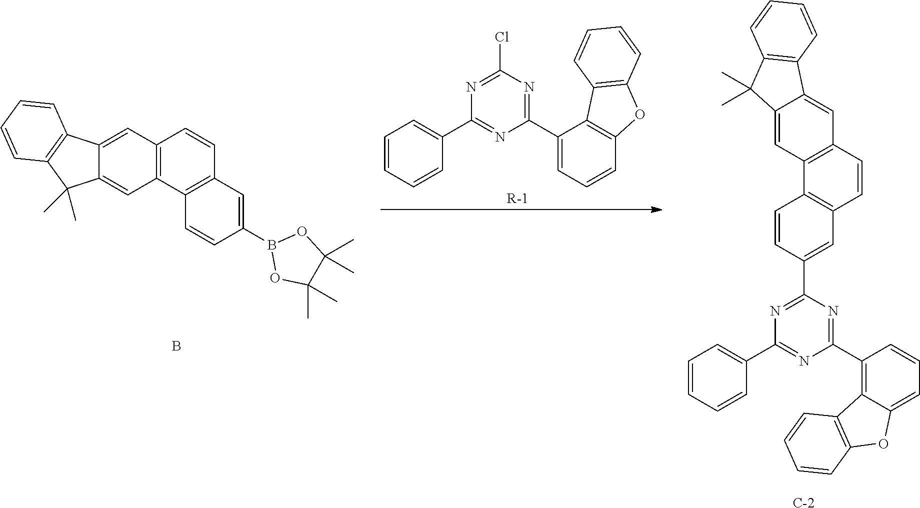

In a flask, compound B (6 g, 14.3 mmol), compound R-1 (5.6 g, 15.5 mmol), potassium carbonate (3.9 g, 28.2 mmol), and Pd(PPh₃)₄ (0.83 g, 0.8 mmol) were dissolved in 70 mL of toluene, 30 mL of ethanol, and 30 mL of water and refluxed at 120° C. for 2 hours. After completion of the reaction, the organic layer was extracted with ethyl acetate, and the residual moisture was removed using magnesium sulfate, followed by dryng. It was then separated by column chromatography to obtain compound C-2 (5 g, yield: 56%).

| Compound | MW | M.P. |
|---|---|---|
| C-2 | 615.74 | 280° C. |

### Example 3: Preparation of Compound C-3

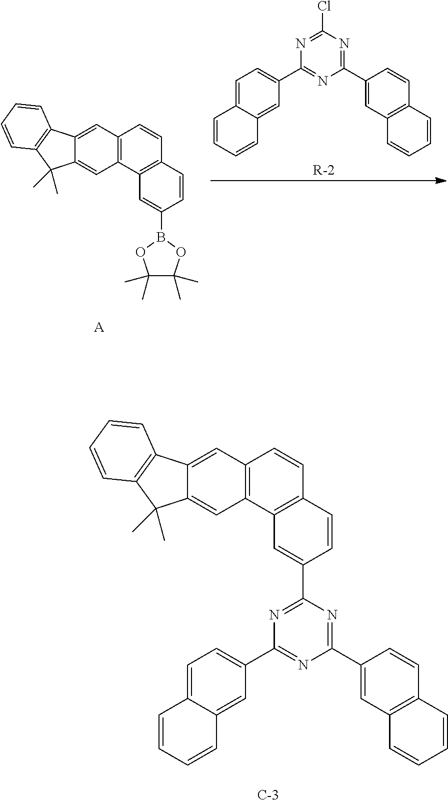

In a flask, compound A (6 g, 14.3 mmol), compound R-2 (6.4 g, 15.7 mmol), potassium carbonate (4 g, 28.5 mmol), and Pd(PPh₃)₄ (0.9 g, 0.8 mmol) were dissolved in 70 mL of toluene, 30 mL of ethanol, and 30 mL of water and refluxed at 120° C. for 2 hours. After completion of the reaction, the organic layer was extracted with ethyl acetate, and the residual moisture was removed using magnesium sulfate, followed by dryng. It was then separated by column chromatography to obtain compound C-3 (1.3 g, yield: 15%).

| Compound | MW | M.P. |
|---|---|---|
| C-3 | 625.78 | 230° C. |

### Example 4: Preparation of Compound C-4

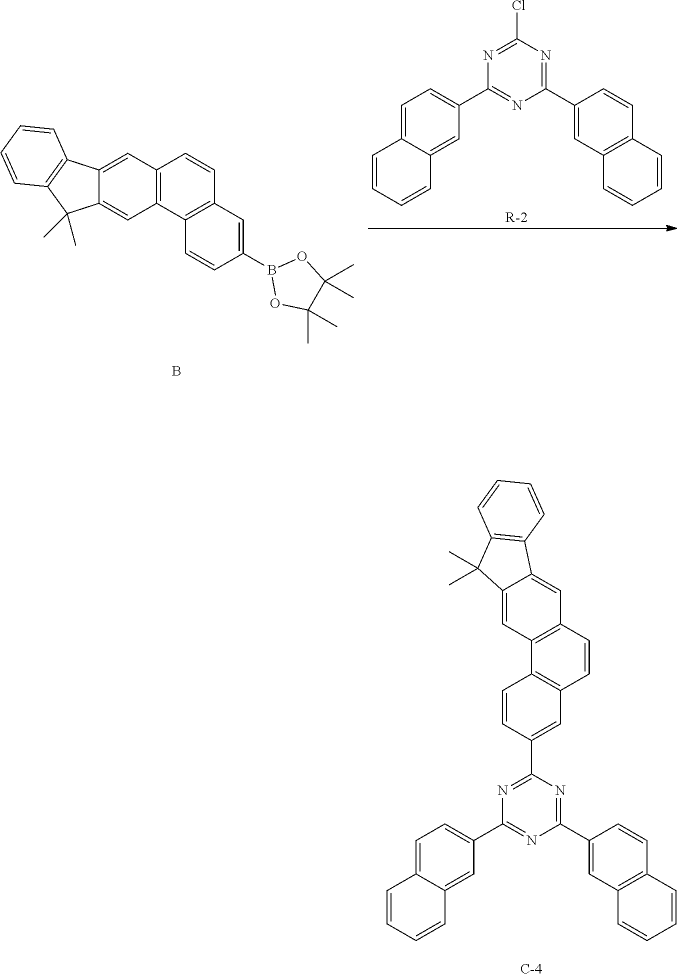

In a flask, compound B (6 g, 14.3 mmol), compound R-2 (6.4 g, 15.7 mmol) potassium carbonate (4 g, 28.4 mmol), and Pd(PPh₃)₄ (0.9 g, 0.8 mmol) were dissolved in 70 mL of toluene, 30 mL of ethanol, and 30 mL of water and refluxed at 120° C. for 2 hours. After completion of the reaction, the organic layer was extracted with ethyl acetate, and the residual moisture was removed using magnesium sulfate, followed by dryng. It was then separated by column chromatography to obtain compound C-4 (3.4 g, yield: 38%).

| Compound | MW | M.P. |
|---|---|---|
| C-4 | 625.78 | 273° C. |

The following examples describe the luminous efficiency and lifespan characteristics of an OLED according to the present disclosure. However, the following examples describe the characteristics of OLEDs according to the present disclosure for a detailed understanding of the present disclosure, but the present disclosure is not limited to the following examples.

### Device Examples 1 and 2: Preparation of OLEDs Comprising a Compound According to the Present Disclosure as a Host Material

OLEDs according to the present disclosure were prepared. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was mounted on a substrate holder of a vacuum vapor deposition apparatus. Compound HI-1 was introduced into a cell of the vacuum vapor deposition apparatus, and compound HT-3 was introduced into another cell. The two materials were evaporated at different rates, and compound HI-1 was deposited in a doping amount of 3 wt % based to the total amount of compound HI-1 and compound HT-3 to form a hole injection layer with a thickness of 10 nm. Subsequently, compound HT-3 was deposited on the hole injection layer to form a first hole transport layer with a thickness of 80 nm. Next, compound HT-4 was introduced into another cell of the vacuum vapor deposition apparatus and was evaporated by applying an electric current to the cell, thereby depositing a second hole transport layer with a thickness of 60 nm on the first hole transport layer. After forming the hole injection layer and the hole transport layers, a light-emitting layer was deposited thereon as follows: Each of the host compound listed in Table 1 below was introduced into the cells of the vacuum vapor deposition apparatus as hosts, and compound D-71 was introduced into another cell as a dopant. The hosts and the dopant were simultaneously evaporated at a different rate, and the dopant was deposited in a doping amount of 3 wt % based on the total amount of the hosts and dopant to form a light-emitting layer with a thickness of 40 nm on the second hole transport layer. Then, compound ET-1 and compound EI-1 were evaporated at a weight ratio of 50:50 as an electron transport material to form an electron transport layer having a thickness of 35 nm on the light-emitting layer. After depositing compound EI-1 as an electron injection layer with a thickness of 2 nm on the electron transport layer, an Al cathode was deposited with a thickness of 80 nm on the electron injection layer by using another vacuum vapor deposition apparatus. Thus, OLEDs were produced. Each compounds used for all the materials were purified by vacuum sublimation under 10 -6 torr.

### Comparative Examples 1 and 2: Preparation of OLEDs Comprising a Comparative Compound as a Host

OLEDs were prepared in the same manner as in Device Example 1, except that the host compound listed in Table 1 below was used as a host of the light-emitting layer.

The luminous color and the time taken for luminance to decrease from 100% to 80% at a luminance of 10,000 nits (lifespan: T80) of the OLED devices of Device Examples 1 and 2 and Comparative Examples 1 and 2 produced as described above, are measured, and the results thereof are shown in the following Table 1.

TABLE 1

| | Host | Light-Emitting Color | Lifespan T80 [hr] |
|---|---|---|---|
| Device | C-2 | Red | 34 |
| Example 1 | | | |
| Device | C-3 | Red | 24 |
| Example 2 | | | |
| Comparative | A-1 | Red | 11 |
| Example 1 | | | |
| Comparative | A-2 | Red | 12 |
| Example 2 | | | |

### Device Examples 3 and 4: Preparation of OLEDs Comprisind a Compound According to the Present Disclosure as an Electron Buffer Material

OLEDs according to the present disclosure were prepared. First, a transparent electrode indium tin oxide (ITO) thin film (10 Ω/sq) on a glass substrate for an OLED (GEOMATEC CO., LTD., Japan) was subjected to an ultrasonic washing with acetone and isopropyl alcohol, sequentially, and then was stored in isopropyl alcohol. The ITO substrate was mounted on a substrate holder of a vacuum vapor deposition apparatus. Compound HI-1 was introduced into a cell of the vacuum vapor deposition apparatus, and compound HT-1 was introduced into another cell. The two materials were evaporated at different rates, and compound HI-1 was deposited in a doping amount of 3 wt % based to the total amount of compound HI-1 and compound HT-1 to form a hole injection layer with a thickness of 10 nm. Subsequently, compound HT-1 was deposited to form a first hole transport layer with a thickness of 80 nm on the hole injection layer. Next, compound HT-5 was introduced into another cell of the vacuum vapor deposition apparatus and was evaporated by applying an electric current to the cell, thereby depositing a second hole transport layer with a thickness of 5 nm on the first hole transport layer. After forming the hole injection layer and the hole transport layers, a light-emitting layer was deposited thereon as follows: compound BH was introduced into the cell of the vacuum vapor deposition apparatus as a host, and compound BD was introduced into another cell as a dopant. The host material and the dopant material were simultaneously evaporated at a different rate, and the dopant was deposited in a doping amount of 2 wt % based on the total amount of the host and dopant to form a light-emitting layer with a thickness of 20 nm on the second hole transport layer. Next, an electron buffer layer having a thickness of 5 nm was deposited on the light-emitting layer by doping the compounds listed in Table 2 below as an electron buffer layer. Subsequently, ET-1 and compound EI-1 were doped at a weight ratio of 50:50 as an electron transport material to form an electron transport layer having a thickness of 30 nm on the electron buffer layer. After depositing compound EI-1 as an electron injection layer with a thickness of 2 nm on the electron transport layer, an Al cathode was deposited with a thickness of 80 nm on the electron injection layer by using another vacuum vapor deposition apparatus, thereby producing OLEDs. Each compounds used for all the materials were purified by vacuum sublimation under10⁻⁶ torr.

### Comparative Examples 3 and 4: Preparation of OLEDs Comprising a Comparative Compound as an Electron Buffer Material

OLEDs were prepared in the same manner as in Device Example 3, except that the compound listed in Table 2 below was used as an electron buffer material.

The luminous color and the time taken for luminance to decrease from 100% to 90% at a luminance of 10,000 nits (lifespan: T90) of the OLEDs of Device Examples 3 and 4 and Comparative Examples 3 and 4 produced as described above, are measured, and the results thereof are shown in the following Table 2.

TABLE 2

| | Electron Buffer | Light-Emitting | Lifespan |
|---|---|---|---|
| | Material | Color | T90 [hr] |
| Device | C-2 | Blue | 90 |
| Example 3 | | | |
| Device | C-3 | Blue | 76 |
| Example 4 | | | |
| Comparative | A-1 | Blue | 30 |
| Example 3 | | | |
| Comparative | A-2 | Blue | 46 |
| Example 4 | | | |

From Tables 1 and 2 above, it can be seen that an organic electroluminescent device including the organic electroluminescent compound according to the present disclosure as a host material or an electron buffer material, exhibits long lifespan characteristics, compared to the conventional organic electroluminescent device.

### Device Examples 5 and 6: Preparation of OLEDs by Co-Deposition of Host Compounds According to the Present Disclosure

In Device Examples 5 and 6, OLEDs were prepared in the same manner as in Device Example 1, except that Compound HT-1 and Compound HT-2 were used as materials for the first hole transport layer and the second hole transport layer, and that each of the first host compound and the second host compound listed in Table 3 below were co-deposited in a 50:50 ratio as the hosts of the light-emitting layer.

The driving voltage, the luminous efficiency, and luminous color at a luminance of 1,000 nits, and the time taken for luminance to decrease from 100% to 95% at a luminance of 10,000 nits (lifespan: T95) of the OLEDs of Device Examples 5 and 6 produced as described above, are measured, and the results thereof are shown in the following Table 3.

TABLE 3

| | | | Driving | Luminous | | |
|---|---|---|---|---|---|---|
| | First | Second | Voltage | Efficiency | Luminous | Lifespan |
| | Host | Host | (V) | (cd/A) | Color | T95 [hr] |
| Device | H-1 | C-1 | 3.1 | 32.6 | Red | 194 |
| Example 5 | | | | | | |
| Device | H-1 | C-2 | 3.0 | 29.4 | Red | 152 |
| Example 6 | | | | | | |

From Table 3 above, it can be seen that an organic electroluminescent device including the compound represented by formula 1 of the present disclosure and the compound represented by formula 2 of the present disclosure as a plurality of host materials, exhibits low driving voltage, high luminous efficiency, and/or long lifespan characteristics, compared to the conventional organic electroluminescent device.

The compounds used in the Device Examples and the Comparative Examples are shown in Table 4 below.

TABLE 4

| | |
|---|---|
| Hole Injection Layer/ Hole Transport Layer | 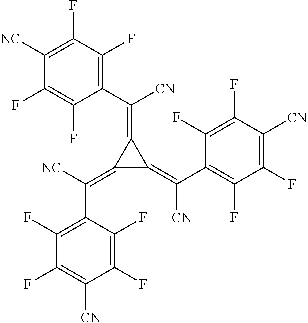 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F] |
| | HI-1 |
| | 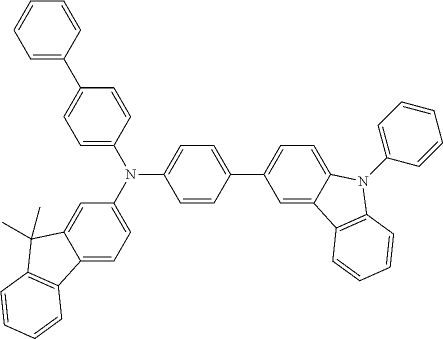 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] |
| | HT-1 |
| | 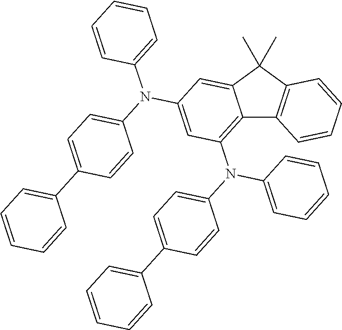 [SMILES: CC1(C)c2ccccc2-c2c(N(c3ccccc3)c3ccc(-c4ccccc4)cc3)cc(N(c3ccccc3)c3ccc(-c4ccccc4)cc3)cc21] |
| | HT-2 |
| | 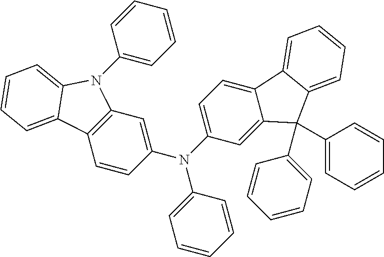 [SMILES: c1ccc(N(c2ccc3c(c2)C(c2ccccc2)(c2ccccc2)c2ccccc2-3)c2ccc3c4ccccc4n(-c4ccccc4)c3c2)cc1] |
| | HT-3 |
| | 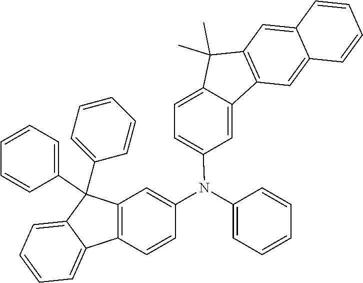 [SMILES: CC1(C)c2ccc(N(c3ccccc3)c3ccc4c(c3)C(c3ccccc3)(c3ccccc3)c3ccccc3-4)cc2-c2cc3ccccc3cc21] |
| | HT-4 |
| | 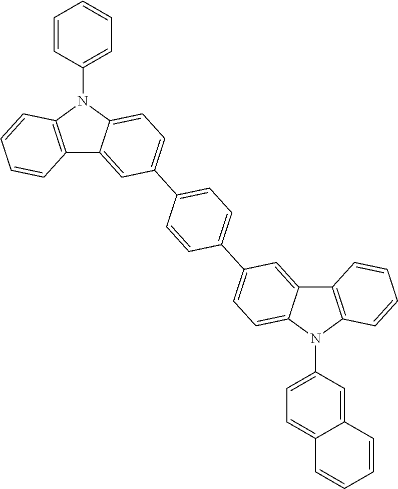 [SMILES: c1ccc(-n2c3ccccc3c3cc(-c4ccc(-c5ccc6c(c5)c5ccccc5n6-c5ccc6ccccc6c5)cc4)ccc32)cc1] |
| | HT-5 |
| Light-emitting Layer/ Electron Buffer Layer | 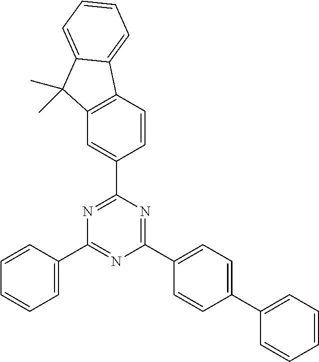 [SMILES: CC1(C)c2ccccc2-c2ccc(-c3nc(-c4ccccc4)nc(-c4ccc(-c5ccccc5)cc4)n3)cc21] |
| | A-1 |
| | 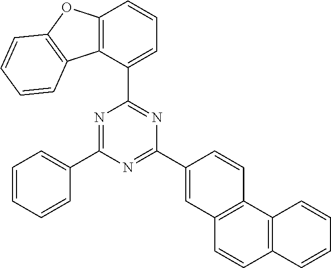 [SMILES: c1ccc(-c2nc(-c3ccc4c(ccc5ccccc54)c3)nc(-c3cccc4oc5ccccc5c34)n2)cc1] |
| | A-2 |
| | 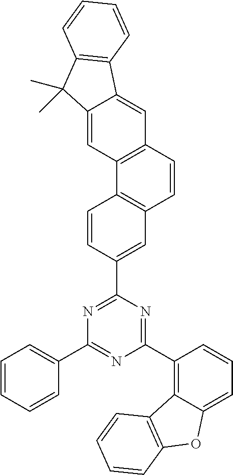 [SMILES: CC1(C)c2ccccc2-c2cc3ccc4cc(-c5nc(-c6ccccc6)nc(-c6cccc7oc8ccccc8c67)n5)ccc4c3cc21] |
| | C-2 |
| | 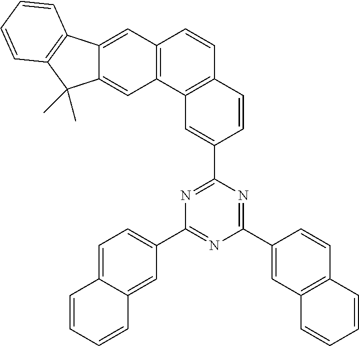 [SMILES: CC1(C)c2ccccc2-c2cc3ccc4ccc(-c5nc(-c6ccc7ccccc7c6)nc(-c6ccc7ccccc7c6)n5)cc4c3cc21] |
| | C-3 |
| | 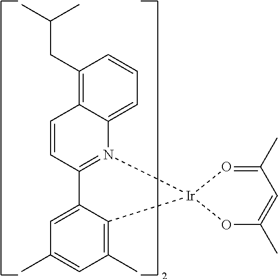 |
| | D-71 |
| | 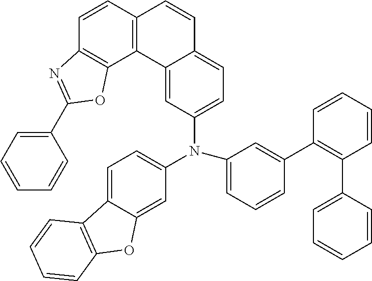 [SMILES: c1ccc(-c2nc3ccc4ccc5ccc(N(c6cccc(-c7ccccc7-c7ccccc7)c6)c6ccc7c(c6)oc6ccccc67)cc5c4c3o2)cc1] |
| | H-1 |
| | 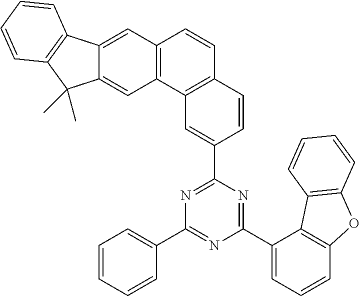 [SMILES: CC1(C)c2ccccc2-c2cc3ccc4ccc(-c5nc(-c6ccccc6)nc(-c6cccc7oc8ccccc8c67)n5)cc4c3cc21] |
| | C-1 |
| | 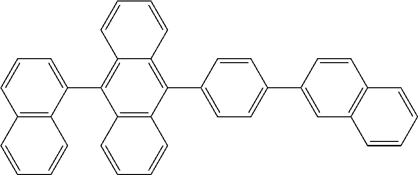 [SMILES: c1ccc2cc(-c3ccc(-c4c5ccccc5c(-c5cccc6ccccc56)c5ccccc45)cc3)ccc2c1] |
| | BH |
| | 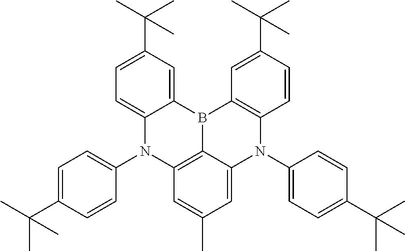 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1] |
| | BD |
| Electron Transport Layer/ Electron Injection Layer | 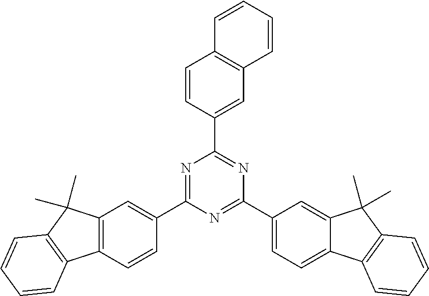 [SMILES: CC1(C)c2ccccc2-c2ccc(-c3nc(-c4ccc5c(c4)C(C)(C)c4ccccc4-5)nc(-c4ccc5ccccc5c4)n3)cc21] |
| | ET-1 |
| | 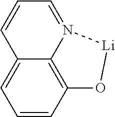 |
| | EI-1 |

1. An organic electroluminescent compound represented by the following formula 1: 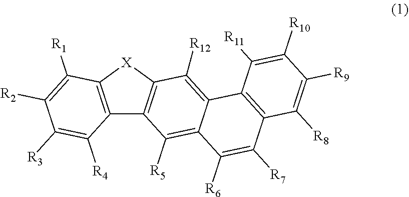
wherein,
X represents —C(R′)(R″)- or —Se—;
R′ and R″ each independently represent a substituted or unsubstituted (C1-C30)alkyl, a substituted or unsubstituted (C6-C30)aryl, or a substituted or unsubstituted (3- to 30-membered)heteroaryl; or may be linked together to form a ring, and R′ and R″ may be the same or different from each other;
R₁ to R₁₂ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of (C3-C30) aliphatic ring and (C6-C30) aromatic ring, or *-L-HAr; or may be linked to an adjacent substituent(s) to form a ring;
with a proviso that any one of R₁ to R₁₂ represents *-L-HAr;
L represents a single bond, a substituted or unsubstituted (C6-C30)arylene, or a substituted or unsubstituted (3- to 30-membered)heteroarylene; and
HAr represents a substituted or unsubstituted (3- to 30-membered)heteroaryl containing at least one nitrogen atom(s).

2. The organic electroluminescent compound according to claim 1, wherein the substituent(s) of the substituted alkyl, the substituted aryl, the substituted arylene, the substituted heteroaryl, the substituted heteroarylene, the substituted cycloalkyl, the substituted alkoxy, the substituted trialkylsilyl, the substituted dialkylarylsilyl, the substituted alkyldiarylsilyl, the substituted triarylsilyl, and the substituted fused ring of an aliphatic ring and an aromatic ring, each independently are at least one selected from the group consisting of deuterium; halogen; cyano; carboxyl; nitro; hydroxyl; phosphine oxide; (C1-C30)alkyl; halo(C1-C30)alkyl; (C2-C30)alkenyl unsubstituted or substituted with at least one (C6-C30)aryl; (C2-C30)alkynyl; (C1-C30)alkoxy; (C1-C30)alkylthio; (C3-C30)cycloalkyl; (C3-C30)cycloalkenyl; a (3- to 7-membered)heterocycloalkyl; (C6-C30)aryloxy; (C6-C30)arylthio; (3- to 30-membered)heteroaryl unsubstituted or substituted with at least one of deuterium and a (C6-C30)aryls; (C6-C30)aryl unsubstituted or substituted with at least one of deuterium, cyano, (C1-C30)alkyl, and (3-to 30-membered)heteroaryl; tri(C1-C30)alkylsilyl; tri(C6-C30)arylsilyl; adi(C1-C30)alkyl(C6-C30)arylsilyl; (C1-C30)alkyldi(C6-C30)arylsilyl; a fused ring of (C3-C30) aliphatic ring and (C6-C30) aromatic ring; (C1-C30)alkylcarbonyl; (C1-C30)alkoxycarbonyl;
(C6-C30)arylcarbonyl; di(C6-C30)arylboronyl; di(C1-C30)alkylboronyl; (C1-C30)alkyl(C6-C30)arylboronyl; (C6-C30)ar(C1-C30)alkyl; and (C1-C30)alkyl(C6-C30)aryl.

3. The organic electroluminescent compound according to claim 1, wherein the formula 1 is represented by any one of the following formulas 1-1 to 1-12: 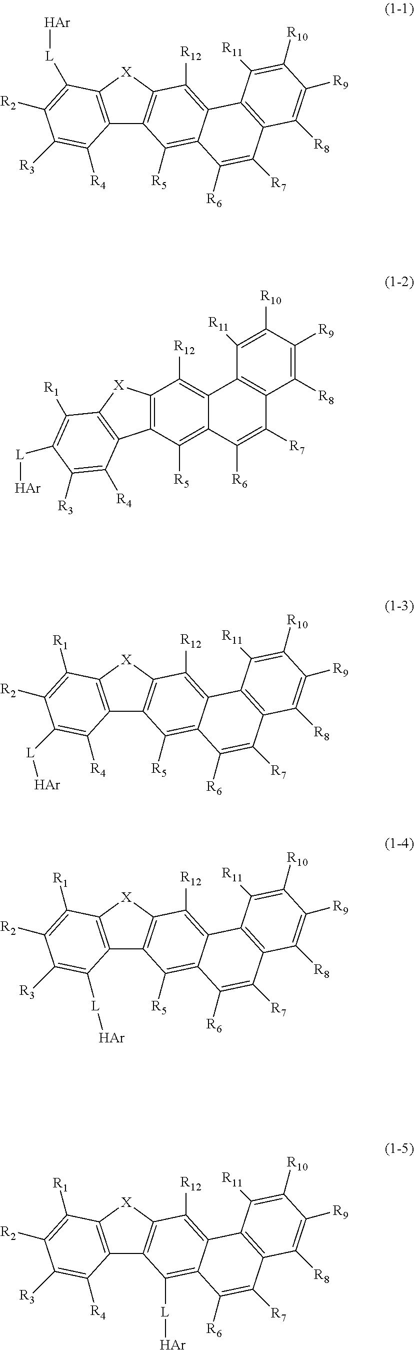 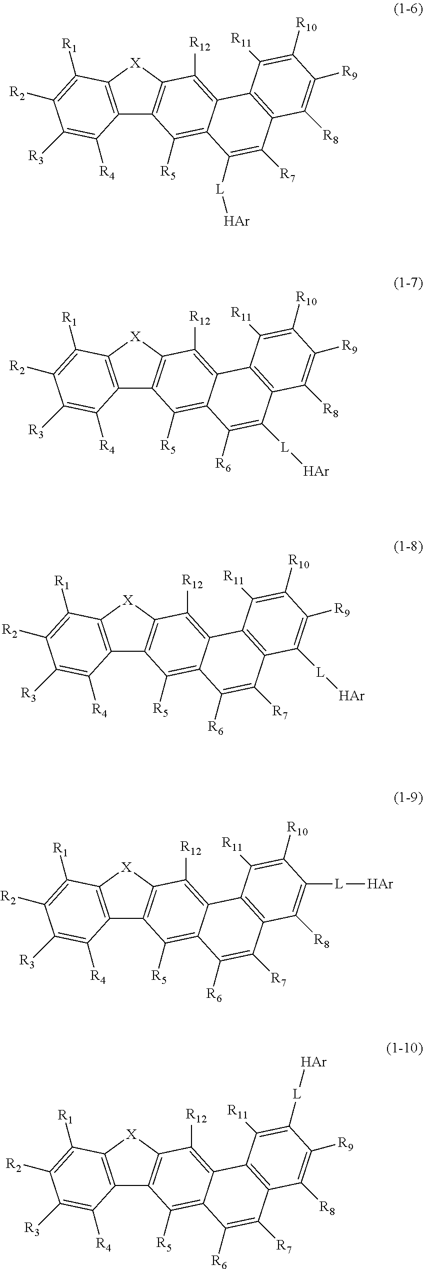 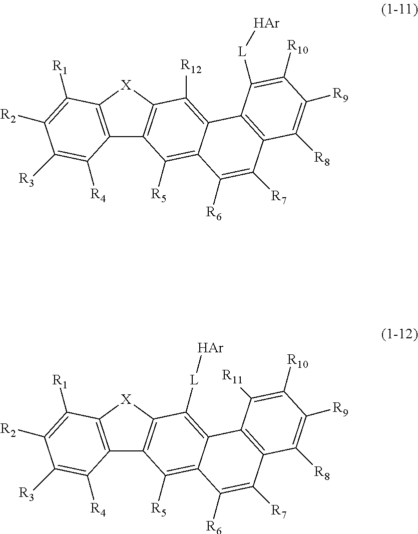
wherein, R₁ to R₁₂, X, L, and HAr are each as defined in claim 1.

4. The organic electroluminescent compound according to claim 1, wherein the HAr represents a substituted or unsubstituted pyridyl, a substituted or unsubstituted pyrimidinyl, a substituted or unsubstituted triazinyl, a substituted or unsubstituted quinazolinyl, a substituted or unsubstituted benzoquinazolinyl, a substituted or unsubstituted quinoxalinyl, a substituted or unsubstituted benzoquinoxalinyl, a substituted or unsubstituted quinolyl, a substituted or unsubstituted benzoquinolinyl, a substituted or unsubstituted isoquinolyl, a substituted or unsubstituted benzoisoquinolyl, a substituted or unsubstituted triazolyl, a substituted or unsubstituted pyrazolyl, a substituted or unsubstituted naphthyridinyl, a substituted or unsubstituted benzofuropyrimidinyl, a substituted or unsubstituted benzothienopyrimidinyl, a substituted or unsubstituted carbazolyl, or a substituted or unsubstituted pyridopyrazinyl.

5. The organic electroluminescent compound according to claim 1, wherein the compound represented by formula 1 is selected from the group consisting of the following compounds:

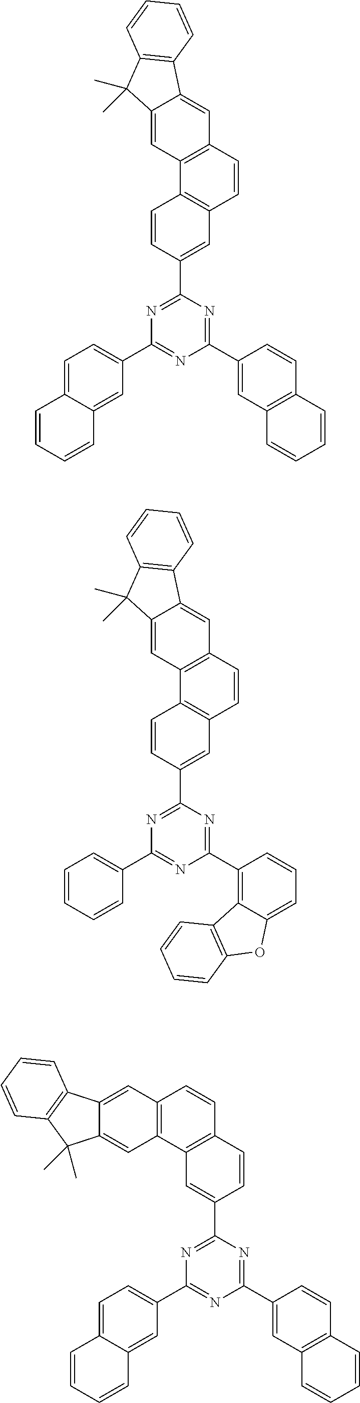 [SMILES: CC1(C)c2ccccc2-c2cc3ccc4cc(-c5nc(-c6ccc7ccccc7c6)nc(-c6ccc7ccccc7c6)n5)ccc4c3cc21 ; CC1(C)c2ccccc2-c2cc3ccc4cc(-c5nc(-c6ccccc6)nc(-c6cccc7oc8ccccc8c67)n5)ccc4c3cc21 ; CC1(C)c2ccccc2-c2cc3ccc4ccc(-c5nc(-c6ccc7ccccc7c6)nc(-c6ccc7ccccc7c6)n5)cc4c3cc21]

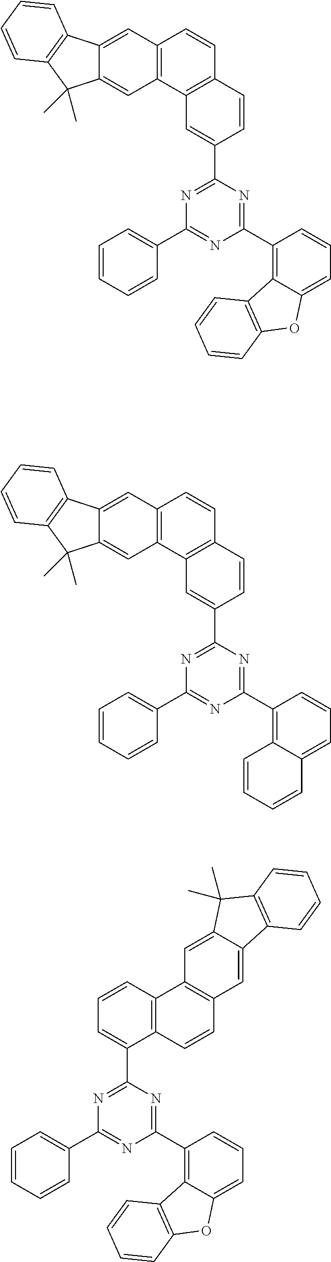 [SMILES: CC1(C)c2ccccc2-c2cc3ccc4ccc(-c5nc(-c6ccccc6)nc(-c6cccc7oc8ccccc8c67)n5)cc4c3cc21 ; CC1(C)c2ccccc2-c2cc3ccc4ccc(-c5nc(-c6ccccc6)nc(-c6cccc7ccccc67)n5)cc4c3cc21 ; CC1(C)c2ccccc2-c2cc3ccc4c(-c5nc(-c6ccccc6)nc(-c6cccc7oc8ccccc8c67)n5)cccc4c3cc21]

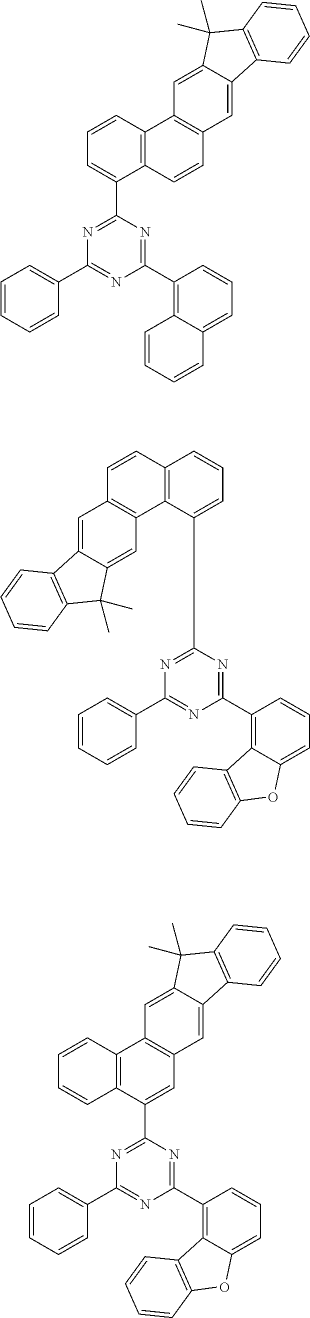 [SMILES: CC1(C)c2ccccc2-c2cc3ccc4c(-c5nc(-c6ccccc6)nc(-c6cccc7ccccc67)n5)cccc4c3cc21 ; CC1(C)c2ccccc2-c2cc3ccc4cccc(-c5nc(-c6ccccc6)nc(-c6cccc7oc8ccccc8c67)n5)c4c3cc21 ; CC1(C)c2ccccc2-c2cc3cc(-c4nc(-c5ccccc5)nc(-c5cccc6oc7ccccc7c56)n4)c4ccccc4c3cc21]

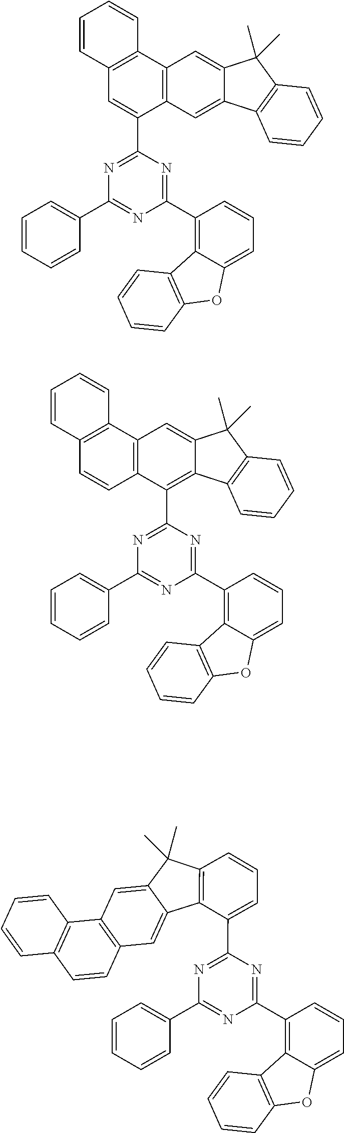 [SMILES: CC1(C)c2ccccc2-c2cc3c(-c4nc(-c5ccccc5)nc(-c5cccc6oc7ccccc7c56)n4)cc4ccccc4c3cc21 ; CC1(C)c2ccccc2-c2c1cc1c(ccc3ccccc31)c2-c1nc(-c2ccccc2)nc(-c2cccc3oc4ccccc4c23)n1 ; CC1(C)c2cc3c(ccc4ccccc43)cc2-c2c(-c3nc(-c4ccccc4)nc(-c4cccc5oc6ccccc6c45)n3)cccc21]

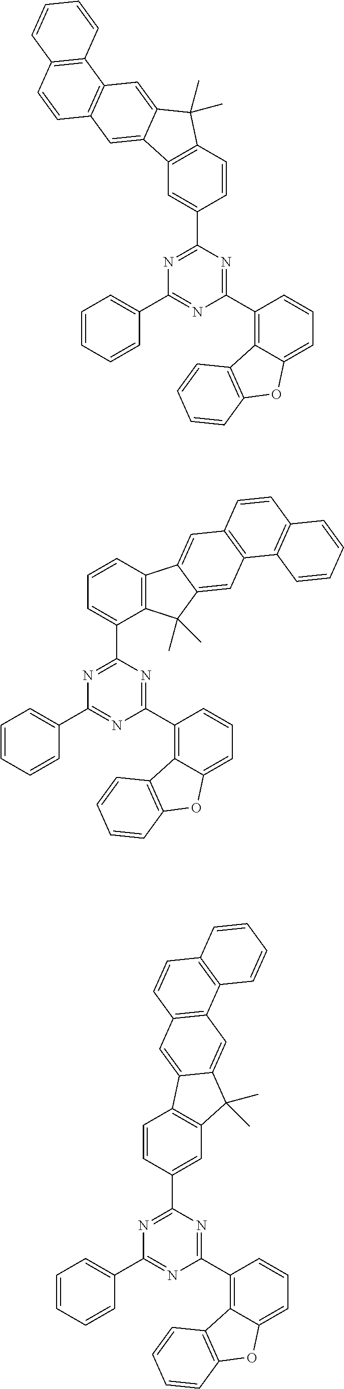 [SMILES: CC1(C)c2ccc(-c3nc(-c4ccccc4)nc(-c4cccc5oc6ccccc6c45)n3)cc2-c2cc3ccc4ccccc4c3cc21 ; CC1(C)c2cc3c(ccc4ccccc43)cc2-c2cccc(-c3nc(-c4ccccc4)nc(-c4cccc5oc6ccccc6c45)n3)c21 ; CC1(C)c2cc(-c3nc(-c4ccccc4)nc(-c4cccc5oc6ccccc6c45)n3)ccc2-c2cc3ccc4ccccc4c3cc21]

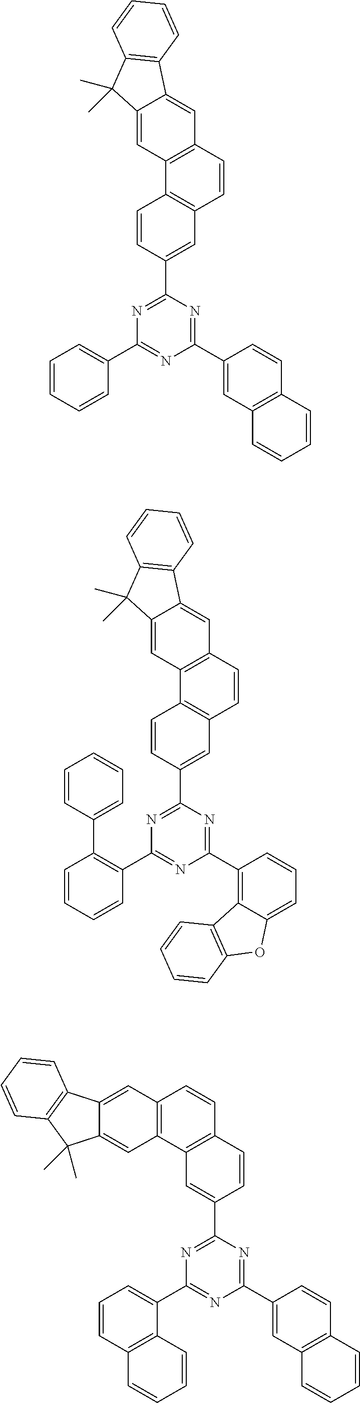 [SMILES: CC1(C)c2ccccc2-c2cc3ccc4cc(-c5nc(-c6ccccc6)nc(-c6ccc7ccccc7c6)n5)ccc4c3cc21 ; CC1(C)c2ccccc2-c2cc3ccc4cc(-c5nc(-c6ccccc6-c6ccccc6)nc(-c6cccc7oc8ccccc8c67)n5)ccc4c3cc21 ; CC1(C)c2ccccc2-c2cc3ccc4ccc(-c5nc(-c6ccc7ccccc7c6)nc(-c6cccc7ccccc67)n5)cc4c3cc21]

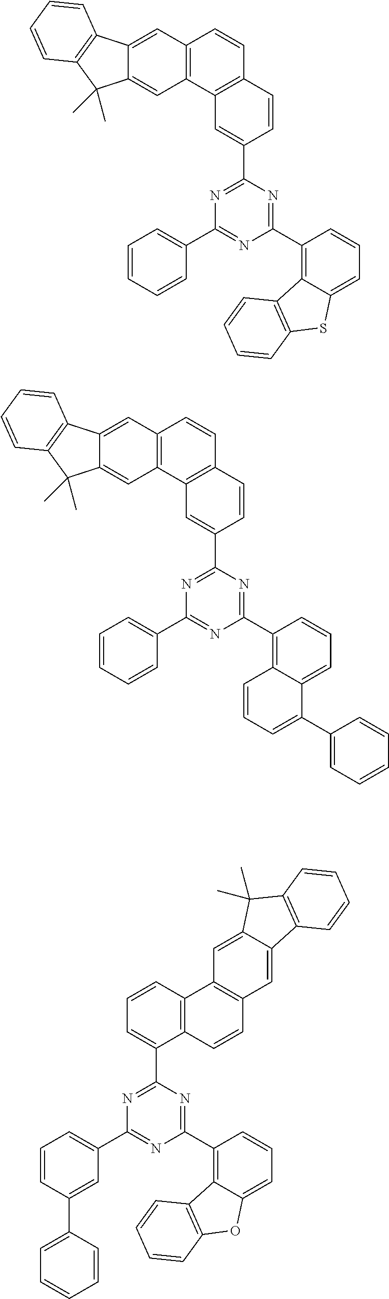 [SMILES: CC1(C)c2ccccc2-c2cc3ccc4ccc(-c5nc(-c6ccccc6)nc(-c6cccc7sc8ccccc8c67)n5)cc4c3cc21 ; CC1(C)c2ccccc2-c2cc3ccc4ccc(-c5nc(-c6ccccc6)nc(-c6cccc7c(-c8ccccc8)cccc67)n5)cc4c3cc21 ; CC1(C)c2ccccc2-c2cc3ccc4c(-c5nc(-c6cccc(-c7ccccc7)c6)nc(-c6cccc7oc8ccccc8c67)n5)cccc4c3cc21]

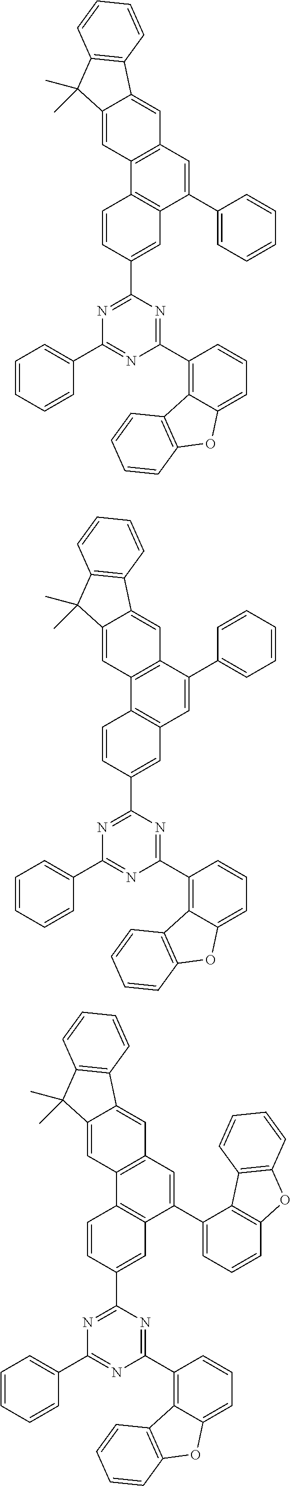

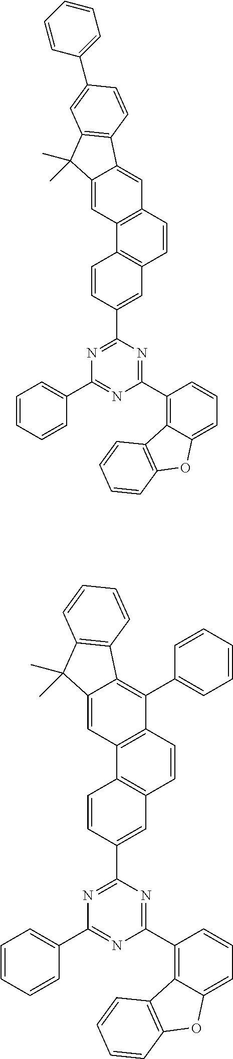 [SMILES: CC1(C)c2cc(-c3ccccc3)ccc2-c2cc3ccc4cc(-c5nc(-c6ccccc6)nc(-c6cccc7oc8ccccc8c67)n5)ccc4c3cc21 ; CC1(C)c2ccccc2-c2c1cc1c(ccc3cc(-c4nc(-c5ccccc5)nc(-c5cccc6oc7ccccc7c56)n4)ccc31)c2-c1ccccc1]

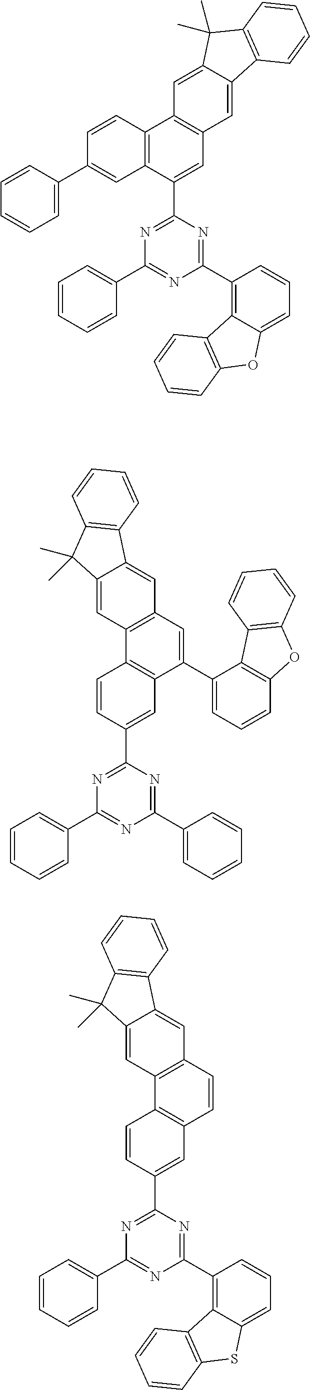 [SMILES: CC1(C)c2ccccc2-c2cc3cc(-c4nc(-c5ccccc5)nc(-c5cccc6oc7ccccc7c56)n4)c4cc(-c5ccccc5)ccc4c3cc21 ; CC1(C)c2ccccc2-c2cc3cc(-c4cccc5oc6ccccc6c45)c4cc(-c5nc(-c6ccccc6)nc(-c6ccccc6)n5)ccc4c3cc21 ; CC1(C)c2ccccc2-c2cc3ccc4cc(-c5nc(-c6ccccc6)nc(-c6cccc7sc8ccccc8c67)n5)ccc4c3cc21]

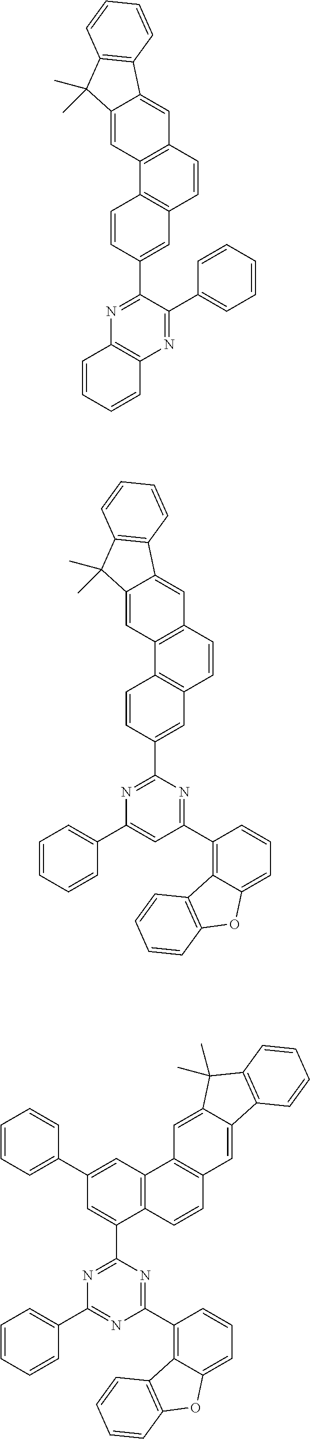 [SMILES: CC1(C)c2ccccc2-c2cc3ccc4cc(-c5nc6ccccc6nc5-c5ccccc5)ccc4c3cc21 ; CC1(C)c2ccccc2-c2cc3ccc4cc(-c5nc(-c6ccccc6)cc(-c6cccc7oc8ccccc8c67)n5)ccc4c3cc21 ; CC1(C)c2ccccc2-c2cc3ccc4c(-c5nc(-c6ccccc6)nc(-c6cccc7oc8ccccc8c67)n5)cc(-c5ccccc5)cc4c3cc21]

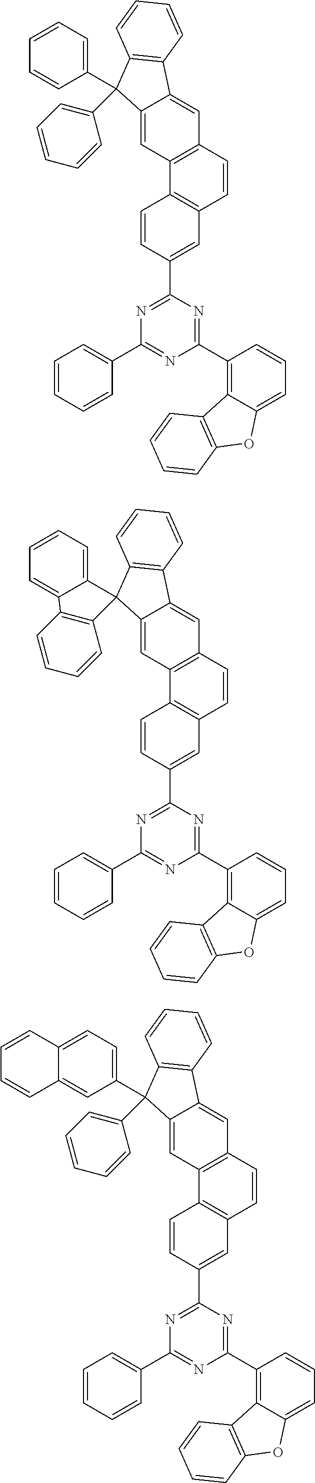

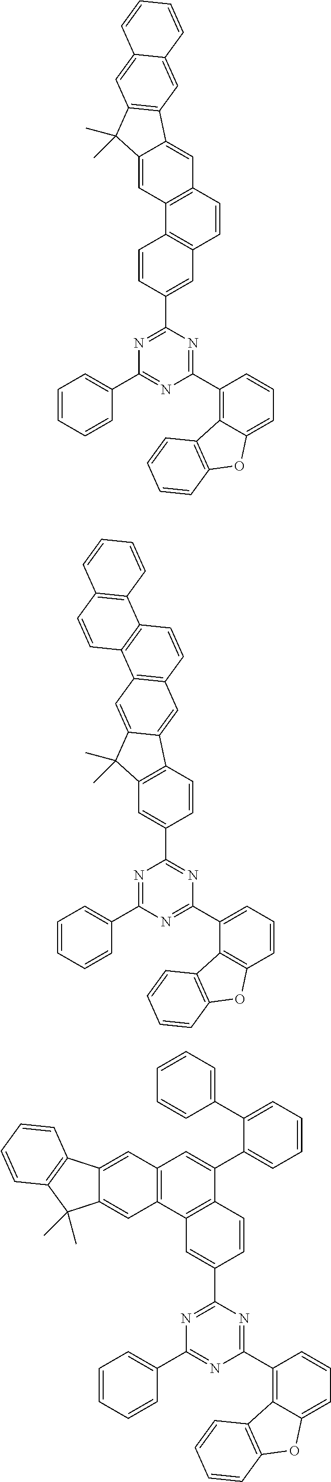

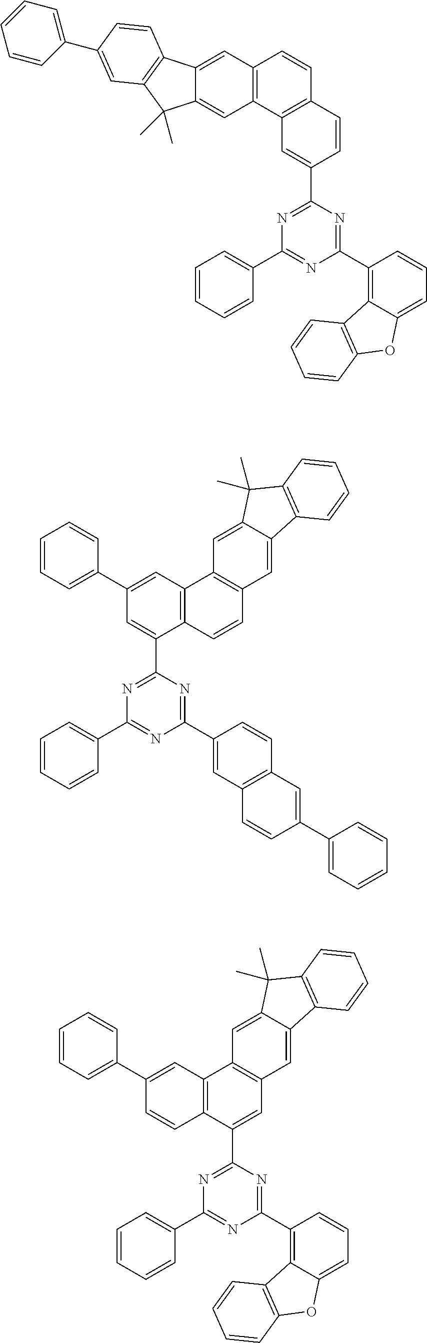 [SMILES: CC1(C)c2cc(-c3ccccc3)ccc2-c2cc3ccc4ccc(-c5nc(-c6ccccc6)nc(-c6cccc7oc8ccccc8c67)n5)cc4c3cc21 ; CC1(C)c2ccccc2-c2cc3ccc4c(-c5nc(-c6ccccc6)nc(-c6ccc7cc(-c8ccccc8)ccc7c6)n5)cc(-c5ccccc5)cc4c3cc21 ; CC1(C)c2ccccc2-c2cc3cc(-c4nc(-c5ccccc5)nc(-c5cccc6oc7ccccc7c56)n4)c4ccc(-c5ccccc5)cc4c3cc21]

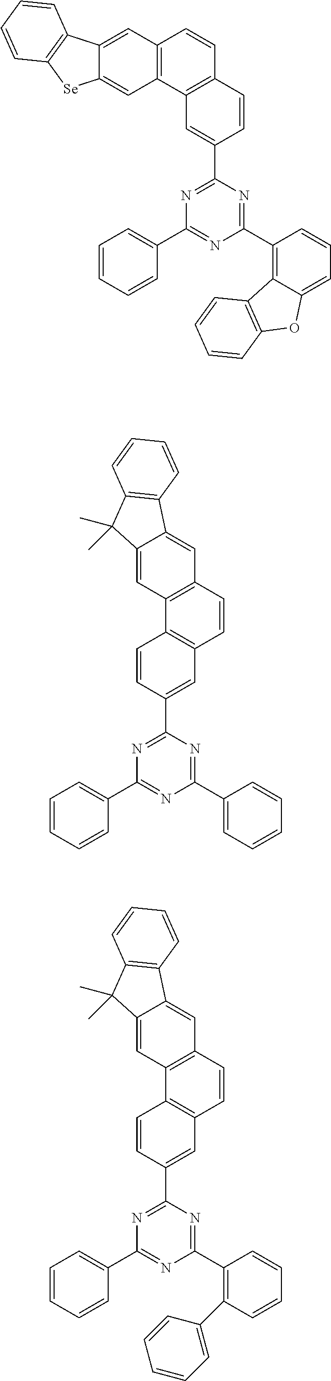 [SMILES: c1ccc(-c2nc(-c3ccc4ccc5cc6c(cc5c4c3)[se]c3ccccc36)nc(-c3cccc4oc5ccccc5c34)n2)cc1 ; CC1(C)c2ccccc2-c2cc3ccc4cc(-c5nc(-c6ccccc6)nc(-c6ccccc6)n5)ccc4c3cc21 ; CC1(C)c2ccccc2-c2cc3ccc4cc(-c5nc(-c6ccccc6)nc(-c6ccccc6-c6ccccc6)n5)ccc4c3cc21]

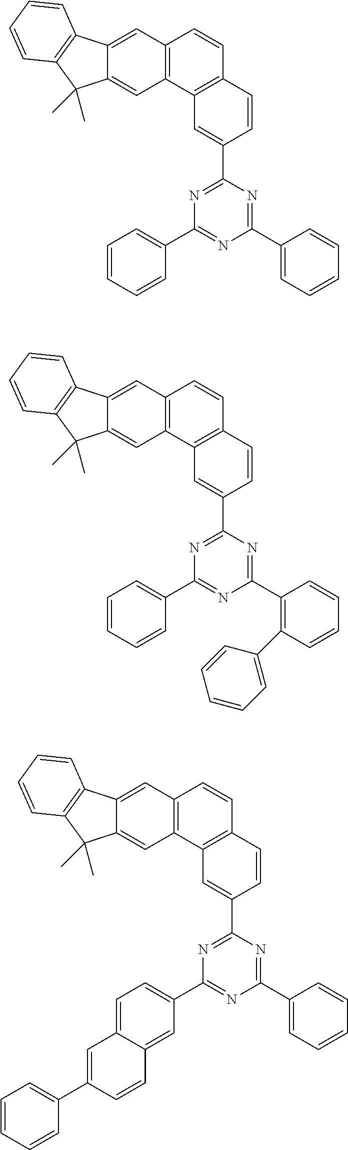 [SMILES: CC1(C)c2ccccc2-c2cc3ccc4ccc(-c5nc(-c6ccccc6)nc(-c6ccccc6)n5)cc4c3cc21 ; CC1(C)c2ccccc2-c2cc3ccc4ccc(-c5nc(-c6ccccc6)nc(-c6ccccc6-c6ccccc6)n5)cc4c3cc21 ; CC1(C)c2ccccc2-c2cc3ccc4ccc(-c5nc(-c6ccccc6)nc(-c6ccc7cc(-c8ccccc8)ccc7c6)n5)cc4c3cc21]

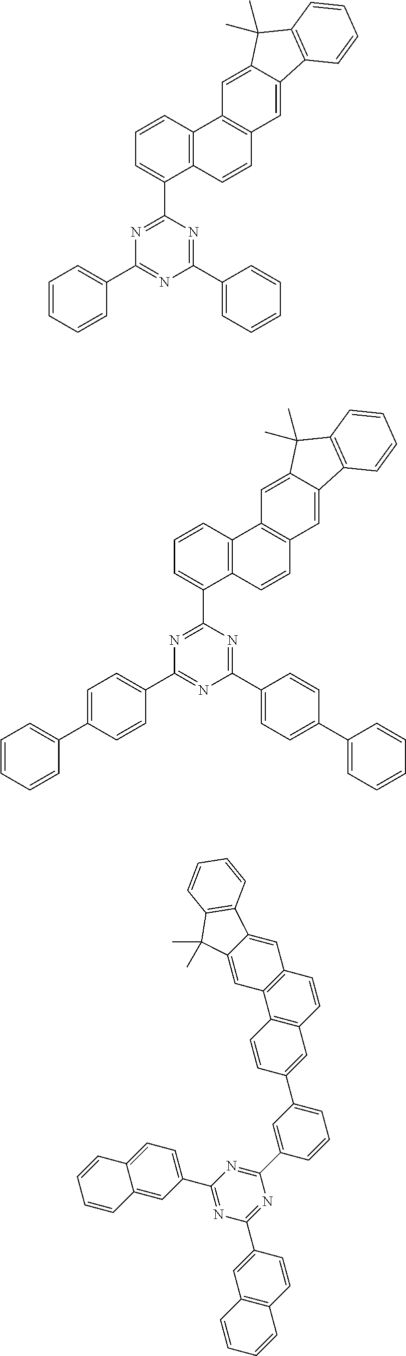 [SMILES: CC1(C)c2ccccc2-c2cc3ccc4c(-c5nc(-c6ccccc6)nc(-c6ccccc6)n5)cccc4c3cc21 ; CC1(C)c2ccccc2-c2cc3ccc4c(-c5nc(-c6ccc(-c7ccccc7)cc6)nc(-c6ccc(-c7ccccc7)cc6)n5)cccc4c3cc21 ; CC1(C)c2ccccc2-c2cc3ccc4cc(-c5cccc(-c6nc(-c7ccc8ccccc8c7)nc(-c7ccc8ccccc8c7)n6)c5)ccc4c3cc21]

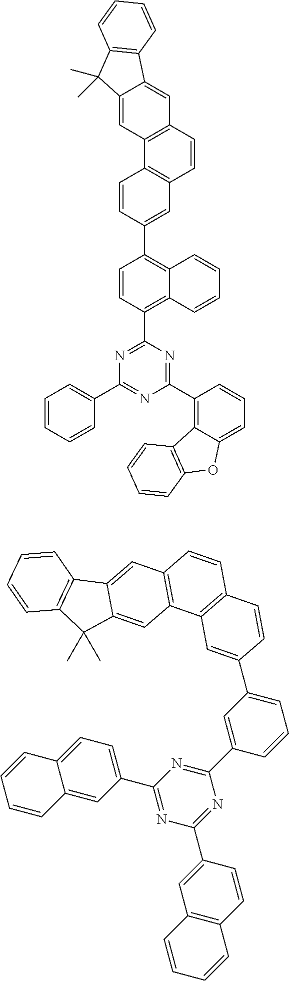 [SMILES: CC1(C)c2ccccc2-c2cc3ccc4cc(-c5ccc(-c6nc(-c7ccccc7)nc(-c7cccc8oc9ccccc9c78)n6)c6ccccc56)ccc4c3cc21 ; CC1(C)c2ccccc2-c2cc3ccc4ccc(-c5cccc(-c6nc(-c7ccc8ccccc8c7)nc(-c7ccc8ccccc8c7)n6)c5)cc4c3cc21]

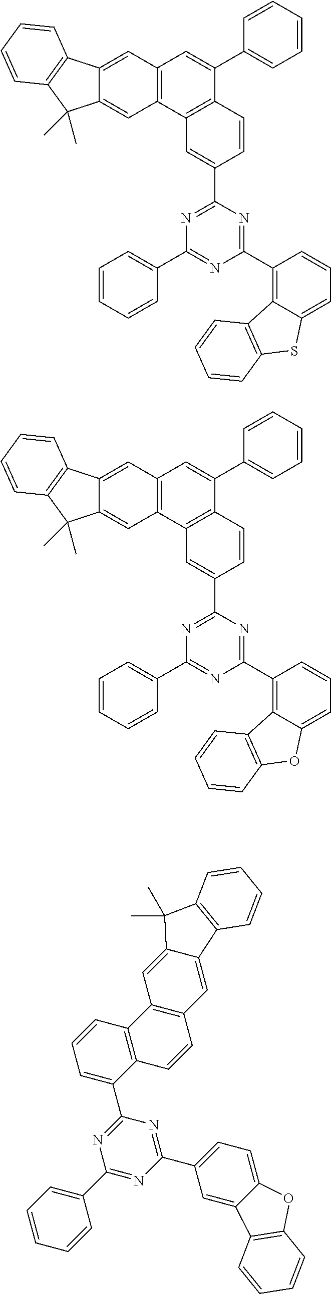

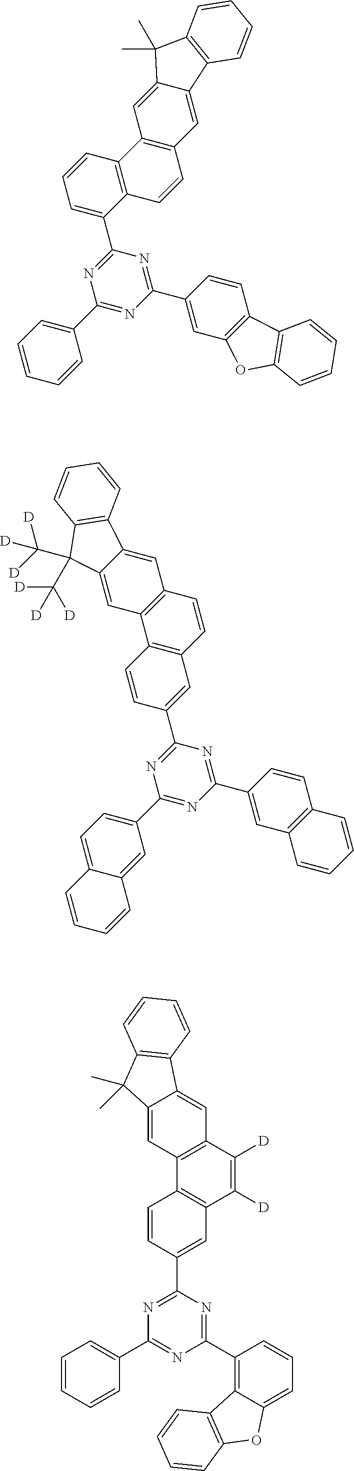 [SMILES: CC1(C)c2ccccc2-c2cc3ccc4c(-c5nc(-c6ccccc6)nc(-c6ccc7c(c6)oc6ccccc67)n5)cccc4c3cc21 ; [2H]C([2H])([2H])C1(C([2H])([2H])[2H])c2ccccc2-c2cc3ccc4cc(-c5nc(-c6ccc7ccccc7c6)nc(-c6ccc7ccccc7c6)n5)ccc4c3cc21 ; [2H]c1c([2H])c2cc3c(cc2c2ccc(-c4nc(-c5ccccc5)nc(-c5cccc6oc7ccccc7c56)n4)cc12)C(C)(C)c1ccccc1-3]

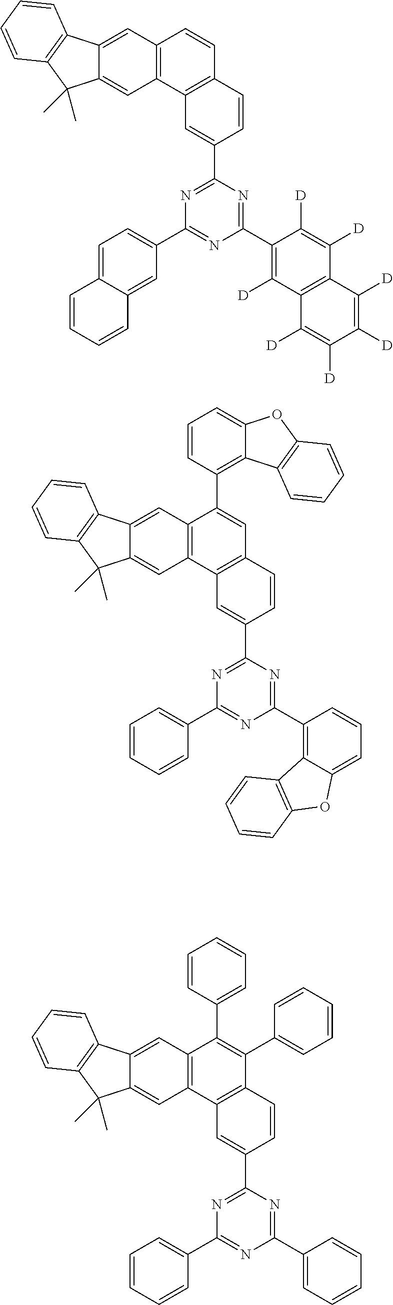 [SMILES: [2H]c1c([2H])c([2H])c2c([2H])c(-c3nc(-c4ccc5ccccc5c4)nc(-c4ccc5ccc6cc7c(cc6c5c4)C(C)(C)c4ccccc4-7)n3)c([2H])c([2H])c2c1[2H] ; CC1(C)c2ccccc2-c2cc3c(-c4cccc5oc6ccccc6c45)cc4ccc(-c5nc(-c6ccccc6)nc(-c6cccc7oc8ccccc8c67)n5)cc4c3cc21 ; CC1(C)c2ccccc2-c2cc3c(-c4ccccc4)c(-c4ccccc4)c4ccc(-c5nc(-c6ccccc6)nc(-c6ccccc6)n5)cc4c3cc21]

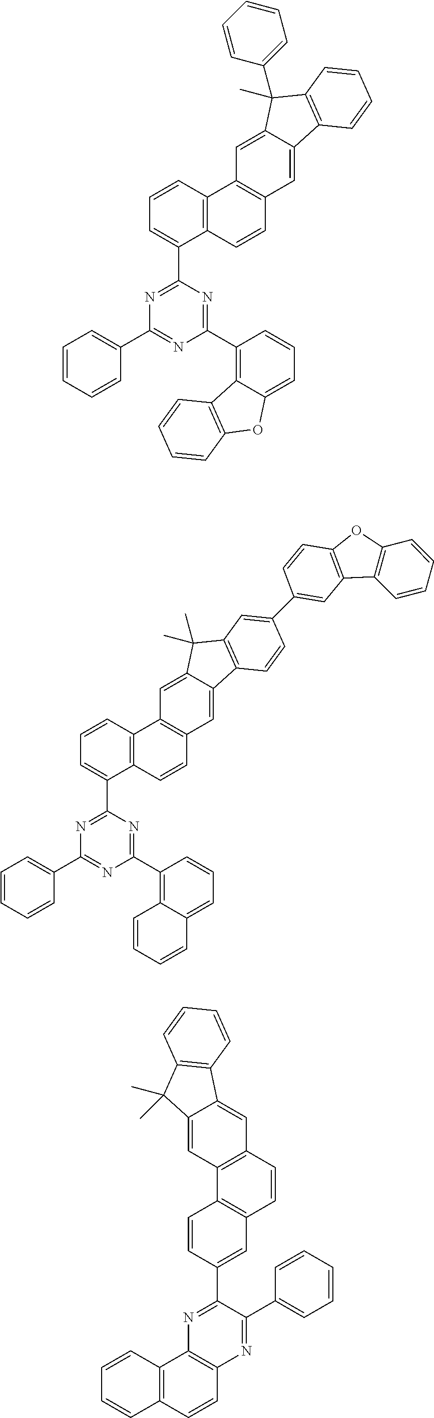 [SMILES: CC1(c2ccccc2)c2ccccc2-c2cc3ccc4c(-c5nc(-c6ccccc6)nc(-c6cccc7oc8ccccc8c67)n5)cccc4c3cc21 ; CC1(C)c2cc(-c3ccc4oc5ccccc5c4c3)ccc2-c2cc3ccc4c(-c5nc(-c6ccccc6)nc(-c6cccc7ccccc67)n5)cccc4c3cc21 ; CC1(C)c2ccccc2-c2cc3ccc4cc(-c5nc6c(ccc7ccccc76)nc5-c5ccccc5)ccc4c3cc21]

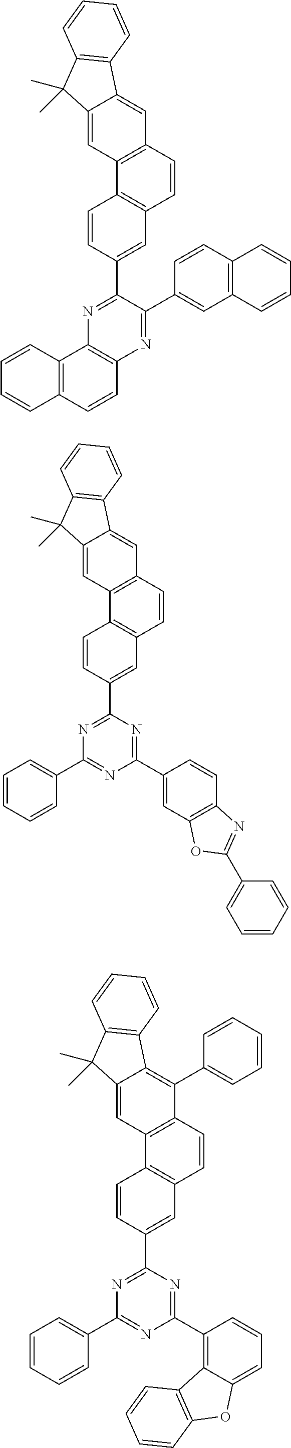

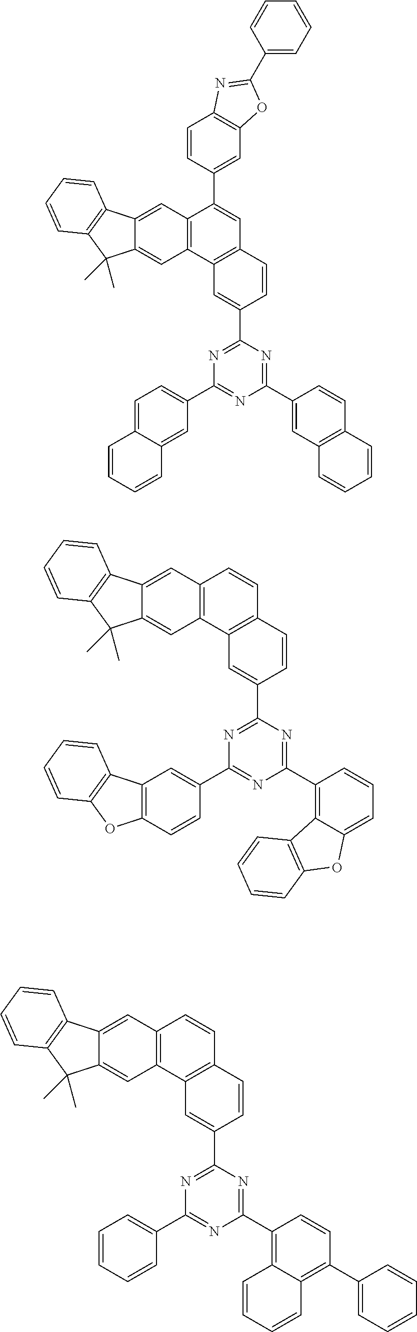 [SMILES: CC1(C)c2ccccc2-c2cc3c(-c4ccc5nc(-c6ccccc6)oc5c4)cc4ccc(-c5nc(-c6ccc7ccccc7c6)nc(-c6ccc7ccccc7c6)n5)cc4c3cc21 ; CC1(C)c2ccccc2-c2cc3ccc4ccc(-c5nc(-c6ccc7oc8ccccc8c7c6)nc(-c6cccc7oc8ccccc8c67)n5)cc4c3cc21 ; CC1(C)c2ccccc2-c2cc3ccc4ccc(-c5nc(-c6ccccc6)nc(-c6ccc(-c7ccccc7)c7ccccc67)n5)cc4c3cc21]

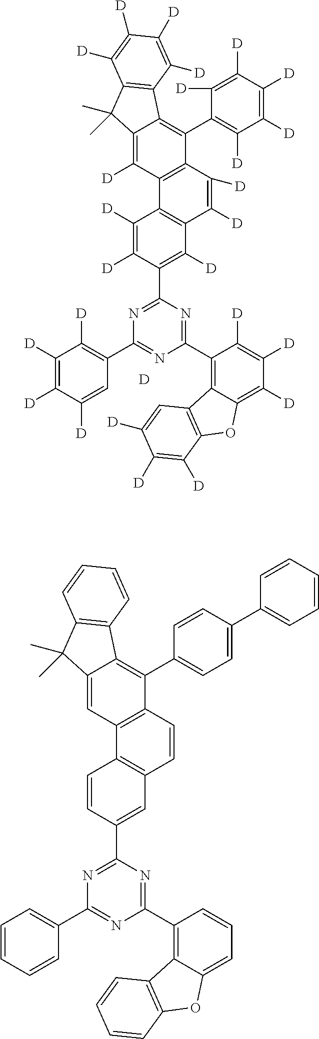

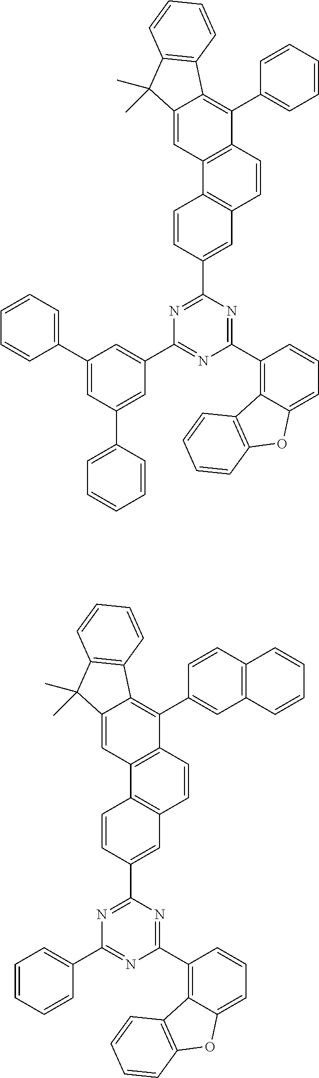 [SMILES: CC1(C)c2ccccc2-c2c1cc1c(ccc3cc(-c4nc(-c5cc(-c6ccccc6)cc(-c6ccccc6)c5)nc(-c5cccc6oc7ccccc7c56)n4)ccc31)c2-c1ccccc1 ; CC1(C)c2ccccc2-c2c1cc1c(ccc3cc(-c4nc(-c5ccccc5)nc(-c5cccc6oc7ccccc7c56)n4)ccc31)c2-c1ccc2ccccc2c1]

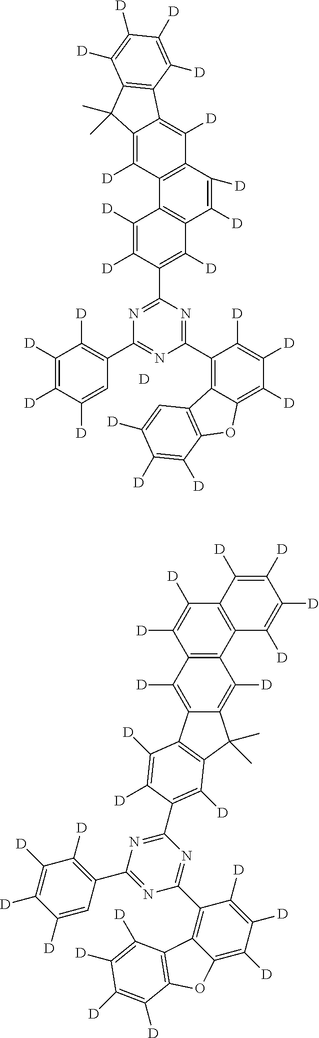

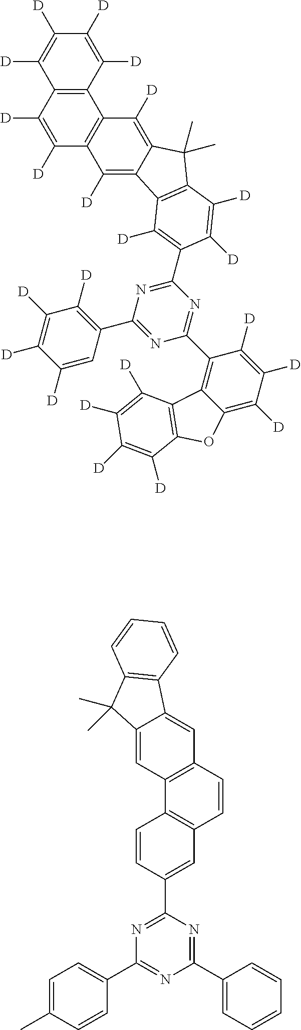 [SMILES: [2H]c1cc(-c2nc(-c3c([2H])c([2H])c4c(c3[2H])-c3c(c([2H])c5c(c3[2H])c([2H])c([2H])c3c([2H])c([2H])c([2H])c([2H])c35)C4(C)C)nc(-c3c([2H])c([2H])c([2H])c4oc5c([2H])c([2H])c([2H])c([2H])c5c34)n2)c([2H])c([2H])c1[2H] ; Cc1ccc(-c2nc(-c3ccccc3)nc(-c3ccc4c(ccc5cc6c(cc54)C(C)(C)c4ccccc4-6)c3)n2)cc1]

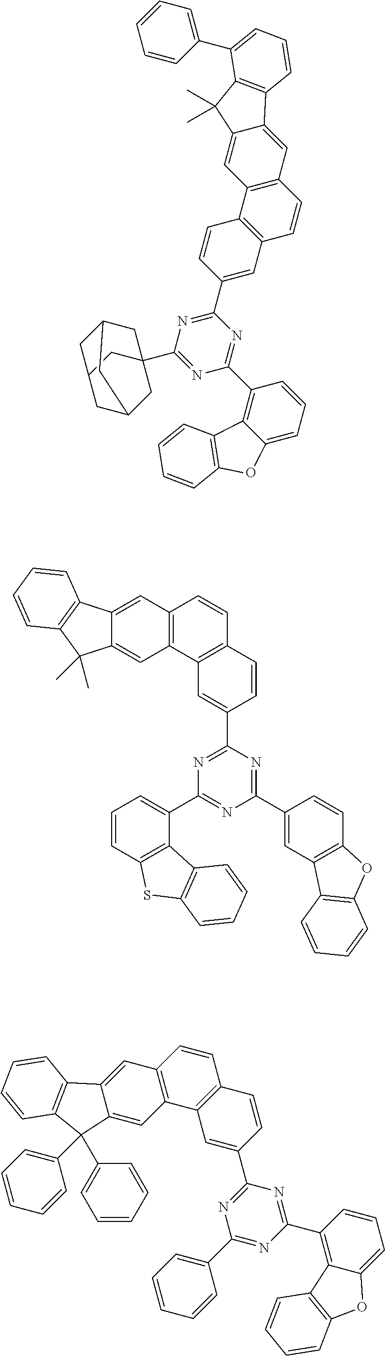 [SMILES: CC1(C)c2cc3c(ccc4cc(-c5nc(-c6cccc7oc8ccccc8c67)nc(C67CC8CC(CC(C8)C6)C7)n5)ccc43)cc2-c2cccc(-c3ccccc3)c21 ; CC1(C)c2ccccc2-c2cc3ccc4ccc(-c5nc(-c6ccc7oc8ccccc8c7c6)nc(-c6cccc7sc8ccccc8c67)n5)cc4c3cc21 ; c1ccc(-c2nc(-c3ccc4ccc5cc6c(cc5c4c3)C(c3ccccc3)(c3ccccc3)c3ccccc3-6)nc(-c3cccc4oc5ccccc5c34)n2)cc1]

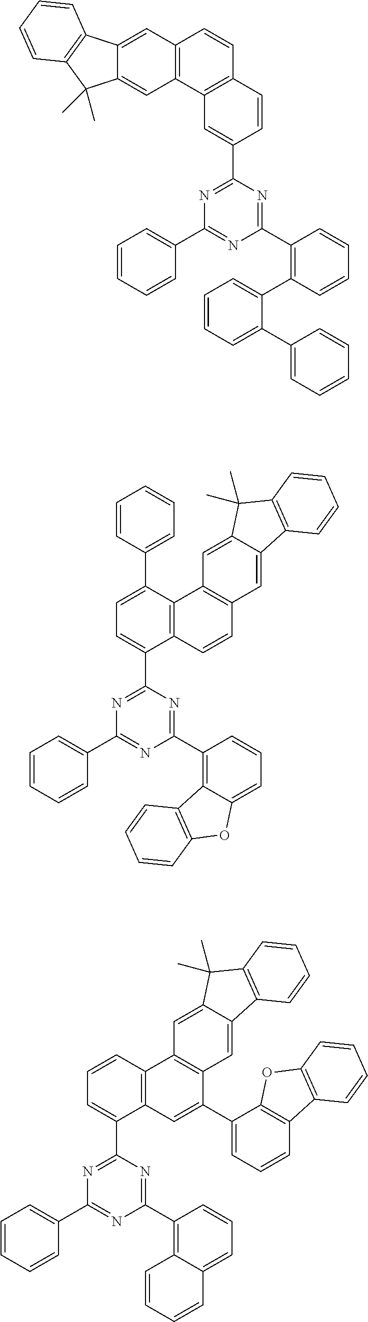 [SMILES: CC1(C)c2ccccc2-c2cc3ccc4ccc(-c5nc(-c6ccccc6)nc(-c6ccccc6-c6ccccc6-c6ccccc6)n5)cc4c3cc21 ; CC1(C)c2ccccc2-c2cc3ccc4c(-c5nc(-c6ccccc6)nc(-c6cccc7oc8ccccc8c67)n5)ccc(-c5ccccc5)c4c3cc21 ; CC1(C)c2ccccc2-c2cc3c(-c4cccc5c4oc4ccccc45)cc4c(-c5nc(-c6ccccc6)nc(-c6cccc7ccccc67)n5)cccc4c3cc21]

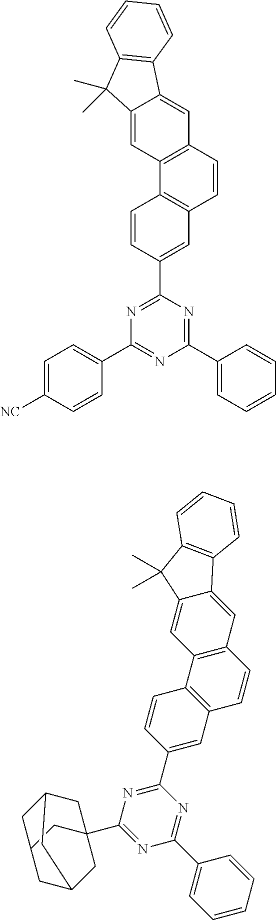 [SMILES: CC1(C)c2ccccc2-c2cc3ccc4cc(-c5nc(-c6ccccc6)nc(-c6ccc(C#N)cc6)n5)ccc4c3cc21 ; CC1(C)c2ccccc2-c2cc3ccc4cc(-c5nc(-c6ccccc6)nc(C67CC8CC(CC(C8)C6)C7)n5)ccc4c3cc21]

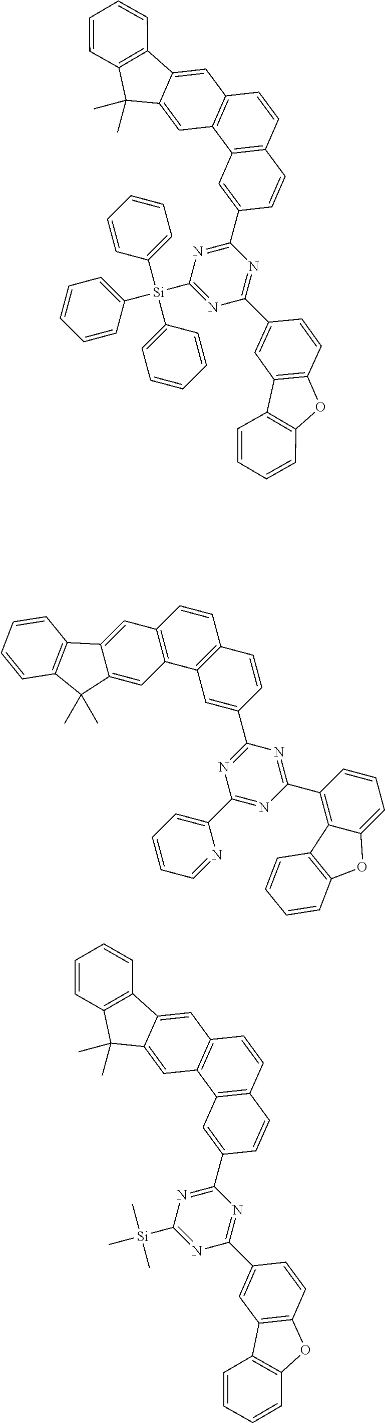 [SMILES: CC1(C)c2ccccc2-c2cc3ccc4ccc(-c5nc(-c6ccc7oc8ccccc8c7c6)nc([Si](c6ccccc6)(c6ccccc6)c6ccccc6)n5)cc4c3cc21 ; CC1(C)c2ccccc2-c2cc3ccc4ccc(-c5nc(-c6ccccn6)nc(-c6cccc7oc8ccccc8c67)n5)cc4c3cc21 ; CC1(C)c2ccccc2-c2cc3ccc4ccc(-c5nc(-c6ccc7oc8ccccc8c7c6)nc([Si](C)(C)C)n5)cc4c3cc21]

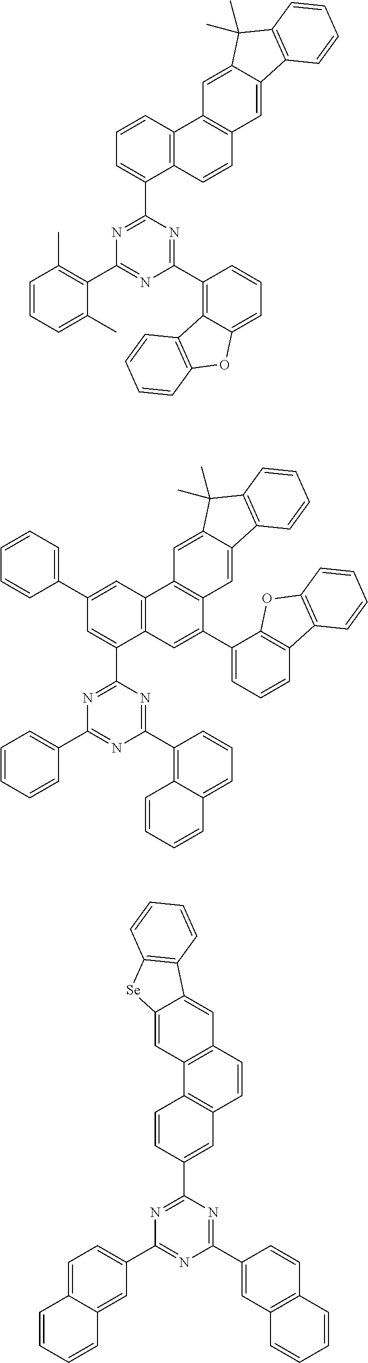 [SMILES: Cc1cccc(C)c1-c1nc(-c2cccc3c2ccc2cc4c(cc23)C(C)(C)c2ccccc2-4)nc(-c2cccc3oc4ccccc4c23)n1 ; CC1(C)c2ccccc2-c2cc3c(-c4cccc5c4oc4ccccc45)cc4c(-c5nc(-c6ccccc6)nc(-c6cccc7ccccc67)n5)cc(-c5ccccc5)cc4c3cc21 ; c1ccc2cc(-c3nc(-c4ccc5ccccc5c4)nc(-c4ccc5c(ccc6cc7c(cc65)[se]c5ccccc57)c4)n3)ccc2c1]

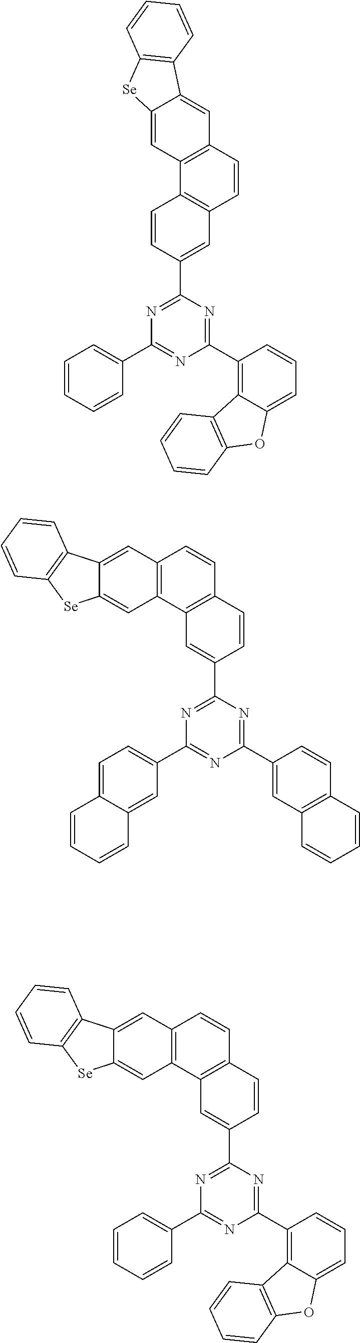 [SMILES: c1ccc(-c2nc(-c3ccc4c(ccc5cc6c(cc54)[se]c4ccccc46)c3)nc(-c3cccc4oc5ccccc5c34)n2)cc1 ; c1ccc2cc(-c3nc(-c4ccc5ccccc5c4)nc(-c4ccc5ccc6cc7c(cc6c5c4)[se]c4ccccc47)n3)ccc2c1 ; c1ccc(-c2nc(-c3ccc4ccc5cc6c(cc5c4c3)[se]c3ccccc36)nc(-c3cccc4oc5ccccc5c34)n2)cc1]

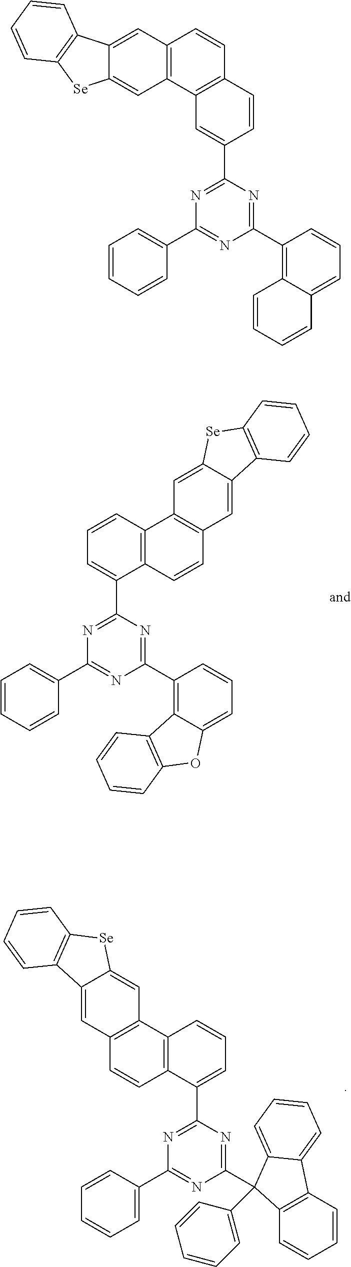 [SMILES: c1ccc(-c2nc(-c3ccc4ccc5cc6c(cc5c4c3)[se]c3ccccc36)nc(-c3cccc4ccccc34)n2)cc1 ; c1ccc(-c2nc(-c3cccc4c3ccc3cc5c(cc34)[se]c3ccccc35)nc(-c3cccc4oc5ccccc5c34)n2)cc1 ; c1ccc(-c2nc(-c3cccc4c3ccc3cc5c(cc34)[se]c3ccccc35)nc(C3(c4ccccc4)c4ccccc4-c4ccccc43)n2)cc1]

6. An organic electroluminescent material comprising the organic electroluminescent compound according to claim 1.

7. The organic electroluminescent material according to claim 6, wherein said organic electroluminescent material is a plurality of host materials, comprising the organic electroluminescent compound as a first host and a compound represented by the following formula 2 as a second host: 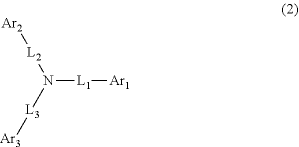
wherein,
L₁ to L3 each independently represent a single bond, a substituted or unsubstituted (C1-C30)alkylene, a substituted or unsubstituted (C6-C30)arylene, a substituted or unsubstituted (3- to 30-membered)heteroarylene, or a substituted or unsubstituted C3-C30)cycloalkylene;
Ar₁ to Ar₃ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of (C3-C30) aliphatic ring and (C6-C30) aromatic ring, or -Lₐ-N(Arₐ)(Ar_{b});
Lₐ each independently represents a single bond, a substituted or unsubstituted (C6-C30)arylene, or a substituted or unsubstituted (3- to 30-membered)heteroarylene;
Arₐ and Ar_{b} each independently represent hydrogen, a substituted or unsubstituted (C1-C30)alkyl, a substituted or unsubstituted (C2-C30)alkenyl, a substituted or unsubstituted fused ring of (C3-C30) aliphatic ring and (C6-C30) aromatic ring, a substituted or unsubstituted (C6-C30)aryl, or a substituted or unsubstituted (3- to 30-membered)heteroaryl; and
provided that the compound where all of L₁ to L3 are a single bond and all of Ar₁ to Ar₃ are hydrogen is excluded.

8. The organic electroluminescent material according to claim 7, wherein the formula 2 is represented by any one of the following formulas 2-1 to 2-12: 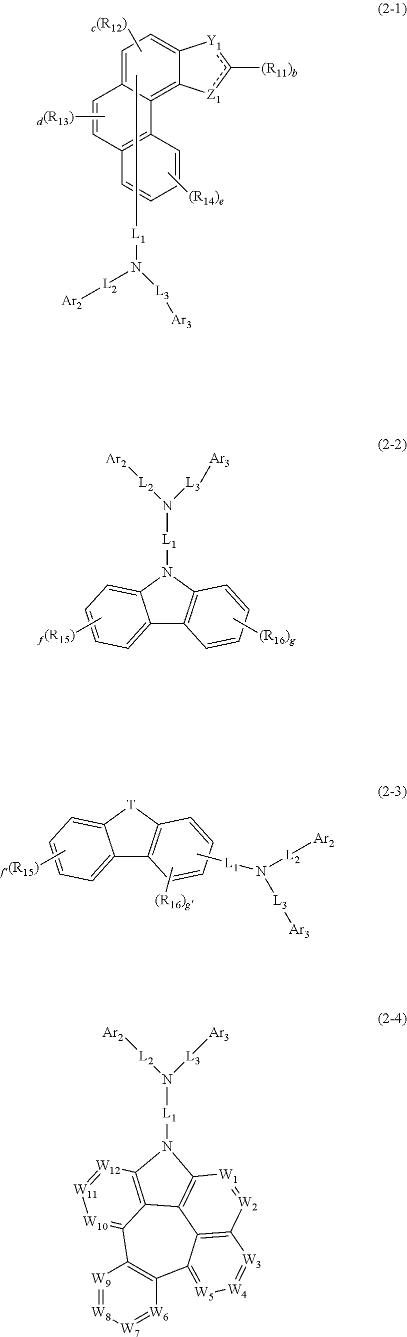 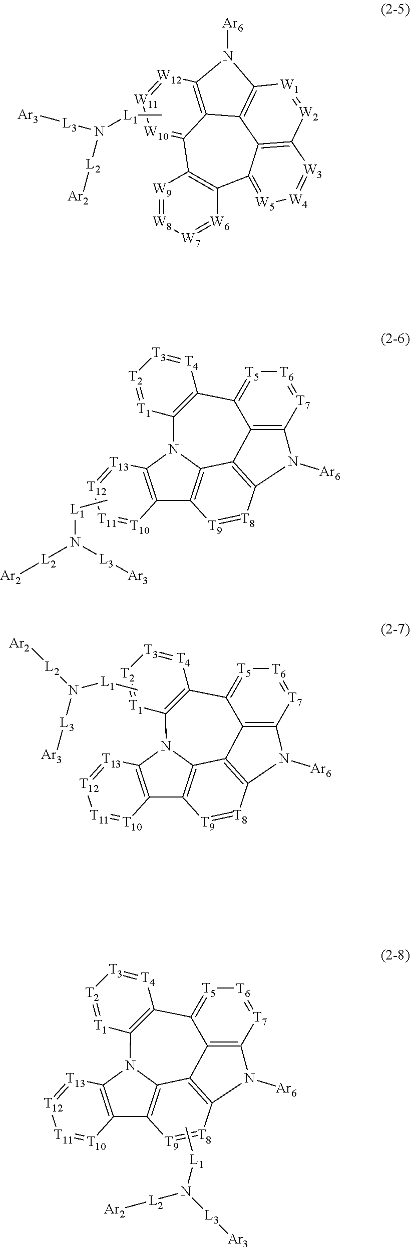 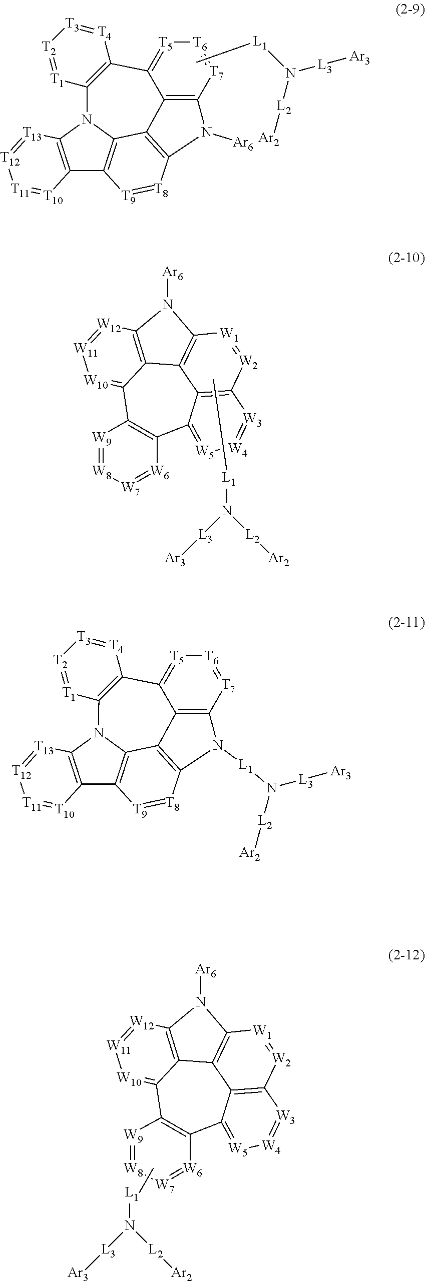
wherein,
Y₁ and Z₁ each independently represent —N═, —N(R₂₁)-, —O—, or —S—, with a proviso that any one of Y₁ and Z₁ represents —N═ and the other represents —N(R₂₁)-, —O—, or —S—;
T represents C(R₂₂)(R₂₃), N(R₂₄), O, or S;
T₁ to T₁₃ and W₁ to W₁₂ each independently represent N or C(V₁);
R₁₁ represents a substituted or unsubstituted (C6-C30)aryl, or substituted or unsubstituted (3- to 30-membered)heteroaryl;
R₁₂ to R₁₆, R₂₁ to R₂₄, and V₁ each independently represent hydrogen, deuterium, halogen, cyano, a substituted or unsubstituted (C1-C30)alkyl, a substituted or unsubstituted (C6-C30)aryl, a substituted or unsubstituted (3- to 30-membered)heteroaryl, a substituted or unsubstituted (C3-C30)cycloalkyl, a substituted or unsubstituted (C1-C30)alkoxy, a substituted or unsubstituted tri(C1-C30)alkylsilyl, a substituted or unsubstituted di(C1-C30)alkyl(C6-C30)arylsilyl, a substituted or unsubstituted (C1-C30)alkyldi(C6-C30)arylsilyl, a substituted or unsubstituted tri(C6-C30)arylsilyl, a substituted or unsubstituted fused ring of a (C3-C30) aliphatic ring and a (C6-C30) aromatic ring, or -L_{c}-N(Arₑ)(Ar_{f}); or may be linked to an adjacent substituent(s) to form a ring;
L_{c} each independently represents single bond, a substituted or unsubstituted (C6-C30)arylene, or substituted or unsubstituted (3- to 30-membered)heteroarylene;
Arₑ and Ar_{f} each independently represent hydrogen, a substituted or unsubstituted (C1-C30)alkyl, a substituted or unsubstituted (C2-C30)alkenyl, a fused ring of substituted or unsubstituted (C3-C30) aliphatic rings and (C6-C30) aromatic rings, a substituted or unsubstituted (C6-C30)aryl, or a substituted or unsubstituted (3- to 30-membered)heteroaryl;
Ar₆ represents a substituted or unsubstituted (C6-C30)aryl or a substituted or unsubstituted (3- to 30-membered)heteroaryl;
b represents 1, c and d each independently represent 1 or 2, e, f, g and f′ each independently represent an integer of 1 to 4, and g′ represents an integer of 1 to 3, when c to g, f′ and g′ are 2 or more, each of R₁₂ to R₁₆ may be the same or different from each other; and
Arₑ, Arₐ, and L₁ to L₃ are as defined in claim 7.

9. The organic electroluminescent material according to claim 7, wherein the compound represented by formula 2 is at least one selected from the group consisting of the following compounds:

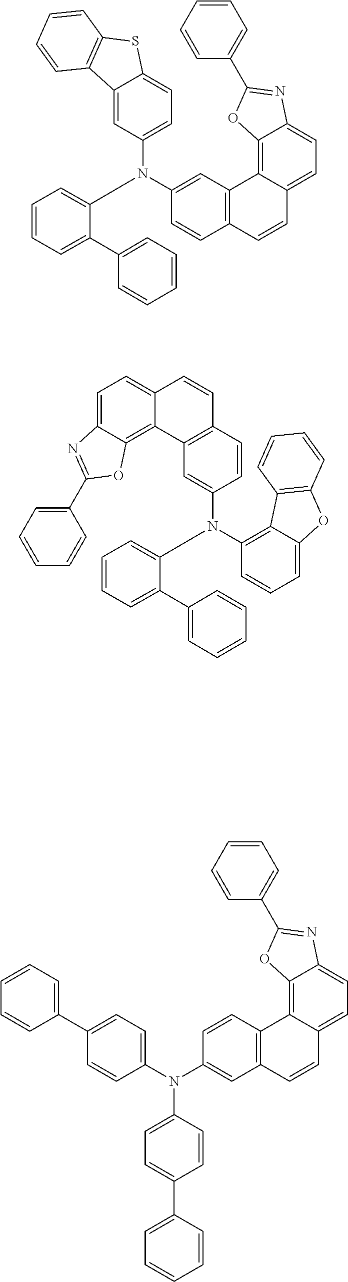 [SMILES: c1ccc(-c2nc3ccc4ccc5ccc(N(c6ccc7sc8ccccc8c7c6)c6ccccc6-c6ccccc6)cc5c4c3o2)cc1 ; c1ccc(-c2nc3ccc4ccc5ccc(N(c6ccccc6-c6ccccc6)c6cccc7oc8ccccc8c67)cc5c4c3o2)cc1 ; c1ccc(-c2ccc(N(c3ccc(-c4ccccc4)cc3)c3ccc4c(ccc5ccc6nc(-c7ccccc7)oc6c54)c3)cc2)cc1]

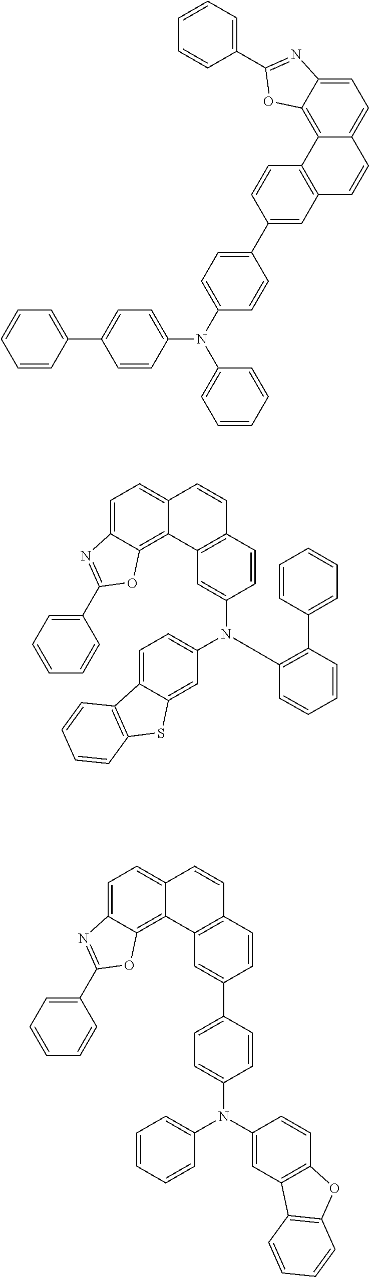 [SMILES: c1ccc(-c2ccc(N(c3ccccc3)c3ccc(-c4ccc5c(ccc6ccc7nc(-c8ccccc8)oc7c65)c4)cc3)cc2)cc1 ; c1ccc(-c2nc3ccc4ccc5ccc(N(c6ccc7c(c6)sc6ccccc67)c6ccccc6-c6ccccc6)cc5c4c3o2)cc1 ; c1ccc(-c2nc3ccc4ccc5ccc(-c6ccc(N(c7ccccc7)c7ccc8oc9ccccc9c8c7)cc6)cc5c4c3o2)cc1]

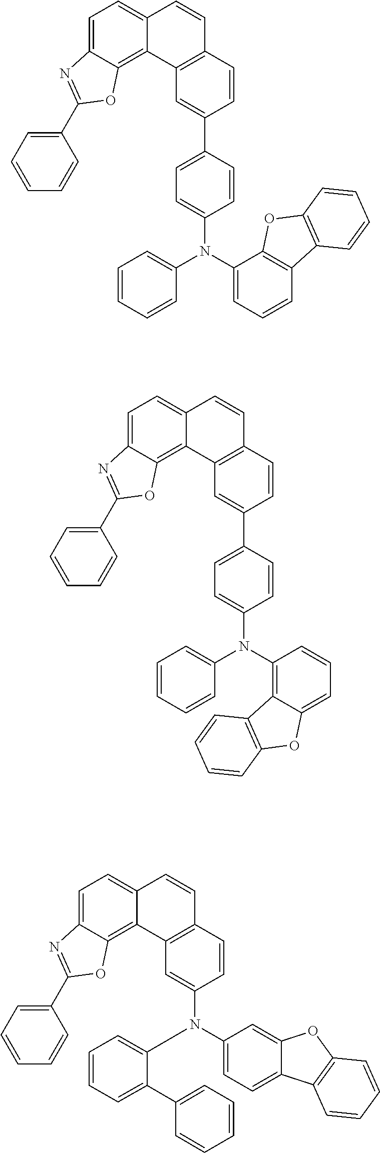 [SMILES: c1ccc(-c2nc3ccc4ccc5ccc(-c6ccc(N(c7ccccc7)c7cccc8c7oc7ccccc78)cc6)cc5c4c3o2)cc1 ; c1ccc(-c2nc3ccc4ccc5ccc(-c6ccc(N(c7ccccc7)c7cccc8oc9ccccc9c78)cc6)cc5c4c3o2)cc1 ; c1ccc(-c2nc3ccc4ccc5ccc(N(c6ccc7c(c6)oc6ccccc67)c6ccccc6-c6ccccc6)cc5c4c3o2)cc1]

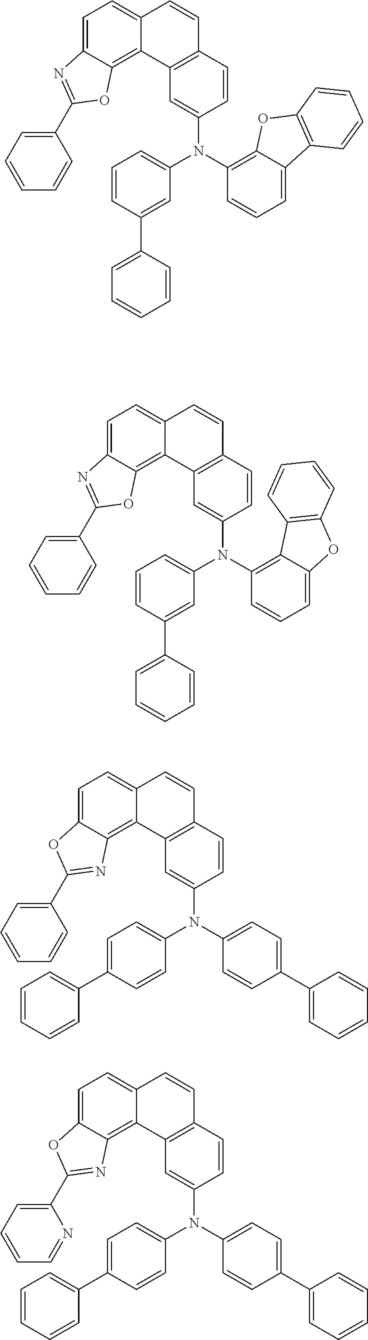 [SMILES: c1ccc(-c2cccc(N(c3ccc4ccc5ccc6nc(-c7ccccc7)oc6c5c4c3)c3cccc4c3oc3ccccc34)c2)cc1 ; c1ccc(-c2cccc(N(c3ccc4ccc5ccc6nc(-c7ccccc7)oc6c5c4c3)c3cccc4oc5ccccc5c34)c2)cc1 ; c1ccc(-c2ccc(N(c3ccc(-c4ccccc4)cc3)c3ccc4ccc5ccc6oc(-c7ccccc7)nc6c5c4c3)cc2)cc1 ; c1ccc(-c2ccc(N(c3ccc(-c4ccccc4)cc3)c3ccc4ccc5ccc6oc(-c7ccccn7)nc6c5c4c3)cc2)cc1]

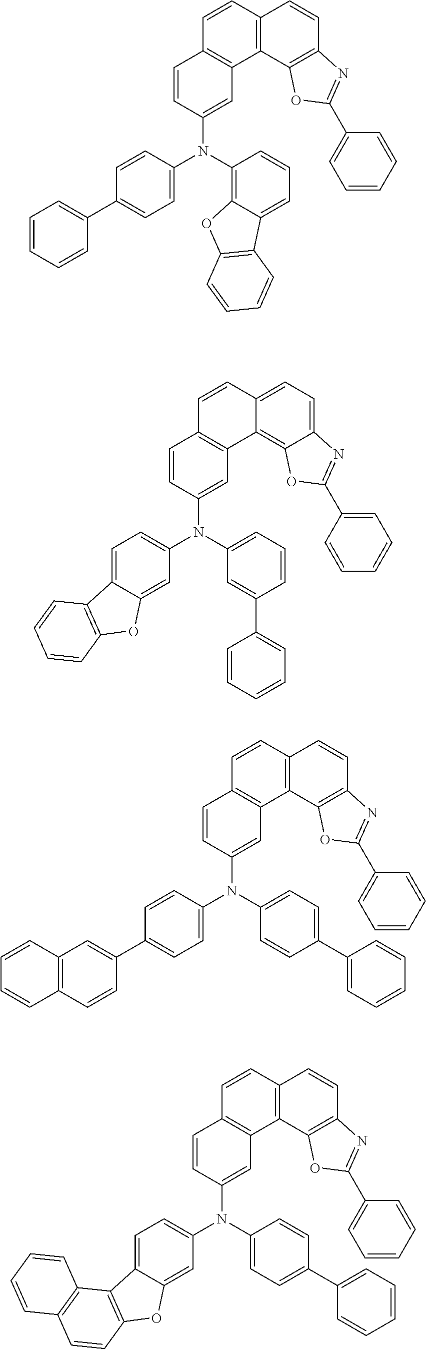

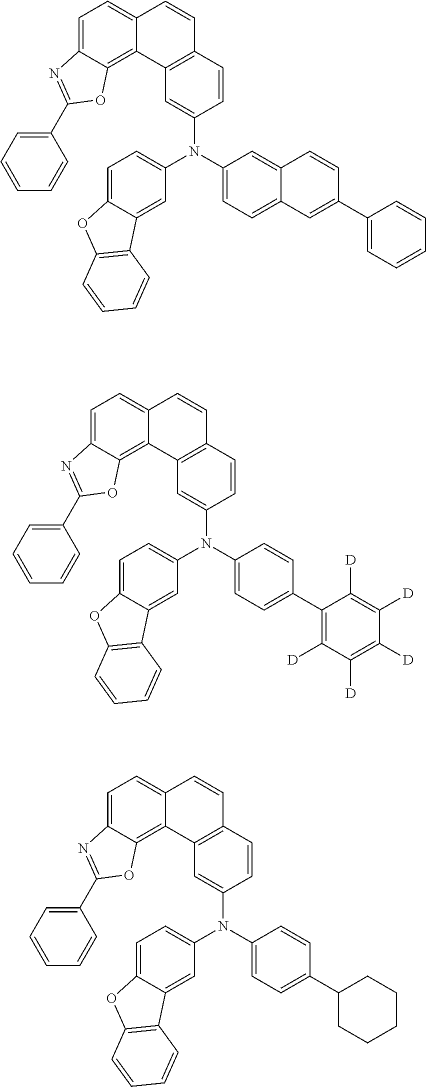 [SMILES: c1ccc(-c2ccc3cc(N(c4ccc5oc6ccccc6c5c4)c4ccc5ccc6ccc7nc(-c8ccccc8)oc7c6c5c4)ccc3c2)cc1 ; [2H]c1c([2H])c([2H])c(-c2ccc(N(c3ccc4oc5ccccc5c4c3)c3ccc4ccc5ccc6nc(-c7ccccc7)oc6c5c4c3)cc2)c([2H])c1[2H] ; c1ccc(-c2nc3ccc4ccc5ccc(N(c6ccc(C7CCCCC7)cc6)c6ccc7oc8ccccc8c7c6)cc5c4c3o2)cc1]

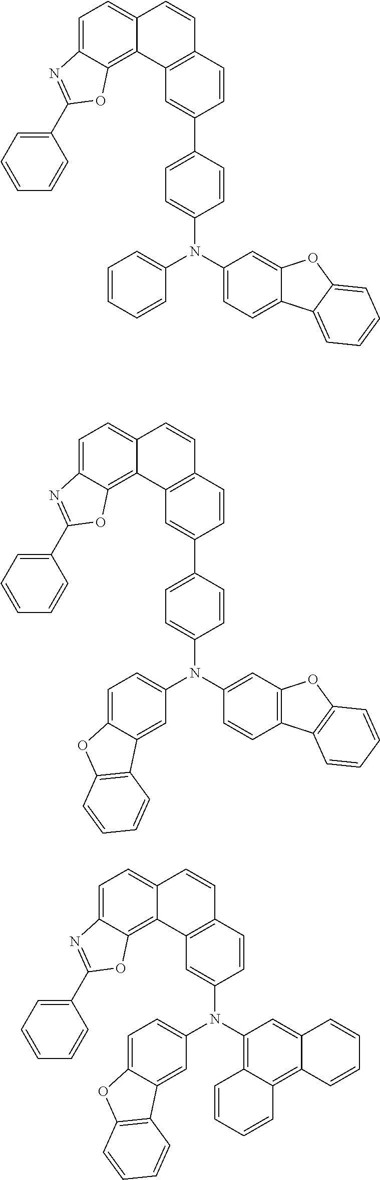

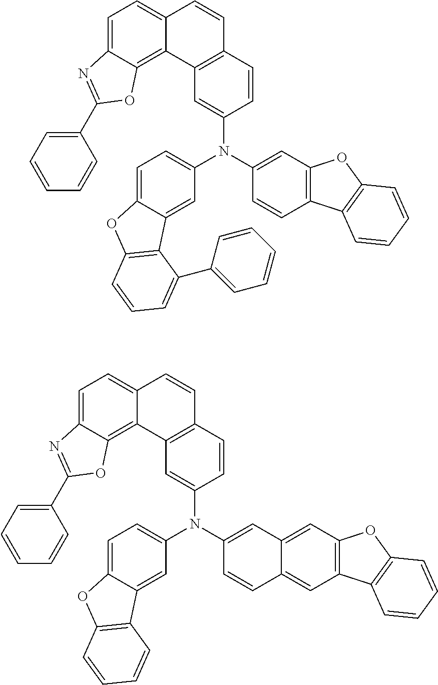 [SMILES: c1ccc(-c2nc3ccc4ccc5ccc(N(c6ccc7c(c6)oc6ccccc67)c6ccc7oc8cccc(-c9ccccc9)c8c7c6)cc5c4c3o2)cc1 ; c1ccc(-c2nc3ccc4ccc5ccc(N(c6ccc7cc8c(cc7c6)oc6ccccc68)c6ccc7oc8ccccc8c7c6)cc5c4c3o2)cc1]

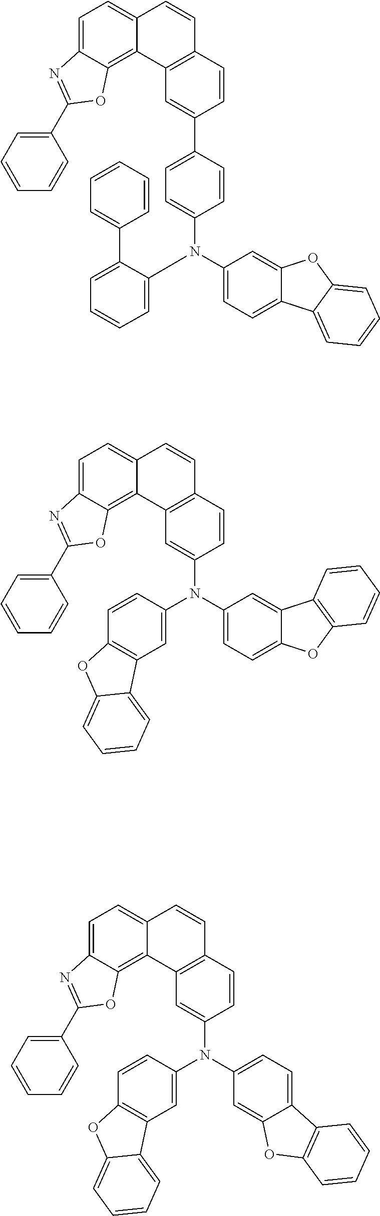 [SMILES: c1ccc(-c2nc3ccc4ccc5ccc(-c6ccc(N(c7ccc8c(c7)oc7ccccc78)c7ccccc7-c7ccccc7)cc6)cc5c4c3o2)cc1 ; c1ccc(-c2nc3ccc4ccc5ccc(N(c6ccc7oc8ccccc8c7c6)c6ccc7oc8ccccc8c7c6)cc5c4c3o2)cc1 ; c1ccc(-c2nc3ccc4ccc5ccc(N(c6ccc7c(c6)oc6ccccc67)c6ccc7oc8ccccc8c7c6)cc5c4c3o2)cc1]

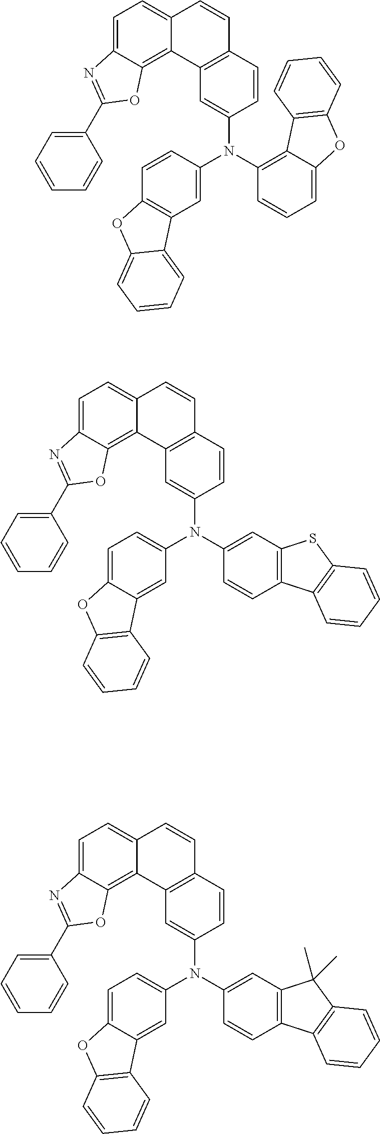 [SMILES: c1ccc(-c2nc3ccc4ccc5ccc(N(c6ccc7oc8ccccc8c7c6)c6cccc7oc8ccccc8c67)cc5c4c3o2)cc1 ; c1ccc(-c2nc3ccc4ccc5ccc(N(c6ccc7c(c6)sc6ccccc67)c6ccc7oc8ccccc8c7c6)cc5c4c3o2)cc1 ; CC1(C)c2ccccc2-c2ccc(N(c3ccc4oc5ccccc5c4c3)c3ccc4ccc5ccc6nc(-c7ccccc7)oc6c5c4c3)cc21]

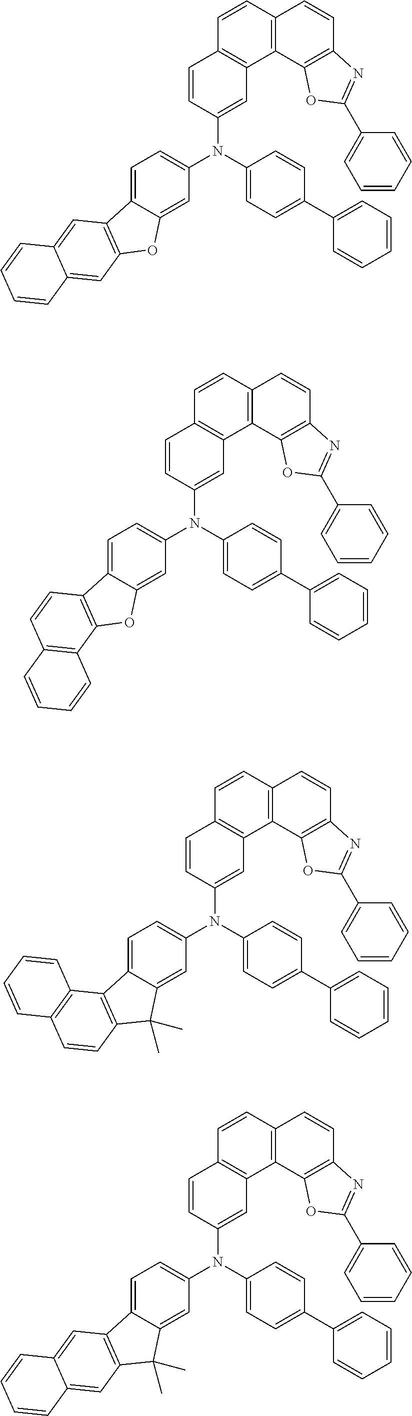 [SMILES: c1ccc(-c2ccc(N(c3ccc4c(c3)oc3cc5ccccc5cc34)c3ccc4ccc5ccc6nc(-c7ccccc7)oc6c5c4c3)cc2)cc1 ; c1ccc(-c2ccc(N(c3ccc4c(c3)oc3c5ccccc5ccc43)c3ccc4ccc5ccc6nc(-c7ccccc7)oc6c5c4c3)cc2)cc1 ; CC1(C)c2cc(N(c3ccc(-c4ccccc4)cc3)c3ccc4ccc5ccc6nc(-c7ccccc7)oc6c5c4c3)ccc2-c2c1ccc1ccccc21 ; CC1(C)c2cc(N(c3ccc(-c4ccccc4)cc3)c3ccc4ccc5ccc6nc(-c7ccccc7)oc6c5c4c3)ccc2-c2cc3ccccc3cc21]

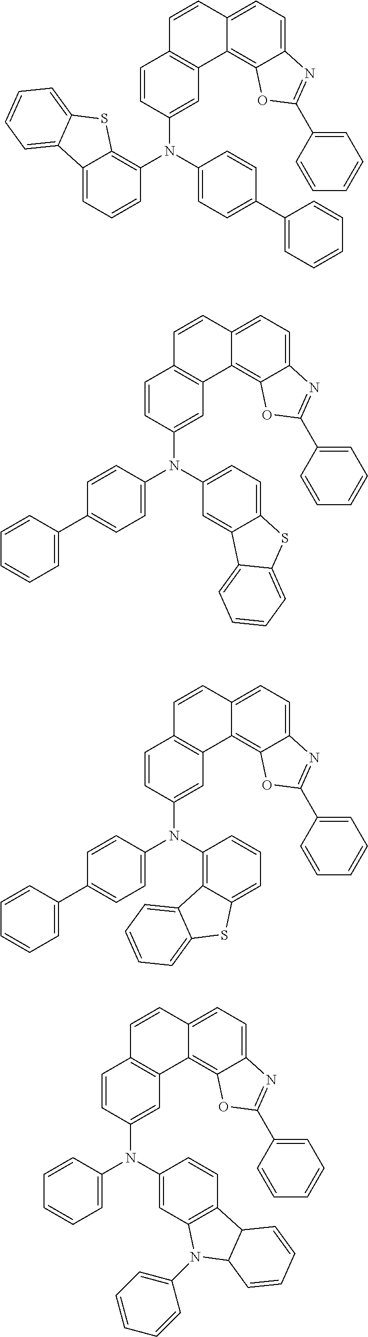

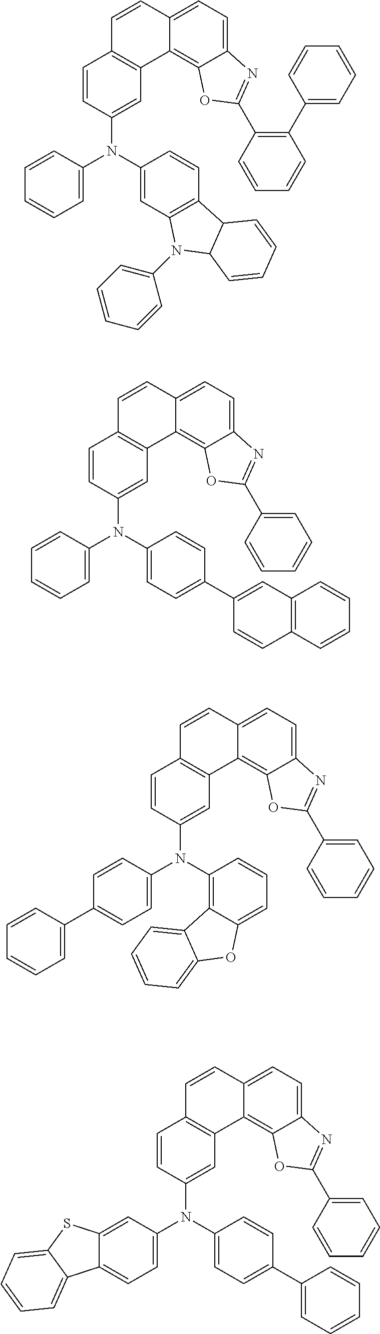 [SMILES: C1=CC2c3ccc(N(c4ccccc4)c4ccc5ccc6ccc7nc(-c8ccccc8-c8ccccc8)oc7c6c5c4)cc3N(c3ccccc3)C2C=C1 ; c1ccc(-c2nc3ccc4ccc5ccc(N(c6ccccc6)c6ccc(-c7ccc8ccccc8c7)cc6)cc5c4c3o2)cc1 ; c1ccc(-c2ccc(N(c3ccc4ccc5ccc6nc(-c7ccccc7)oc6c5c4c3)c3cccc4oc5ccccc5c34)cc2)cc1 ; c1ccc(-c2ccc(N(c3ccc4c(c3)sc3ccccc34)c3ccc4ccc5ccc6nc(-c7ccccc7)oc6c5c4c3)cc2)cc1]

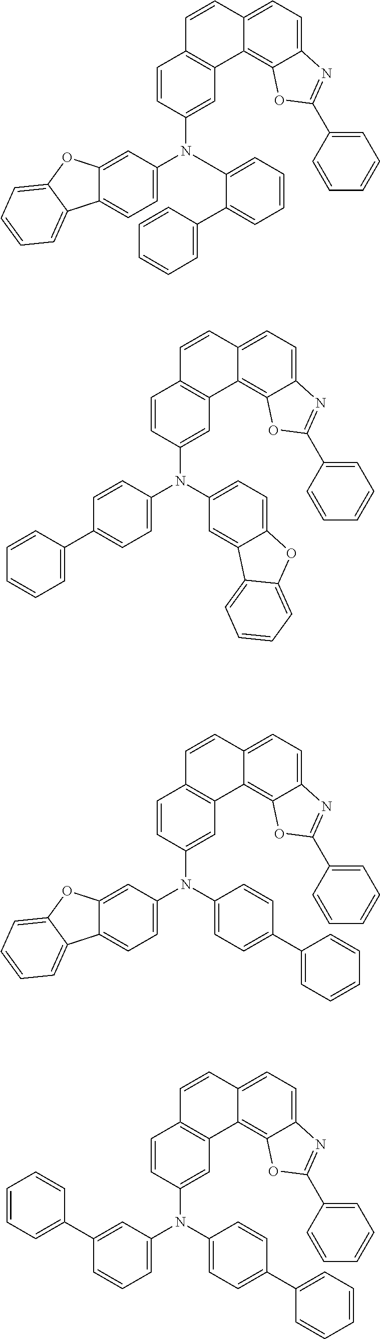 [SMILES: c1ccc(-c2nc3ccc4ccc5ccc(N(c6ccc7c(c6)oc6ccccc67)c6ccccc6-c6ccccc6)cc5c4c3o2)cc1 ; c1ccc(-c2ccc(N(c3ccc4oc5ccccc5c4c3)c3ccc4ccc5ccc6nc(-c7ccccc7)oc6c5c4c3)cc2)cc1 ; c1ccc(-c2ccc(N(c3ccc4c(c3)oc3ccccc34)c3ccc4ccc5ccc6nc(-c7ccccc7)oc6c5c4c3)cc2)cc1 ; c1ccc(-c2ccc(N(c3cccc(-c4ccccc4)c3)c3ccc4ccc5ccc6nc(-c7ccccc7)oc6c5c4c3)cc2)cc1]

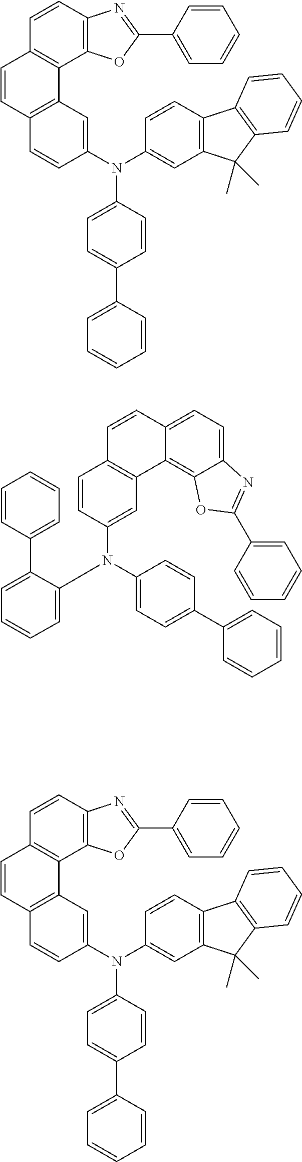 [SMILES: CC1(C)c2ccccc2-c2ccc(N(c3ccc(-c4ccccc4)cc3)c3ccc4ccc5ccc6nc(-c7ccccc7)oc6c5c4c3)cc21 ; c1ccc(-c2ccc(N(c3ccc4ccc5ccc6nc(-c7ccccc7)oc6c5c4c3)c3ccccc3-c3ccccc3)cc2)cc1 ; CC1(C)c2ccccc2-c2ccc(N(c3ccc(-c4ccccc4)cc3)c3ccc4ccc5ccc6nc(-c7ccccc7)oc6c5c4c3)cc21]

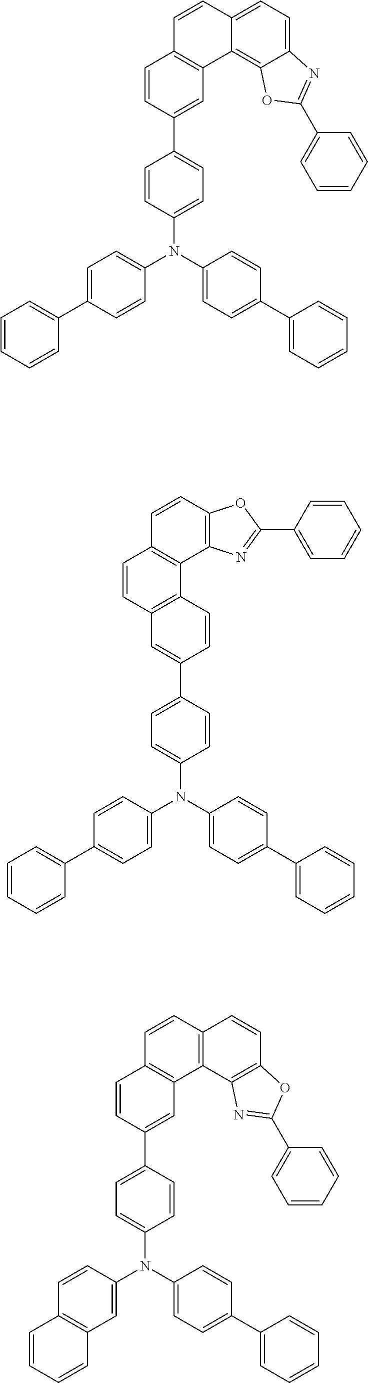 [SMILES: c1ccc(-c2ccc(N(c3ccc(-c4ccccc4)cc3)c3ccc(-c4ccc5ccc6ccc7nc(-c8ccccc8)oc7c6c5c4)cc3)cc2)cc1 ; c1ccc(-c2ccc(N(c3ccc(-c4ccccc4)cc3)c3ccc(-c4ccc5c(ccc6ccc7oc(-c8ccccc8)nc7c65)c4)cc3)cc2)cc1 ; c1ccc(-c2ccc(N(c3ccc(-c4ccc5ccc6ccc7oc(-c8ccccc8)nc7c6c5c4)cc3)c3ccc4ccccc4c3)cc2)cc1]

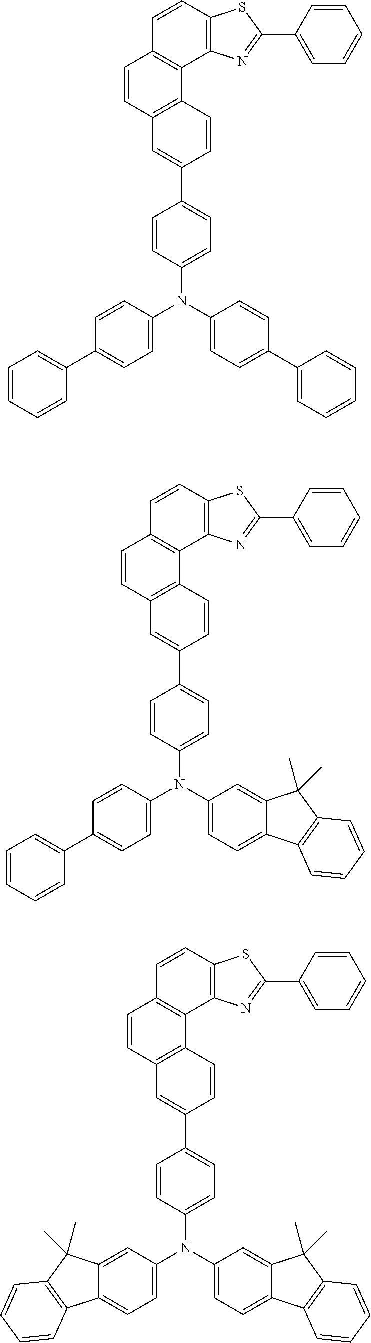 [SMILES: c1ccc(-c2ccc(N(c3ccc(-c4ccccc4)cc3)c3ccc(-c4ccc5c(ccc6ccc7sc(-c8ccccc8)nc7c65)c4)cc3)cc2)cc1 ; CC1(C)c2ccccc2-c2ccc(N(c3ccc(-c4ccccc4)cc3)c3ccc(-c4ccc5c(ccc6ccc7sc(-c8ccccc8)nc7c65)c4)cc3)cc21 ; CC1(C)c2ccccc2-c2ccc(N(c3ccc(-c4ccc5c(ccc6ccc7sc(-c8ccccc8)nc7c65)c4)cc3)c3ccc4c(c3)C(C)(C)c3ccccc3-4)cc21]

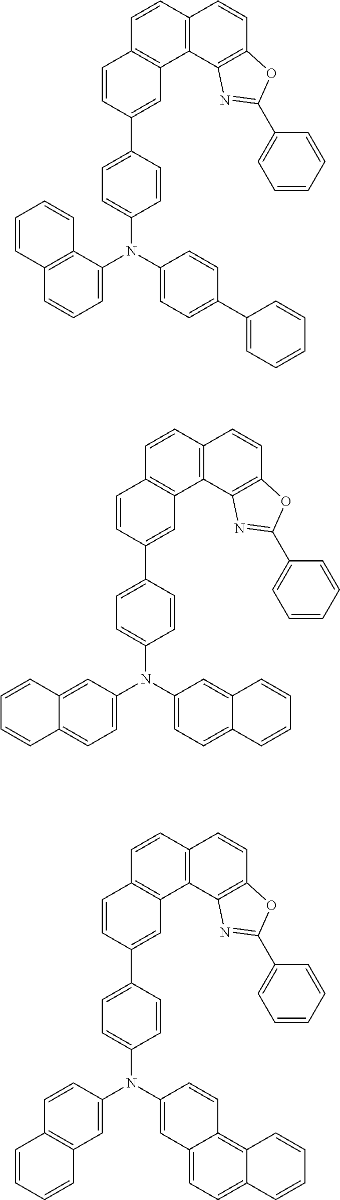 [SMILES: c1ccc(-c2ccc(N(c3ccc(-c4ccc5ccc6ccc7oc(-c8ccccc8)nc7c6c5c4)cc3)c3cccc4ccccc34)cc2)cc1 ; c1ccc(-c2nc3c(ccc4ccc5ccc(-c6ccc(N(c7ccc8ccccc8c7)c7ccc8ccccc8c7)cc6)cc5c43)o2)cc1 ; c1ccc(-c2nc3c(ccc4ccc5ccc(-c6ccc(N(c7ccc8ccccc8c7)c7ccc8c(ccc9ccccc98)c7)cc6)cc5c43)o2)cc1]

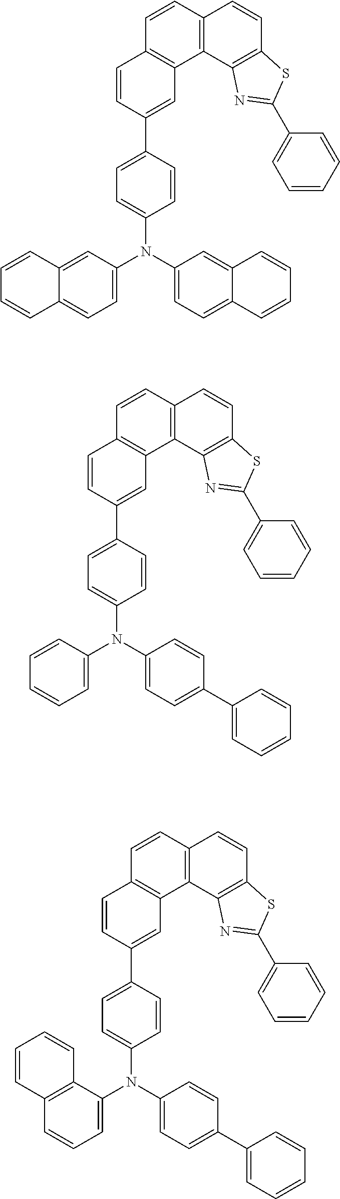 [SMILES: c1ccc(-c2nc3c(ccc4ccc5ccc(-c6ccc(N(c7ccc8ccccc8c7)c7ccc8ccccc8c7)cc6)cc5c43)s2)cc1 ; c1ccc(-c2ccc(N(c3ccccc3)c3ccc(-c4ccc5ccc6ccc7sc(-c8ccccc8)nc7c6c5c4)cc3)cc2)cc1 ; c1ccc(-c2ccc(N(c3ccc(-c4ccc5ccc6ccc7sc(-c8ccccc8)nc7c6c5c4)cc3)c3cccc4ccccc34)cc2)cc1]

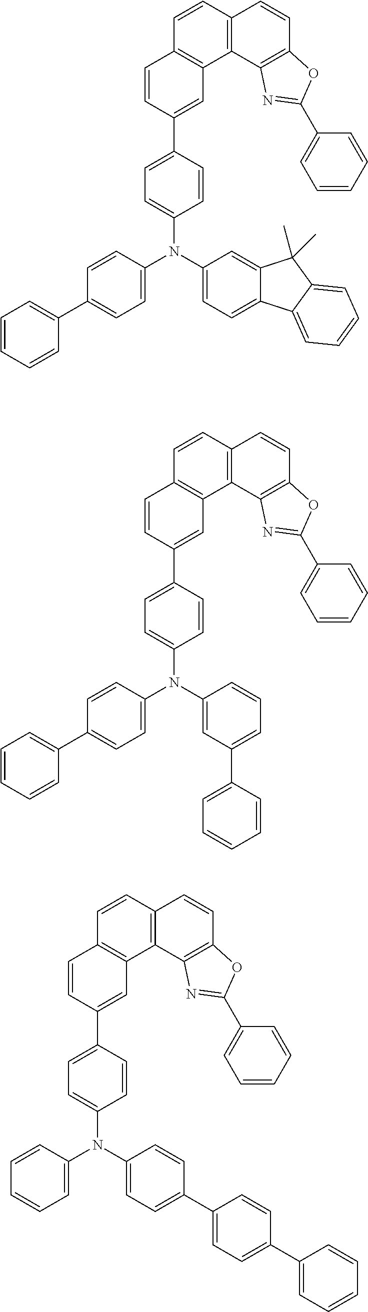 [SMILES: CC1(C)c2ccccc2-c2ccc(N(c3ccc(-c4ccccc4)cc3)c3ccc(-c4ccc5ccc6ccc7oc(-c8ccccc8)nc7c6c5c4)cc3)cc21 ; c1ccc(-c2ccc(N(c3ccc(-c4ccc5ccc6ccc7oc(-c8ccccc8)nc7c6c5c4)cc3)c3cccc(-c4ccccc4)c3)cc2)cc1 ; c1ccc(-c2ccc(-c3ccc(N(c4ccccc4)c4ccc(-c5ccc6ccc7ccc8oc(-c9ccccc9)nc8c7c6c5)cc4)cc3)cc2)cc1]

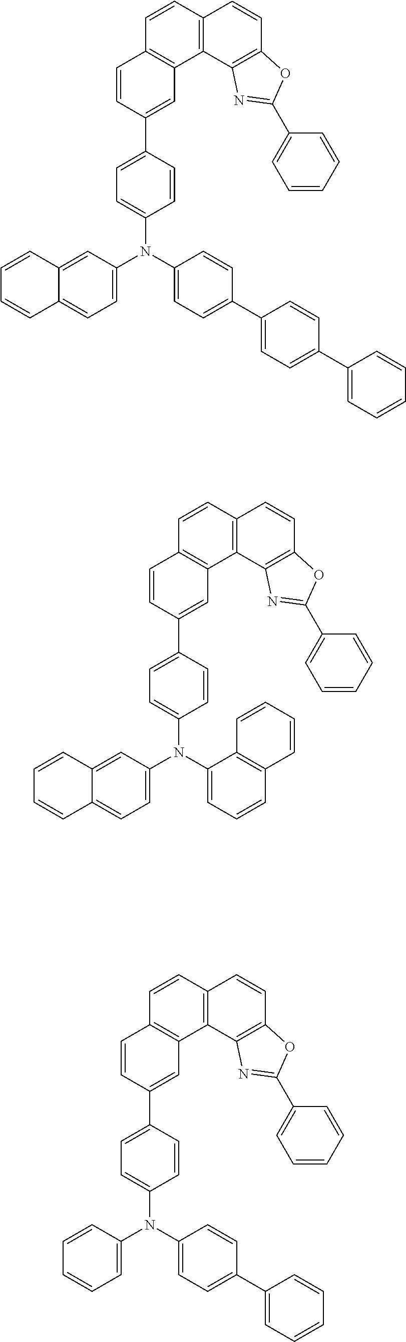 [SMILES: c1ccc(-c2ccc(-c3ccc(N(c4ccc(-c5ccc6ccc7ccc8oc(-c9ccccc9)nc8c7c6c5)cc4)c4ccc5ccccc5c4)cc3)cc2)cc1 ; c1ccc(-c2nc3c(ccc4ccc5ccc(-c6ccc(N(c7ccc8ccccc8c7)c7cccc8ccccc78)cc6)cc5c43)o2)cc1 ; c1ccc(-c2ccc(N(c3ccccc3)c3ccc(-c4ccc5ccc6ccc7oc(-c8ccccc8)nc7c6c5c4)cc3)cc2)cc1]

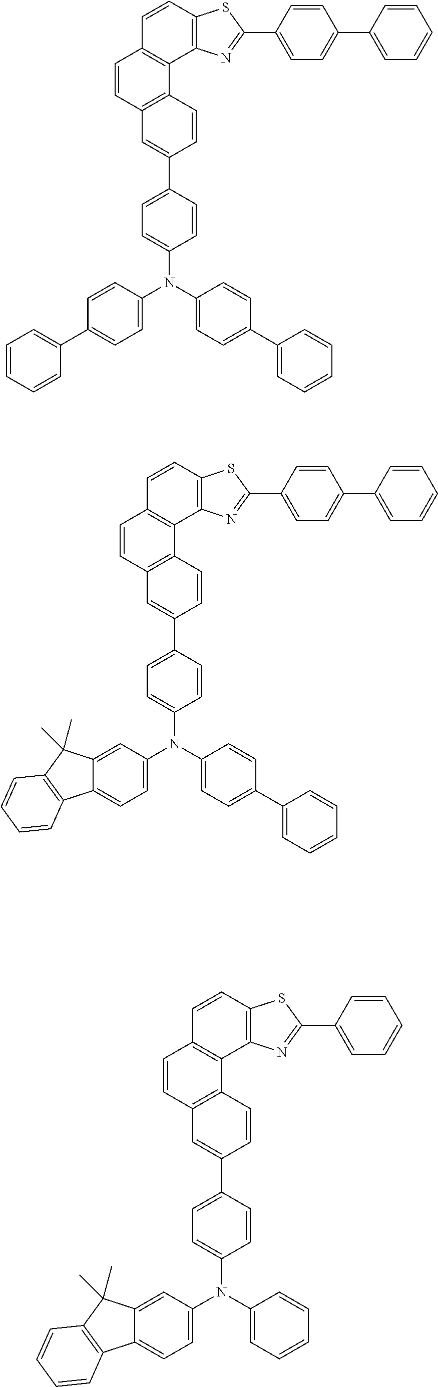 [SMILES: c1ccc(-c2ccc(-c3nc4c(ccc5ccc6cc(-c7ccc(N(c8ccc(-c9ccccc9)cc8)c8ccc(-c9ccccc9)cc8)cc7)ccc6c54)s3)cc2)cc1 ; CC1(C)c2ccccc2-c2ccc(N(c3ccc(-c4ccccc4)cc3)c3ccc(-c4ccc5c(ccc6ccc7sc(-c8ccc(-c9ccccc9)cc8)nc7c65)c4)cc3)cc21 ; CC1(C)c2ccccc2-c2ccc(N(c3ccccc3)c3ccc(-c4ccc5c(ccc6ccc7sc(-c8ccccc8)nc7c65)c4)cc3)cc21]

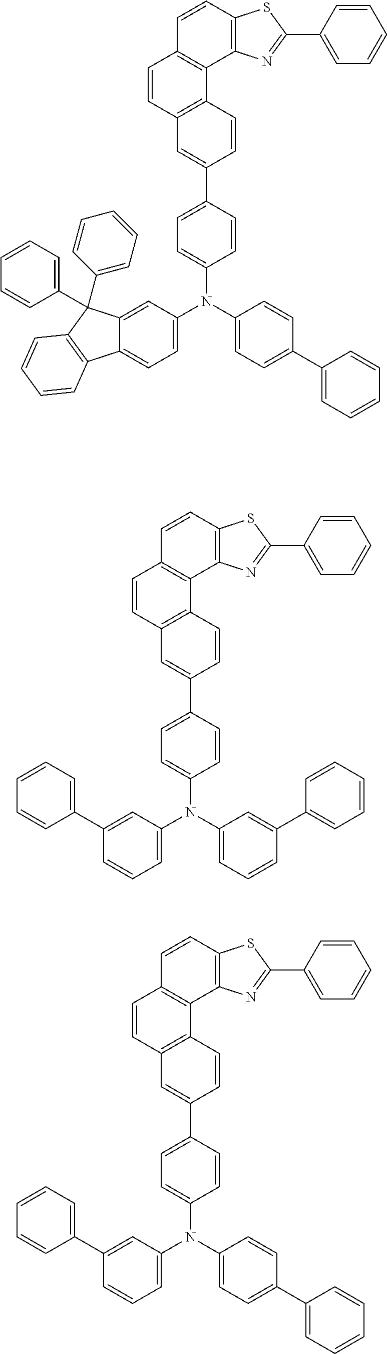 [SMILES: c1ccc(-c2ccc(N(c3ccc(-c4ccc5c(ccc6ccc7sc(-c8ccccc8)nc7c65)c4)cc3)c3ccc4c(c3)C(c3ccccc3)(c3ccccc3)c3ccccc3-4)cc2)cc1 ; c1ccc(-c2cccc(N(c3ccc(-c4ccc5c(ccc6ccc7sc(-c8ccccc8)nc7c65)c4)cc3)c3cccc(-c4ccccc4)c3)c2)cc1 ; c1ccc(-c2ccc(N(c3ccc(-c4ccc5c(ccc6ccc7sc(-c8ccccc8)nc7c65)c4)cc3)c3cccc(-c4ccccc4)c3)cc2)cc1]

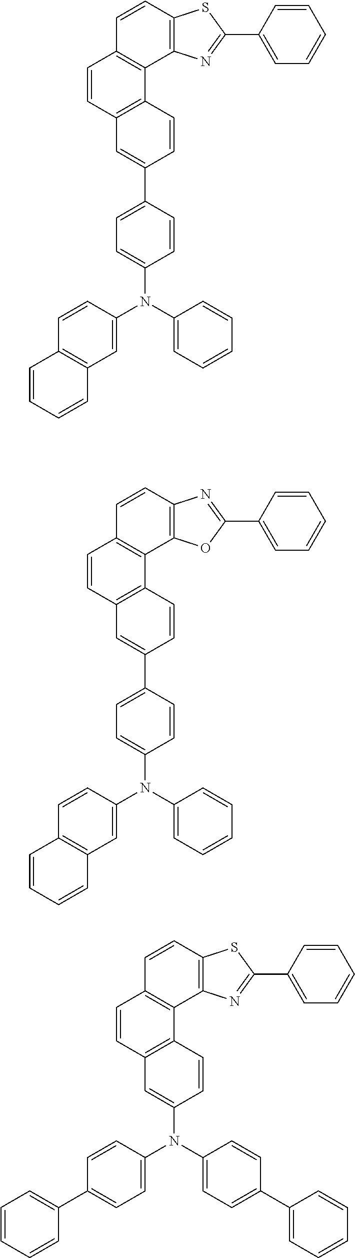 [SMILES: c1ccc(-c2nc3c(ccc4ccc5cc(-c6ccc(N(c7ccccc7)c7ccc8ccccc8c7)cc6)ccc5c43)s2)cc1 ; c1ccc(-c2nc3ccc4ccc5cc(-c6ccc(N(c7ccccc7)c7ccc8ccccc8c7)cc6)ccc5c4c3o2)cc1 ; c1ccc(-c2ccc(N(c3ccc(-c4ccccc4)cc3)c3ccc4c(ccc5ccc6sc(-c7ccccc7)nc6c54)c3)cc2)cc1]

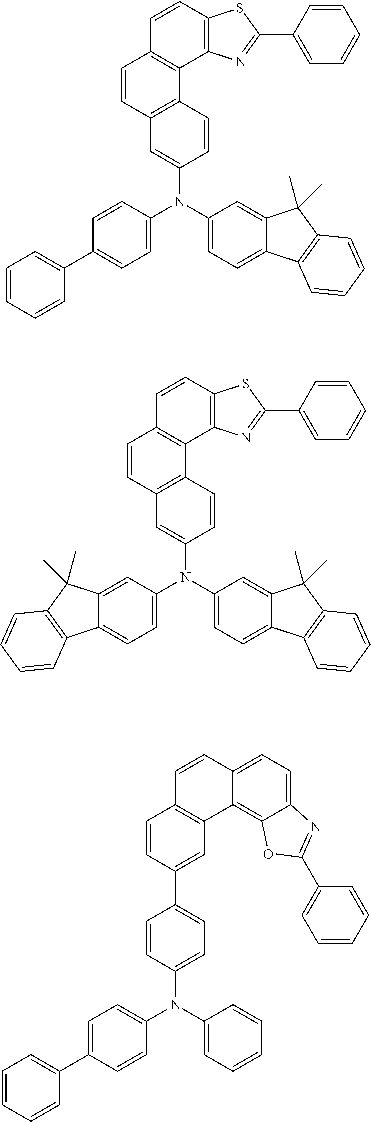 [SMILES: CC1(C)c2ccccc2-c2ccc(N(c3ccc(-c4ccccc4)cc3)c3ccc4c(ccc5ccc6sc(-c7ccccc7)nc6c54)c3)cc21 ; CC1(C)c2ccccc2-c2ccc(N(c3ccc4c(c3)C(C)(C)c3ccccc3-4)c3ccc4c(ccc5ccc6sc(-c7ccccc7)nc6c54)c3)cc21 ; c1ccc(-c2ccc(N(c3ccccc3)c3ccc(-c4ccc5ccc6ccc7nc(-c8ccccc8)oc7c6c5c4)cc3)cc2)cc1]

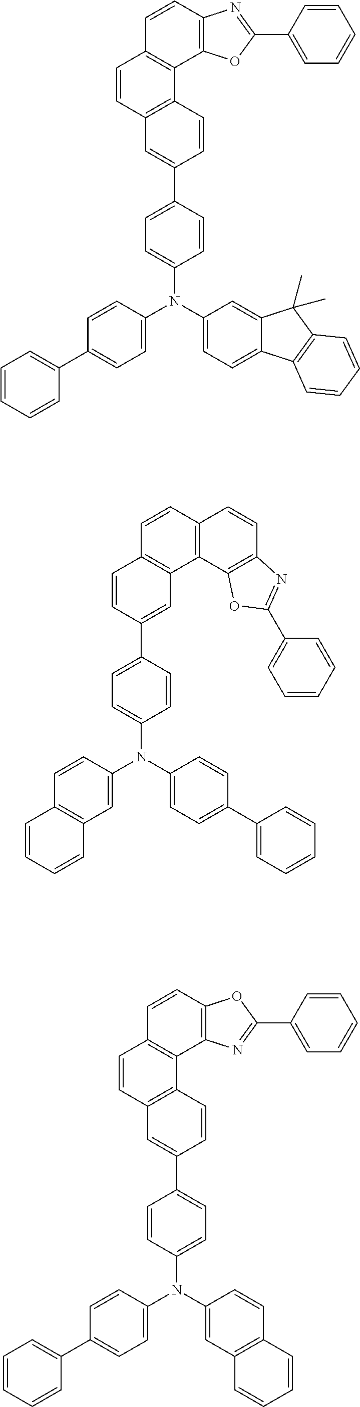 [SMILES: CC1(C)c2ccccc2-c2ccc(N(c3ccc(-c4ccccc4)cc3)c3ccc(-c4ccc5c(ccc6ccc7nc(-c8ccccc8)oc7c65)c4)cc3)cc21 ; c1ccc(-c2ccc(N(c3ccc(-c4ccc5ccc6ccc7nc(-c8ccccc8)oc7c6c5c4)cc3)c3ccc4ccccc4c3)cc2)cc1 ; c1ccc(-c2ccc(N(c3ccc(-c4ccc5c(ccc6ccc7oc(-c8ccccc8)nc7c65)c4)cc3)c3ccc4ccccc4c3)cc2)cc1]

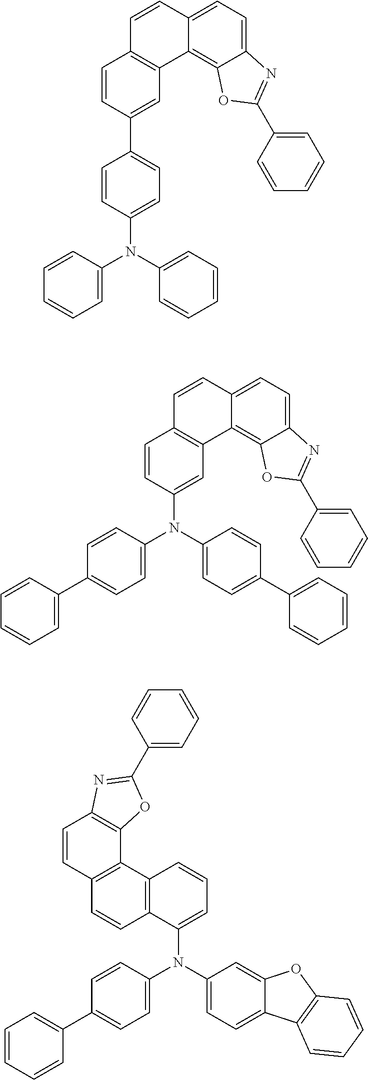 [SMILES: c1ccc(-c2nc3ccc4ccc5ccc(-c6ccc(N(c7ccccc7)c7ccccc7)cc6)cc5c4c3o2)cc1 ; c1ccc(-c2ccc(N(c3ccc(-c4ccccc4)cc3)c3ccc4ccc5ccc6nc(-c7ccccc7)oc6c5c4c3)cc2)cc1 ; c1ccc(-c2ccc(N(c3ccc4c(c3)oc3ccccc34)c3cccc4c3ccc3ccc5nc(-c6ccccc6)oc5c34)cc2)cc1]

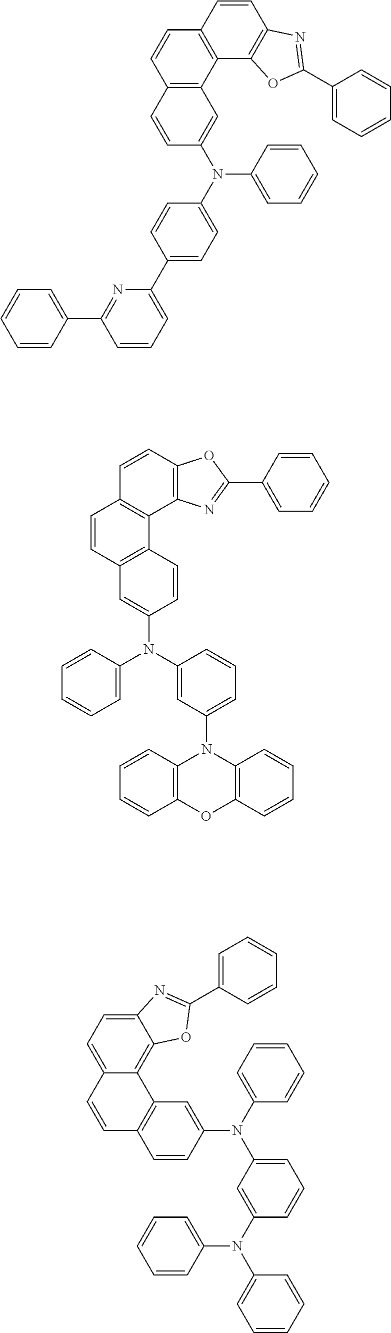 [SMILES: c1ccc(-c2cccc(-c3ccc(N(c4ccccc4)c4ccc5ccc6ccc7nc(-c8ccccc8)oc7c6c5c4)cc3)n2)cc1 ; c1ccc(-c2nc3c(ccc4ccc5cc(N(c6ccccc6)c6cccc(N7c8ccccc8Oc8ccccc87)c6)ccc5c43)o2)cc1 ; c1ccc(-c2nc3ccc4ccc5ccc(N(c6ccccc6)c6cccc(N(c7ccccc7)c7ccccc7)c6)cc5c4c3o2)cc1]

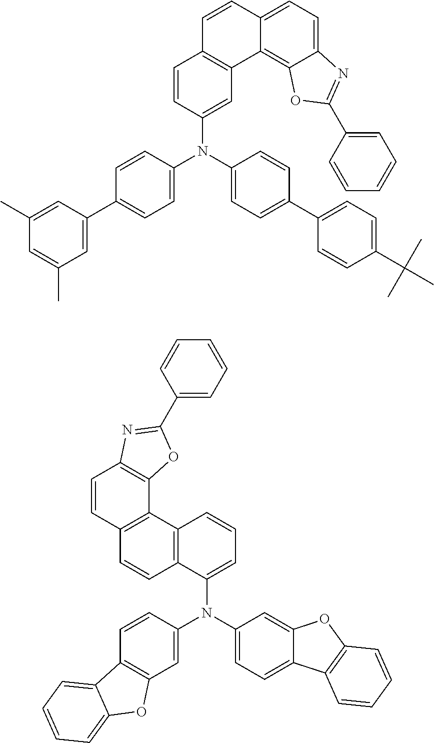 [SMILES: Cc1cc(C)cc(-c2ccc(N(c3ccc(-c4ccc(C(C)(C)C)cc4)cc3)c3ccc4ccc5ccc6nc(-c7ccccc7)oc6c5c4c3)cc2)c1 ; c1ccc(-c2nc3ccc4ccc5c(N(c6ccc7c(c6)oc6ccccc67)c6ccc7c(c6)oc6ccccc67)cccc5c4c3o2)cc1]

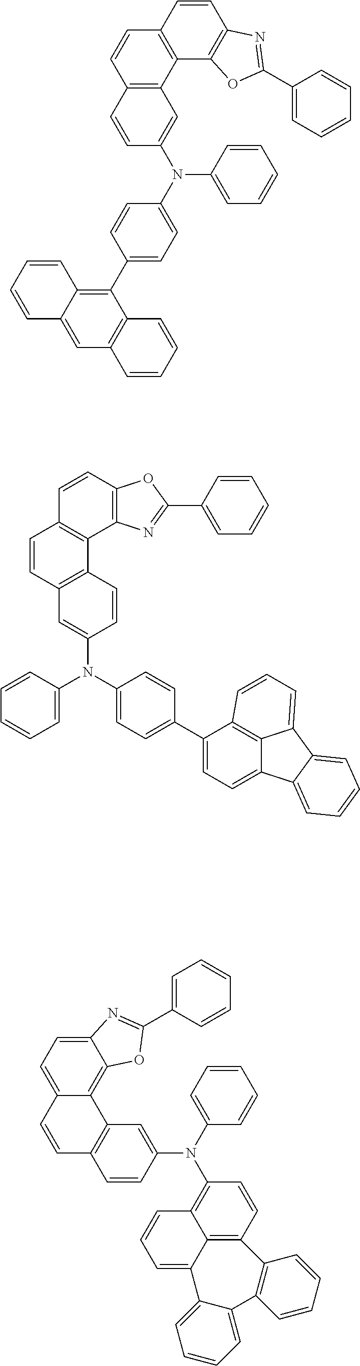 [SMILES: c1ccc(-c2nc3ccc4ccc5ccc(N(c6ccccc6)c6ccc(-c7c8ccccc8cc8ccccc78)cc6)cc5c4c3o2)cc1 ; c1ccc(-c2nc3c(ccc4ccc5cc(N(c6ccccc6)c6ccc(-c7ccc8c9c(cccc79)-c7ccccc7-8)cc6)ccc5c43)o2)cc1 ; c1ccc(-c2nc3ccc4ccc5ccc(N(c6ccccc6)c6ccc7c8c(cccc68)-c6ccccc6-c6ccccc6-7)cc5c4c3o2)cc1]

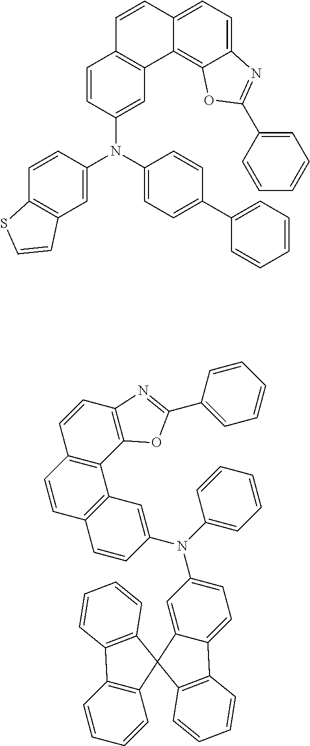 [SMILES: c1ccc(-c2ccc(N(c3ccc4sccc4c3)c3ccc4ccc5ccc6nc(-c7ccccc7)oc6c5c4c3)cc2)cc1 ; c1ccc(-c2nc3ccc4ccc5ccc(N(c6ccccc6)c6ccc7c(c6)C6(c8ccccc8-c8ccccc86)c6ccccc6-7)cc5c4c3o2)cc1]

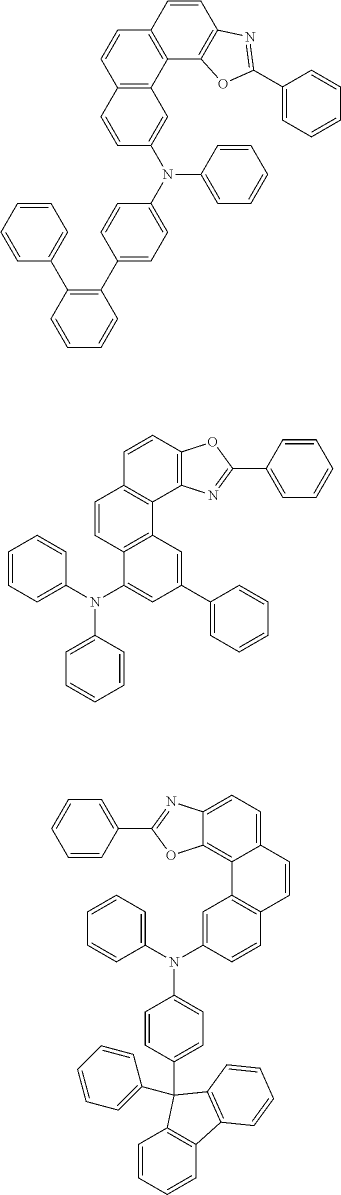 [SMILES: c1ccc(-c2nc3ccc4ccc5ccc(N(c6ccccc6)c6ccc(-c7ccccc7-c7ccccc7)cc6)cc5c4c3o2)cc1 ; c1ccc(-c2cc(N(c3ccccc3)c3ccccc3)c3ccc4ccc5oc(-c6ccccc6)nc5c4c3c2)cc1 ; c1ccc(-c2nc3ccc4ccc5ccc(N(c6ccccc6)c6ccc(C7(c8ccccc8)c8ccccc8-c8ccccc87)cc6)cc5c4c3o2)cc1]

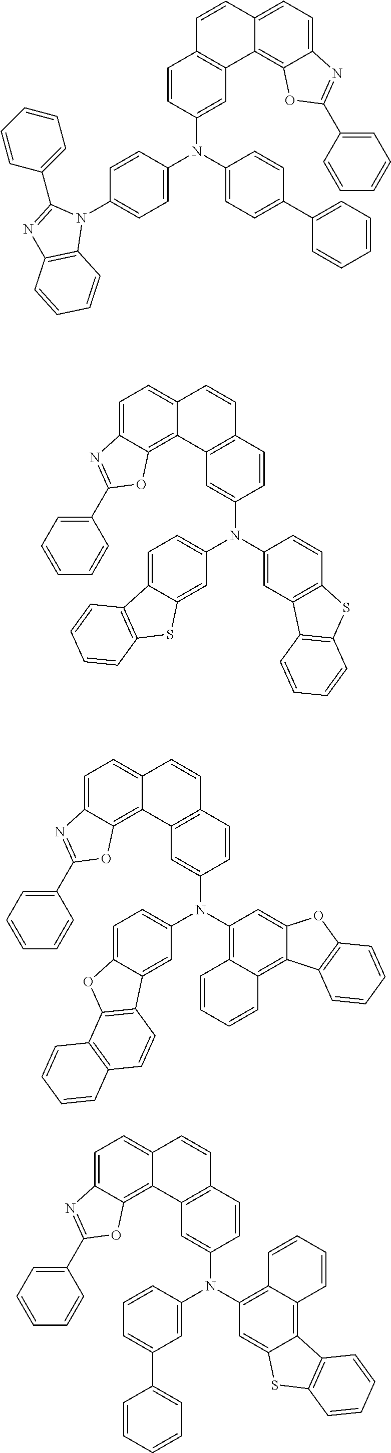

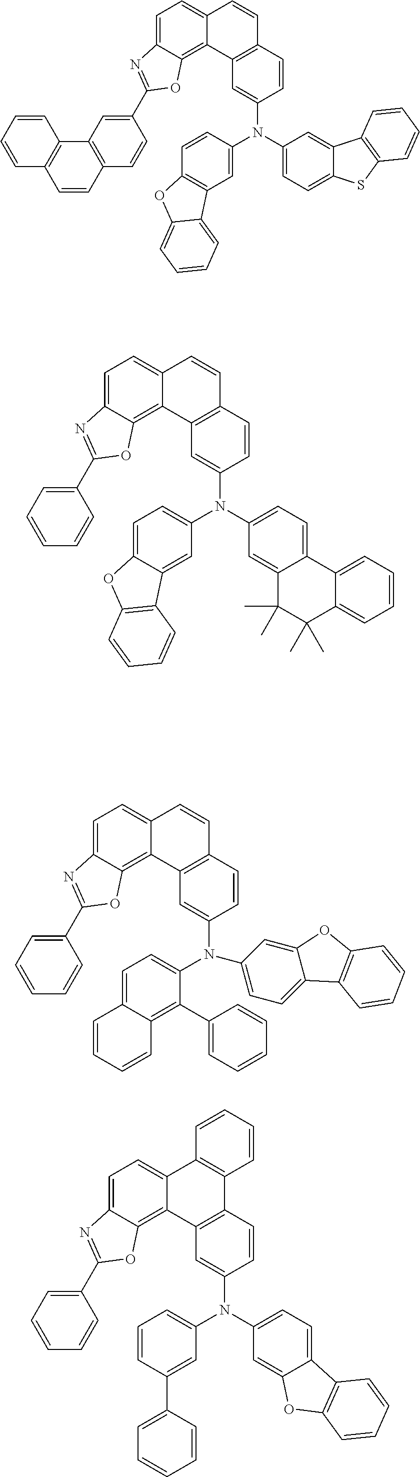

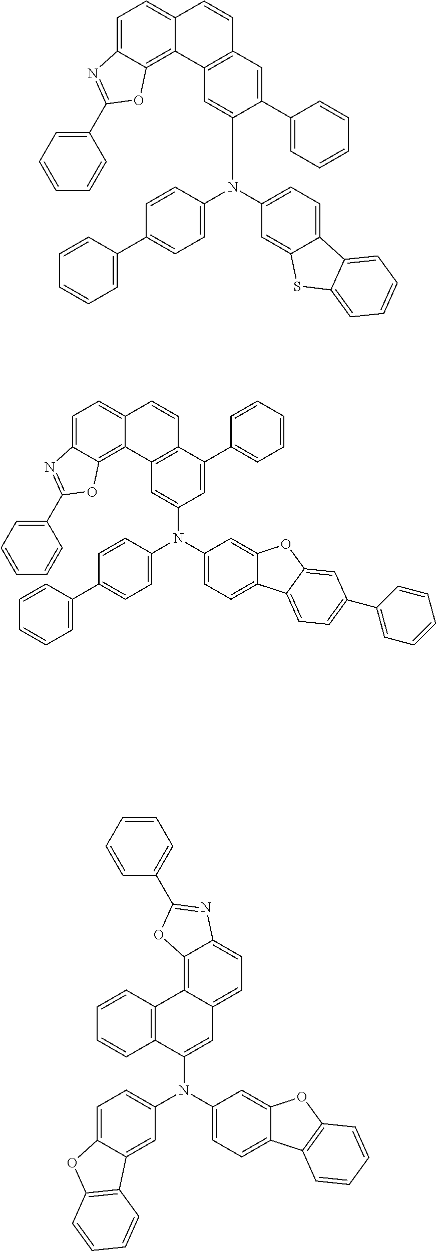 [SMILES: c1ccc(-c2ccc(N(c3ccc4c(c3)sc3ccccc34)c3cc4c(ccc5ccc6nc(-c7ccccc7)oc6c54)cc3-c3ccccc3)cc2)cc1 ; c1ccc(-c2ccc(N(c3ccc4c(c3)oc3cc(-c5ccccc5)ccc34)c3cc(-c4ccccc4)c4ccc5ccc6nc(-c7ccccc7)oc6c5c4c3)cc2)cc1 ; c1ccc(-c2nc3ccc4cc(N(c5ccc6c(c5)oc5ccccc56)c5ccc6oc7ccccc7c6c5)c5ccccc5c4c3o2)cc1]

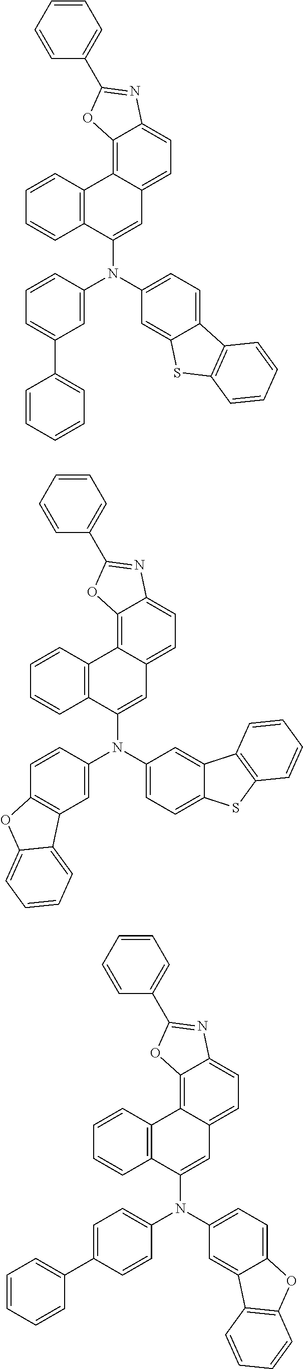

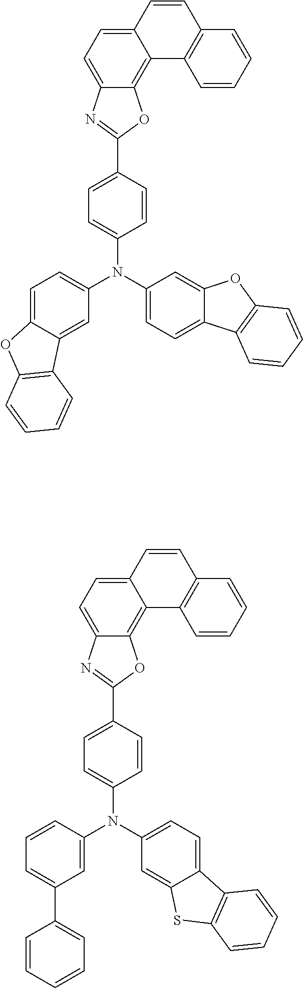 [SMILES: c1ccc2c(c1)ccc1ccc3nc(-c4ccc(N(c5ccc6c(c5)oc5ccccc56)c5ccc6oc7ccccc7c6c5)cc4)oc3c12 ; c1ccc(-c2cccc(N(c3ccc(-c4nc5ccc6ccc7ccccc7c6c5o4)cc3)c3ccc4c(c3)sc3ccccc34)c2)cc1]

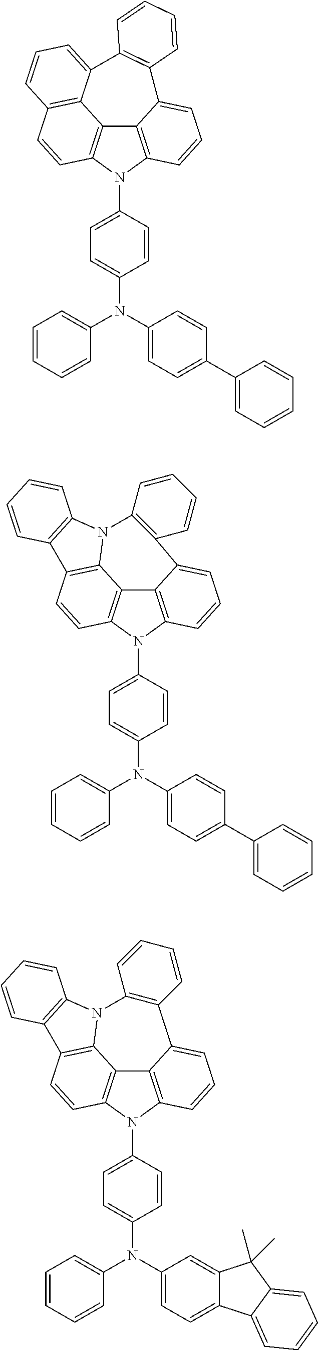 [SMILES: c1ccc(-c2ccc(N(c3ccccc3)c3ccc(-n4c5cccc6c5c5c7c(cccc7ccc54)-c4ccccc4-6)cc3)cc2)cc1 ; c1ccc(-c2ccc(N(c3ccccc3)c3ccc(-n4c5cccc6c7ccccc7n7c8ccccc8c8ccc4c(c65)c87)cc3)cc2)cc1 ; CC1(C)c2ccccc2-c2ccc(N(c3ccccc3)c3ccc(-n4c5cccc6c7ccccc7n7c8ccccc8c8ccc4c(c65)c87)cc3)cc21]

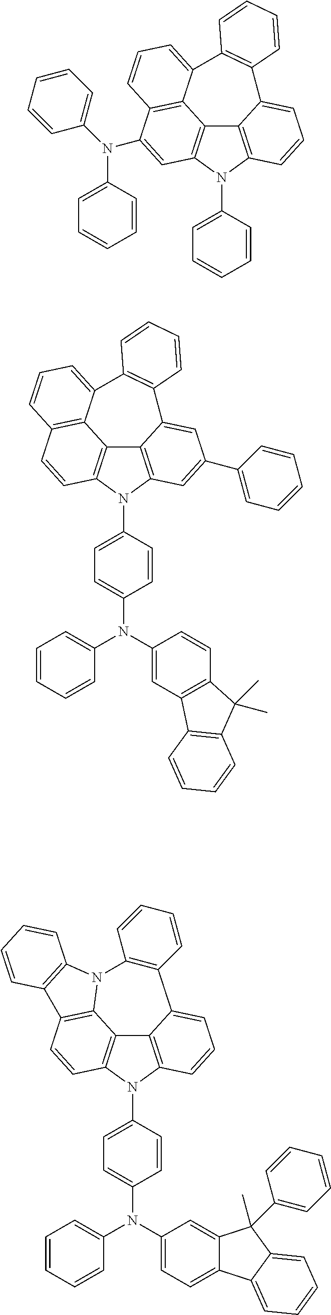 [SMILES: c1ccc(N(c2ccccc2)c2cc3c4c5c(cccc25)-c2ccccc2-c2cccc(c24)n3-c2ccccc2)cc1 ; CC1(C)c2ccccc2-c2cc(N(c3ccccc3)c3ccc(-n4c5cc(-c6ccccc6)cc6c5c5c7c(cccc7ccc54)-c4ccccc4-6)cc3)ccc21 ; CC1(c2ccccc2)c2ccccc2-c2ccc(N(c3ccccc3)c3ccc(-n4c5cccc6c7ccccc7n7c8ccccc8c8ccc4c(c65)c87)cc3)cc21]

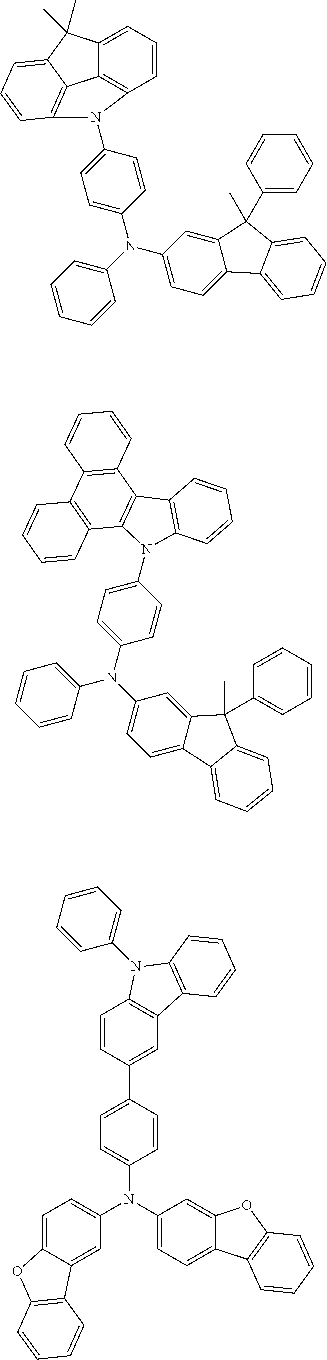 [SMILES: CC1(C)c2cccc3c2c2c1cccc2n3-c1ccc(N(c2ccccc2)c2ccc3c(c2)C(C)(c2ccccc2)c2ccccc2-3)cc1 ; CC1(c2ccccc2)c2ccccc2-c2ccc(N(c3ccccc3)c3ccc(-n4c5ccccc5c5c6ccccc6c6ccccc6c54)cc3)cc21 ; c1ccc(-n2c3ccccc3c3cc(-c4ccc(N(c5ccc6c(c5)oc5ccccc56)c5ccc6oc7ccccc7c6c5)cc4)ccc32)cc1]

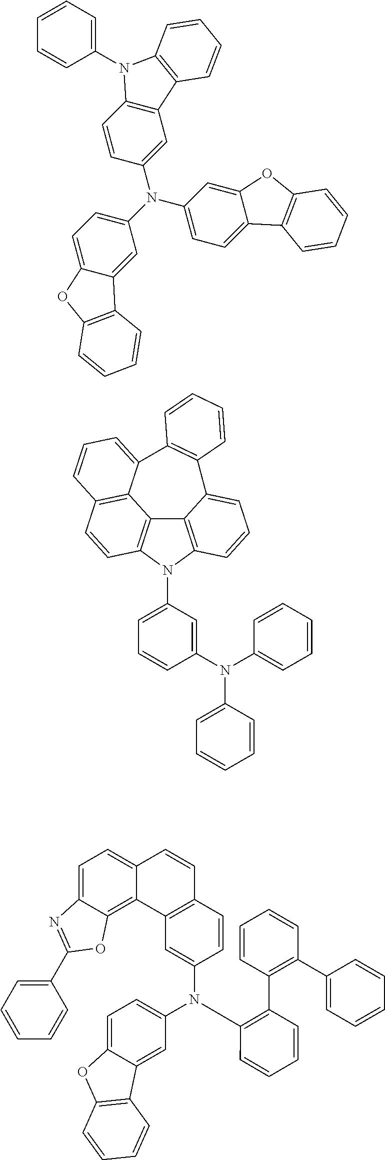 [SMILES: c1ccc(-n2c3ccccc3c3cc(N(c4ccc5c(c4)oc4ccccc45)c4ccc5oc6ccccc6c5c4)ccc32)cc1 ; c1ccc(N(c2ccccc2)c2cccc(-n3c4cccc5c4c4c6c(cccc6ccc43)-c3ccccc3-5)c2)cc1 ; c1ccc(-c2nc3ccc4ccc5ccc(N(c6ccc7oc8ccccc8c7c6)c6ccccc6-c6ccccc6-c6ccccc6)cc5c4c3o2)cc1]

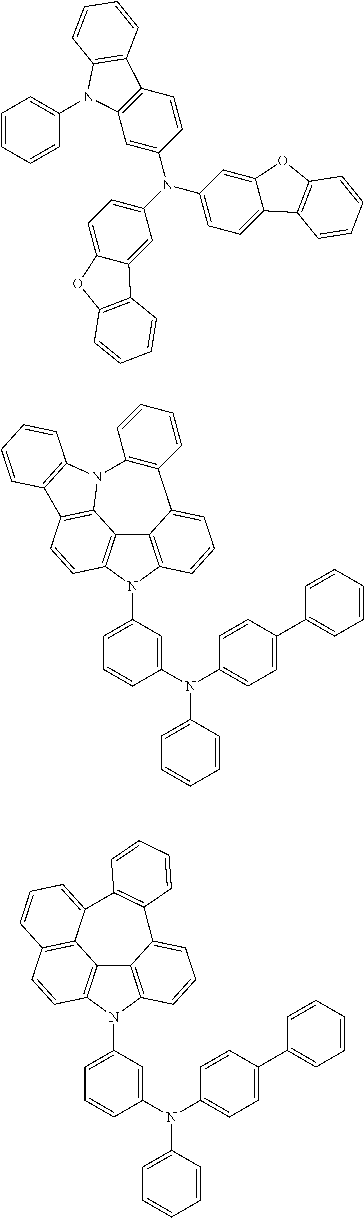

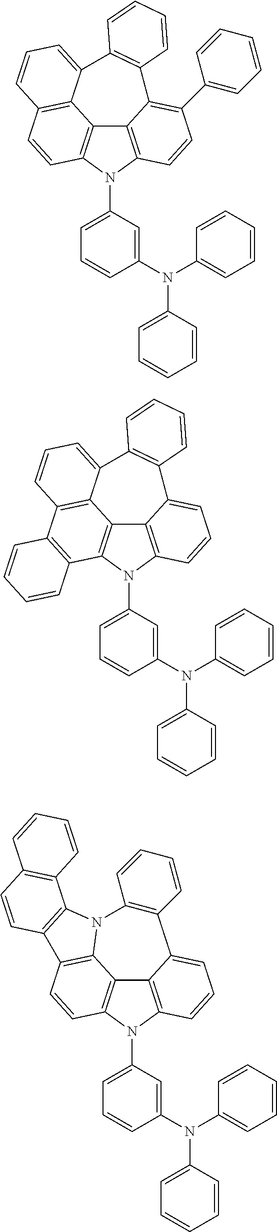

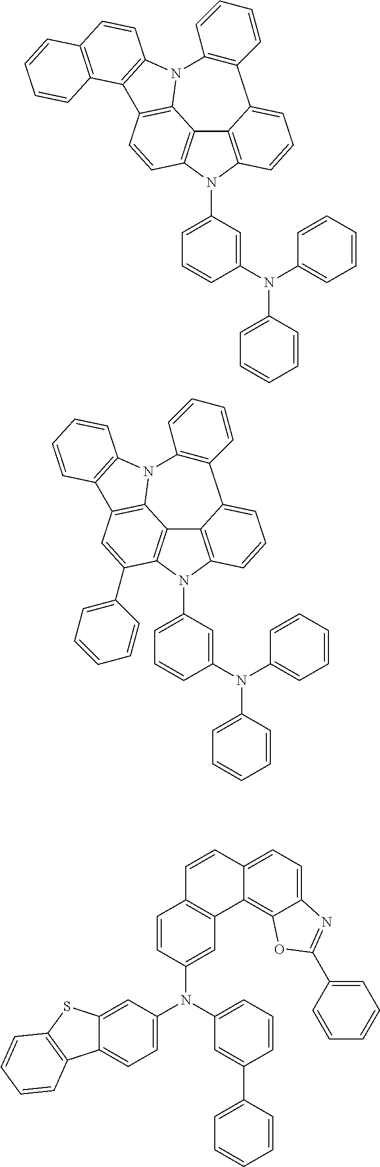

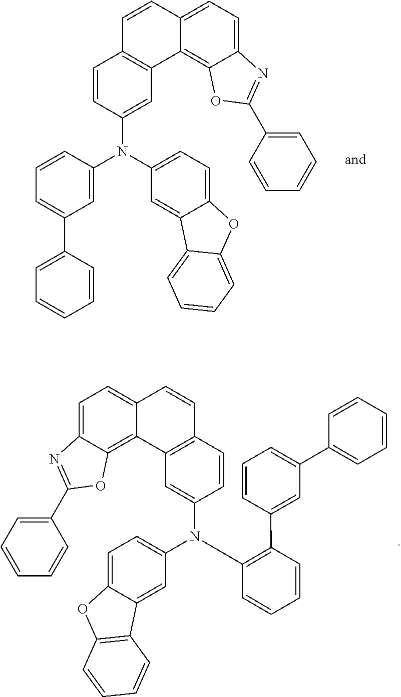 [SMILES: c1ccc(-c2cccc(N(c3ccc4oc5ccccc5c4c3)c3ccc4ccc5ccc6nc(-c7ccccc7)oc6c5c4c3)c2)cc1 ; c1ccc(-c2cccc(-c3ccccc3N(c3ccc4oc5ccccc5c4c3)c3ccc4ccc5ccc6nc(-c7ccccc7)oc6c5c4c3)c2)cc1]

10. An organic electroluminescent device comprising the organic electroluminescent compound according to claim 1.

11. An organic electroluminescent device comprising the organic electroluminescent material according to claim 7.
